(19) United States
(12) Patent Application Publication
(10) Pub. No.: US 2008/0171783 A1
(43) Pub. Date: Jul. 17, 2008

(54) COMPOSITIONS AND METHODS FOR TREATING HYPERPROLIFERATIVE DISEASE

(75) Inventors: Dale Russell Cameron, Richmond (CA); Maria Marta Guarna, Vancouver (CA); Luba Metlitskaia, North Vancouver (CA)

Correspondence Address: DARBY & DARBY P.C., P.O. BOX 770, Church Street Station, New York, NY, 10008-0770, US

(73) Assignee: Migenix Inc., Vancouver (CA)

(21) Appl. No.: 11/966,666

(22) Filed: Dec. 28, 2007

Related U.S. Application Data
(60) Provisional application No. 60/882,541, filed on Dec. 28, 2006.

(51) Int. Cl. A61K 31/404; A61K 31/164; A61K 31/135; A61K 31/27; A61P 43/00; A61K 31/17; A61K 31/341

(52) U.S. Cl. 514/419; 514/625; 514/656; 514/480; 514/598; 514/471

(57) ABSTRACT

The present disclosure provides amide-based, non-nucleoside compounds having an inhibitory activity against endogenous polymerases, such as polymerase alpha and polymerase gamma. This disclosure further provides uses of treating hyperproliferative diseases or disorders, such as benign or malignant neoplasms, and more specifically cancers that are sensitive to inhibition of polymerase alpha and polymerase gamma.

### CROSS-REFERENCE TO RELATED APPLICATION

This application claims the benefit of U.S. Provisional Patent Application No. 60/882,541, filed Dec. 28, 2006, which application is incorporated herein by reference in its entirety.

### TECHNICAL FIELD

The present disclosure relates generally to agents for treating hyperproliferative diseases or disorders and, more specifically, to amide-based compounds and compositions thereof for use in treating cancer, such as cancer, which is sensitive to inhibitors of certain endogenous polymerases including polymerase alpha or polymerase gamma.

### BACKGROUND

Cancers are a leading cause of death in animals and humans. Despite advances in the field of cancer treatment, the leading therapies to date are surgery, radiation and chemotherapy. Many types of chemotherapeutic agents have been shown to be effective against cancers and tumor cells, but not all types of cancers and tumors respond to these agents. Unfortunately, many of these agents are also toxic for normal cells. Chemotherapeutic approaches can be used to treat cancers that are metastasized or ones that are particularly aggressive, and such cytocidal or cytostatic agents work best on cancers with rapidly dividing cells. The bulk of therapies available to oncologists today include hormones (such as estrogen, progesterone, testosterone), some antibiotics, alkylating agents, and anti-metabolites.

Ideally, chemotherapeutic agents that have specificity for certain hyperproliferative cells (e.g., cancer) while not affecting normal cells would be most desirable. Unfortunately, none have been found and instead agents that target rapidly dividing cells (both tumor and normal) have been used. The development of compounds that would specifically target hyperproliferative cells or that are cytotoxic to hyperproliferative cells while exerting mild effects on normal cells would be desirable.

### SUMMARY

Briefly, the present disclosure provides methods of using non-nucleoside compounds that inhibit endogenous polymerases to inhibit uncontrolled growth of a hyperproliferative cell. For example, use against hyperproliferative cells involved in various different diseases or disorders, such as cancer, autoimmunity, premalignant lesions, or the like.

In one aspect, the present disclosure provides methods for treating a hyperproliferative disease with a compound having a structure of formula (IV):

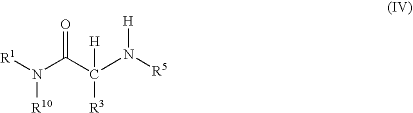

or a stereoisomer, prodrug or pharmaceutically acceptable salt thereof, wherein:

R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

R⁵ is selected from —C(═O)NR¹⁰R⁹, —C(═O)(NR¹⁰)SO₂R⁹, —C(═S)NR¹⁰R⁹, or —C(═NR¹⁰)NR¹⁰R⁹; and

each R¹⁰ is independently selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl. Preferably, R³ is not hydrogen.

In a further embodiment, the present disclosure provides use of a compound having a structure of formula (IV), as defined herein, in any of the methods described in the instant disclosure, wherein R³ is not hydrogen or wherein R³ has an ionizable nitrogen.

In a further aspect, any of the compounds of this disclosure can be administered parenterally, such as by injection intravenously, intra-arterially, or intratumorally.

In another aspect, the present disclosure provides methods for treating a hyperproliferative disease or disorder, comprising administering to a subject in need thereof a therapeutically effective amount of a compound having a structure of formula (III):

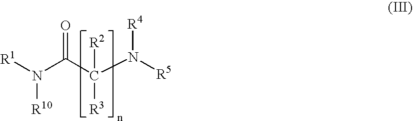

or a stereoisomer, prodrug or pharmaceutically acceptable salt thereof, wherein:

n is 1-5;

R¹ is selected from (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R², R³, R⁴, and R⁹ are (i) independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ and R⁹ are as defined above;

or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R⁹ are as defined above;

or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R², R³ and R⁹ are as defined above;

and wherein at least one, but not more than two of R², R³, R⁴ and R⁵, is hydrogen, provided that R¹ is not an amino acid when R⁴ and R⁵ are both H.

In a further aspect, the present disclosure provides methods for treating a hyperproliferative disease or disorder, comprising administering to a subject in need thereof a therapeutically effective amount of a compound having a structure of formula (I):

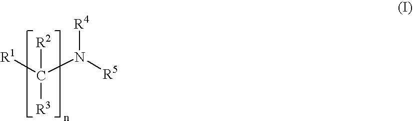

or a stereoisomer, prodrug or pharmaceutically acceptable salt thereof, wherein:

n is 1-5;

R¹ is selected from —C(═O)R⁹, —C(═S)R⁹, —C(═NR¹⁰)R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —S(═O)R⁹, —SO₂R⁹, provided that R⁹ is not H, and that R⁹ does not form an ester with the carbonyl group to which it is bonded when R¹ is —C(═O)R⁹;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;

and wherein at least one, but not more than two, of R², R³, R⁴ and R⁵ is hydrogen, provided that R¹ is not an amino acid when R⁴ and R⁵ are both H.

### BRIEF DESCRIPTION OF THE DRAWINGS

FIG. 1 shows specific exemplary compounds of structures (I)-(X) for use in treating hyperproliferative diseases or disorders.

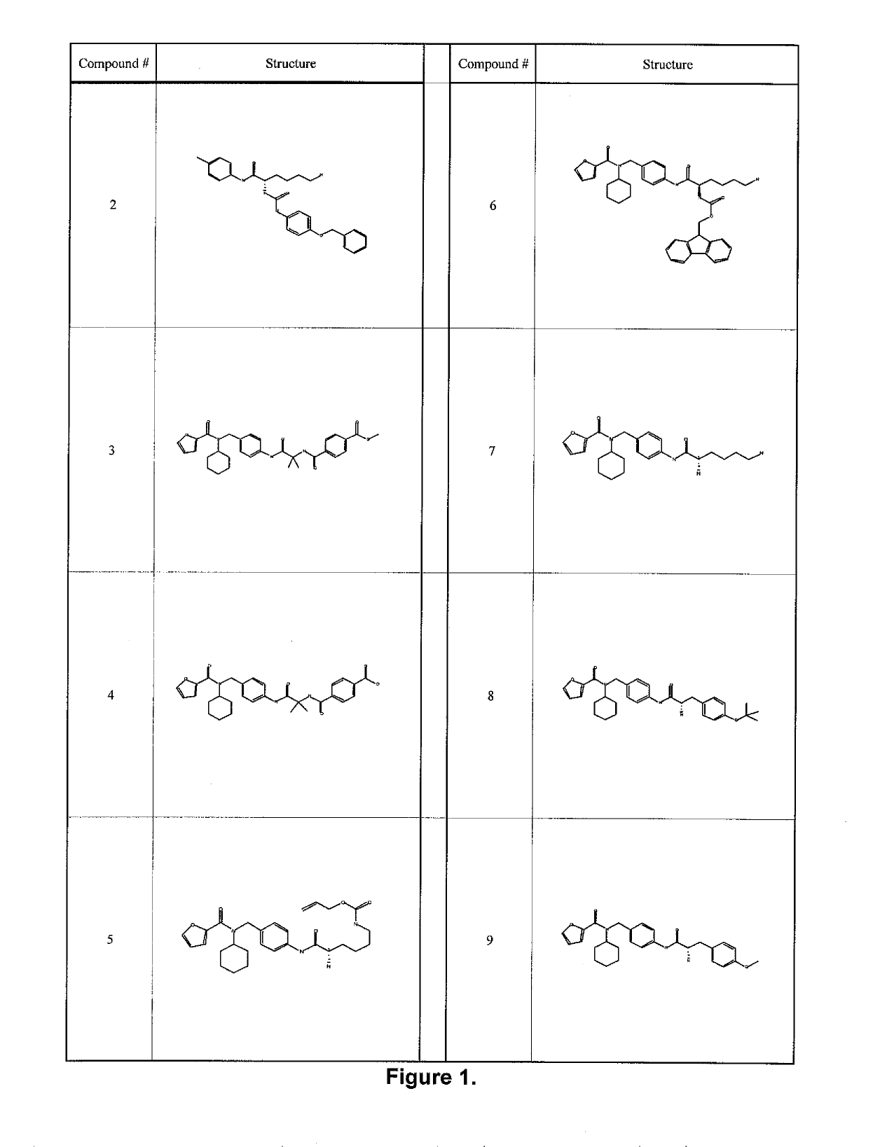

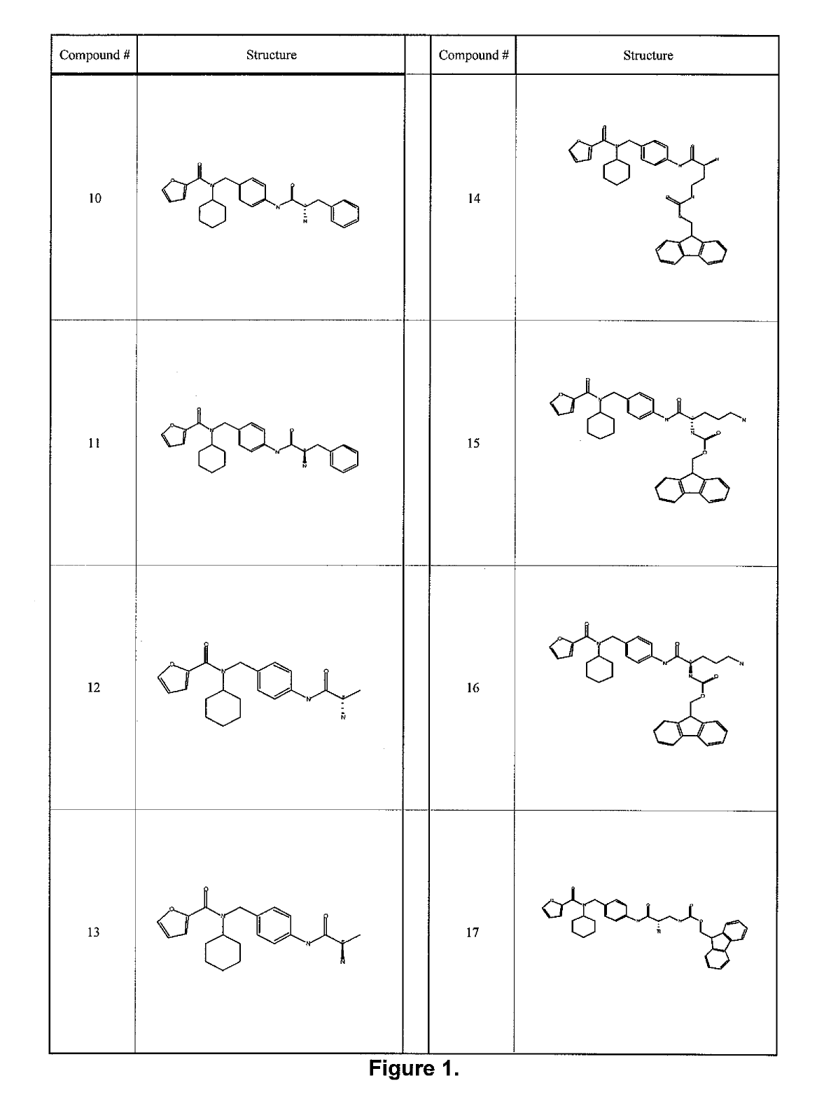

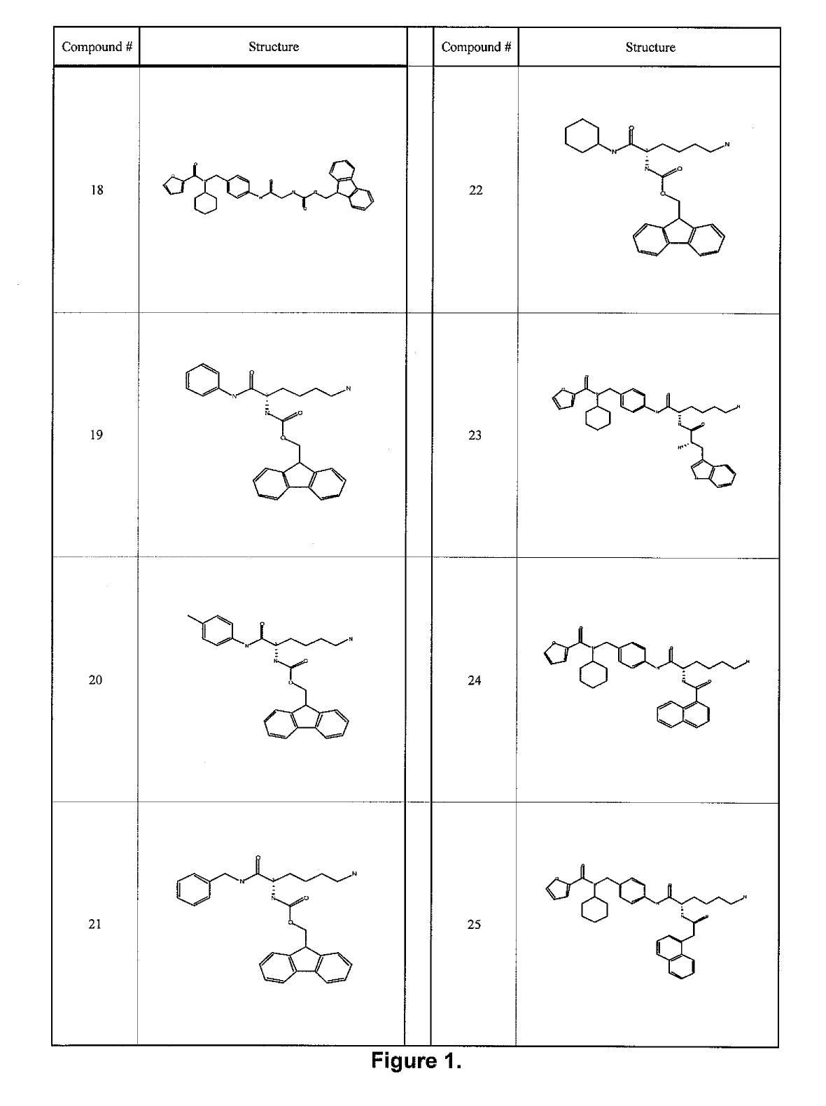

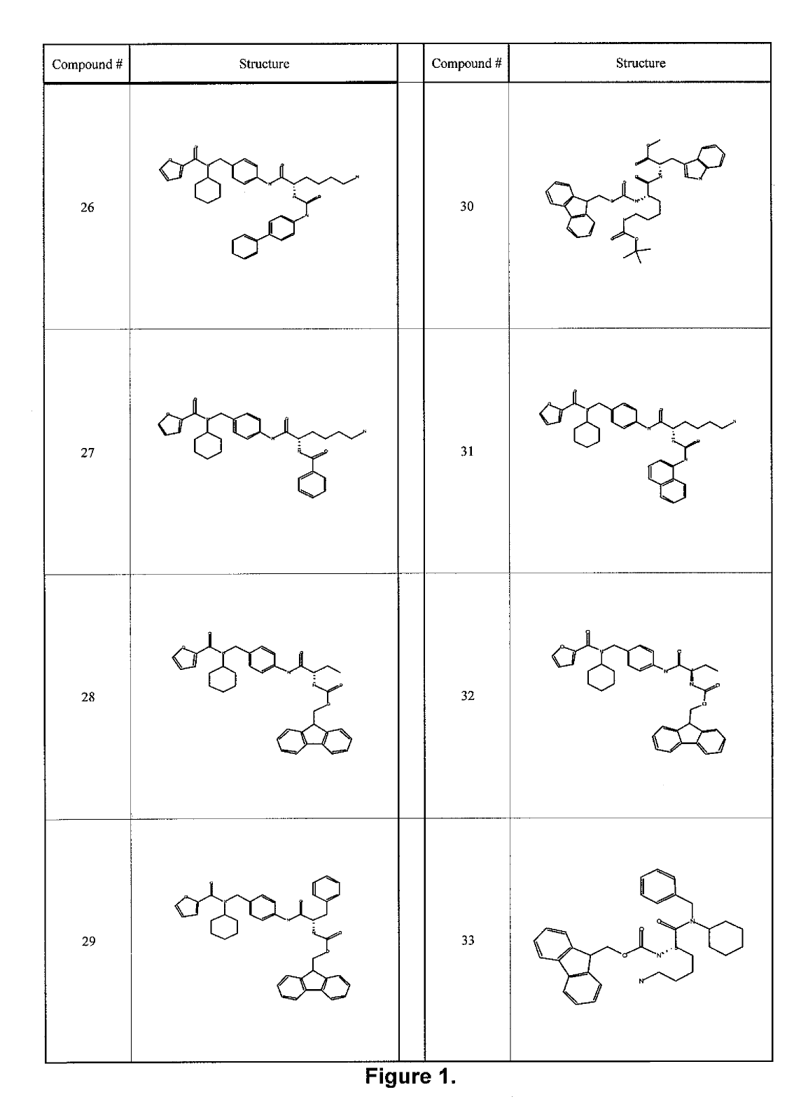

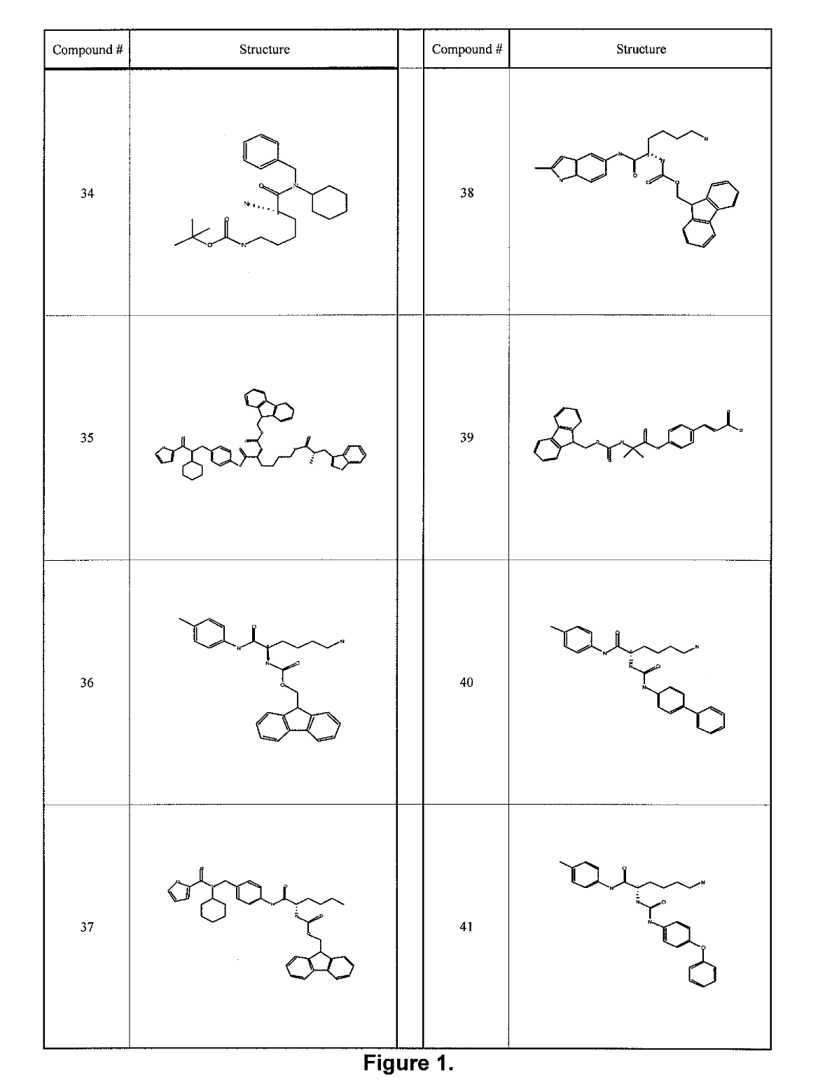

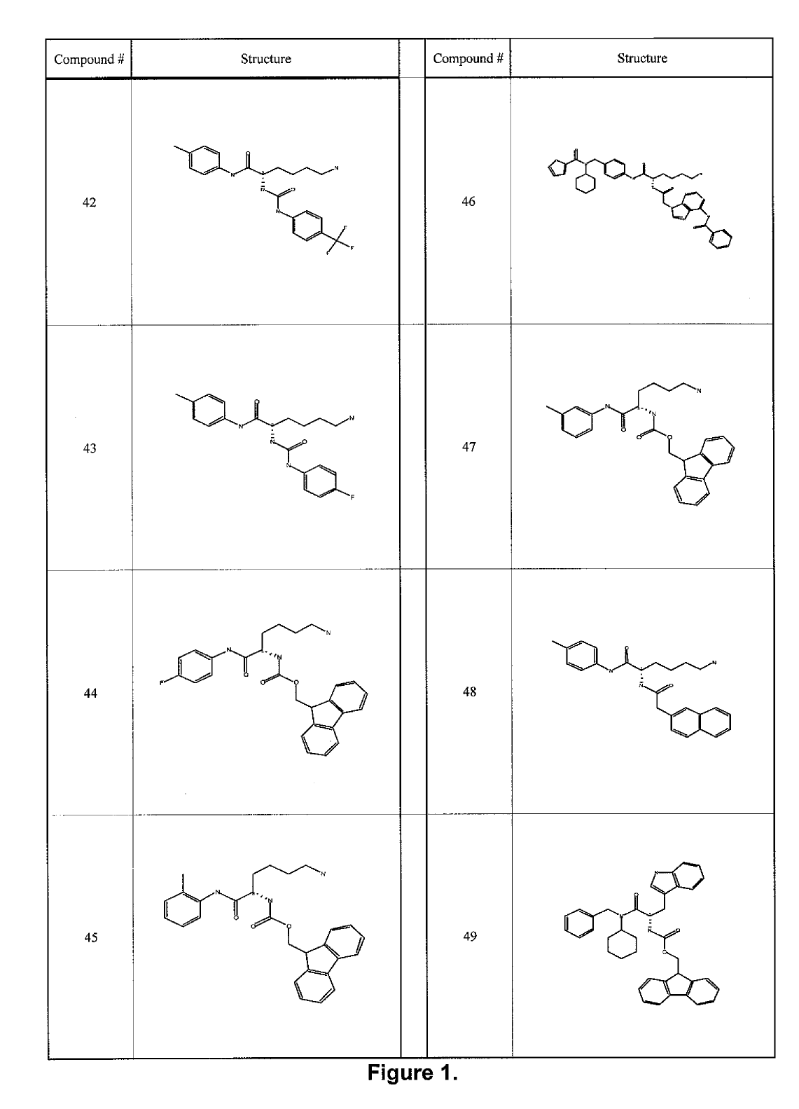

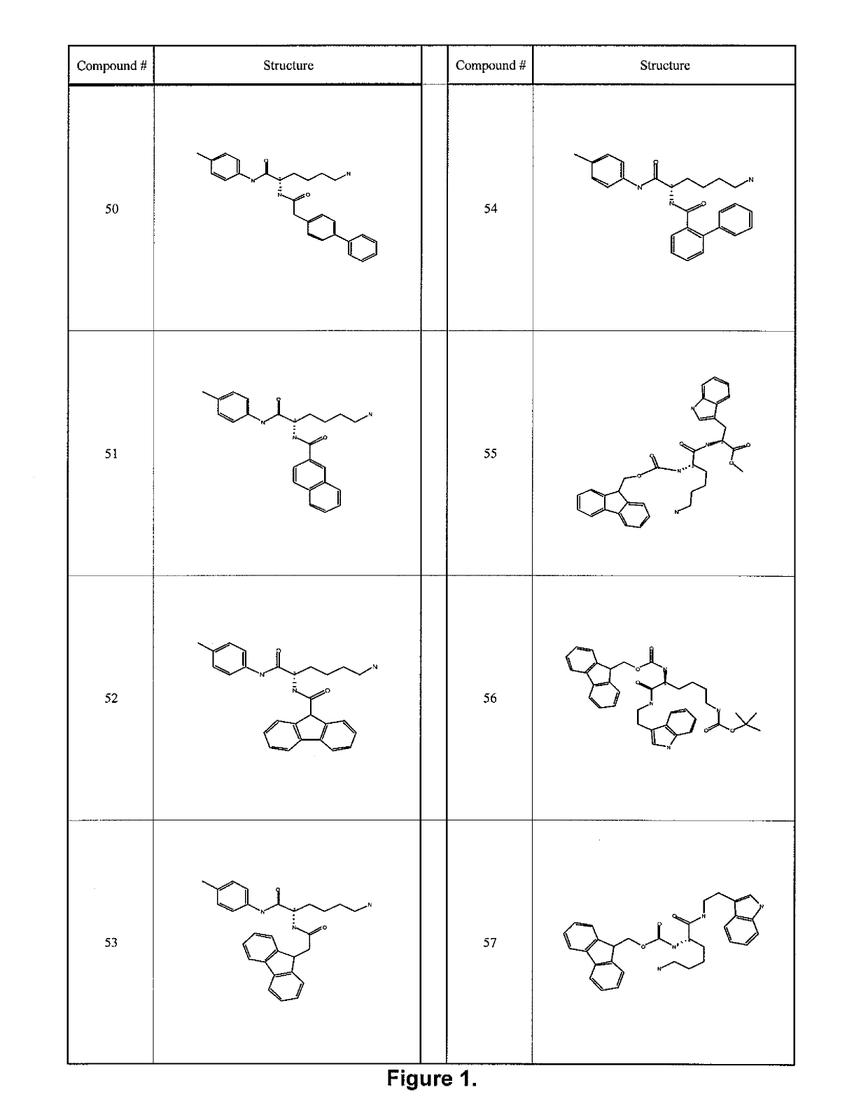

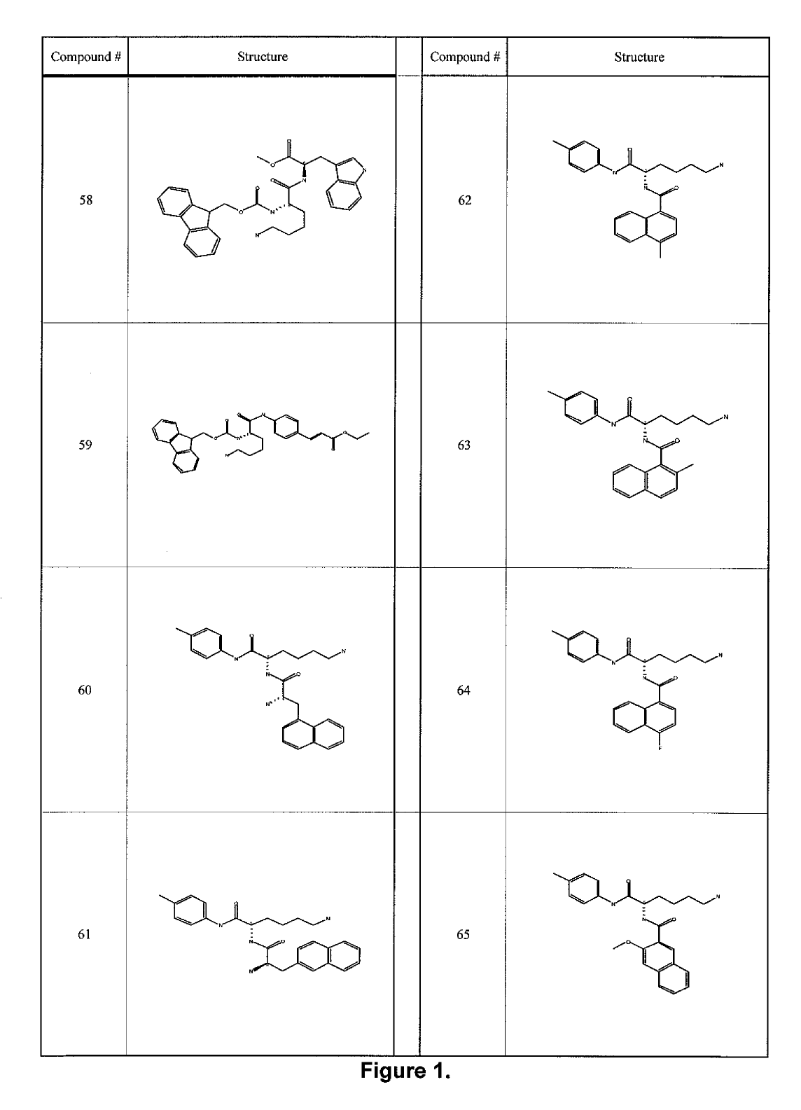

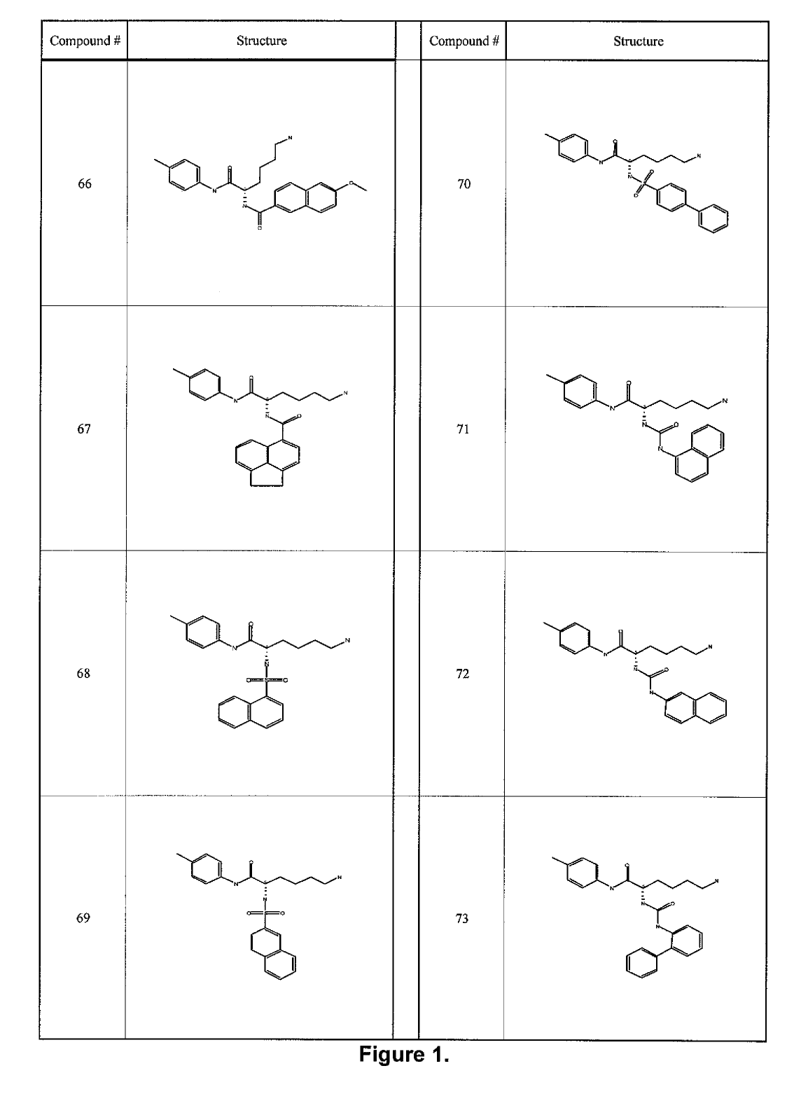

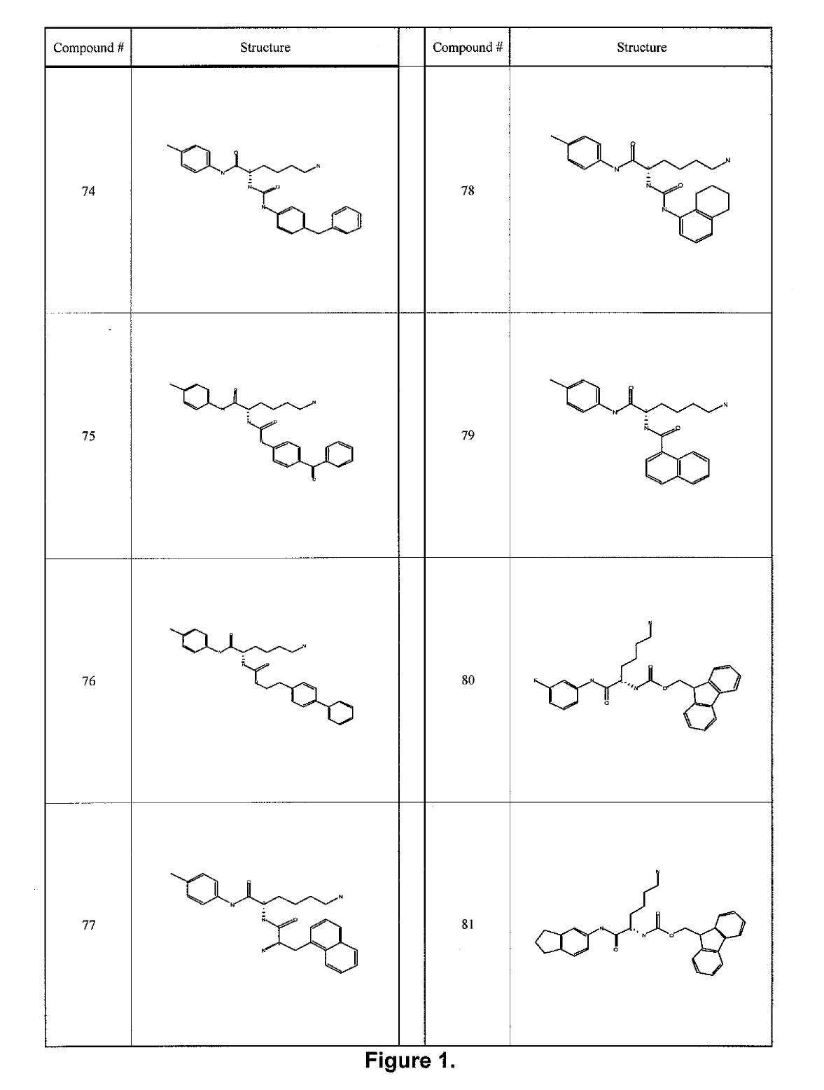

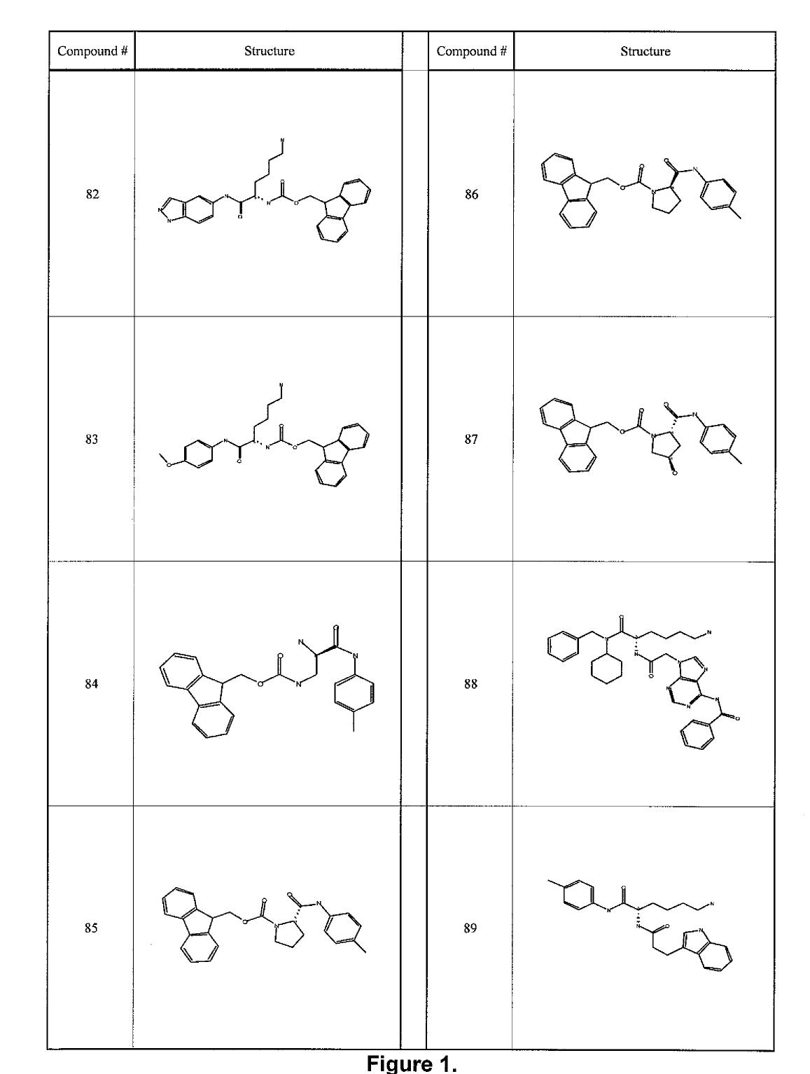

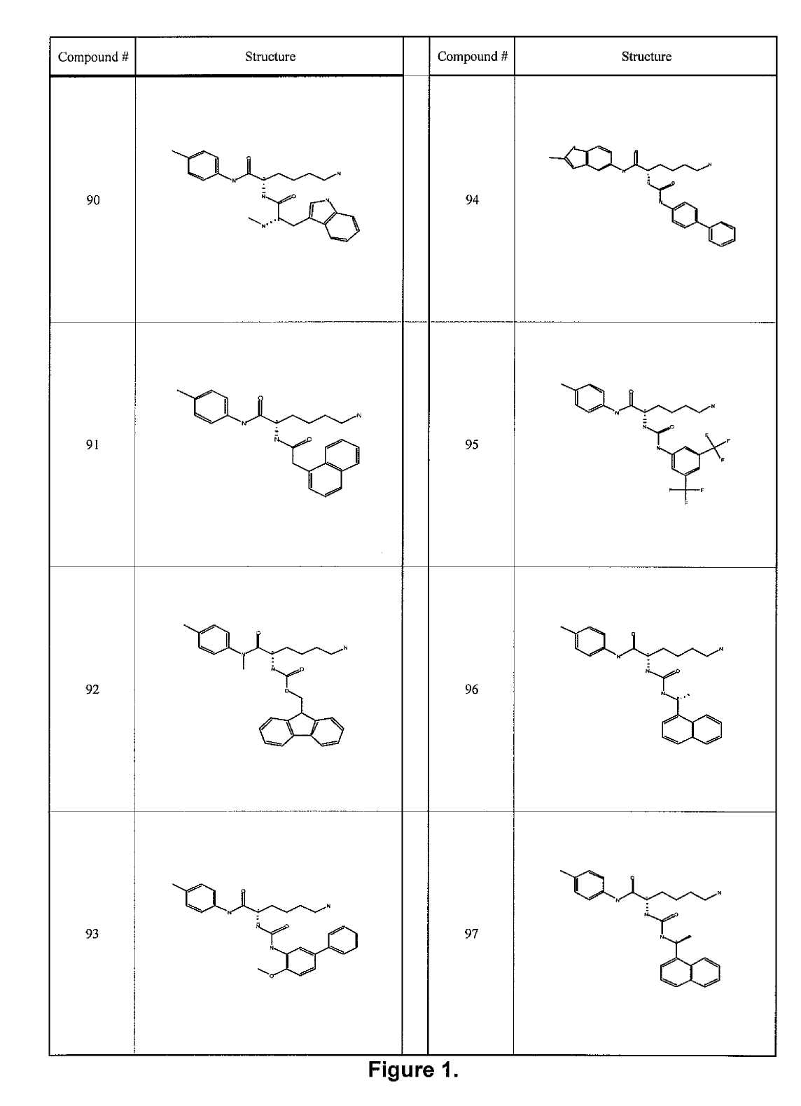

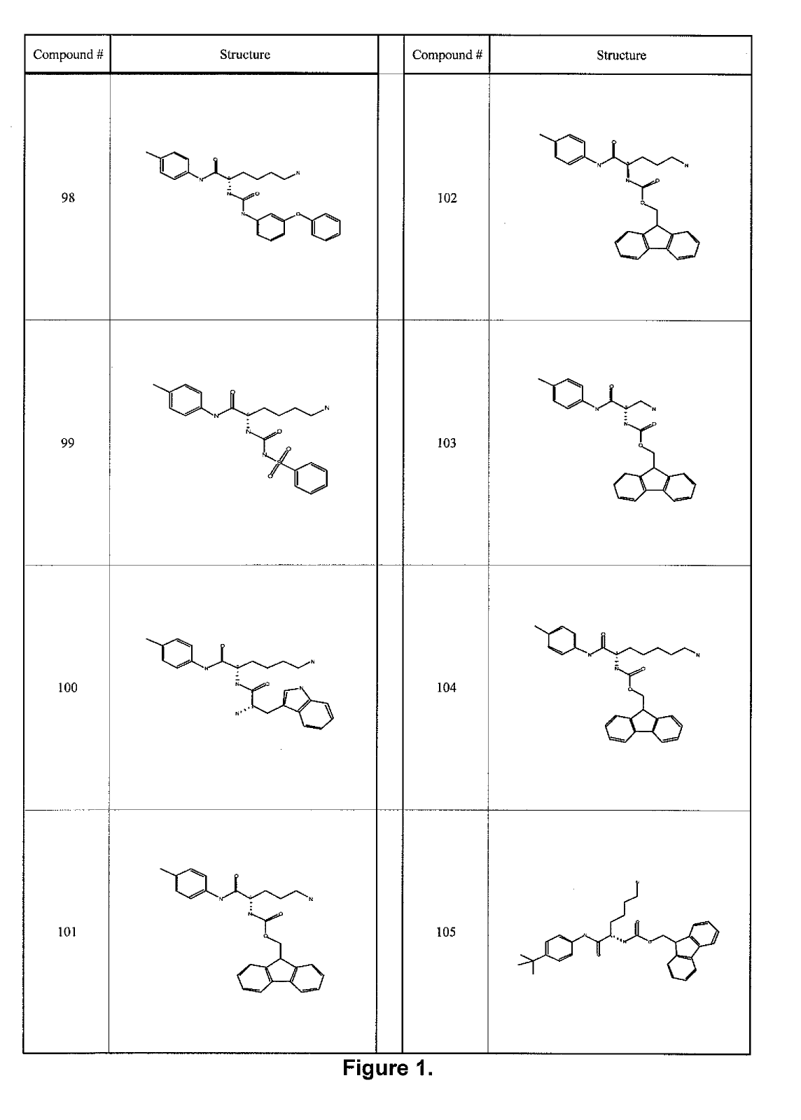

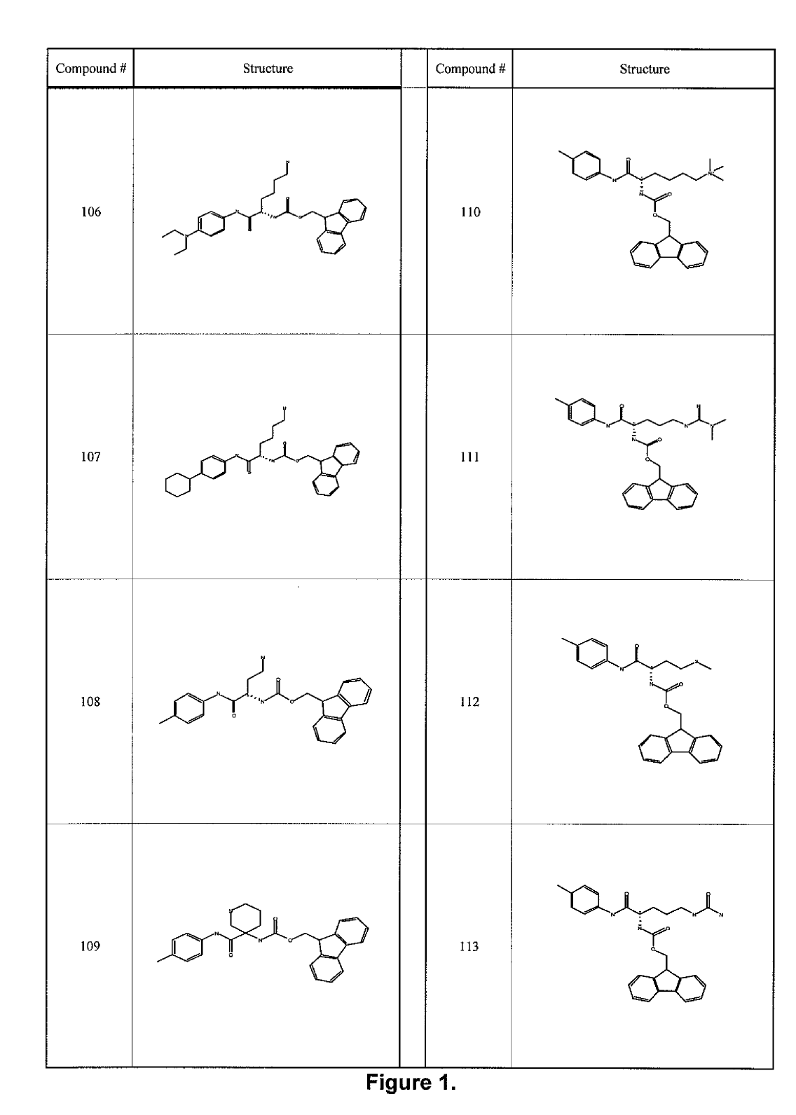

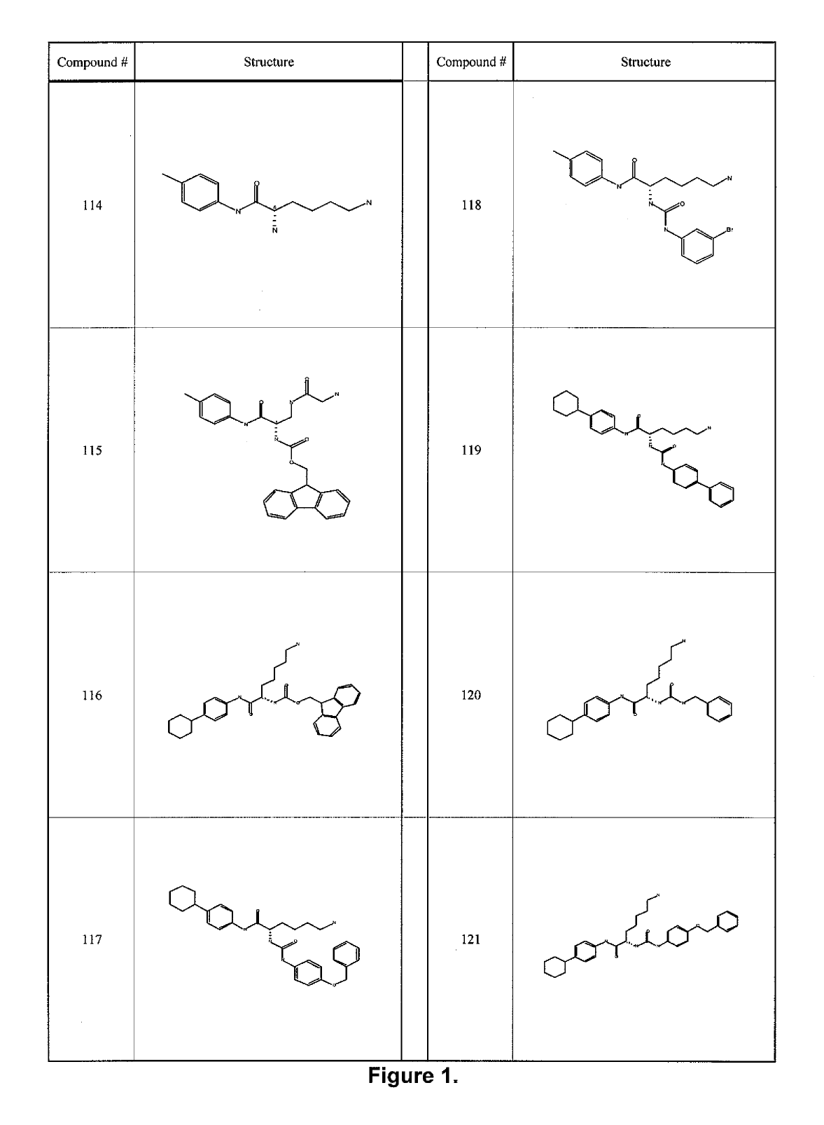

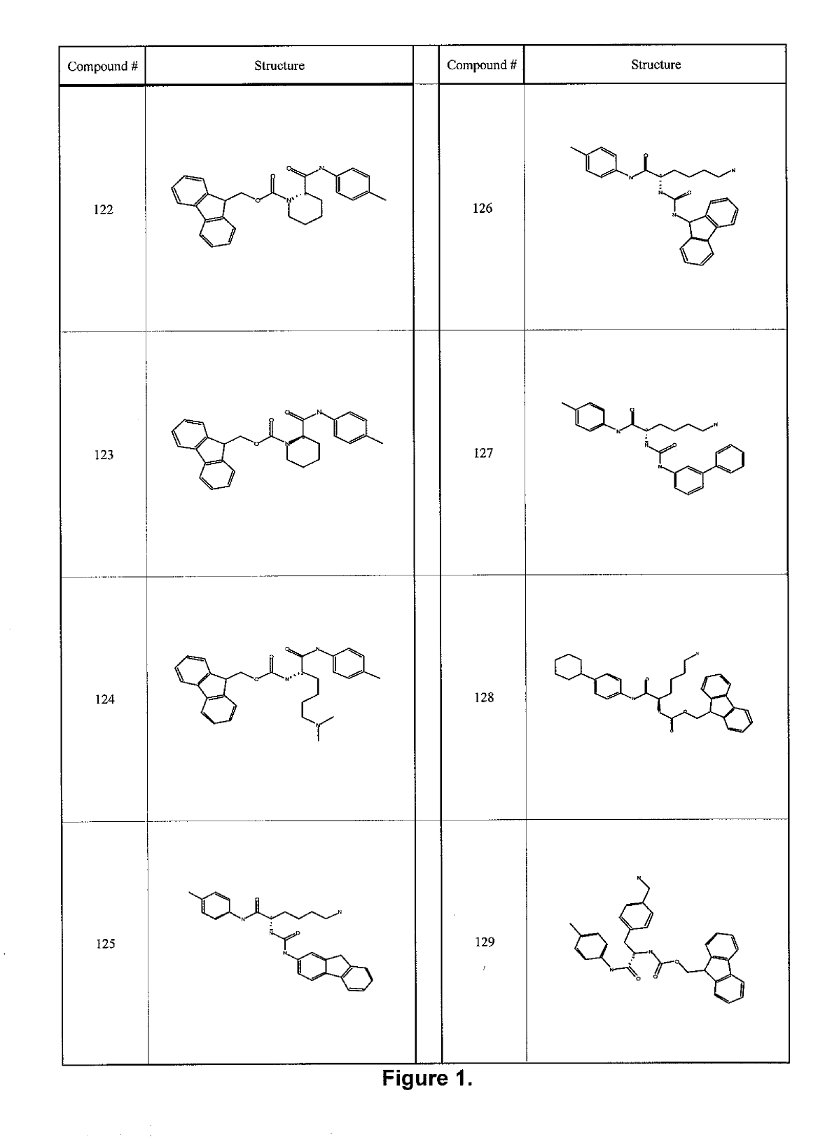

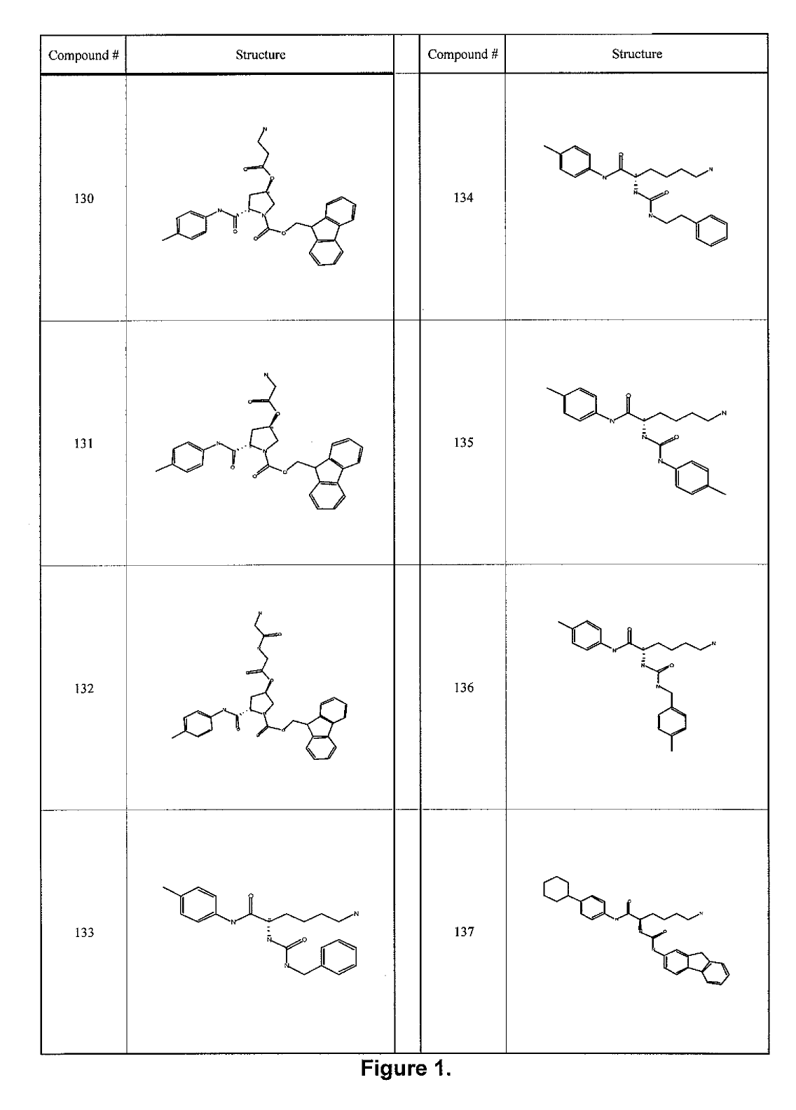

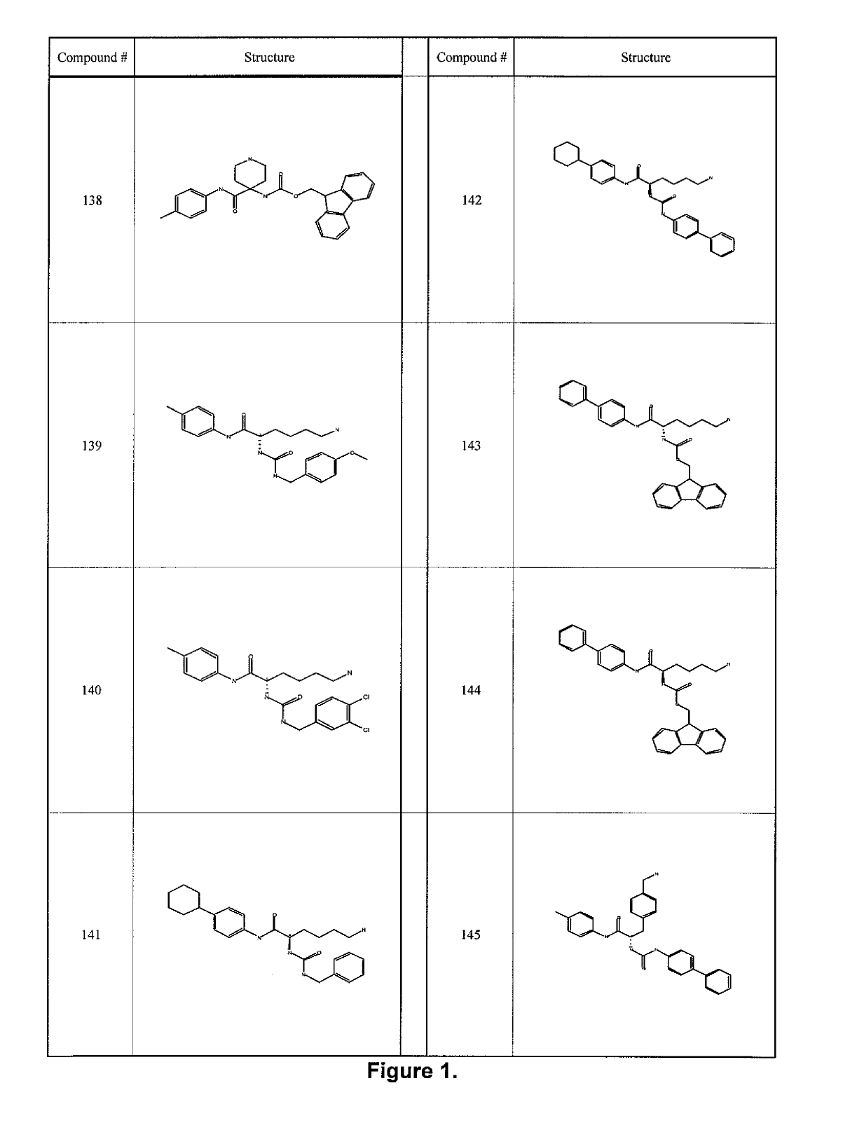

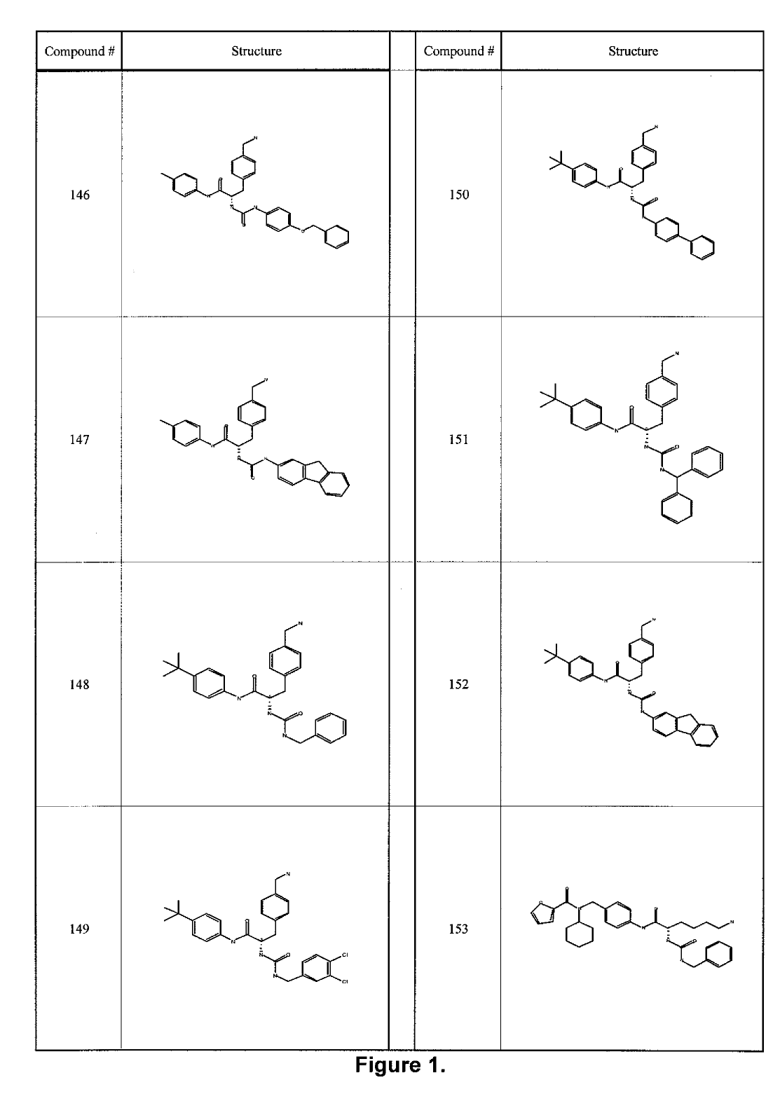

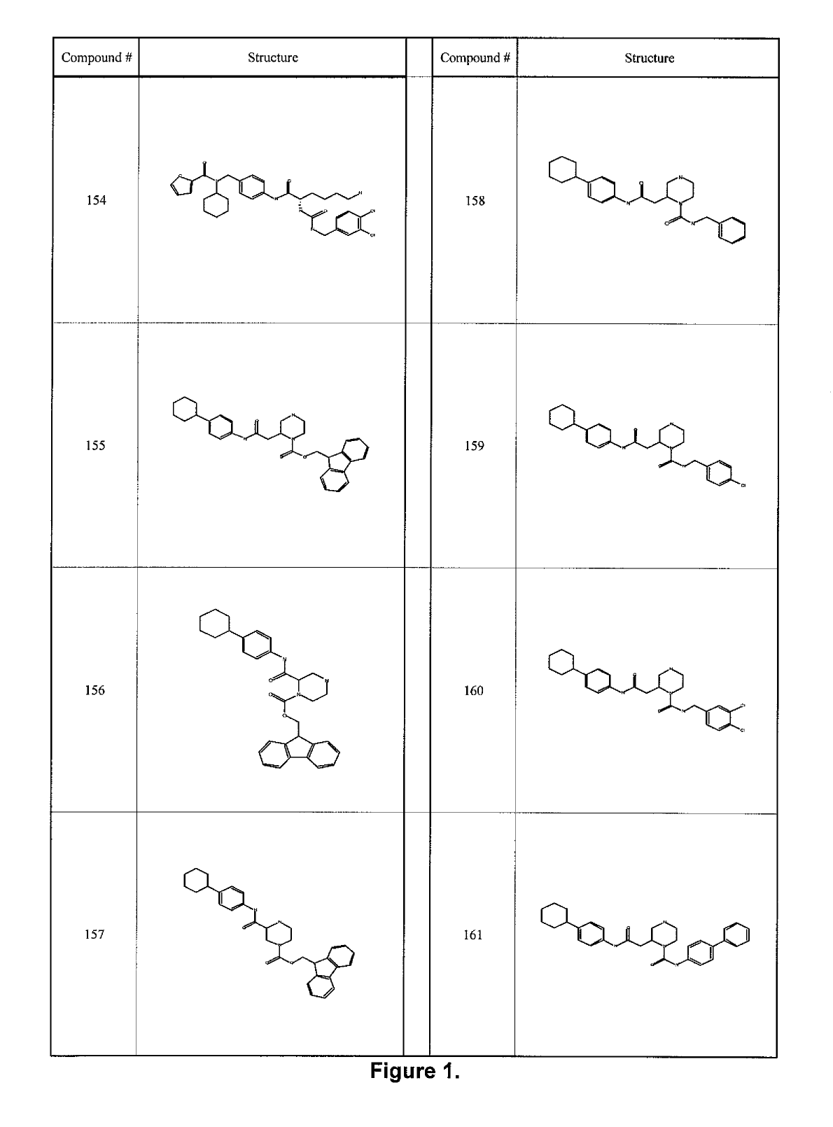

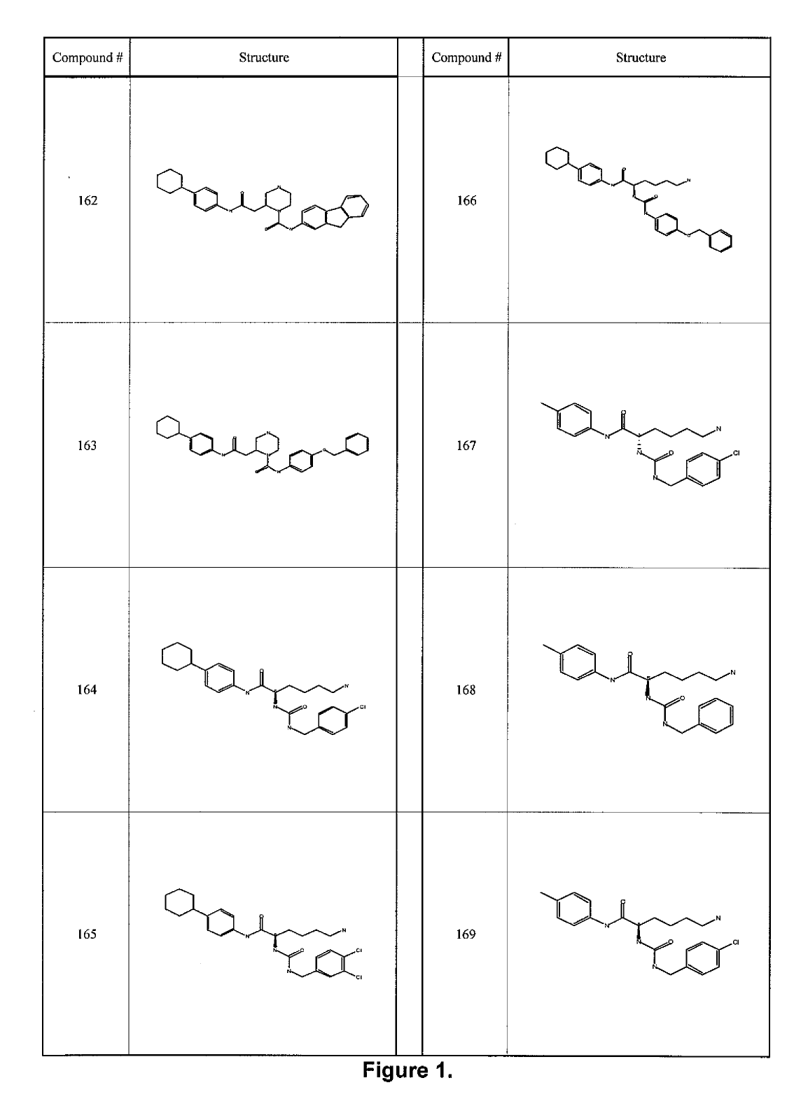

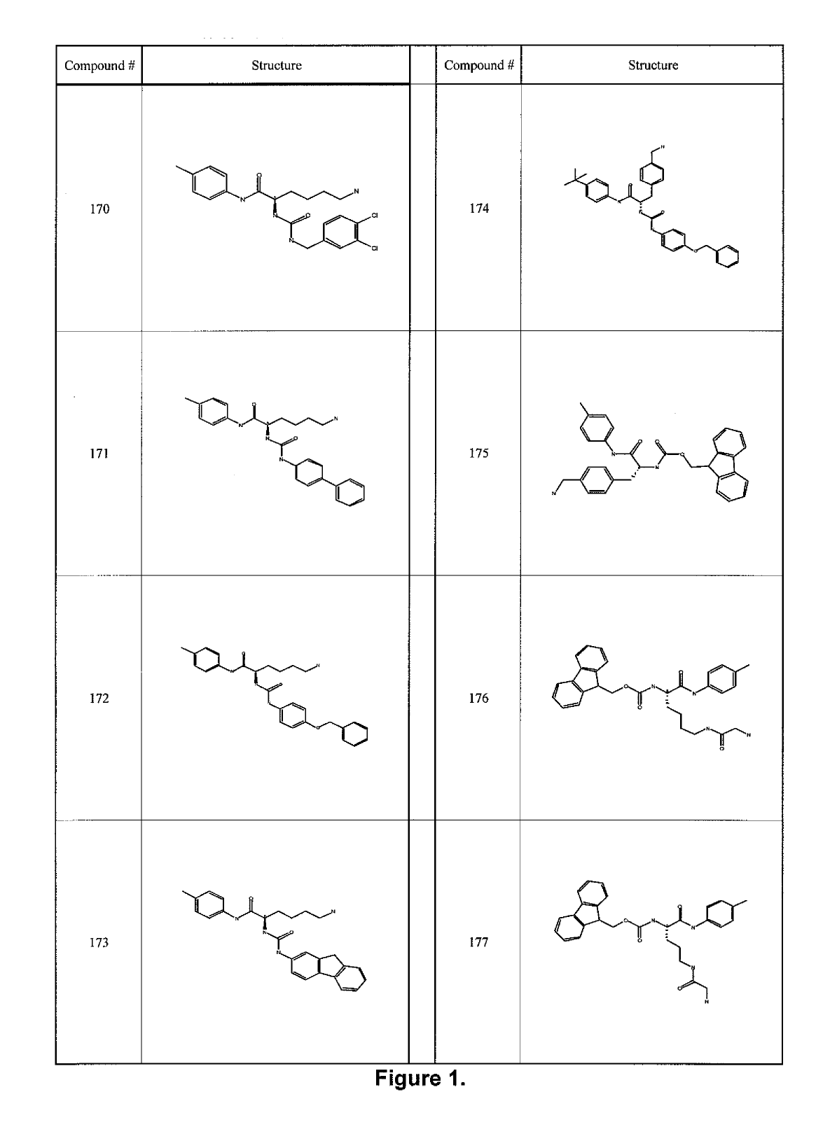

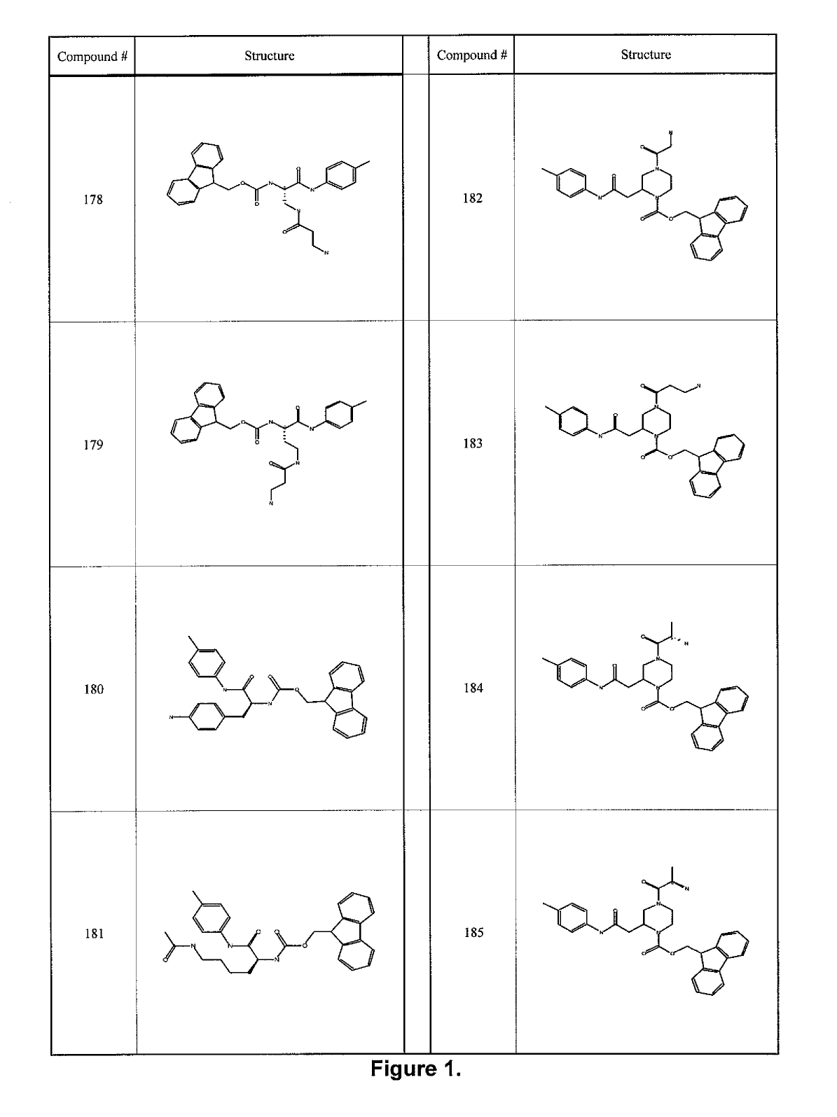

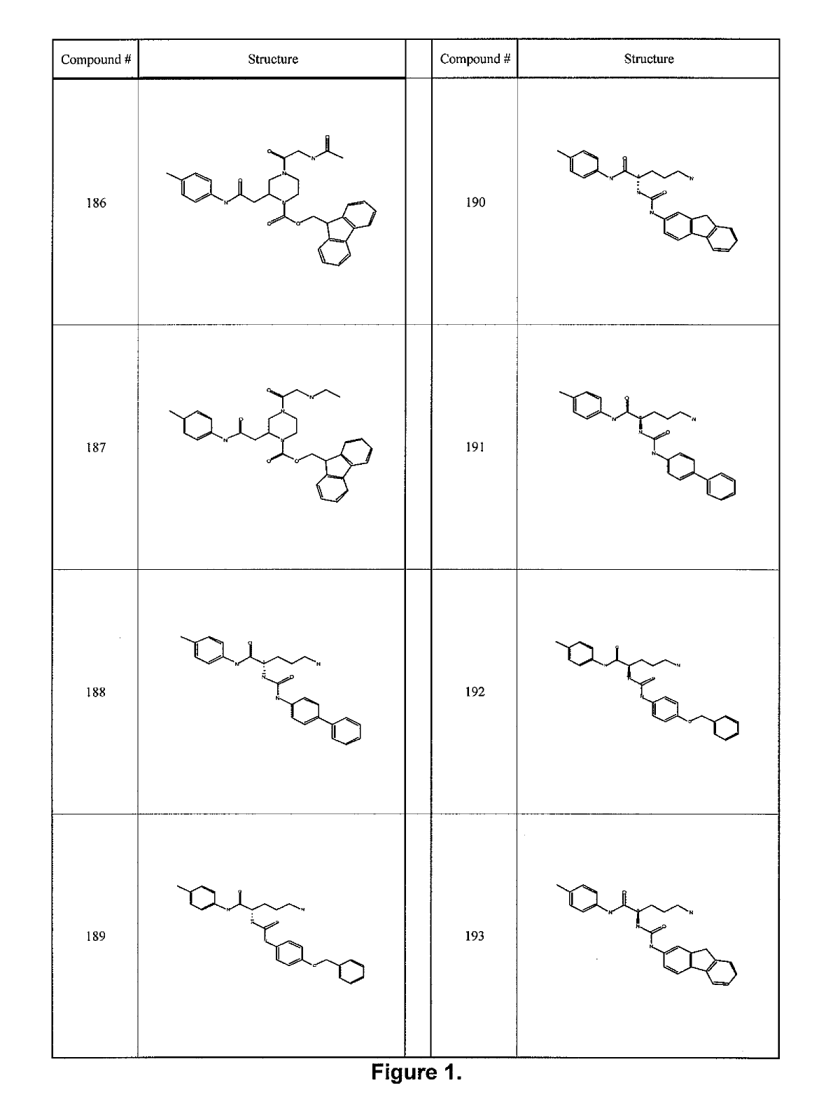

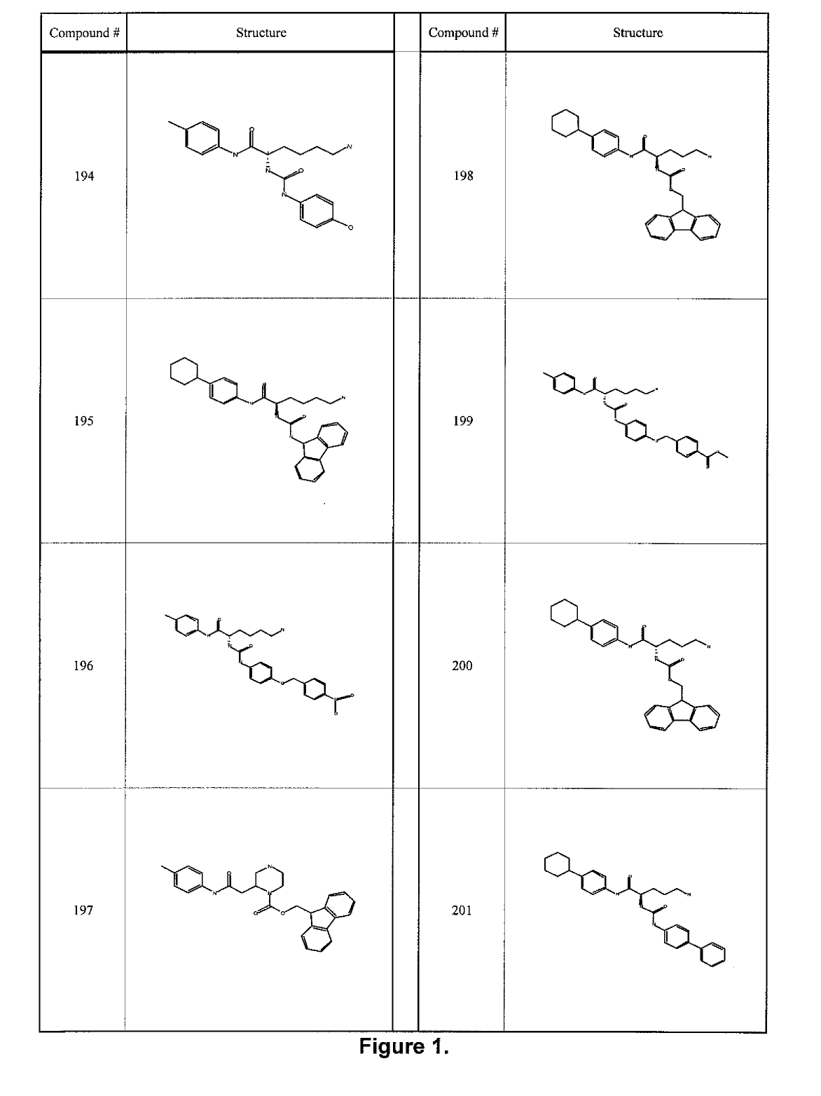

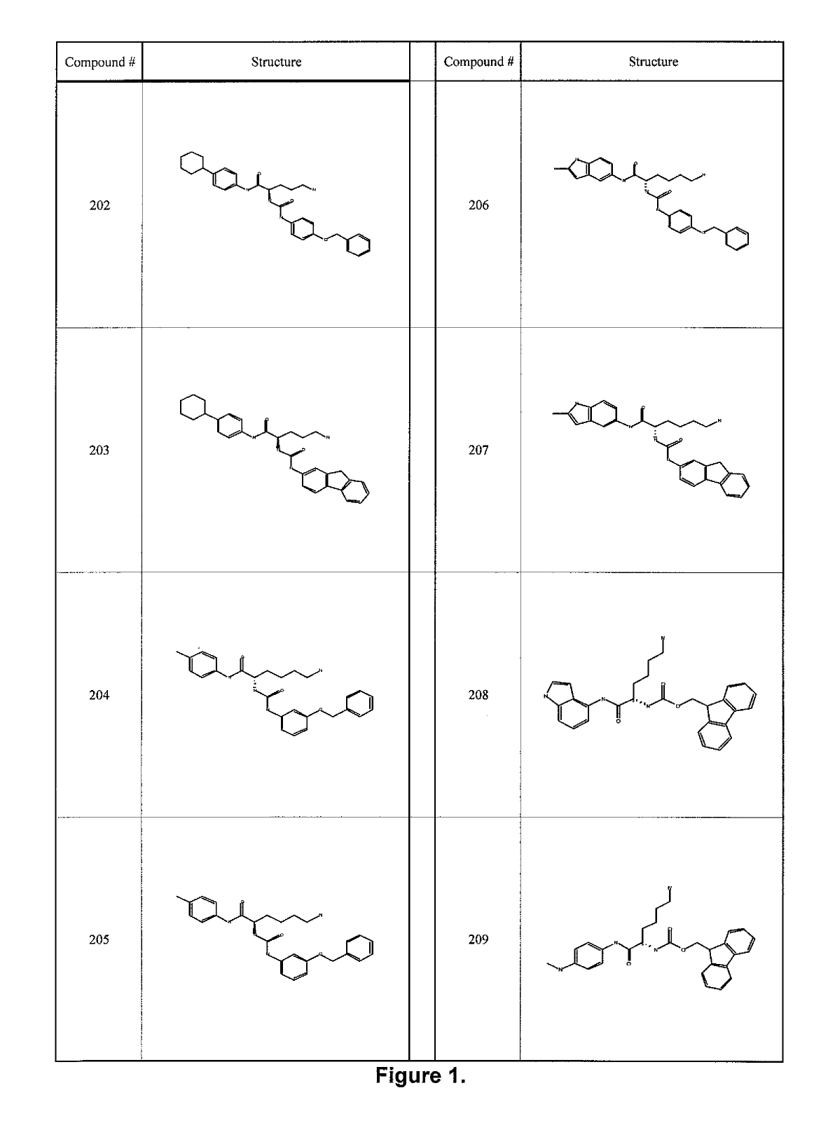

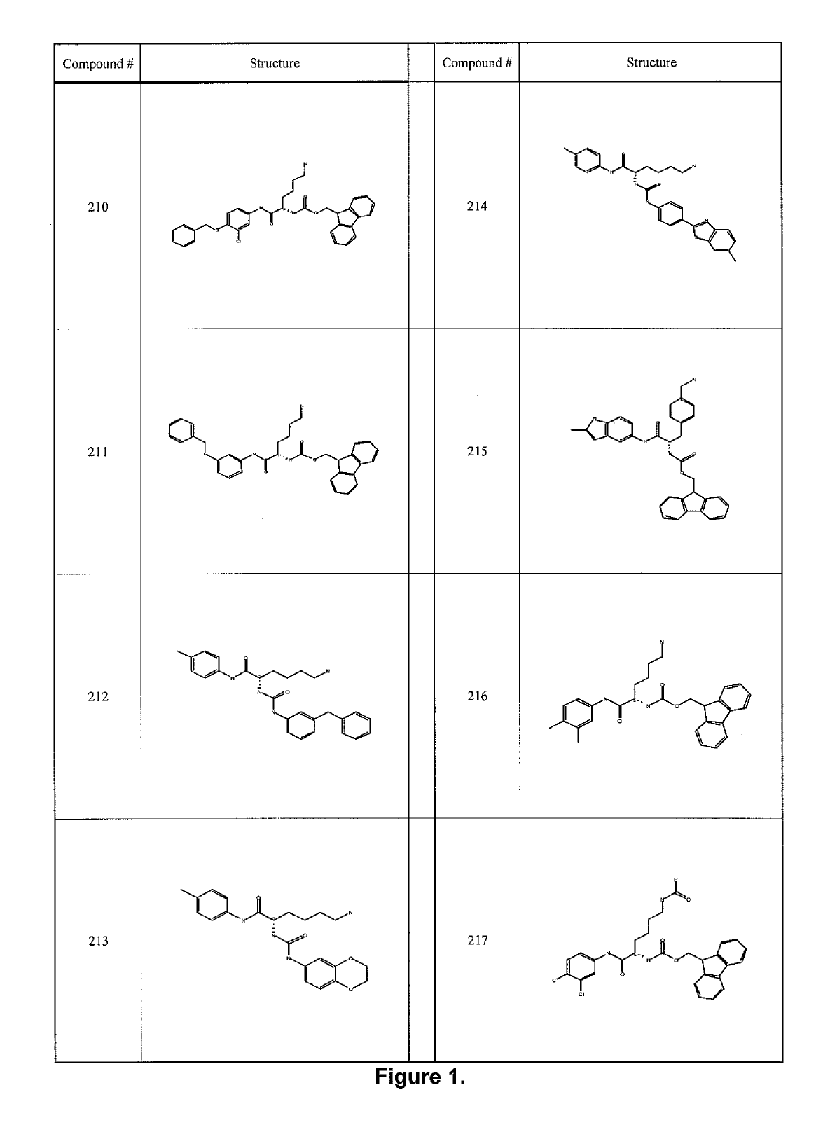

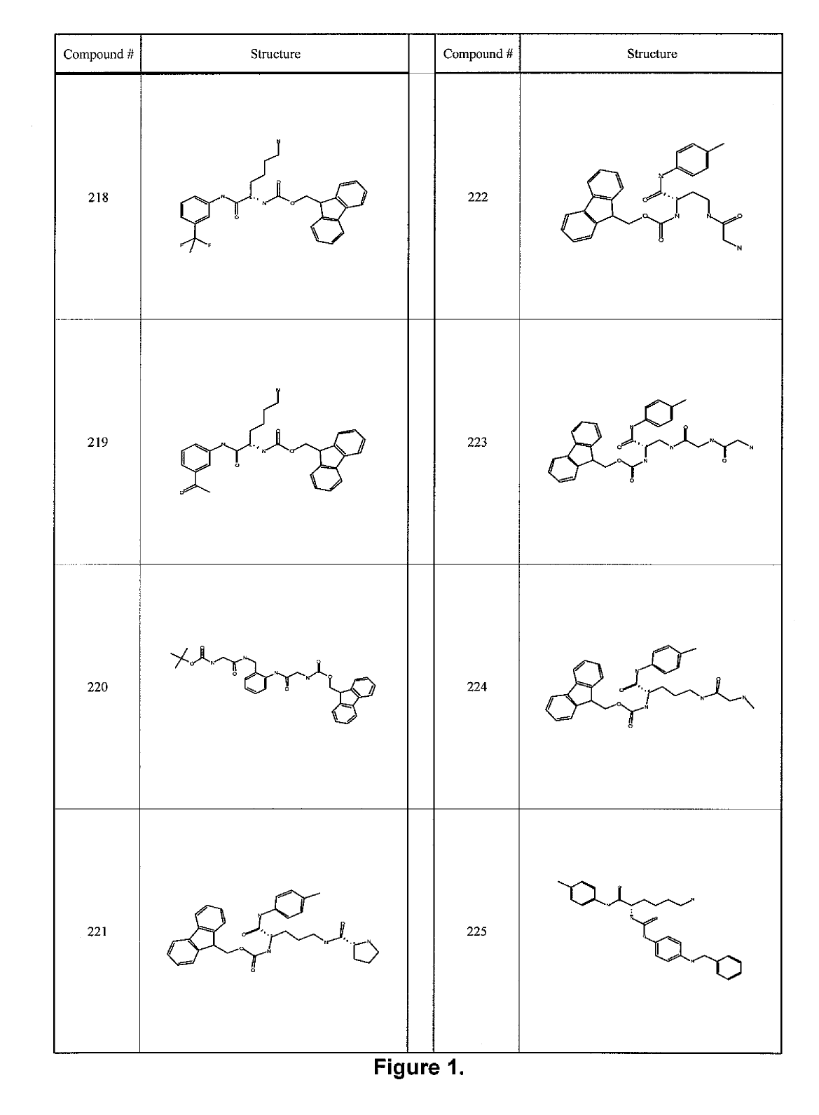

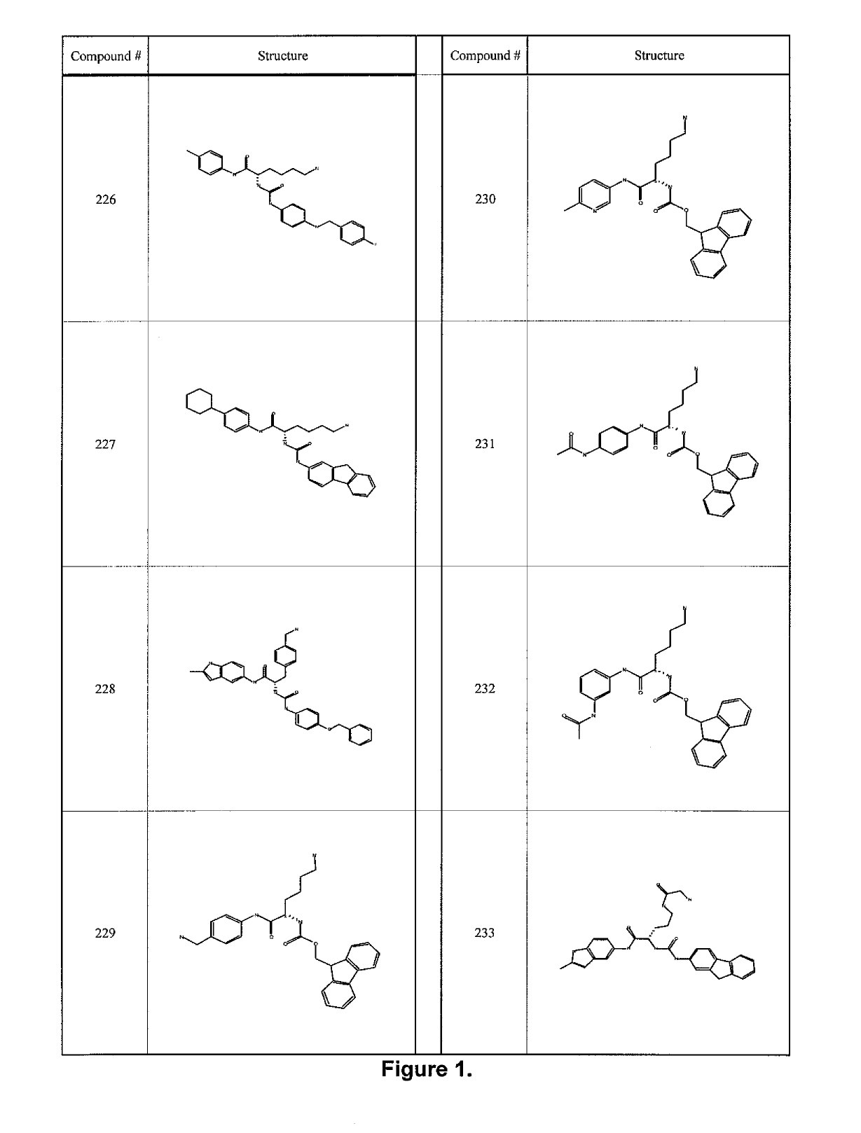

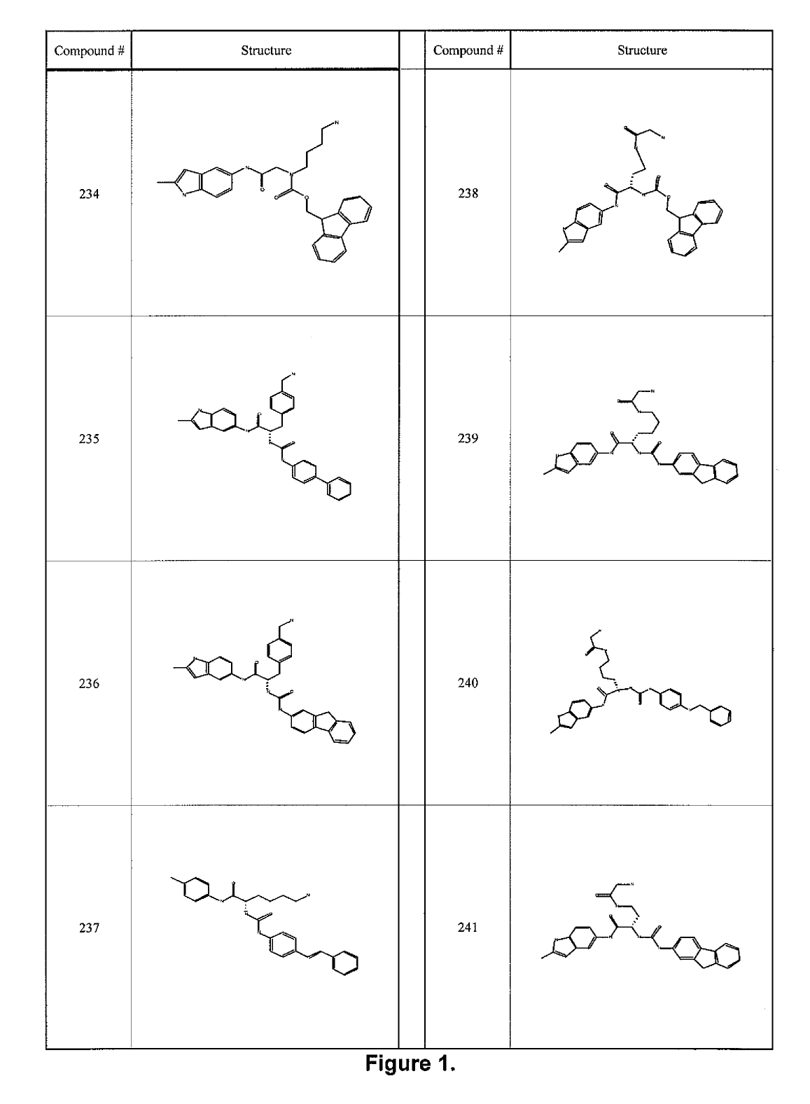

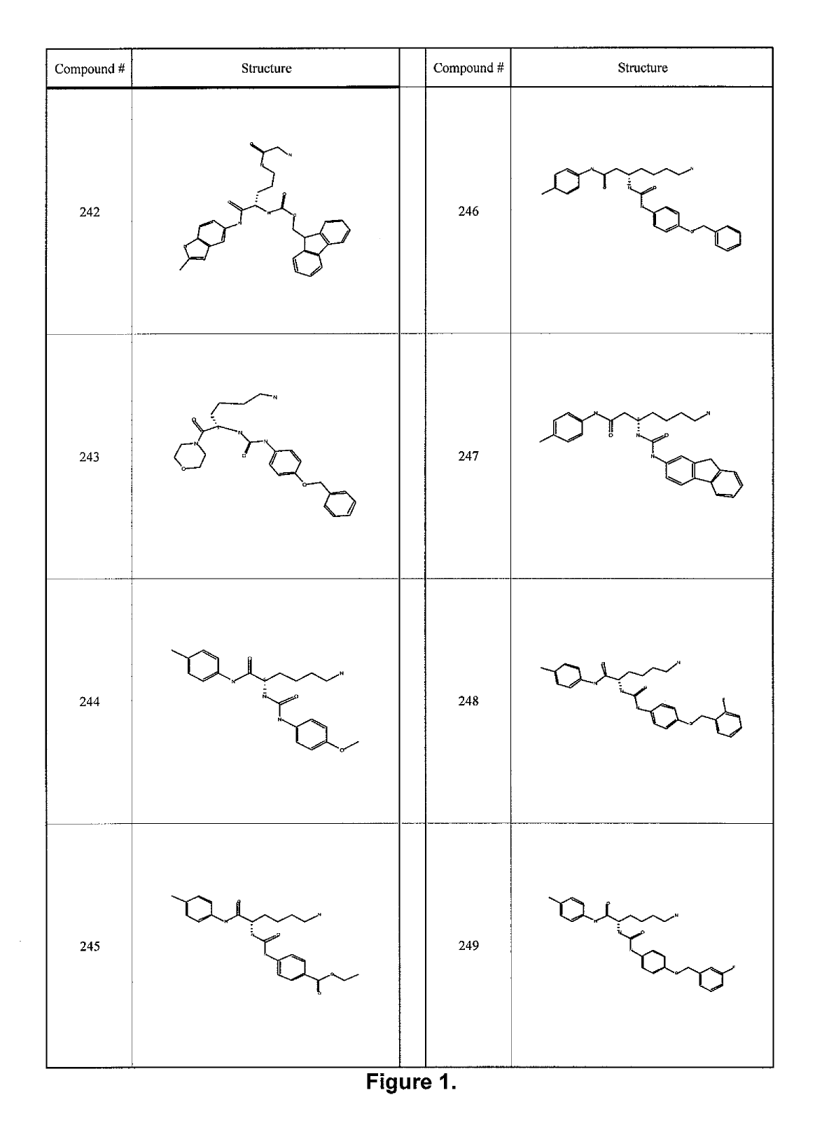

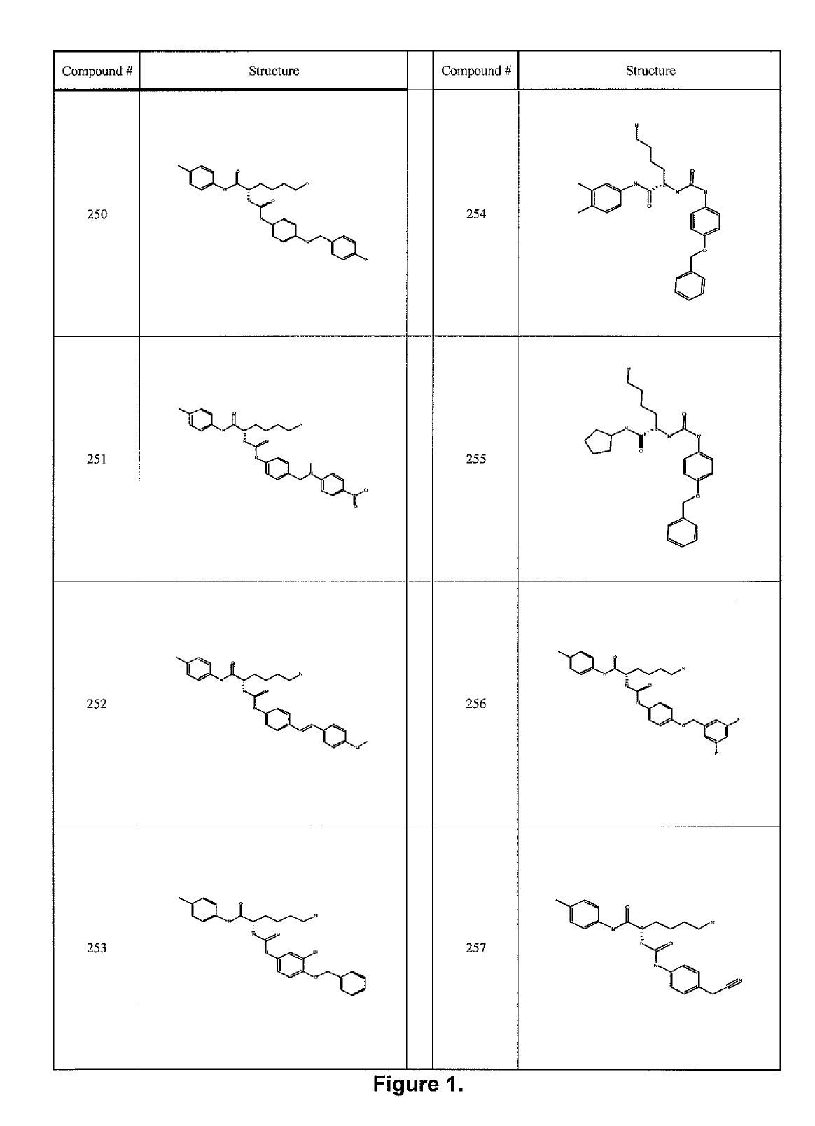

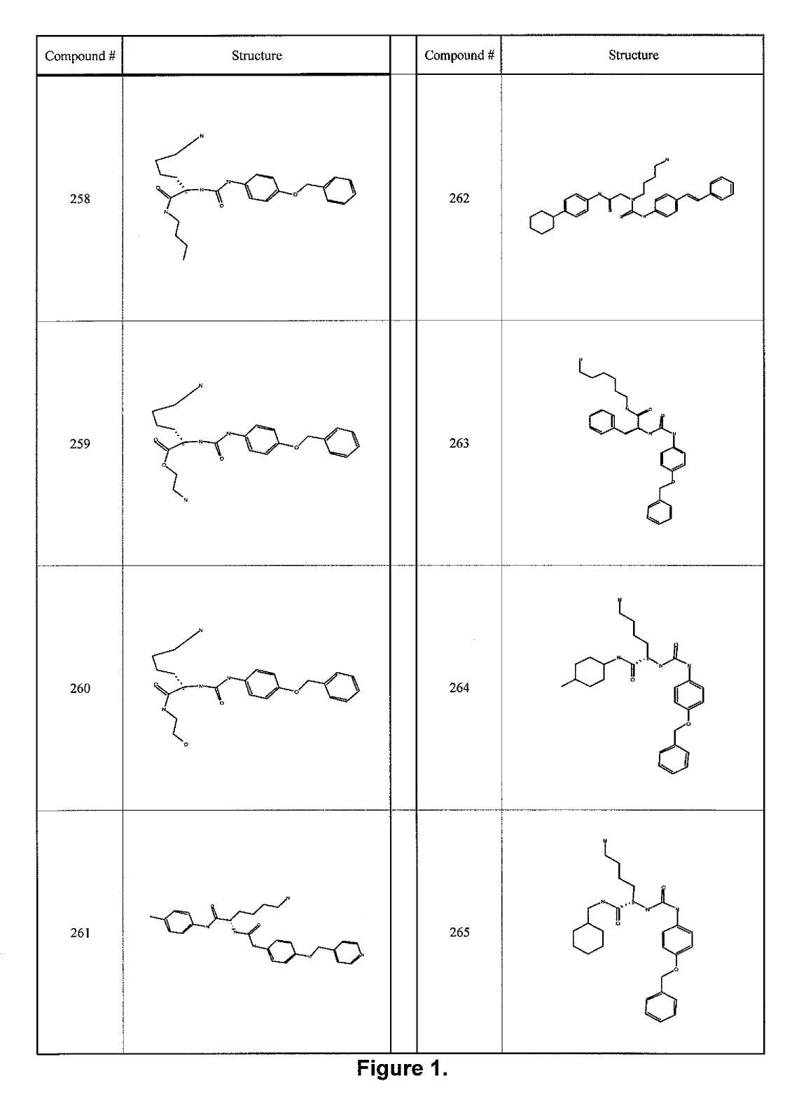

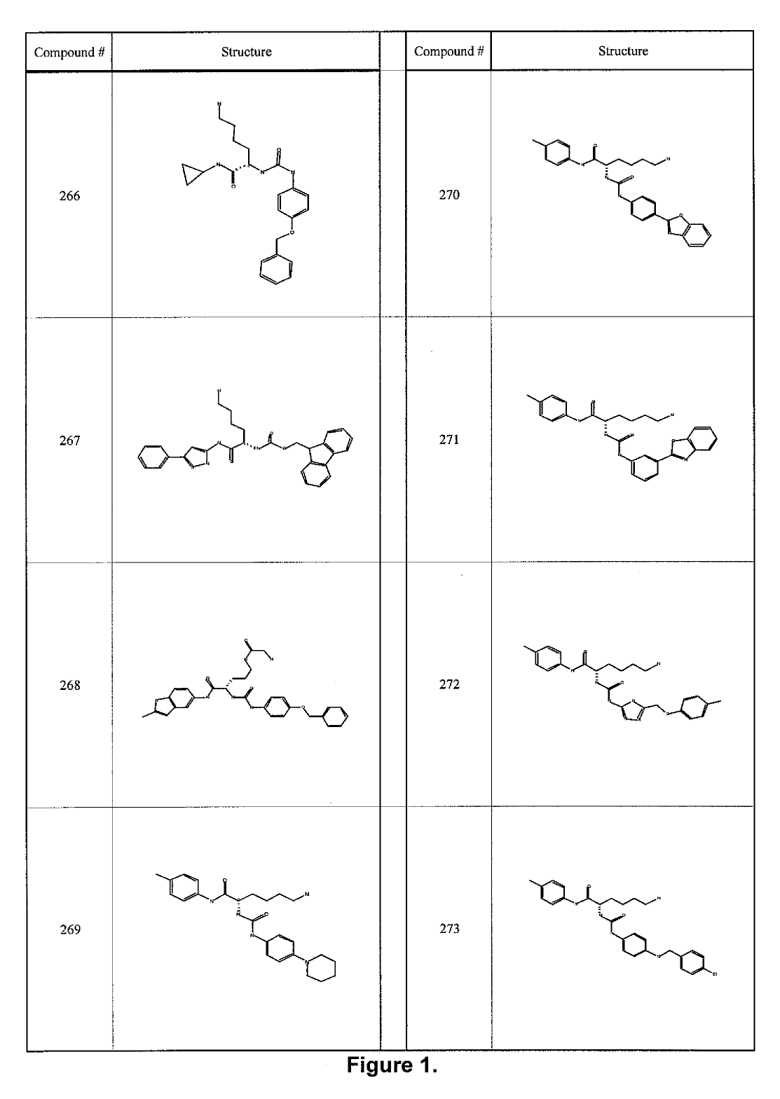

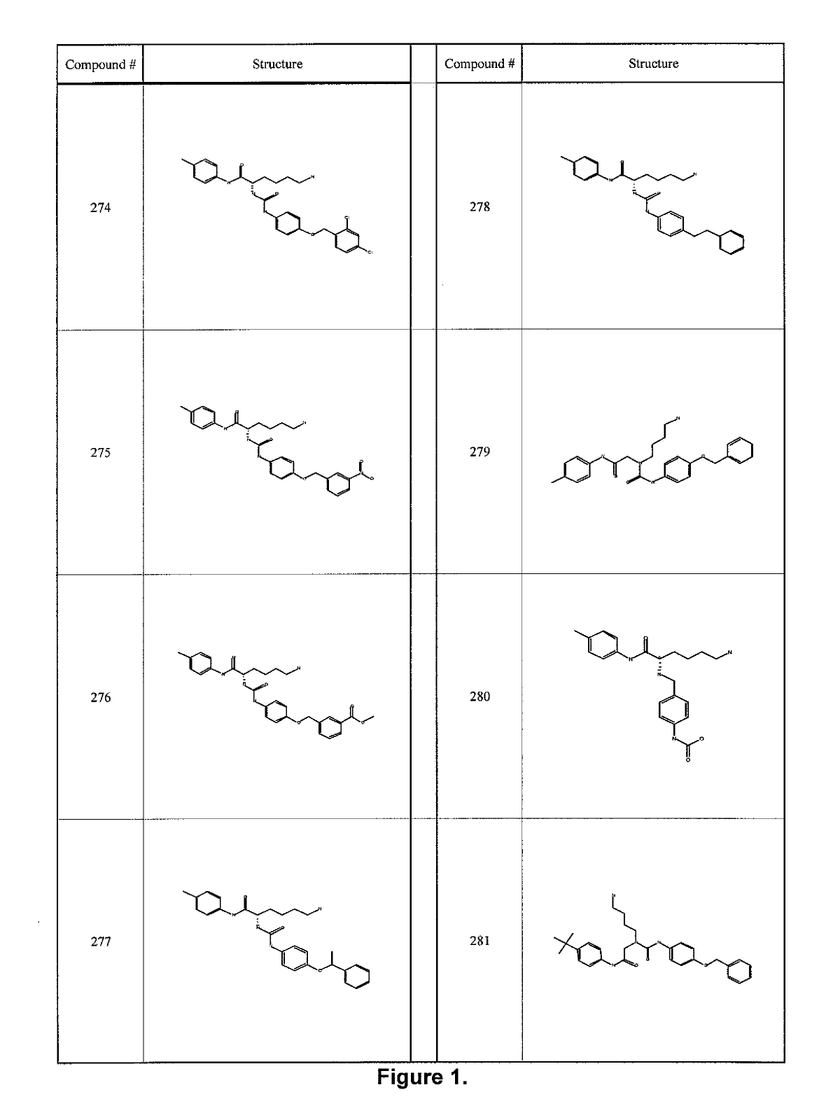

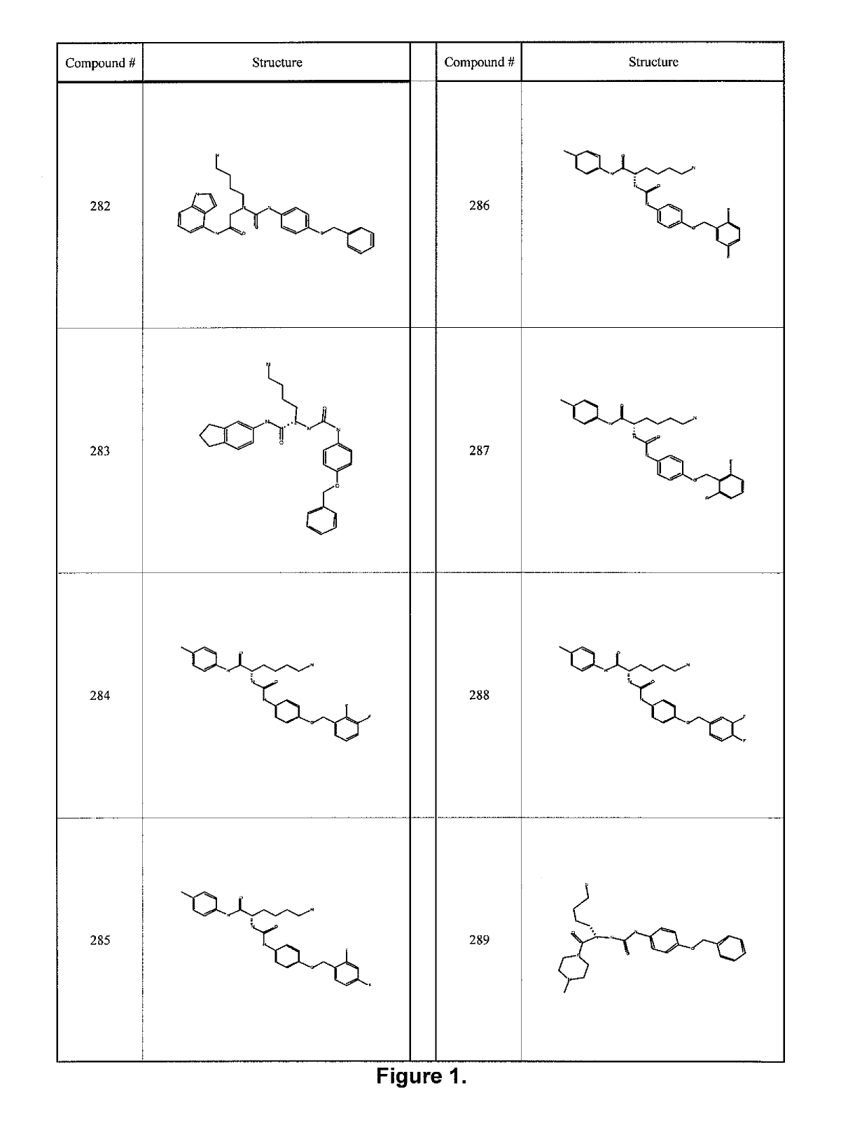

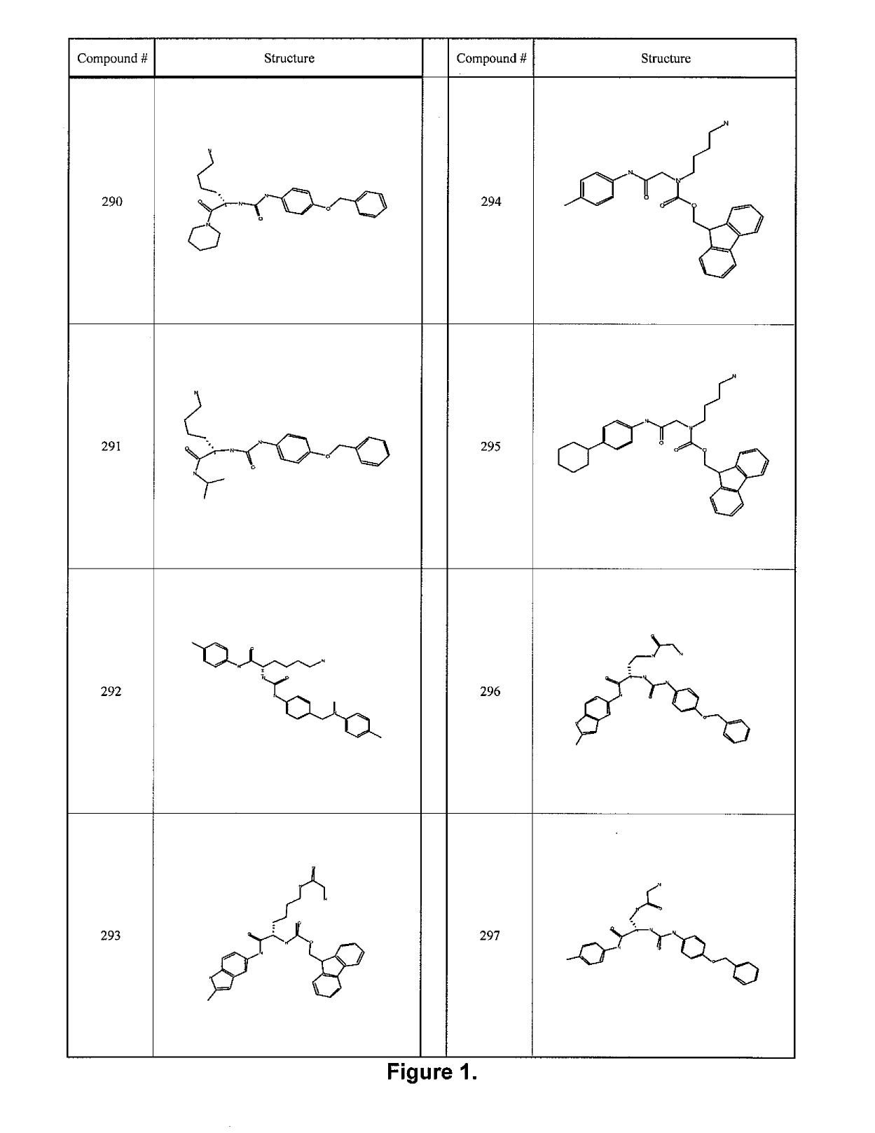

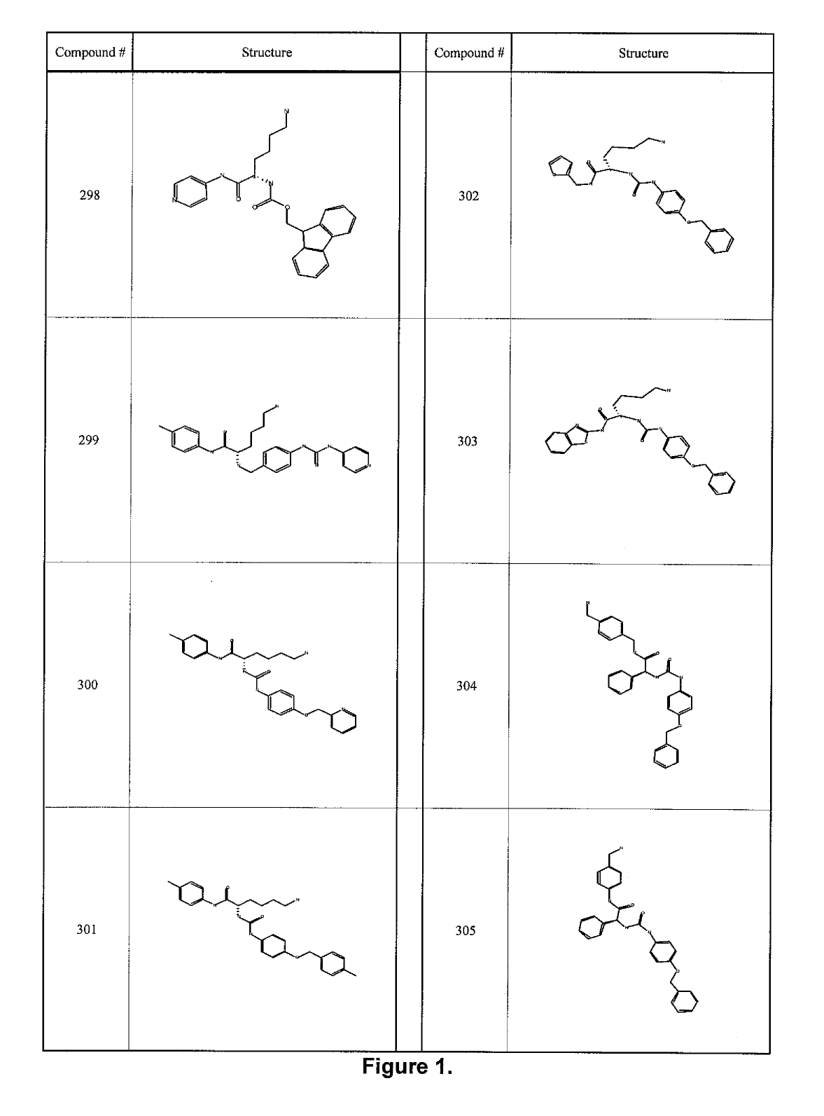

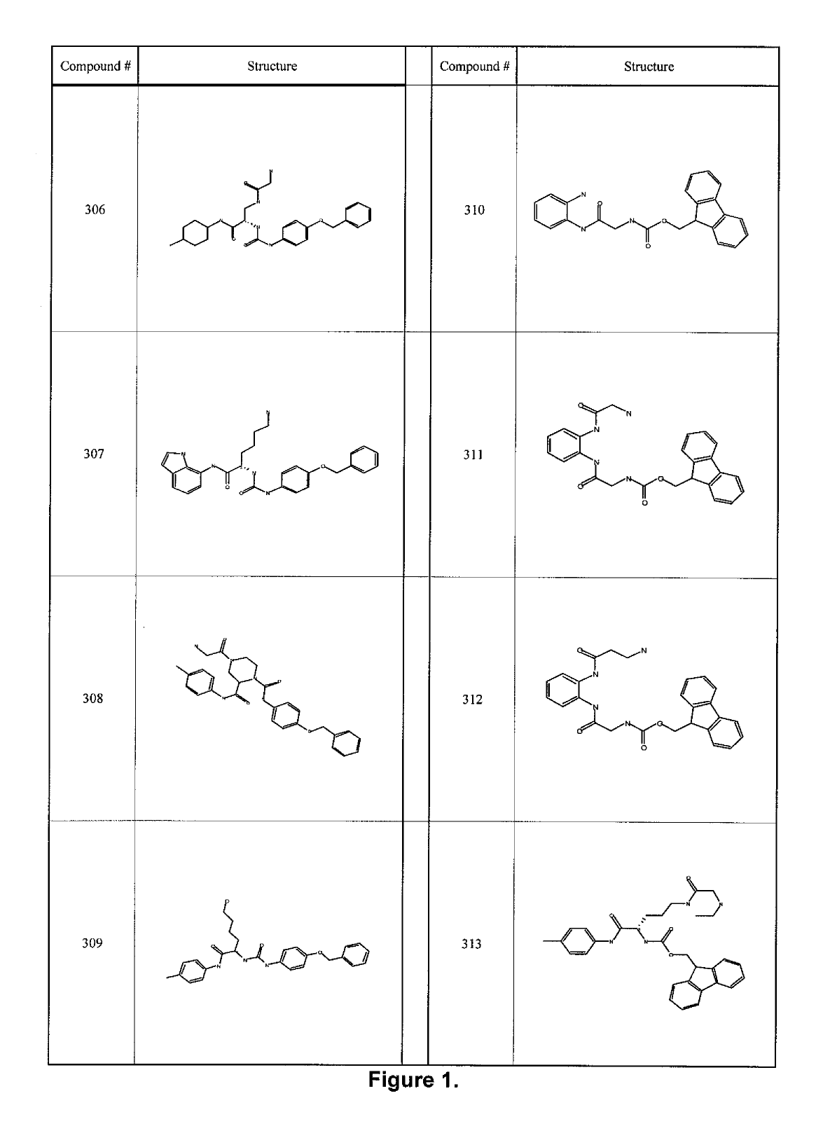

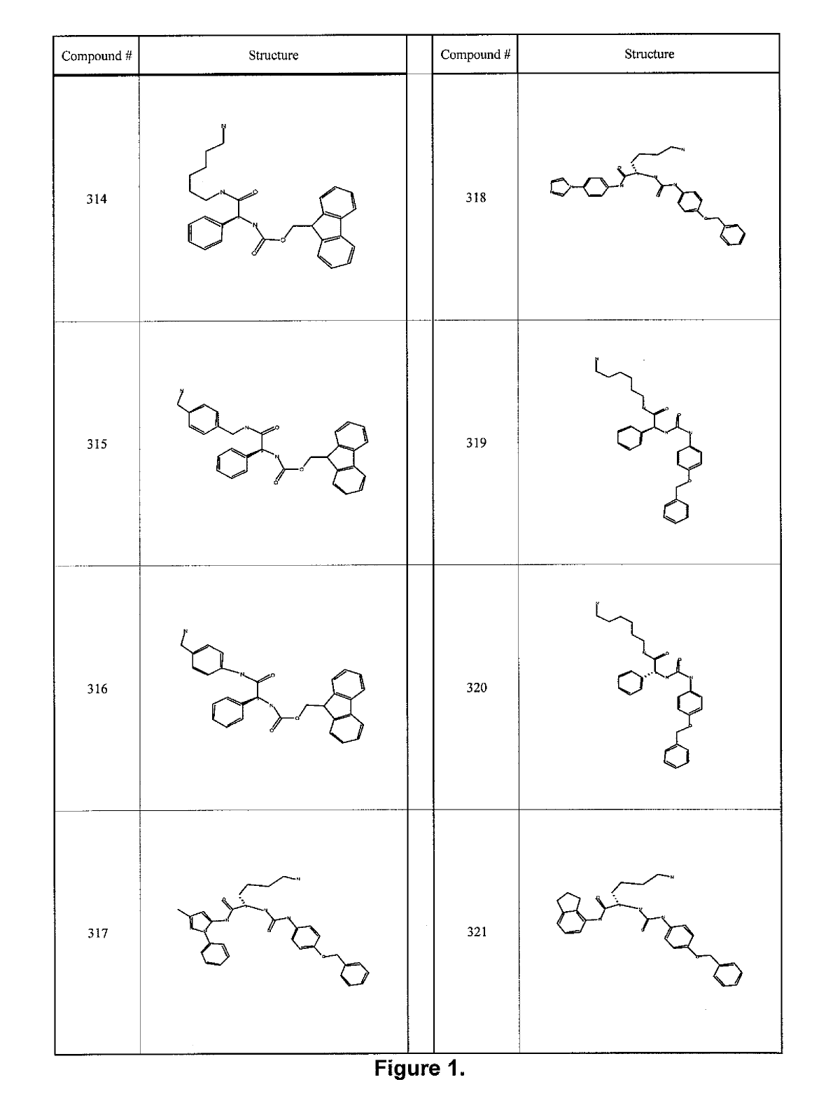

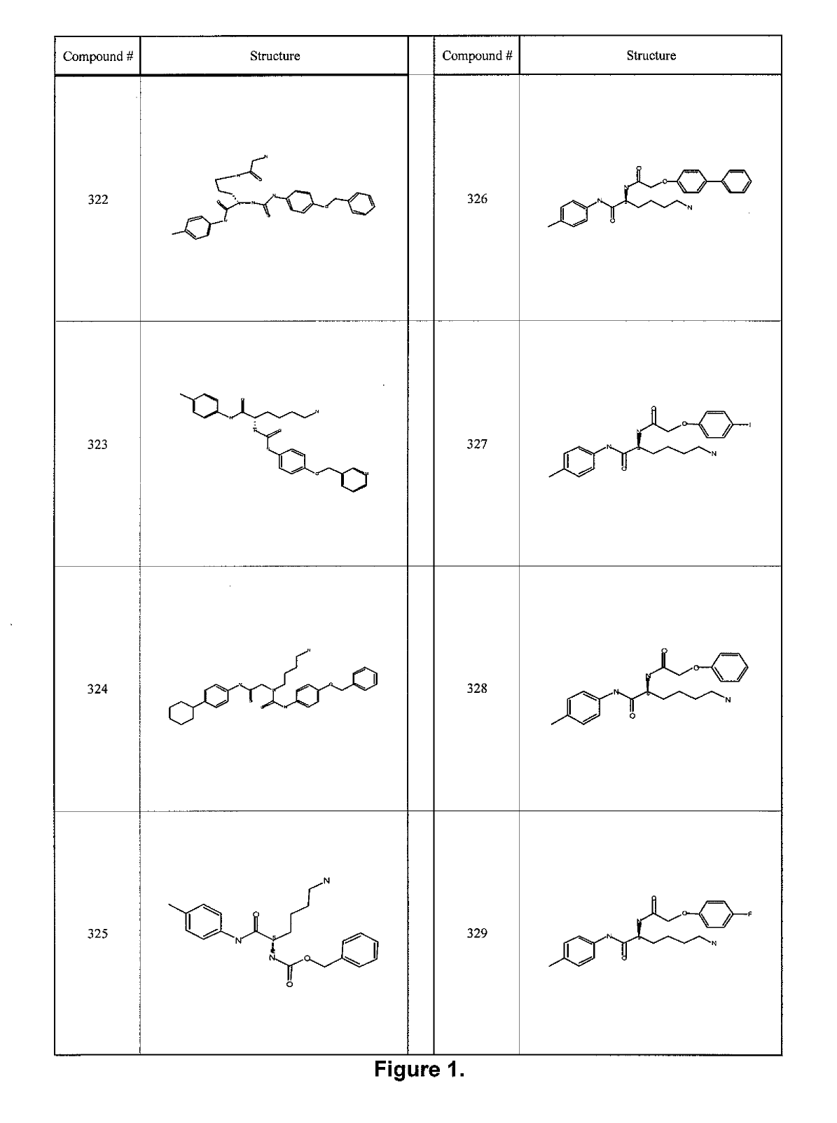

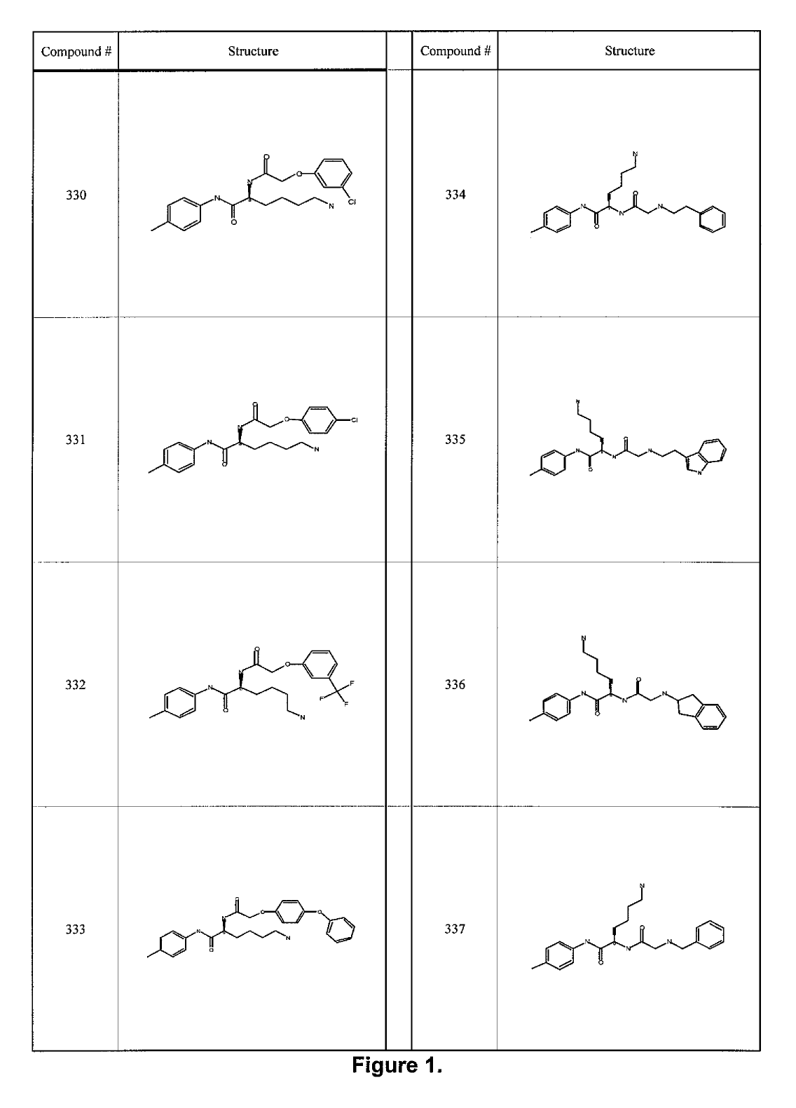

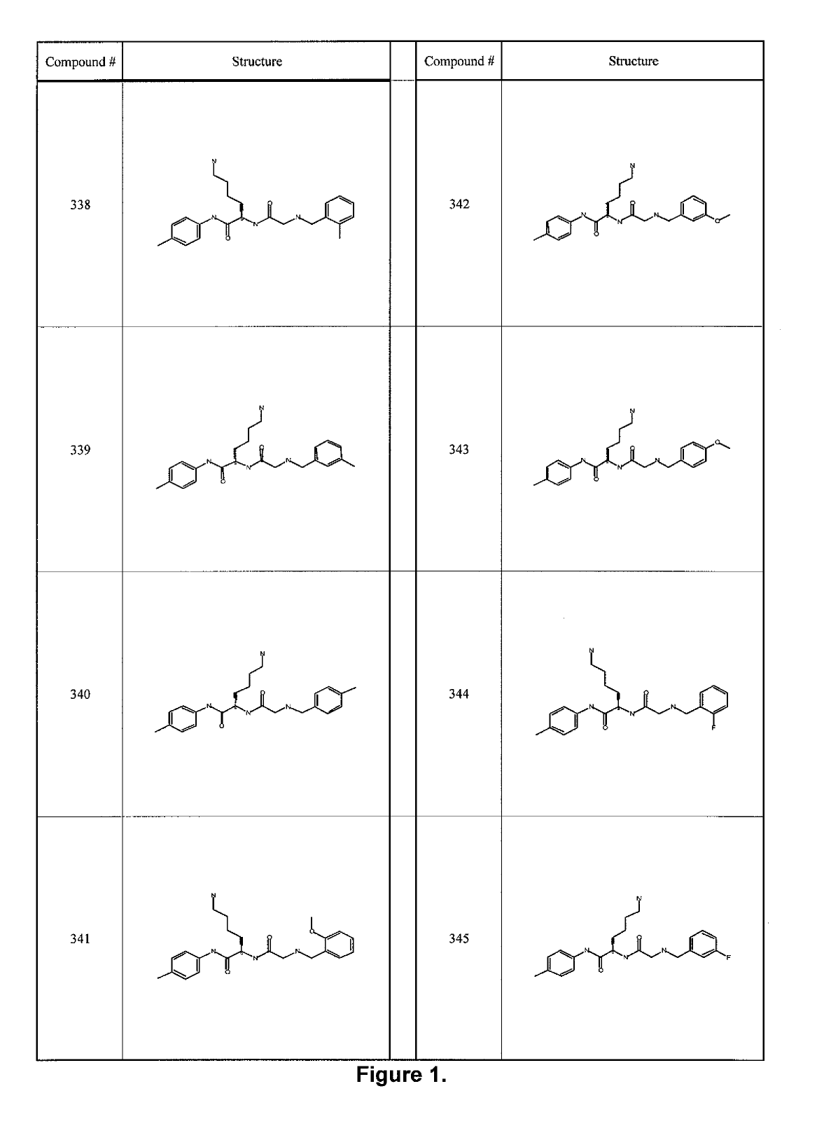

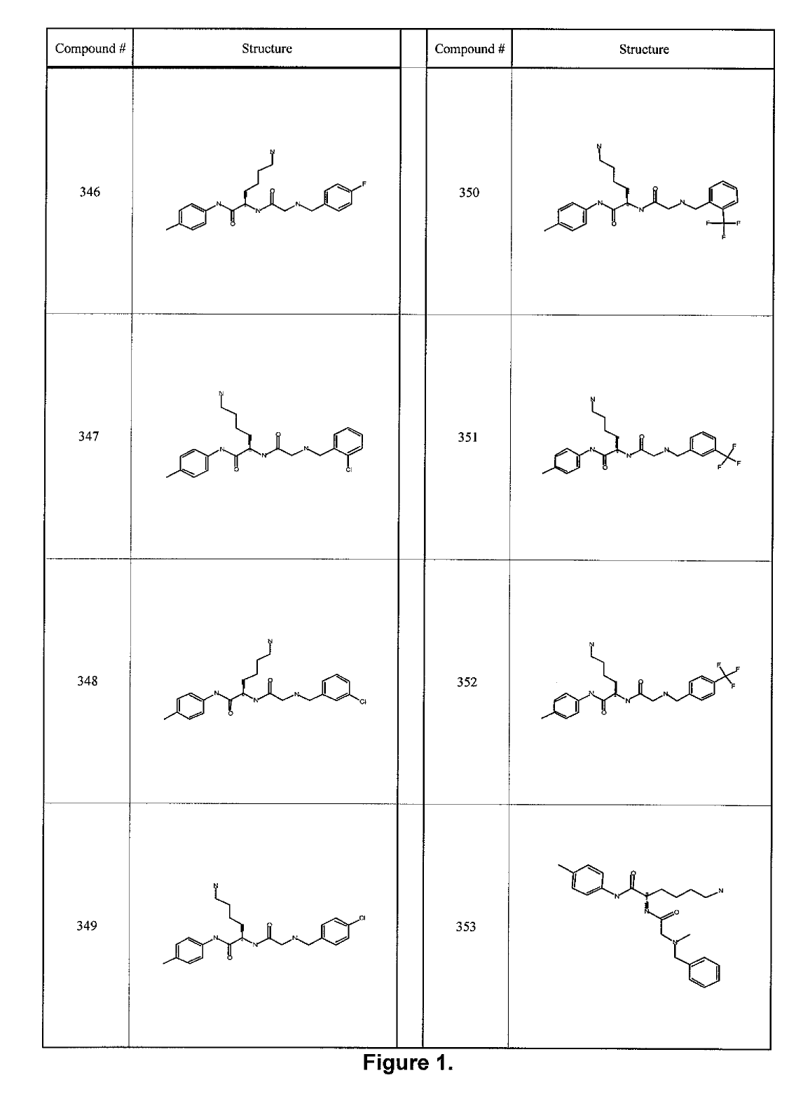

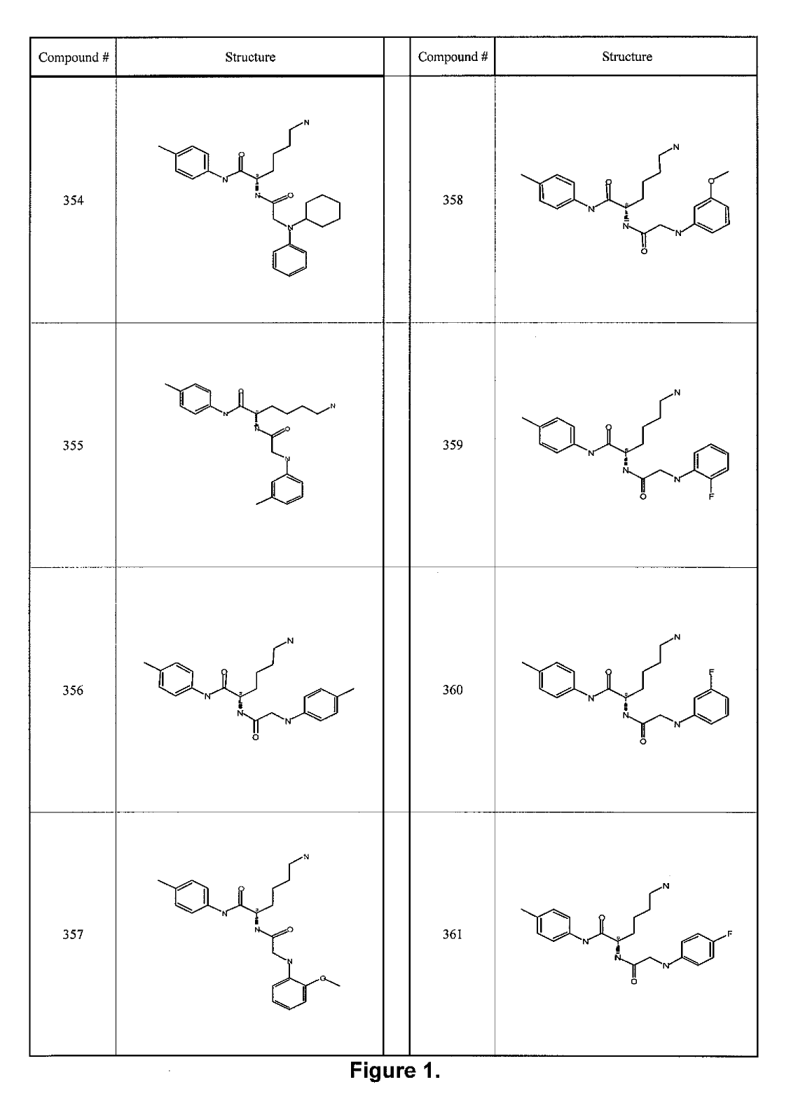

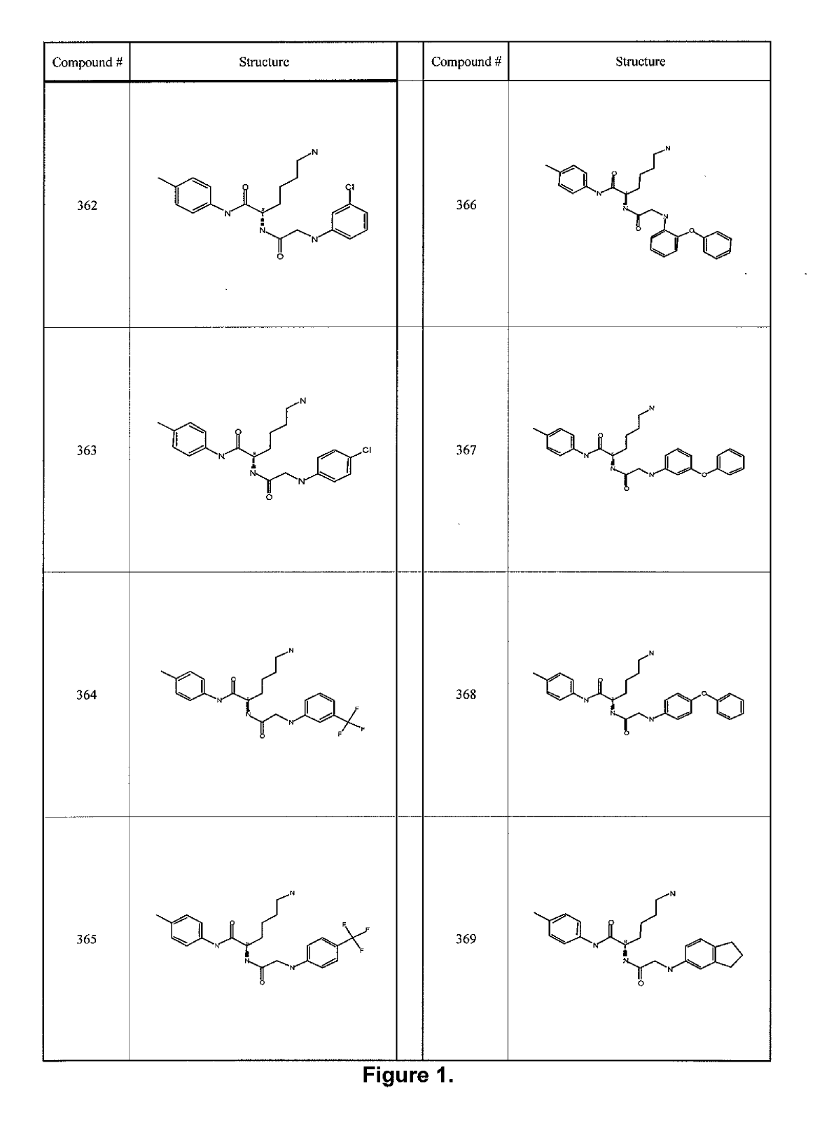

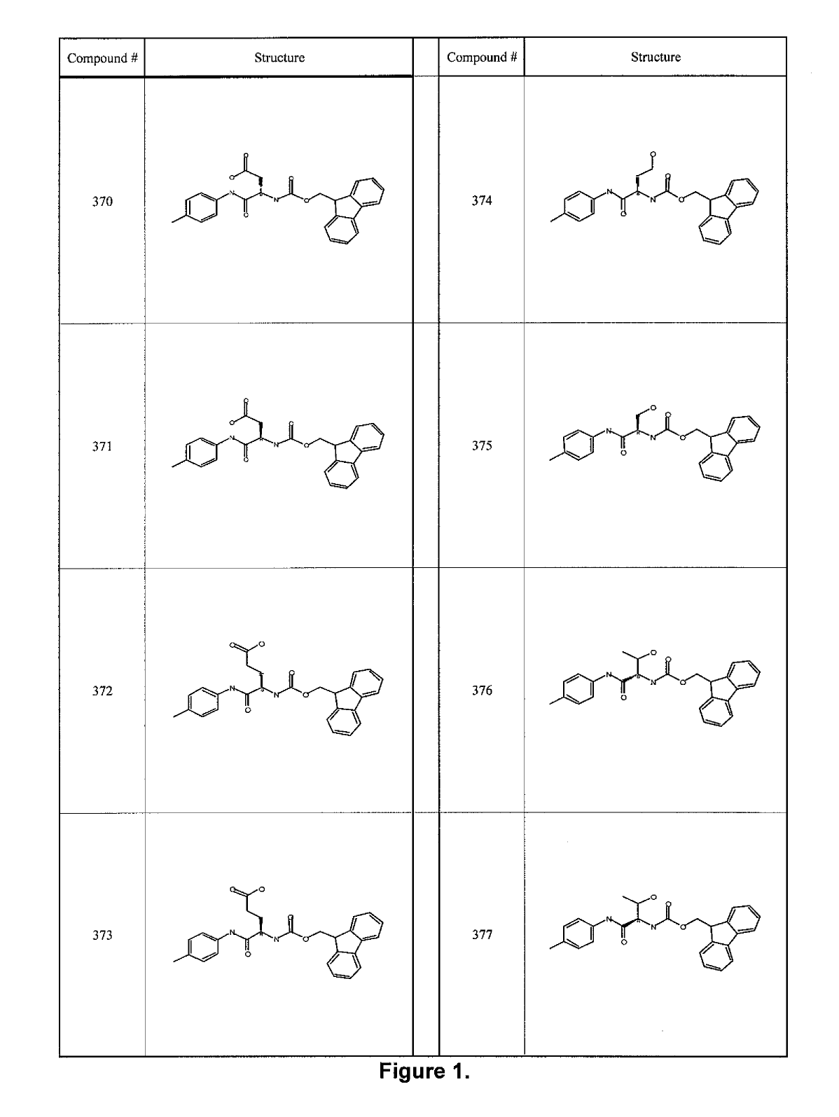

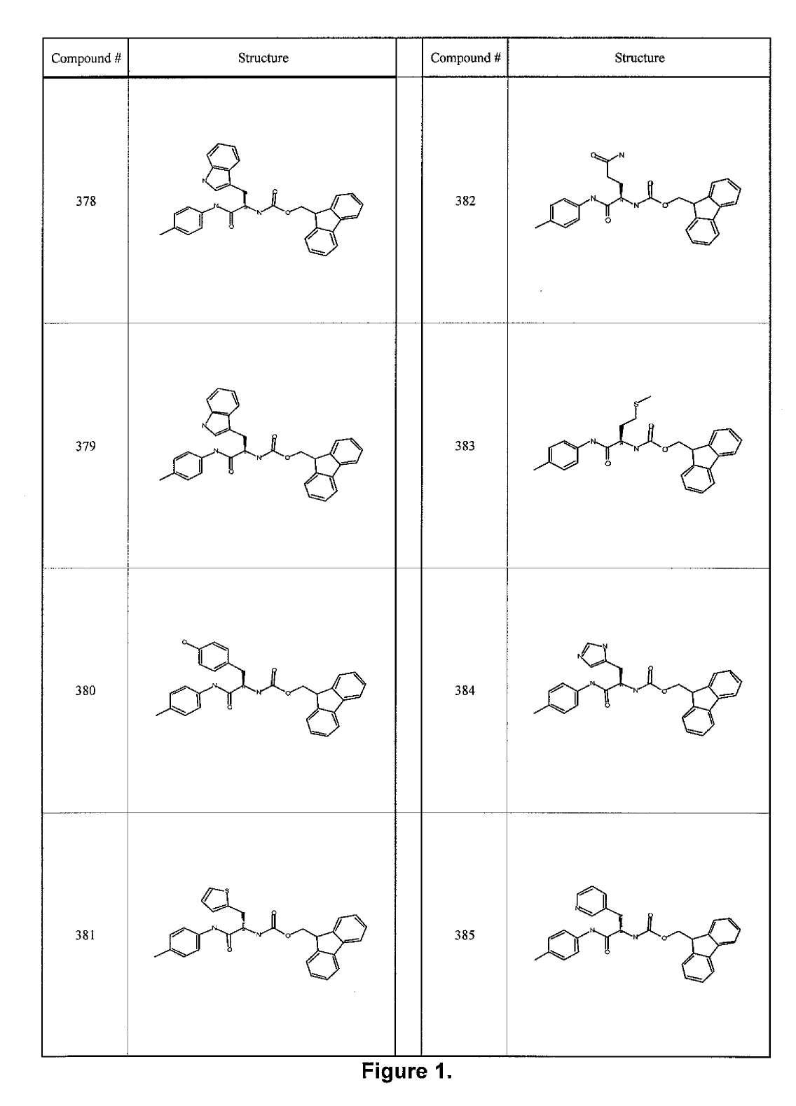

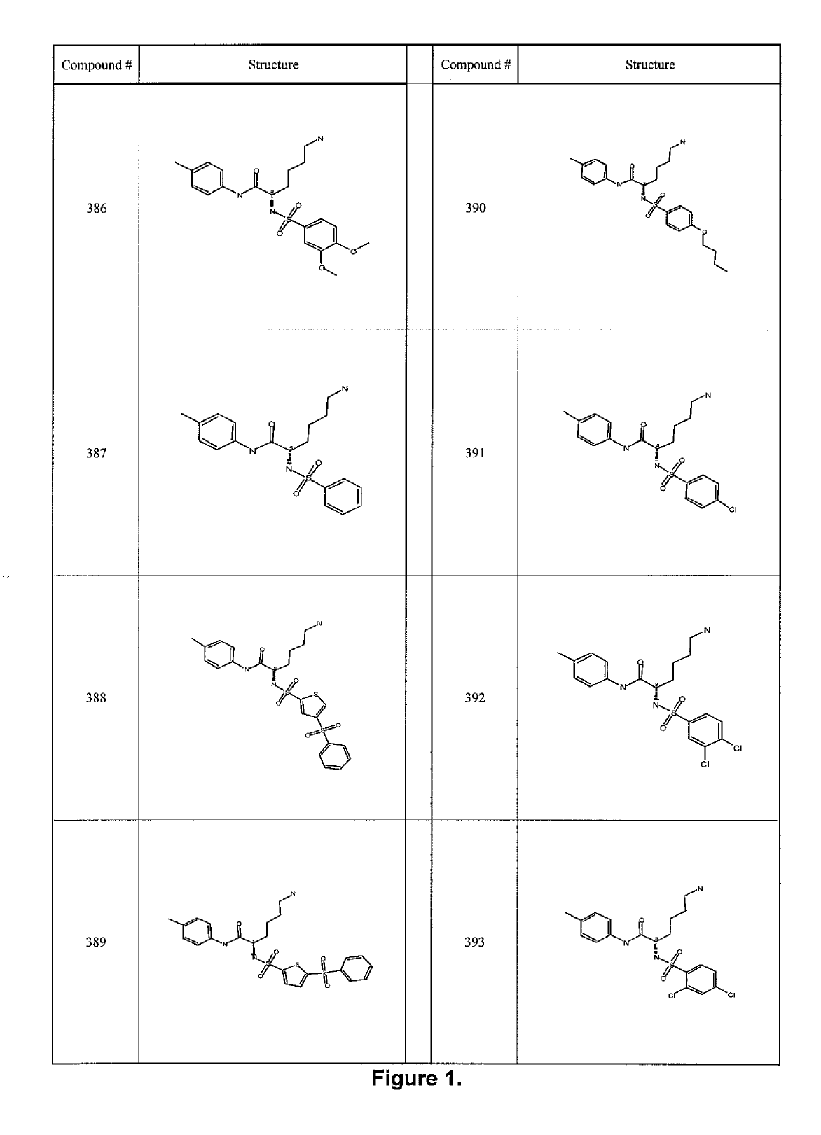

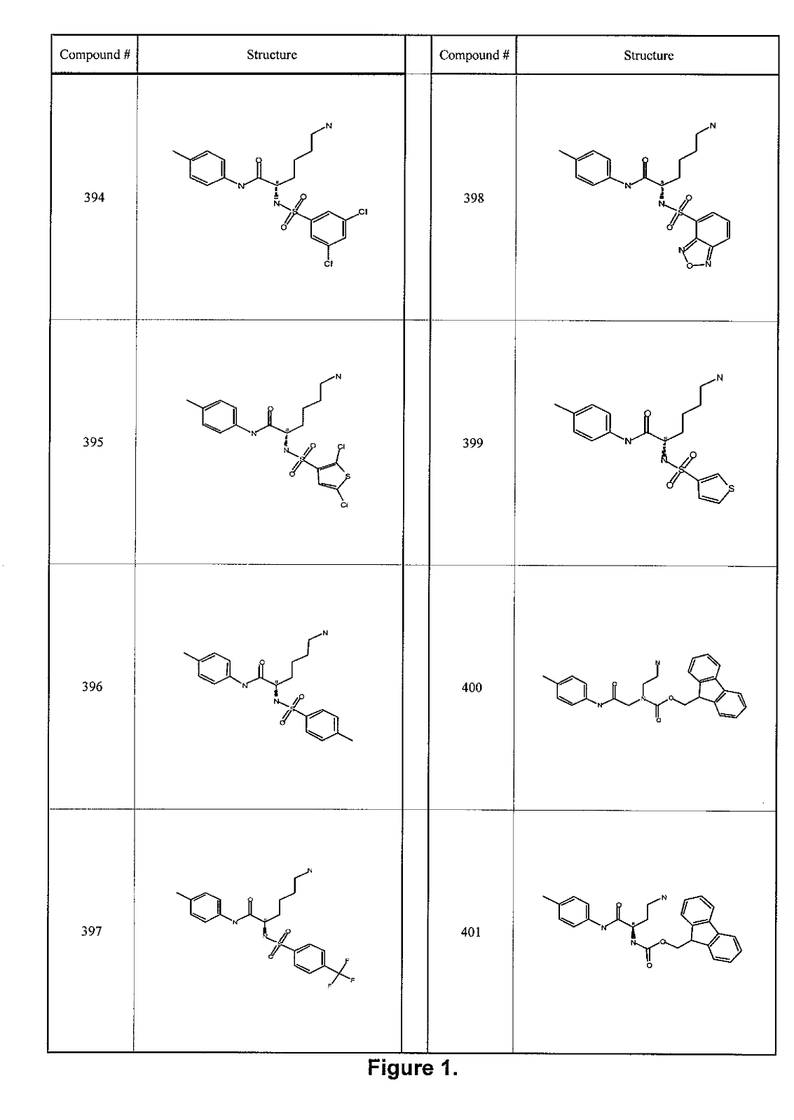

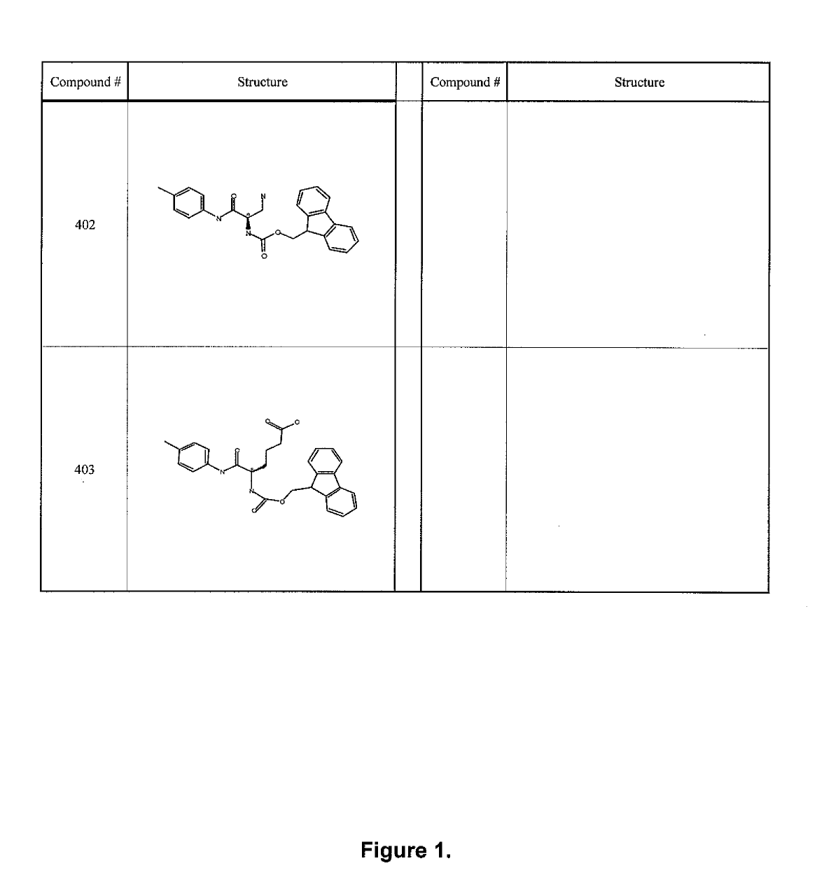

### DETAILED DESCRIPTION

As set forth herein, the present disclosure provides non-nucleoside compounds for use as inhibitors of an endogenous polymerase for treating hyperproliferative diseases or disorders, such as cancer, neoplasms, or autoimmunity, and more specifically, diseases or disorders that are sensitive to inhibition of polymerase alpha or polymerase gamma.

The compounds disclosed herein have an amide-based core structure and an unusually high inhibitory activity against polymerases, such as polymerase alpha or polymerase gamma. In one embodiment of the present disclosure, provided are methods for treating a hyperproliferative disease or disorder using compounds having a structure of formula (I):

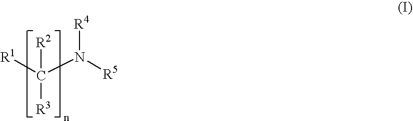

or a stereoisomer, prodrug or pharmaceutically acceptable salt thereof, wherein:

n is 1-5;

R¹ is selected from —C(═O)R⁹, —C(═S)R⁹, —C(═NR¹⁰)R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —S(═O)R⁹, —SO₂R⁹, provided that R⁹ is not H, and that R⁹ does not form an ester with the carbonyl group to which it is bonded when R¹ is —C(═O)R⁹;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆₁C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ of is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;

and wherein at least one, but not more than three, of R², R³, R⁴ and R⁵ is hydrogen, provided that R¹ is not an amino acid when R⁴ and R⁵ are both H. Preferably, not more than two of R², R³, R⁴ and R⁵ are hydrogen.

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” or “consisting essentially of” mean±15% of a range or value. within an acceptable error range for the particular value as determined by one of ordinary skill in the art, which will depend in part on how the value is measured or determined (e.g., the limitations of the measurement system), or the degree of precision required for a particular purpose. For example, “about” can mean within 1, or more than 1 standard deviation, per the practice in the art, or “about” can mean±20% of a given a range or value, unless otherwise indicated, or “about” can mean within an order of magnitude or within 5-fold or within 2-fold of a value (e.g., with respect to a biological system or process). The use of the alternative (e.g., “or”) should be understood to mean either one, both or any combination thereof of the alternatives. As used herein, the indefinite articles “a” and “an” refer to one or to more than one (i.e., at least one) of the grammatical object of the article. By way of example, “a component” means one component or a plurality of components.

“Hyperproliferative disease or disorder,” as used herein, refers to any of a number of diseases that are characterized by abnormal cell division or inappropriate cell division leading to pathogenesis. For example, neoplasia involves rapid abnormal cell division and tissue growth, and can continue after the stimulus, which initiated the abnormal growth, has stopped. Neoplasms have a partial or a complete lack of structural organization and functional coordination with normal tissue, which form a distinct mass of tissue that can either be benign (benign tumor) or malignant (cancer). Malignancies can occur in virtually any tissue (e.g., breast, prostate, pancreas, bone, liver, colon, lung, etc.) and can be localized or spread from its primary site to other places in the body (i.e., metastatic). Benign neoplasms are generally not locally invasive or metastatic, but can lead in certain circumstances to severe disease and even death, due to altered tissue function or tumor growth compressing or damaging adjacent critical structures (e.g., benign brain tumors). Several nonmalignant diseases are characterized by hyperproliferating cells and are amenable to treatment with the compositions and methods of this disclosure. For example, these can include premalignant lesions (e.g., polyps, actinic keratosis, adenomatous hyperplasia, prostatic intraepithelial neoplasia, cervical dysplasia, Bowen's disease, Barrett's syndrome), psoriasis, arthritis, osteoarthritis, rheumatoid arthritis, vascular disease (e.g., atherosclerosis, arteriosclerosis, vascular stenosis, restenosis following angioplasty or stenting, and instent restenosis), hemangiomas, inflammatory bowel disease, leiomyomas, pulmonary fibrosis, benign diseases of the eye, oral hairy leukoplakia, adenomas, lipomas, fibromas, keloids, or the like.

“Alkyl” refers to a saturated or unsaturated, branched, straight-chain or cyclic monovalent hydrocarbon group derived by the removal of one hydrogen atom from a single carbon atom of a parent alkane, alkene or alkyne. Representative alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, and the like.

The term “Alkyl” is specifically intended to include straight- or branched-hydrocarbons having from 1 to 12, or 1 to 8, or 1 to 6, or 1 to 4 carbon atoms. The alkyls may have any degree or level of saturation, such as groups having exclusively single carbon-carbon bonds, groups having one or more double carbon-carbon bonds, groups having one or more triple carbon-carbon bonds and groups having mixtures of single, double and triple carbon-carbon bonds. The expressions “alkanyl,” “alkenyl,” and “alkynyl” are used when a specific level of saturation is intended. The expression “lower alkyl” refers to alkyl groups comprising from 1 to 8 carbon atoms. The alkyl group may be substituted or unsubstituted.

“Alkanyl” refers to a saturated branched, straight-chain or cyclic alkyl group. Representative alkanyl groups include methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, and the like. The alkanyl group may be substituted or unsubstituted.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Representative alkenyl groups include ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, and the like. The alkenyl group may be substituted or unsubstituted.

“Alkynyl” refers to an unsaturated branched, straight-chain or cyclic alkyl group having at least one carbon-carbon triple bond derived by the removal of one hydrogen atom from a single carbon atom of a parent alkyne. Representative alkynyl groups include ethynyl; propynyls such as prop-1-yn-1-yl, prop-2-yn-1-yl, etc.; butynyls such as but-1-yn-1-yl, but-1-yn-3-yl, but-3-yn-1-yl, and the like. The alkynyl group may be substituted or unsubstituted.

“Alkyldiyl” refers to a saturated or unsaturated, branched, straight-chain or cyclic divalent hydrocarbon group derived by the removal of one hydrogen atom from each of two different carbon atoms of a parent alkane, heteroalkane, alkene, heteroalkene, alkyne or heteroalkyne, or by the removal of two hydrogen atoms from a single carbon atom of a parent alkane, heteroalkane, alkene, heteroalkene, alkyne or heteroalkyne. The two monovalent radical centers or each valency of the divalent radical center can form bonds with the same or different atoms. Representative alkyldiyl groups include methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyldiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. The nomenclature of alkanyldiyl, alkenyldiyl and/or alkynyldiyl, as well as heterocompounds thereof, is used when specific levels of saturation are intended. In certain embodiments, the alkyldiyl group is (C₁-C₄) alkyldiyl. In further embodiments, the alkyldiyl group is a saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra). The alkyldiyl group may be substituted or unsubstituted.

“Alkyleno” refers to a straight-chain alkyldiyl group having two terminal monovalent radical centers derived by the removal of one hydrogen atom from each of the two terminal carbon atoms of straight-chain parent alkane, heteroalkane, alkene, heteroalkene, alkyne or heteroalkyne. Representative alkyleno groups include methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc. When specific levels of saturation are intended, the nomenclature alkano, alkeno and/or alkyno is used. In certain embodiments, the alkyleno group is (C₁-C₆) or (C₁-C₄) alkyleno. In further embodiments, the alkyleno group is a straight-chain saturated alkano groups, e.g., methano, ethano, propano, butano, and the like. The alkynyl group may be substituted or unsubstituted.

“Heteroalkyl, Heteroalkanyl, Heteroalkenyl, Heteroalkanyl, Heteroalkyldiyl and Heteroalkyleno” refer to alkyl, alkanyl, alkenyl, alkynyl, alkyldiyl and alkyleno groups, respectively, in which one or more of the carbon atoms (and any associated hydrogen atoms) are each independently replaced with the same or different heteroatoms or heteroatomic groups. Representative heteroatoms or heteroatomic groups include —O—, —S—, —Se—, —O—O—, —S—S—, —O—S—, —O—S—O—, —O—NR⁶—, —NR⁶—, —NR⁶—NR⁶—, ═N—N═, —N═N—, —N═N—NR⁶—, —PH—, —P(O)₂—, —O—P(O)₂—, —SH₂—, —S(O)₂—, —SnH₂— and the like, and combinations thereof, such as —NR⁶—S(O)₂—; wherein each R⁶ is independently selected from the group consisting of hydrogen, alkyl, alkanyl, alkenyl, alkynyl, aryl, arylalkyl, heteroaryl and heteroarylalkyl, as described herein.

“Aryl” refers to a monovalent aromatic hydrocarbon group derived by the removal of one hydrogen atom from a single carbon atom of a parent aromatic ring system. Representative aryl groups include groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, the aryl group is (C₅-C₁₈) or (C₅-C₁₂) aryl. Other representative aryls are cyclopentadienyl, phenyl, biphenyl, and naphthyl. The aryl group may be substituted or unsubstituted.

“Arylalkyl” refers to an acyclic alkyl group in which one of the hydrogen atoms bonded to a carbon atom, such as a terminal or sp³ carbon atom, is replaced with an aryl group. Representative arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The nomenclature of arylalkanyl, arylalkenyl and/or arylalkynyl is used when specific alkyl moieties are intended. In certain embodiments, the arylalkyl group is (C₆-C₂₀) arylalkyl, e.g., the alkanyl, alkenyl or alkynyl moiety of the arylalkyl group is (C₁-C₆) or (C₂-C₆), and the aryl moiety is (C₅-C₁₄). In further embodiments, the arylalkyl group is (C₆-C₁₄), e.g., the alkanyl, alkenyl or alkynyl moiety of the arylalkyl group is (C₁-C₄) or (C₂-C₄), and the aryl moiety is (C₅-C₁₀). The arylalkyl group may be substituted or unsubstituted.

“Heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). Representative heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group is a 5-14 membered or a 5-10 membered heteroaryl. In further embodiments, heteroaryl groups are those derived from thiophene, pyrrole, furan, benzothiophene, benzofuran, indole, pyridine, pyrimidine, quinoline, imidazole, oxazole and pyrazine. The heteroaryl group may be substituted or unsubstituted.

“Heteroalicyclic” refers to a monocyclic or fused ring group having in the ring(s) one or more atoms selected from, for example, nitrogen, oxygen and sulfur. The rings may also have one or more double bonds. However, the rings do not necessarily have a completely conjugated π-electron system. The heteroalicyclic ring may be substituted or unsubstituted. When substituted, the substituted group(s) may be selected independently from alkyl, aryl, haloalkyl, halo, hydroxy, alkoxy, mercapto, cyano, sulfonamidyl, aminosulfonyl, acyl, acyloxy, nitro, and substituted amino.

“Heteroarylalkyl” refers to an acyclic alkyl group (including heteroalkyl groups, substituted or not substituted) in which one of the hydrogen atoms bonded to a carbon atom, such as a terminal or sp³ carbon atom, is replaced with an aryl or a heteroaryl group. The “heteroarylalkyl” can encompass any combination of “aryl”, “heteroaryl,” “alkyl” and “heteroalkyl,” such as heteroarylalkyl, heteroalkylaryl, heteroarylheteroalkyl, and the like. A “heteroarylalkyl” can be substituted or not substituted. The nomenclature heteroarylalkanyl, heteroarylakenyl and/or heterorylalkynyl are used when specific alkyl moieties are intended. In certain embodiments, the heteroarylalkyl group is a 5-20 membered heteroarylalkyl, e.g., the alkanyl, alkenyl or alkynyl moiety of the heteroarylalkyl is 1-6 membered and the heteroaryl moiety is a 5-14-membered heteroaryl. In further embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

The various heteroaryls, such as indole-, naphthalene-, pyridine-, thiophene- and furan-groups, can include the various position isomers when in the form of a heteroarylalkyl. For example, if the alkyl includes a carbonyl group, the heteroarylalkyls can be indole-3-carbonyl, indole-5-carbonyl, naphthalene-1-carbonyl, naphthalene-2-carbonyl, nicotinoyl, isonicotinoyl, N-methyl-dihydro-pyridine-3-carbonyl, thiophene-2-carbonyl, thiophene-3-carbonyl, furan-2-carbonyl and furan-3-carbonyl. The indole, naphthalene, pyridine, thiophene and furan groups can be optionally further substituted, as indicated herein.

“Acyl” group refers to the C(═O)—R″ group, where R″ is selected preferably from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heteroalicyclic (bonded through a ring carbon) optionally substituted with one or more alkyl, haloalkyl, alkoxy, halo and substituted amino groups. Acyl groups include aldehydes, ketones, acids, acid halides, esters and amides. Preferred acyl groups are carboxy groups, e.g., acids and esters. Esters include amino acid ester derivatives. The acyl group may be attached to a compound's backbone at either end of the acyl group, i.e., via the C or the R″. Where the acyl group is attached via the R″, then C will bear another substituent, such as hydrogen, alkyl, and the like.

“Halogen” or “halo” refers to fluoro (F), chloro (Cl), bromo (Br), iodo (I). As used herein, —X refers to independently any halogen.

Sulphur (S) atom may be present in several compounds of this disclosure, and when present, the S atom can be at any oxidation state (e.g., S, SO, SO₂).

As used herein, “ionizable nitrogen” refers to a nitrogen containing substituent wherein the nitrogen is capable of taking on a positive charge within a pH range of about 4 to about 9.

As used herein, “amino acid” refers to a natural (those occurring in nature) amino acid, a substituted natural amino acid, a non-natural amino acid, a substituted non-natural amino acid, or any combination thereof. The designations for natural amino acids are herein set forth as either the standard one- or three-letter code. Natural polar amino acids include asparagine (Asp or N) and glutamine (Gln or Q); as well as basic amino acids such as arginine (Arg or R), lysine (Lys or K), histidine (H is or H), and derivatives thereof; and acidic amino acids such as aspartic acid (Asp or D) and glutamic acid (Glu or E), and derivatives thereof. Natural hydrophobic amino acids include tryptophan (Trp or W), phenylalanine (Phe or F), isoleucine (Ile or I), leucine (Leu or L), methionine (Met or M), valine (Val or V), and derivatives thereof; as well as other non-polar amino acids such as glycine (Gly or G), alanine (Ala or A), proline (Pro or P), and derivatives thereof. Natural amino acids of intermediate polarity include serine (Ser or S), threonine (Thr or T), tyrosine (Tyr or Y), cysteine (Cys or C), and derivatives thereof. Unless specified otherwise, any amino acid described herein may be in either the D- or L-configuration. A capital letter indicates an L-enantiomer amino acid; a small letter indicates a D-enantiomer amino acid.

Other exemplary amino acids include cinnamic acids (such as aminocinnamic acids, amino-trans-cinnamic acids, amino-cis-cinnamic acids, o-amino-cinnamic acids, m-amino-cinnamic acids, p-amino-cinnamic acids, o-amino-trans-cinnamic acid, m-amino-trans-cinnamic acid, p-amino-trans-cinnamic acid, o-amino-cis-cinnamic acid, m-amino-cis-cinnamic acid, p-amino-cis-cinnamic acid), phenylglycine (Phg), 2,3-diaminobutyric acid (Dab), 2,4-diaminobutyric acid (gDab), 2,3-diaminopropionic acid (Dap), β-methylaspartate (MeAsp), cyclohexylalanine (β-Cha), norleucine (Nle), norvaline (Nvl), isonipecotic acid (Ina), pipecolic acid (homoproline) (Pip or hPro), phenylacetic acids (such as aminophenylacetic acids, diaminophenylacetic acids, triaminophenylacetic acids, o-amino-phenylacetic acid, m-amino-phenylacetic acid, p-amino-phenylacetic acid (Apa), o,o-diamino-phenylacetic acid, o,m-diamino-phenylacetic acid, o,p-diamino-phenylacetic acid, m,m-diamino-phenylacetic acid, m,p-diamino-phenylacetic acid, o,o,m-triamino-phenylacetic acid, o,o,p-triamino-phenylacetic acid, o,m,p-triamino-phenylacetic acid, m,m,p-triamino-phenylacetic acid, o,m,m-triamino-phenylacetic acid, o,o,m-triamino-phenylacetic acid), phenylpropanoic acids (such as aminophenylpropanoic acids, diaminophenylpropanoic acids, triaminophenylpropanoic acids, o-amino-phenylpropanoic acid, m-amino-phenylpropanoic acid, p-amino-phenylpropanoic acid, o,o-diamino-phenylpropanoic acid, o,m-diamino-phenylpropanoic acid, o,p-diamino-phenylpropanoic acid, m,m-diamino-phenylpropanoic acid, m,p-diamino-phenylpropanoic acid, o,o,m-triamino-phenylpropanoic acid, o,o,p-triamino-phenylpropanoic acid, o,m,p-triamino-phenylpropanoic acid, m,m,p-triamino-phenylpropanoic acid, o,m,m-triamino-phenylpropanoic acid, o,o,m-triamino-phenylpropanoic acid), 2-aminobutyric acid (Abu), sarcosine (Sar or N-methyl glycine), 6-aminohexanoic acid (Ahx), para-fluoro-Phenylalanine (p-F-Phe), γ-amino-butyric acid (GABA), benzoic acids (such as aminobenzoic acids, diaminobenzoic acids, triaminobenzoic acids, o-amino-benzoic acid, m-amino-benzoic acid, p-aminobenzoic acid (PABA), o,o-diamino-benzoic acid, o,m-diamino-benzoic acid, o,p-diamino-benzoic acid, m,m-diamino-benzoic acid, m,p-diamino-benzoic acid, o,o,m-triamino-benzoic acid, o,o,p-triamino-benzoic acid, o,m,p-triamino-benzoic acid, m,m,p-triamino-benzoic acid, o,m,m-triamino-benzoic acid, o,o,m-triamino-benzoic acid), hydrazinobenzoic acids (such as dihydrazinobenzoic acids, trihydrazinobenzoic acids, o-hydrazino-benzoic acid, m-hydrazino-benzoic acid, p-hydrazino-benzoic acid, o,o-dihydrazino-benzoic acid, o,m-dihydrazino-benzoic acid, o,p-dihydrazino-benzoic acid, m,m-dihydrazino-benzoic acid, m,p-dihydrazino-benzoic acid, o,o,m-trihydrazino-benzoic acid, o,o,p-trihydrazino-benzoic acid, o,m,p-trihydrazino-benzoic acid, m,m,p-trihydrazino-benzoic acid, o,m,m-trihydrazino-benzoic acid, o,o,m-trihydrazino-benzoic acid), homophenylalanine (homoPhe or hPhe), β-cyanoAlanine (β-cyano-Ala), methyl or ethyl aryl ethers of tyrosine (Tyr(Me) or Tyr(Et), respectively), aminoisobutyric acid (Aib, which is also known as α,α-dimethylglycine), S-methylcysteine (MeCys), N,N′-dimethyl-arginine ((Me)₂Arg), hydroxyProline (Hyp), citruline (Cit), N,N,N-trimethyllysine or N,N,N,—(CH₃)₃-lysine or γ,γ,γ-trimethyllysine ((Me)₃Lys), homolysine (homoLys or hLys), 5-aminopentanoic acid or aminovaleric acid (5-Ava), (S)-3-Benzo[b]thiophen-3-yl-aminopropanoic acid (L-BBTA), pyroglutamic acid (pGlu), aminothiazole acetic acids, 2-amino-thiazol-4-yl acetic acid, aminoheptanoic acids, aminooctanoic acids, aminononanoic acids, aminodecanoic acids, aminoundecanoic acids, aminododecanoic acids, 7-aminoheptanoic acid, 8-aminooctanoic acid, 9-aminononanoic acid, 10-aminodecanoic acid, 1-aminoundecanoic acid, 12-aminododecanoic acid, 3- or 4-mercaptoproline derivatives, N⁵-acetyl-N⁵-hydroxy-L-ornithine, α-N-hydroxyamino acids, and the like. An compound disclosed herein may include any one or a combination of the above-noted amino acids or any one or a combination of the above-noted amino acids optionally substituted.

“Substituted” refers to a group in which one or more hydrogen atoms are each independently replaced with the same or different substituent(s). Representative substituents include —X, —R⁶, —O—, ═O, —OR, —SR⁶, —S—, ═S, —NR⁶R⁶, ═NR⁶, CX₃, —CF₃, —CN, —OCN, —SCN, —NO, —NO₂, ═N₂, —N₃, —S(═O)₂O—, —S(═O)₂OH, —S(═O)₂R⁶, —OS(═O)₂O—, —OS(═O)₂OH, —OS(═O)₂R⁶, —P(═O)(O⁻)₂, —P(═O)(OH)(O⁻), —OP(═O)₂(O⁻), —C(—O)R⁶, —C(═S)R⁶, —C(═O)OR⁶, —C(═O)O⁻, —C(═S)OR⁶, —NR⁶—C(═O)—N(R⁶)₂, —NR⁶—C(═S)—N(R⁶)₂, and —C(═NR⁶)NR⁶R⁶, wherein each X is independently a halogen; and each R⁶ is independently hydrogen, halogen, alkyl, aryl, arylalkyl, arylaryl, arylheteroalkyl, heteroaryl, heteroarylalkyl, NR⁷R⁷, —C(—O)R⁷, and —S(═O)₂R⁷; and each R⁷ is independently hydrogen, alkyl, alkanyl, alkynyl, aryl, arylalkyl, arylheteralkyl, arylaryl, heteroaryl or heteroarylalkyl. Aryl containing substituents, whether or not having one or more substitutions, may be attached in a para (p-), meta (m-) or ortho (o-) conformation, or any combination thereof.

The term “independently” means that a substituent can be the same or different for each item described.

In addition, it should be understood that individual compounds or groups of compounds derived from the various combinations of the structures and substituents described herein are provided by the present disclosure to the same extent as if each compound or group of compounds was set forth individually. Thus, selection of particular structures or particular substituents is within the scope of the present disclosure.

In one embodiment of formula (I), a carbonyl group can be a bridge between R¹ and the core structure—these compounds can be used for treating hyperproliferative diseases or disorders and have a structure of formula (II):

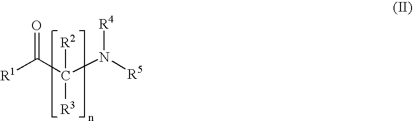

or a stereoisomer, prodrug or pharmaceutically acceptable salt thereof, wherein:

n is 1-5;

R¹ is selected from (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups, provided that R¹ does not form an ester with the carbonyl group to which it is bonded;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In another embodiment of formula (I), R¹ is bonded to the core structure via a —NR¹⁰C(═O). These compounds can be used for treating hyperproliferative diseases or disorders and have a structure of formula (III):

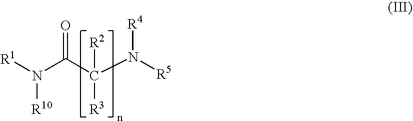

or a stereoisomer, prodrug or pharmaceutically acceptable salt thereof, wherein:

n is 1-5;

R¹ is selected from (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R², R³, R⁴, and R⁹ are (i) independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ and R⁹ are as defined above;

or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R⁹ are as defined above;

or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R², R³ and R⁹ are as defined above;

and wherein at least one, but not more than three, of R², R³, R⁴ and R⁵ is hydrogen, provided that R¹ is not an amino acid when R⁴ and R⁵ are both H. Preferably, not more than two of R², R³, R⁴ and R⁵ is hydrogen

In some embodiments, R² is H and R⁴ is H.

In some embodiments, R² is H, R⁴ is H, and n is 1.

In some embodiments, R² is H, R⁴ is H, n is 1, and R⁵ is selected from —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, and —SO₂R⁹.

In some embodiments, n is 1 and R⁵ is selected from —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, and —SO₂R⁹. In some of these embodiments, the R⁹ substituent of R⁵ is selected from alkyl substituted with one or more of the same or different substituted or unsubstituted aryl groups, (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, the R⁹ substituent of R⁵ is selected from —(CH₂)ₘ—R¹¹ and —R¹¹ where m is an integer in the range from 1 to 4 and R¹¹ is selected from (C₅-C₁₈) aryl optionally substituted with one or more of the same or different R¹⁰ groups, (C₆-C₂₀), arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, R¹¹ is selected from (C₅-C₁₈) aryl optionally substituted with one or more of the same or different R¹⁰ groups. In some of these embodiments, R¹¹ is selected from the following:

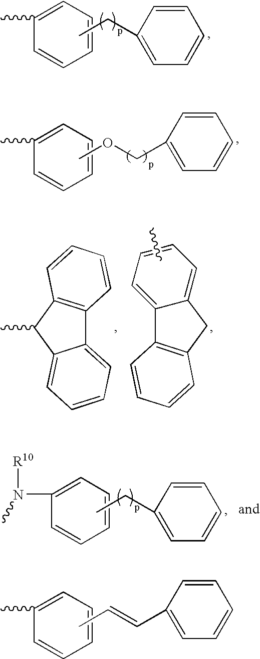

where p is an integer in the range of 0 to 4 and where each phenyl group is optionally substituted with 1 to 3 of the same or different R¹⁰ groups. It will be understood that a bond extending from within a phenyl ring indicates that the attachment of that substituent can be at any open site on the phenyl ring (e.g., ortho, meta, or para to another substituent.) In some of these embodiments, R⁹ is —R¹¹ and in other embodiments R⁹ is —CH₂R¹¹. In some of these embodiments, p is 0 and in other embodiments p is 1. In some of the embodiments, none of the phenyl groups or R¹¹ are substituted by additional R¹⁰ groups.

In some embodiments, R² is H, R⁴ is H, n is 1, and R⁵ is selected from —C(═O)R⁹, —CO₂R⁹, —C(═O)NR¹⁰R⁹, and —C(═O)(NR¹⁰)SO₂R⁹. In some of these embodiments, the R⁹ substituent of R⁵ is selected from alkyl substituted with one or more of the same or different substituted or unsubstituted aryl groups, (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, the R⁹ substituent of R⁵ is selected from —(CH₂)ₘ—R¹¹ and —R¹¹ where m is an integer in the range from 1 to 4 and R¹¹ is selected from (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, R¹¹ is selected from (C₅-C₁₈) aryl optionally substituted with one or more of the same or different R¹⁰ groups. In some of these embodiments, R¹¹ is selected from the following:

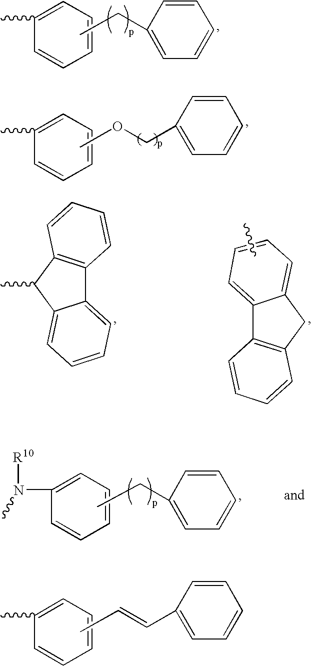

where p is an integer in the range of 0 to 4 and where each phenyl group is optionally substituted with 1 to 3 of the same or different R¹⁰ groups. In some of these embodiments, R⁹ is —R¹¹ and in other embodiments R⁹ is —CH₂R¹¹. In some of these embodiments, p is 0 and in other embodiments p is 1. In some of the embodiments, none of the phenyl groups or R¹¹ are substituted by additional R¹⁰ groups.

In some of the preceding embodiments, R³ is —(CR⁹R¹⁰)ᵣNR⁹R¹⁰ where r is an integer in the range of 1 to 10 and each R⁹ and R¹⁰ is independent and as defined above. In some of these embodiments, R² is H. In some of these embodiments, R³ is —(CR⁹R¹⁰)ᵣNH₂, —(CH₂)ᵣNR⁹R¹⁰, or —(CH₂)ᵣNH₂. In some of these embodiments, r is an integer in the range of 2 to 5. In some of these embodiments, r is 3 or 4.

In some of the preceding embodiments, R¹ is (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, the R¹⁰ substituent attached to the same nitrogen as R¹ is H. In some of these embodiments, R¹ is (C₅-C₁₈) aryl optionally substituted with one or more of the same or different R¹⁰ groups.

Each of the preceding embodiments, with corresponding selection of substituents for one or more of R¹, R², R³, R⁴, R⁵, R⁹, R¹⁰, and R¹¹, can be applied to compounds for each of the formulas (I) to (X) where such selection of each particular substituent is not already provided in the description of those compounds.

In certain aspects, the instant disclosure provides compounds that can be used for treating hyperproliferative diseases or disorders and that have a structure of formula (IV):

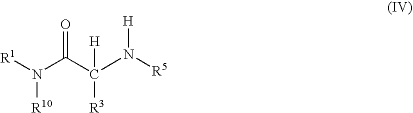

or a stereoisomer, prodrug or pharmaceutically acceptable salt thereof, wherein:

R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

R⁵ is selected from —C(═O)NR¹⁰R⁹, —C(═O)(NR¹⁰)SO₂R⁹, —C(═S)NR¹⁰R⁹, or —C(═NR¹⁰)NR¹⁰R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl. Preferably, R¹ and R³ are not hydrogen.

In some of these embodiments, the R⁹ substituent of R⁵ is selected from alkyl substituted with one or more of the same or different substituted or unsubstituted aryl groups, (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, the R⁹ substituent of R⁵ is selected from —(CH₂)ₘ—R¹¹ and —R¹¹ where m is an integer in the range from 1 to 4 and R¹¹ is selected from (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, R¹¹ is selected from (C₅-C₁₈) aryl optionally substituted with one or more of the same or different R¹⁰ groups. In some of these embodiments, R¹¹ is selected from the following:

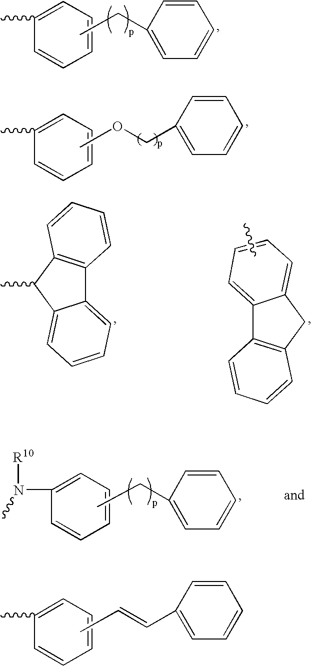

where p is an integer in the range of 0 to 4 and where each phenyl group is optionally substituted with 1 to 3 of the same or different R¹⁰ groups. In some of these embodiments, R⁹ is —R¹¹ and in other embodiments R⁹ is —CH₂R¹¹. In some of these embodiments, p is 0 and in other embodiments p is 1. In some of the embodiments, none of the phenyl groups or R¹¹ are substituted by additional R¹⁰ groups.

In some of the preceding embodiments, R³ is —(CR⁹R¹⁰)ᵣNR⁹R¹⁰ where r is an integer in the range of 1 to 10 and each R⁹ and R¹⁰ is independent and as defined above. In some of these embodiments, R² is H. In some of these embodiments, R³ is —(CR⁹R¹⁰)ᵣNH₂, —(CH₂)ᵣNR⁹R¹⁰, or —(CH₂)ᵣNH₂. In some of these embodiments, r is an integer in the range of 2 to 5. In some of these embodiments, r is 3 or 4.

In some of the preceding embodiments, R¹ is (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups. In some of these embodiments, the R¹⁰ substituent attached to the same nitrogen as R¹ is H. In some of these embodiments, R¹ is (C₅-C₁₈) aryl optionally substituted with one or more of the same or different R¹⁰ groups.

In a further embodiment, provided is a compound having a structure of formula (IV) as defined herein, wherein R³ is not hydrogen, or wherein R³ has an ionizable nitrogen.

In a further aspect, the instant disclosure provides compounds that can be used for treating hyperproliferative diseases or disorders and that have a structure of formula (V):

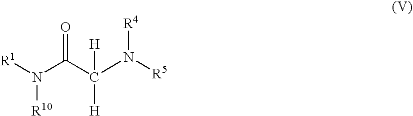

or a stereoisomer, prodrug or pharmaceutically acceptable salt thereof, wherein:

R¹, R⁴ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ and R⁴ are not hydrogen;

R⁵ is selected from —C(═O)NR¹⁰R⁹, —C(═O)(NR¹⁰)SO₂R⁹, —C(═S)NR¹⁰R⁹, or —C(═NR¹⁰)NR¹⁰R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

In a further embodiment, provided is a compound having a structure of formula (V) as defined herein, wherein R⁴ has an ionizable nitrogen

In a further aspect, provided are compounds that can be used for treating hyperproliferative diseases or disorders and that have a structure of formula (VI):

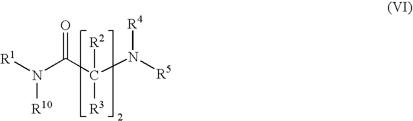

or a stereoisomer, prodrug or pharmaceutically acceptable salt thereof, wherein:

R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

(i) R², R³ and R⁴ are each independently the same or different substituent as defined for R⁹; or (ii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In a further embodiment, provided is a compound that can be used for treating hyperproliferative diseases or disorders and that have a structure of formula (VI) as defined herein, wherein the compounds are further defined by a structure of formula (VII):

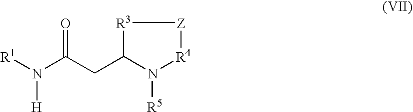

wherein R³ and R⁴ are each independently selected from —CH₂— or —(CH₂)₂—; Z is —N(R⁹)—; and R¹, R⁵, R⁹, and R¹⁰ are as defined herein for structure (VI). In one embodiment, there is provided a compound of structure (VII), wherein the R⁹ has an ionizable nitrogen. In another embodiment, there is provided a compound of structure (VII), wherein the R⁹ has an ionizable nitrogen, R³ is —CH₂— and R⁴ is —(CH₂)₂

In a further aspect, the instant disclosure provides compounds that can be used for treating hyperproliferative diseases or disorders and that have a structure of formula (VIII):

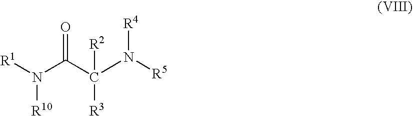

or a stereoisomer, prodrug or pharmaceutically acceptable salt thereof, wherein:

R¹ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₆-C₂₀) heteroarylalkenyl optionally substituted with one or more of the same or different R¹⁰ groups; provided that R¹ is not hydrogen;

(i) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (ii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iii) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, or —SO₂R⁹; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In a further embodiment, the instant disclosure provides a compound of structure (VIII) as defined herein, wherein at least one of R², R³ or R⁴ has an ionizable nitrogen. In another embodiment, provided is a compound of structure (VIII) as defined herein, wherein R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent. In still another embodiment, provided is a compound of structure (VIII) as defined herein, wherein R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent.

In a further aspect, the instant disclosure provides compounds that can be used for treating hyperproliferative diseases or disorders and that have a structure of formula (IX):

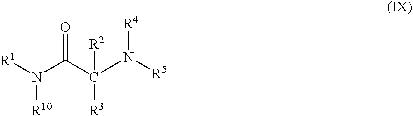

or a stereoisomer, prodrug or pharmaceutically acceptable salt thereof, wherein:

R¹ is the same as R⁹ provided an ionizable nitrogen is present;

(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;

R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;

R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;

and wherein at least one but not more than three of R², R³, R⁴ and R⁵ is hydrogen, provided that R¹ is not an amino acid when R⁴ and R⁵ are both H.

In a further aspect, the instant disclosure provides compounds that can be used for treating hyperproliferative diseases or disorders and that have a structure of formula (X):

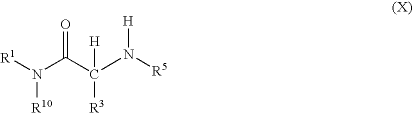

or a stereoisomer, prodrug or pharmaceutically acceptable salt thereof, wherein:

R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ is not hydrogen;

R⁵ is selected from H, —C(═O)R⁹, —C(═S)R⁹, —C(═NR¹⁰)R⁹, or —CO₂R⁹;

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl; and

R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

In a further embodiment, the instant disclosure provides a compound having a structure of formula (X) as defined herein, wherein R³ is not hydrogen or wherein R³ has an ionizable nitrogen.

The compounds for use in treating hyperproliferative diseases or disorders of the instant disclosure can be utilized as a free acid, free base, or in the form of acid or base addition salts (e.g., pharmaceutically acceptable salts). “Pharmaceutically acceptable salt” refers to a salt of a compound of this disclosure that is pharmaceutically acceptable and that possesses the desired pharmacological activity of the parent compound. Such salts may include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

In another aspect, the compounds for use in treating hyperproliferative diseases or disorders of the instant disclosure may be in the form of a prodrug. “Prodrug” as used herein refers to a compound that can be converted into the parent compound in vivo. Prodrugs often are useful because, in some situations, they may be easier to administer than the parent compound. For example, the prodrug may be more bioavailable by oral administration or for cellular uptake than a parent compound. The prodrug may also have improved solubility in pharmaceutical compositions over the parent compound or an extended half-life in vivo. An example of a prodrug can be a compound as described herein that is administered as an ester (a “prodrug”) to, for example, facilitate transmittal across a cell membrane (when water solubility is detrimental to mobility across such as membrane). Once in the cell, the prodrug may then be metabolically hydrolyzed to a more water soluble form where water solubility is beneficial. Alternatively, a prodrug compound may be converted into its metabolite before entry into a cell. Other representative examples of prodrugs include acetate, formate, and benzoate derivatives of alcohol and amine functional groups that would be converted into hydroxy or amine groups. In certain embodiments, such a prodrug compound may be inactive (or less active) until converted into a metabolite (i.e., parent compound or derivative thereof). In other embodiments, a prodrug compound may be remain active (or have substantially similar activity to the parent compound) before being converted into a metabolite.

“Structurally pure” refers to a compound composition in which a substantial percentage, e.g., on the order of 95% to 100% and preferably ranging from about 95%, 96%, 97%, 98%, 99% or more, of the individual molecules comprising the composition each contain the same number and types of atoms attached to each other in the same order and with the same bonds. As used herein, “structurally pure” is not intended to distinguish different geometric isomers or different optical isomers from one another. For example, as used herein a mixture of cis- and trans-but-2,3-ene is considered structurally pure, as is a racemic mixture. When compositions are intended to include a substantial percentage of a single geometric isomer and/or optical isomer, the nomenclature “geometrically pure” and “optically or enantiomerically pure,” respectively, are used.

The phrase “structurally pure” is also not intended to discriminate between different tautomeric forms or ionization states of a molecule, or other forms of a molecule that result as a consequence of equilibrium phenomena or other reversible interconversions. Thus, a composition of, for example, an organic acid is structurally pure even though some of the carboxyl groups may be in a protonated state (—CO₂H) and others may be in a deprotonated state (—CO₂⁻). Likewise, a composition comprising a mixture of keto and enol tantomers, unless specifically noted otherwise, is considered structurally pure.

The compounds of this disclosure for use in treating hyperproliferative diseases or disorders may contain a chiral center on any of the substituents and these can exist in the form of two optical isomers (the (+) and (−) isomers, also referred to as the (R) and (S) isomers). All such enantiomers and mixtures thereof, including racemic mixtures, are included within the scope of this disclosure. A single optical isomer (or enantiomer) can be obtained by methods known in the art, such as by chiral HPLC or other chiral chromatography, enzymatic resolutions, use of chiral auxiliaries, selective crystallization, or any combination thereof. In certain embodiments, some of the crystalline forms of the compounds of this disclosure may exist as polymorphs, which are included within the scope of this disclosure. In further embodiments, some of the compounds of this disclosure may form solvates with solvents (e.g., water, organic solvents), which are included within the scope of this disclosure.

In certain embodiments, the present disclosure provides compounds for use in treating hyperproliferative diseases or disorders in the form of a single enantiomer that is at least 90%, 95%, 97% or at least 99% free of a corresponding enantiomer. In one embodiment, the single enantiomer is in the (+) form and is at least 90%, at least 95%, at least 97% or at least 99% free of a corresponding (−) enantiomer. In another embodiment, the single enantiomer is in the (−) form and is at least 90%, at least 95%, at least 97% or at least 99%, free of a corresponding (+) enantiomer.

### Methods of Synthesis

The compounds of this disclosure may be synthesized via several different synthetic routes using commercially available starting materials or starting materials prepared by conventional synthetic or biosynthetic methods. For example, the synthesis may be carried out in solution or in solid phase. An exemplary synthetic approach in solution is illustrated in Scheme (I) (see Examples 2-325), as follows:

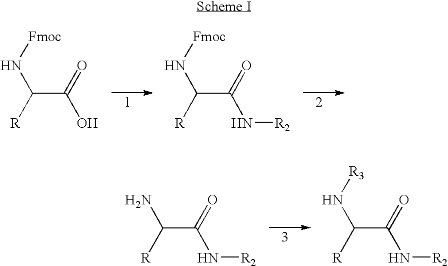

Prior to using Scheme I, a compound of interest is coupled with an appropriate protective group, such as t-Boc or Fmoc, generate a protected reactant. While Scheme I illustrates the use of an Fmoc protecting group, a person having ordinary skill in the art will recognize that other protecting groups may be employed. In addition, in some instances, a parent compound may include other or additional functionalities that may require protection. Groups suitable for protecting a wide variety of different functionalities, as well as conditions for their removal, are well known and will be apparent to those of ordinary skill in the art. Specific guidance for selectively protecting a wide variety of functionalities may be found, for example, in Greene & Wuts, Protective Groups in Organic Synthesis, 3^{rd} edition, 1999 (“Greene & Wuts”). Preferred protecting groups are those that may be easily removed. Exemplary groups for protecting primary amines are tert-butyloxycarbonyl (“t-Boc”), 9-fluorenylmethoxycarbonyl (“Fmoc”) benzyloxycarbonyl (“Z”), and allyloxycarbonyl (Alloc).

In a first step of Scheme I, a protected reactant is subjected to an amidation reaction, or other nucleophilic substitution, to provide a protected intermediate (or mixture of intermediates) in reasonable crude yields. Purification of this protected intermediate by chromatography is optional. Reaction conditions for coupling amines with carboxylic acids to yield amide linkages are known to those of ordinary skill in the art and may be found in any compendium of standard synthetic methods or literature related to the synthesis of peptides and proteins. See e.g., March, J., Advanced Organic Chemistry; Reactions, Mechanisms and Structure, 4^{th} ed., 1992; Larock, Comprehensive Organic Transformations, VCH, New York, 1999; Bodanzsky, Principles of Peptide Synthesis, Springer Verlag, 1984; Bodanzsky, Practice of Peptide Synthesis, Springer Verlag, 1984; Lloyd-Williams et al., Chemical Approaches to the Synthesis of Peptides and Proteins, CRC Press, 1997 (see especially pp. 105-114); and Atherton & Sheppard, Solid Phase Peptide Synthesis: A Practical Approach, IRL Press, 1989). Alternative reactive groups can be utilized, such as isocyanate (which would yield a urea) and others exemplified herein, in methods known in the art. In a second step, deprotection provides a free amine use in a third step of coupling with an appropriate electrophile, such as an isocyanate or acyl chloride, to provide a crude final product.

The derivative compounds of this disclosure may be isolated and purified using standard techniques, such as high-pressure liquid chromatography (HPLC), fast protein liquid chromatography (FPLC), counter current extraction, centrifugation, filtration, precipitation, ion exchange chromatography, gel electrophoresis, affinity chromatography, flash chromatography, and the like. Specific methods of isolation are provided in the Examples section below. Standard characterization and purity analysis known in the art can be used to verify final products and intermediates.

An exemplary solid-phase synthetic approach is illustrated in Scheme (II) (see Examples 326-403), as follows:

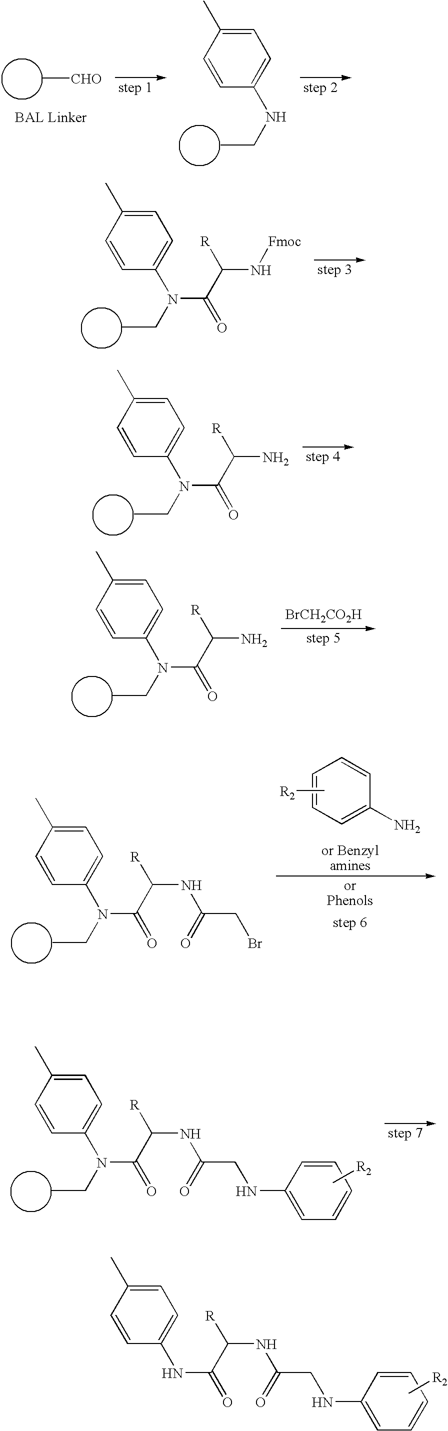

### Step 1. Reductive Amination

Used in these procedures were SynPhase™ polystyrene D-series Lanterns derivatized with backbone amide linker (BAL) with a nominal loading capacity if 35 μmol available from Mimotopes (code MIL1018). The working volume of solvent per Lantern was 0.7 mL.

A stock solution was prepared of sodium cyanoborohydride (0.1 M) in acetic acid/dimethylformamide (DMF) (1:99). The appropriate amines (1.0 M) were dispensed into glass vials or Schott bottles, to which the sodium cyanoborohydride stock solution was added. The Lanterns were added and the mixture heated for 17 h at 60° C. For certain types of amines, a gelatinous substance was formed. Lanterns were pushed into the jelly using tweezers prior to heating.

The reaction solution was removed and the Lanterns washed with methanol (3×5 min), DMF (3×5 min), methanol (3×5 min) and dichloromethane (DCM) (3×5 min). Lanterns were air-dried in a fumehood for 48 h, which were then ready for the next reaction.

Step 2. Acylation of p-Toluidine Mounted on BAL Lanterns with α-Fmoc-Lys(NBoc)

Fmoc-Lys(NBoc) acid (5.6 g, 12 mmol) was dissolved in DCM (60 mL) as a 0.2 M solution in a Schott bottle. Diisopropylcarbodiimide (DIC) (1.88 g, 12 mmol) was added, the mixture was shaken, and then let to stand for 15 min at room temperature. Diisopropyl urea precipitated as a fine colorless solid over this time.

BAL double Lanterns (100) from step 1 were added to solution, shaken to make sure all Lanterns were immersed in solution, and then left to stand overnight at room temperature. The supernatant phase was removed from the Lanterns by aspiration followed by a washing procedure to remove excess reagents. The Lanterns were washed in DCM (2× rapid). Further, longer washing with DCM (3×15 min) followed, after which the Lanterns were air-dried. An Fmoc loading test on two lanterns gave an average loading of 32.5 μmole/Lantern. Proceeding to step 7 retains the Fmoc protection for the isolation of some of the final compounds below (see, e.g., Example 403).

Step 3. Acylation of BAL-p-Toluidine-Lys(Boc) Lanterns with Bromoacetic Acid

The double Lanterns from Step 2 (98) were immersed in freshly prepared piperidine/DMF (1:4) for 30 min at room temperature to effect Fmoc deprotection. After aspiration of the supernatant phase, the Lanterns were washed with DMF (2× rapid) and then further washed with DMF for a longer period (3×10 min). The still wet Lanterns after the final aspiration were used below.

Bromoacetic acid (3.8 g, 27.5 mmol) was dissolved in DMF (55 mL) to make a 0.5 M solution in a Schott bottle. Diisopropyl carbodiimide (4.39 mL, 28.05 mmol) was transferred by pipette and the mixture was shaken and then left to stand for 15 min at room temperature. The deprotected Lanterns (98) were added with some shaking to ensure complete immersion and the mixture was left standing overnight at room temperature.

The supernatant reaction mixture was removed by aspiration and the Lanterns were washed with N,N-dimethylacetamide (DMA) (2× rapid) followed by DMA (2×10 min), DCM (1×10 min), and finally DMA (1×10 min). The reactive derivatized Lanterns were used in the following steps 4-6 without further drying, and protected from the atmosphere as rapidly as possible.

Step 4. Reaction of BAL-p-Toluidine-Lys(BOC) Bromoacetyl Lanterns with Phenols

Phenol solutions (0.25 M) in DMA (105 mL) with anhydrous potassium carbonate (0.25 M) were prepared in screw-capped glass vials. After capping, the mixtures were heated to 40° C. for 30 min, and then cooled to room temperature before placing three lanterns from step 3 into the phenol solutions ensuring complete immersion of the Lanterns. The mixtures were shaken gently at room temperature for 19 h.

A small section of one of the Lanterns from each reaction was excised with a razor blade or scalpel and each section was washed separately in a labeled vial with DMA (2×10 min) followed by DCM (2×10 min). The still wet sections were cleaved as described in Step 7, samples were removed from the TFA/DCM solutions, and diluted in acetonitrile for LC and liquid chromatography/mass spectroscopy (LC/MS) analysis. Phenols that had not completely reacted by substitution with the solid-phase bound bromoacetyl group were heated either at 40° C. or 60° C., depending on the rate of substitution observed. Sampling of the solid phase reaction was repeated as described and as necessary.

At the completion of the reactions, they were aspirated to remove reagents and the Lanterns were then washed with DMA (2×10 min), water (1×10 min), DMA (2×10 min), and finally DCM (4×10 min) while still in their original reaction tubes. The still wet Lanterns were then subjected to cleavage as described in Step 7. Samples were prepared for final LC and LC/MS analysis from the intermediate water/acetonitrile solutions.

Step 5. Reaction of BAL-p-Toluidine-Lys(Boc) Bromoacetyl Lanterns with Primary Amines.

Amine solutions (2 M) in DMA (1.5 mL) were prepared in screw-capped glass vials. Three lanterns from step 3 were placed into the solutions ensuring complete immersion of the Lanterns. The mixtures were shaken gently at room temperature for 19 h.

A small section of one of the Lanterns from each reaction was excised with a razor blade or scalpel and each section was washed separately in a labeled vial with DMA (2×10 min) and then DCM (2×10 min). The still wet sections were cleaved as described in Step 7. Samples were removed from the TFA/DCM solutions and diluted in acetonitrile for LC and LC/MS analysis. All of the primary amines were found to have reacted to completion.

At the completion of the reactions, they were aspirated to remove reagents and the Lanterns were then washed with DMA (2×10 min) and then DCM (3×10 min) while still in their original reaction tubes. The still wet Lanterns were then subjected to cleavage as in step 7. Samples were prepared for final LC and LC/MS analysis from the intermediate water/acetonitrile solutions.

Step 6. Reaction of BAL-p-Toluidine-Lys(Boc) Bromoacetyl Lanterns with Secondary Amines and Anilines

Solutions of aniline or secondary amine (2 M) in DMA (1.5 mL) were prepared in screw-capped glass vials. Three lanterns from Step 3 were placed into the solutions ensuring complete immersion of the Lanterns. The mixtures were heated at room temperature, 60° C. or 80° C., until complete.

The progress of the reaction was determined by analyzing a small sample of the cleaved residue by LC/MS. A small section of one of the Lanterns from each reaction was excised with a razor blade or scalpel and each section was washed separately in a labeled vial with DMA (2×10 min) and then DCM (2×10 min). The still wet sections were cleaved as described in Step 7. Samples were removed from the TFA/DCM solutions and diluted in acetonitrile for LC and LC/MS analysis. When reactions were determined to be incomplete, the temperature of the reaction and/or the reaction time was increased.

Lanterns were allowed to soak in DCM, and the DCM was analyzed by LC/MS. A 150 μL aliquot of the DCM wash solution was placed in an MS vial and allowed to stand in a fumehood so that the DCM would evaporate. The solvent was replaced with neat acetonitrile. Samples were injected (10 μL loop) and if the Lanterns were washed properly, the UV trace of the chromatogram should show no peaks of significance. The presence of a sharp peak was an indication that some non-solid, bound impurities may remain on the surface of the Lantern. Further washing with DMF and DMSO was used to remove the impurities.

### Step 7. BAL Cleavage Reaction

The BAL linker amide products are cleaved with freshly prepared mixed TFA and DCM (1:4) cleavage reagent solution. The Lanterns to be cleaved were placed in glass vials and sufficient cleavage solution was added to cover the Lanterns. The vials were capped and allowed to stand for 1 h at room temperature. The Lanterns were removed, washed with methanol, and discarded. The reaction solutions were evaporated under a stream of nitrogen or placed in a centrifugal evaporator certified to handle TFA vapor. The residues were dissolved in neat acetonitrile and samples were then transferred into pre-weighed 10 mL plastic tubes or equivalent and frozen in liquid nitrogen. The samples were lyophilized by freeze-drying.

These and other methods known in the art can be used to synthesize the compounds of the instant disclosure. Those of ordinary skill in the art will appreciate that many of the amide-based compounds of the instant disclosure may exhibit the phenomena of tautomerism, conformational isomerism, geometric isomerism or optical isomerism. As the formula drawings within the specification and claims can represent only one of the possible tautomeric, conformational isomeric, optical isomeric or geometric isomeric forms, it should be understood that this disclosure encompasses any tautomeric, conformational isomeric, optical isomeric or geometric isomeric forms of the compounds having one or more of the utilities described herein, as well as mixtures of these various different forms. In addition, although the exact optical configurations of the chiral centers of the various illustrated amide-based compounds are not all specified, it is to be understood that the structural illustrations are intended to be a short-hand for describing these compounds, and are not intended to be limiting.

### Anti-Polymerase Activity

The non-nucleoside, amide-based compounds of structure (I)-(X) can be tested for anti-polymerase activity (e.g., for use in treating hyperproliferative diseases or disorders) in various assays, including, for example, enzyme- and cell-based assays, as are known in the art and described herein (see Examples 404-406). The IC₅₀ range of an anti-polymerase compound of this disclosure can vary from about 1 μM to about 75 μM (see Tables 2 and 3). In certain embodiments, the compounds of structure (I)-(X) inhibit or functionally alter an endogenous polymerase, such as polymerase alpha or polymerase gamma. In certain embodiments, the compounds of structure (I)-(X) that inhibit or functionally alter polymerase alpha or polymerase gamma do not inhibit or functionally alter polymerase beta. In one embodiment, the polymerase inhibited or functionally altered is a polymerase alpha. In another embodiment, the endogenous polymerase inhibited or functionally altered is a polymerase gamma.

The compounds of structure (I)-(X) were analyzed using a polymerase assay as described herein, and activities were verified using in vitro screening assays as described herein or as known in the art. In certain embodiments, the present disclosure provides methods for identifying amide-based anti-polymerase compounds and methods for diagnosing the presence of a hyperproliferative disease or disorder, such as cancer, neoplasm, or autoimmunity.

The compounds described herein may be useful research tools for in vitro and cell-based assays to study the biological mechanisms of cell growth and replication, and to identify other anti-polymerase compounds. In one embodiment, a method is provided for identifying anti-polymerase compounds, comprising contacting a host cell with a candidate anti-polymerase compound, such as an anti-polymerase compound of structure (I)-(X), for a time sufficient to inhibit nucleic acid replication, and identifying a candidate anti-polymerase compound that inhibits (prevents, slows, abrogates, interferes with) cell growth or replication. In certain embodiments, the methods described herein may be used to identify a test compound that acts synergistically when combined with a chemotherapeutic agent (e.g., nucleoside-based chemotherapeutic). In another embodiment, a method is provided for identifying cells suspected of being associated with or having a hyperproliferative disease or disorder, comprising contacting a host cell suspected of being associated with or having a hyperproliferative disease or disorder with an anti-polymerase compound of structure (I)-(X) under conditions and for a time sufficient to inhibit cell growth or replication, and identifying cells associated with or having a hyperproliferative disease or disorder. In certain embodiments, the hyperproliferative disease or disorder may be cancer, neoplasm, or autoimmunity. The assays described herein may be used to determine the therapeutic value of a candidate compound or combination, may be used for diagnostic purposes (e.g., detect the presence of a hyperproliferative disease or disorder), and may be useful for determining dosage parameters that would be useful in treating a subject in need thereof.

### Activity Against Cancer Cell Lines

Activity of the compounds described herein is demonstrated for cancer. In particular, activity of the compounds disclosed herein is demonstrated for leukemia, non-small cell lung cancer, colon cancer, CNS (central nervous system) cancer, melanoma, ovarian cancer, renal cancer, prostate cancer, and breast cancer. The non-nucleoside, amide-based compounds of structure (I)-(X) can have activity against cancer cell lines (e.g., for use in treating hyperproliferative diseases or disorders) as determined in various assays, as are known in the art and described herein (see, for example, Examples 407 and 408 below). The activity range of an anti-polymerase compound of this disclosure can vary from about 100 nM to about 75 μM (see Tables 5 through 8). In certain embodiments, the compounds of structure (I)-(X) inhibit growth of at least certain cell lines representing a variety of cancer cells such as CCRF-CEM, CEM-SS, Jurkat, THP-1, HL-60(TB), K-562, MOLT-4, RPMI-8226 and SR, representing leukemia. In certain embodiments, the compounds of structure (I)-(X) inhibit growth of at least certain cell lines representing a variety of cancer cells such as A549/ATCC, EKVX, HOP-62, HOP-92, NCI-H226, NCI-H23, NCI-H322M, NCI-H460 and NCI-H522, representing non-small cell lung cancer. In certain embodiments, the compounds of structure (I)-(X) inhibit growth of at least certain cell lines representing a variety of cancer cells such as Caco-2, DLD-1, HCC-2998, HCT-116, HCT-15, HT29, KM12 and SW-620, representing colon cancer. In certain embodiments, the compounds of structure (I)-(X) inhibit growth of at least certain cell lines representing a variety of cancer cells such as SF-268, SF-295, SF-539, SNB-19, SNB-74 and U251, representing Central nervous system (CNS) cancers. In certain embodiments, the compounds of structure (I)-(X) inhibit growth of at least certain cell lines representing a variety of cancer cells such as LOX IMVI, MALME-3M, M14, SK-MEL-28, SK-MEL-5, UACC-257 and UACC-62, representing melanoma. In certain embodiments, the compounds of structure (I)-(X) inhibit growth of at least certain cell lines representing a variety of cancer cells such as IGORV1, OVCAR-3, OVCAR-4, OVCAR-5, and SK-OV-3, representing ovarian cancer. In certain embodiments, the compounds of structure (I)-(X) inhibit growth of at least certain cell lines representing a variety of cancer cells such as 786-0, A498, ACHN, CAKI-1, SN12C, TK-10, and UO-31, representing renal cancers. In certain embodiments, the compounds of structure (I)-(X) inhibit growth of at least certain cell lines representing a variety of cancer cells such as PC-3 and DU-145, representing prostate cancer. In certain embodiments, the compounds of structure (I)-(X) inhibit growth of at least certain cell lines representing a variety of cancer cells such as MCF7, NCI/ADR-RES, MDA-MB-231/ATCC, HS 578T, MDA-MB-435, BT-549, T-47D, and MDA-MB-468, representing breast cancers.

### Compositions and Therapeutic Uses

The present disclosure provides amide-based compounds and compositions thereof for use in treating hyperproliferative diseases or disorders, as described herein. In particular, the present disclosure provides methods for using such compounds or compositions in reducing or inhibiting the activity of an endogenous polymerase, such as polymerase alpha or polymerase gamma. The reduction or inhibition of polymerase activity may be accomplished by administering a therapeutically effective amount of an amide-based compound having any of the structural forms described herein, or composition thereof, such that a hyperproliferative disease or disorder is treated.

Pharmaceutical compositions comprising compounds of structure (I)-(X) for use in treating hyperproliferative diseases or disorders may be manufactured by means of conventional mixing, dissolving, granulating, dragee making, levigating, emulsifying, encapsulating, entrapping or lyophilizing processes. Pharmaceutical compositions may be formulated in conventional manner using one or more physiologically acceptable carriers, diluents, excipients or auxiliaries that facilitate formulating active compounds of structure (I)-(X) into preparations that can be used pharmaceutically. A single anti-polymerase compound of structure (I)-(X), a plurality of anti-polymerase compounds of structure (I)-(X), or anti-polymerase compounds of structure (I)-(X) in combination with one or more biologically active agents (e.g., chemotherapeutic agents.) may be formulated with a pharmaceutically acceptable carrier, diluent or excipient to generate pharmaceutical compositions of the instant disclosure. The combination therapies may be conveniently formulated together or separately in pharmaceutical formulations comprising a combination as defined herein together with a pharmaceutically acceptable carrier or carriers. The individual components of the combinations above may be administered either simultaneously or sequentially, either in separate or combined pharmaceutical formulations, each in similar or different dosage forms, each by similar or different dosage schedules as appropriately determined by those skilled in the art.

Examples of tumor cells or cancers that may be treated using the methods of this disclosure include breast cancer (e.g., breast cell carcinoma), ovarian cancer (e.g., ovarian cell carcinoma), renal cell carcinoma (RCC), melanoma (e.g., metastatic malignant melanoma), hepatocellular carcinoma (HCC), prostate cancer, gum cancer, tongue cancer, gastrointestinal cancer, colon cancer, lung cancer (including small cell lung cancer and non-small cell lung cancer), bone cancer, osteosarcoma, retinoblastoma, astrocytoma, glioblastoma, neuroblastoma, brain cancer, rhabdomyosarcoma, leiomyosarcoma, chondrosarcoma, pancreatic cancer, skin cancer, fibrosarcoma, chronic or acute leukemias including acute lymphocytic leukemia (ALL), adult T-cell leukemia (T-ALL), acute myeloid leukemia, chronic myeloid leukemia, acute lymphoblastic leukemia, chronic lymphocytic leukemia, hairy cell leukemia, meningeal leukemia, lymphangiosarcoma, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), Castleman's disease, Kaposi's Sarcoma, hemangiosarcoma, multiple myeloma, Waldenstrom's macroglobulinemia and other B-cell lymphomas, nasopharangeal carcinomas, head or neck cancer, myxosarcoma, liposarcoma, cutaneous or intraocular malignant melanoma, rectal cancer, cancer of the anal region, stomach cancer, testicular cancer, uterine cancer, carcinoma of the fallopian tubes, carcinoma of the endometrium, cervical carcinoma, vaginal carcinoma, vulvar carcinoma, transitional cell carcinoma, esophageal cancer, malignant gastrinoma, small intestine cancer, cholangiocellular carcinoma, adenocarcinoma, endocrine system cancer, thyroid gland cancer, parathyroid gland cancer, adrenal gland cancer, sarcoma of soft tissue, urethral, penile cancer, malignant teratoma, solid tumors of childhood, bladder cancer, kidney or ureter cancer, carcinoma of the renal pelvis, malignant meningioma, neoplasm of the central nervous system (CNS), tumor angiogenesis, spinal axis tumor, pituitary adenoma, epidermoid cancer, squamous cell cancer, environmentally induced cancers including those induced by asbestos, e.g., mesothelioma, and combinations of these cancers.

For example, a compound of structure (I)-(X) may be used in combination with one or more other adjunctive therapies, such as a nucleoside-based chemotherapeutic agent. Chemotherapeutic agents may be any known or subsequently discovered agents. For example, known types of chemotherapeutic agents include anthracyclines, alkylating agents, alkyl sulfonates, aziridines, ethylenimines, methylmelamines, nitrogen mustards, nitrosoureas, antibiotics, antimetabolites, folic acid analogs, purine analogs, pyrimidine analogs, enzymes, podophyllotoxins, platinum-containing agents, interferons, and interleukins.

Exemplary known chemotherapeutic agents include busulfan, improsulfan, piposulfan, benzodepa, carboquone, meturedepa, uredepa, altretamine, triethylenemelamine, triethylenephosphoramide, triethylenethiophosphoramide, trimethylolomelamine, chlorambucil, chlornaphazine, cyclophosphamide, estramustine, ifosfamide, mechlorethaamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, dacarbazine, mannomustine, mitobronitol, mitolactol, pipobroman, aclacinomycins, actinomycin F(1), anthramycin, azaserine, bleomycin, cactinomycin, carubicin, carzinophilin, chromomycin, dactinomycin, daunorubicin, daunomycin, 6-diazo-5-oxo-1-norleucine, doxorubicin, epirubicin, mitomycin C, mycophenolic acid, nogalamycin, olivomycin, peplomycin, plicamycin, porfiromycin, puromycin, streptonigrin, streptozocin, tubercidin, ubenimex, zinostatin, zorubicin, denopterin, methotrexate, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, fluororacil, tegafur, L-asparaginase, pulmozyme, aceglatone, aldophosphamide glycoside, aminolevulinic acid, amsacrine, bestrabucil, bisantrene, carboplatin, cisplatin, defofamide, demecolcine, diaziquone, elformithine, elliptinium acetate, etoglucid, etoposide, flutamide, gallium nitrate, hydroxyurea, interferon-alpha, interferon-beta, interferon-gamma, interleukin-2, lentinan, lonidamine, mitoguazone, mitoxantrone, mopidamol, nitracrine, pentostatin, phenamet, pirarubicin, podophyllinic acid, 2-ethylhydrazide, procarbazine, razoxane, sizofuran, spirogermanium, paclitaxel, tamoxifen, teniposide, tenuazonic acid, triaziquone, 2,2′,2″-trichlorotriethylamine, urethan, vidarabine, vinblastine, vincristine, and vindesine. In certain embodiments, an compound of structure (I)-(X) is administered in combination with an interferon, such as interferon-α or known as pegylated interferon, such as peginterferon α-2b (Peg-Intron; Schering-Plough) or peginterferon α-2a (Pegasys®; Hoffmann-La Roche).

Pharmaceutically acceptable carriers, diluents or excipients for therapeutic use are well known in the pharmaceutical art, and are described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992). In certain embodiments, compounds of any of structures (I)-(X) may be formulated with a pharmaceutically or physiologically acceptable carrier, diluent or excipient that is aqueous, such as water or a mannitol solution (e.g., about 1% to about 20%), hydrophobic carrier (e.g., oil or lipid), or a combination thereof (e.g., oil and water emulsions). In certain embodiments, any of the pharmaceutical compositions described herein are sterile.

The formulations of the present disclosure having an amount of one or more compounds of any of structures (I)-X), with or without another chemotherapeutic agent, sufficient to treat or prevent a hyperproliferative disease or disorder are, for example, suitable for topical (e.g., creams, ointments, skin patches, eye drops, ear drops, shampoos) application or administration. Other exemplary routes of administration include oral, parenteral, sublingual, bladder wash-out, vaginal, rectal, enteric, suppository, nasal, or inhalation. The term parenteral, as used herein, includes subcutaneous, intravenous, intramuscular, intraarterial, intraabdominal, intraperitoneal, intraarticular, intraocular or retrobulbar, intraaural, intrathecal, intracavitary, intracelial, intraspinal, intrapulmonary or transpulmonary, intrasynovial, intratumoral, or intraurethral injection or infusion techniques. The pharmaceutical compositions of the present disclosure are formulated so as to allow the compounds of any of structures (I)-(X) contained therein to be bioavailable upon administration of the composition to a subject. The level of anti-polymerase compound in serum and other tissues after administration can be monitored by various well-established techniques, such as chromatographic- or antibody-based (e.g., ELISA) assays. In certain embodiments, compounds of any of structures (I)-(X) are formulated for topical application to a target site on a subject in need thereof, such as an animal or a human. In other embodiments, compounds of any of structures (I)-(X) derivatives are formulated for parenteral administration to a subject in need thereof (e.g., having a hyperproliferative disease or disorder, such as cancer, neoplasm, or autoimmunity), such as a non-human animal or a human.

Proper formulation is generally dependent upon the route of administration chosen, as is known in the art. For example, in exemplary embodiments for topical administration, the compounds of any of structures (I)-(X) may be formulated as solutions, gels, ointments, creams, suspensions, pastes, and the like. Systemic formulations are another embodiment, which includes those designed for administration by injection, e.g. subcutaneous, intravenous, intra-arterial, intratumoral, intramuscular, intrathecal or intraperitoneal injection, as well as those designed for transdermal, transmucosal, oral, intranasal, or pulmonary administration. In one embodiment, the systemic formulation is sterile. In embodiments for injection, the compounds of any of structures (I)-(X) may be formulated in an aqueous solution, preferably in a physiologically compatible solution or buffer such as Hank's solution, Ringer's solution, mannitol solutions or physiological saline buffer. In certain embodiments, any of the compositions described herein may contain formulatory agents, such as suspending, stabilizing or dispersing agents. Alternatively, the compounds of any of structures (I)-(X) may be in solid form (e.g., powder) for constitution with a suitable vehicle (e.g., sterile pyrogen-free water) before use. In embodiments for transmucosal administration, penetrants, solubilizers or emollients appropriate to the barrier to be permeated may be used in the formulation. For example, 1-dodecylhexahydro-2H-azepin-2-one (Azone®), oleic acid, propylene glycol, menthol, diethyleneglycol ethoxyglycol monoethyl ether (Transcutol®), polysorbate polyethylenesorbitan monolaurate (Tween®-20), and the drug 7-chloro-1-methyl-5-phenyl-3H-1,4-benzodiazepin-2-one (Diazepam), isopropyl myristate, and other such penetrants, solubilizers or emollients generally known in the art may be used in any of the compositions of the instant disclosure.

In other embodiments, the compounds of any of structures (I)-(X) can be formulated with a pharmaceutically acceptable carrier in the form of tablets, pills, dragees, capsules, liquids, gels, syrups, slurries, suspensions and the like, for oral ingestion by a subject or patient to be treated. In certain embodiments for oral solid formulations, such as powders, capsules or tablets, suitable excipients include fillers, such as sugars (e.g., lactose, sucrose, mannitol, sorbitol); cellulose preparations such as maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP); granulating agents; or binding agents.

Optionally, disintegrating agents may be added, such as cross-linked polyvinylpyrrolidone, agar, or alginic acid (or a salt thereof, such as sodium alginate). If desired, solid dosage forms may be sugar-coated or enteric-coated using standard techniques. In some embodiments for oral liquid preparations, such as suspensions, elixirs or solutions, suitable carriers, excipients or diluents include water, glycols, oils, alcohols, or combinations thereof. Additionally, flavoring agents, preservatives, viscosity-increasing agents, humectants, coloring agents, or the like, may be added. In embodiments for oral or buccal administration, the compositions may take the form of, for example, tablets or lozenges, formulated as is known in the art and described herein.

In embodiments for administration by inhalation, the compounds for use according to the present disclosure may be formulated for convenient delivery in the form of drops for intranasal administration, or in the form of an aerosol spray from pressurized packs or nebulizer having a suitable propellant (e.g., dichlorodifluoromethane, trichlorofluoromethane, dichlorotetrafluoroethane, carbon dioxide or other suitable gas). In certain embodiments, the drops or aerosol composition is sterile. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base, such as lactose or starch.

In other embodiments, the compounds of any of structures (I)-(X) may be formulated into rectal or vaginal compositions, such as suppositories or retention enemas, e.g., containing conventional suppository bases, such as cocoa butter or other glycerides.

In addition to the formulations described herein, the compounds of this disclosure may also be formulated as a depot preparation. For example, compounds of any of structures (I)-(X) can be in the form of the slow-release formulation such that they can provide activity over time. Such long-acting formulations may be administered by implantation (for example, subcutaneously or intramuscularly) or by intramuscular or intratumoral injection. In certain embodiments, the compounds may be formulated with suitable a polymer (including poly(lactides), poly(glycolides), poly(caprolactones), and blends thereof), a hydrophobic material, (including a physiologically acceptable oil, which can be in the form of an emulsion), an ion exchange resin, or as sparingly soluble derivatives (such as a sparingly soluble salt).

Alternatively, other pharmaceutical delivery systems may be employed. In certain embodiments, the compounds are formulated with liposomes or emulsions as delivery vehicles. Certain organic solvents, such as dimethylsulfoxide (DMSO), may also be employed. Additionally, the compounds of any of structures (I)-(X) may be delivered using a sustained-release system, such as semipermeable matrices of solid or semi-solid polymers (e.g., thermopaste) containing the therapeutic agent. Sustained-release capsules may, depending on their chemical nature, release the compounds for a few hours, a few days, a few weeks, or for up to about 100 days.

As certain of the carboxyl groups of the compounds of any of structures (I)-(X) are acidic, or the substituents R¹, R², R³, R⁴, and R⁵ may include acidic or basic substituents, the compounds of any of structures (I)-(X) may be included in any of the above-described formulations as a free acid, a free base, or as a pharmaceutically acceptable salt. Pharmaceutically acceptable salts are those salts that substantially retain the anti-polymerase activity of the free acid or base, and which are prepared by reaction with a base or acid, respectively. Suitable acids and bases are well known to those of ordinary skill in the art and are described herein. Exemplary pharmaceutical salts may tend to be more soluble in aqueous and other protic solvents than is the corresponding free base or acid form.

Compounds of any of structures (I)-(X) can be provided in dosage amounts and intervals, which can be adjusted on a case-by-case basis, to provide plasma levels of one or more of the anti-polymerase compounds sufficient to maintain a therapeutic effect. Exemplary clinical dosages for administration by injection may range from about 0.1 to about 200 mg/kg/day, or range from about 1.5 to about 15 mg/kg/day. In certain embodiments, therapeutically effective serum levels may be achieved by administering a single dose or as a single daily dose or multiple doses each day over a specified time period. That is, the desired dose may be conveniently provided in divided doses administered at appropriate intervals, for example, two, three, four or more doses per day, or one dose per day, one dose per two days, etc. In other embodiments, therapeutically effective serum levels may also be achieved by administering at less frequent dosing schedules such as, for example, once every two days, twice a week, once a week or at longer intervals between dosing, or any combination thereof. For example, combination administration schedules may be utilized to reach therapeutically effective does, such as multiple does on one or more days followed by less frequent dosing such as, for example, once every two days, twice a week or once a week, or longer.

The compositions of this disclosure may be administered to a subject as a single dosage unit form (e.g., a tablet, capsule, injection or gel), or the compositions may be administered, as described herein, as a plurality of dosage unit forms (e.g., in aerosol or injectable form, tablet, capsule), or in any combination thereof. For example, the formulations provided herein may be sterilized and packaged in single-use, plastic laminated pouches or plastic tubes of dimensions selected to provide for routine, measured dispensing. In one example, the container may have dimensions anticipated to dispense 0.5 mL of the composition (e.g., a drop, gel or injection form) to a subject, or to a limited area of a target surface on or in a subject (e.g., benign or malignant tumor), to treat a hyperproliferative disease or disorder.

In cases of local administration or selective uptake, the effective local concentration of compounds of any of structures (I)-(X) may not relate to plasma concentration. A person having ordinary skill in the art will be able to optimize therapeutically effective local dosages without undue experimentation. The amount of an active compound of structure (I)-(X) administered will be dependent upon, among other factors, the subject being treated, the subject's weight, the severity of the affliction, the manner of administration and the judgment of the prescribing physician.

The compositions of this disclosure may be provided in various forms, depending on the amount and number of different pharmaceutically acceptable excipients present. For example, the compositions may be in the form of a solid, a semi-solid, a liquid, a lotion, a cream, an ointment, a cement, a paste, a gel, or an aerosol. In one embodiment, the formulation is in the form of a liquid or a gel. Pharmaceutically acceptable excipients suitable for use in the formulation compositions as described herein may optionally include, for example, a viscosity-increasing agent, a buffering agent, a solvent, a humectant, a preservative, a chelating agent (e.g., EDTA or EGTA), an oleaginous compound, an emollient, an antioxidant, an adjuvant, or the like. Exemplary buffering agents suitable for use with the compounds of any of structures (I)-(X) or compositions thereof include monocarboxylate or dicarboxylate compounds (such as acetate, fumarate, lactate, malonate, succinate, or tartrate). Exemplary preservatives include benzoic acid, benzyl alcohol, phenoxyethanol, methylparaben, propylparaben, and the like. The function of each of these excipients is not mutually exclusive within the context of the present disclosure. For example, glycerin may be used as a solvent or as a humectant or as a viscosity-increasing agent.

The present disclosure provides a method for treating a hyperproliferative disease or disorder, such as a benign or malignant tumor, in a host comprising administering a therapeutically effective amount of a compound of any of structure (I)-(X). In one embodiment, the hyperproliferative disease or disorder being treated is cancer. The therapy may be repeated intermittently while the hyperproliferative disease or disorder is detectable or even when not detectable.

Treatment, as provided by the present disclosure, can encompass preventative or maintenance administration of any compound or combination described herein. For example, effective treatment of a hyperproliferative disease or disorder may include a cure of such a disease or disorder (e.g., eradication of the pathology); a sustained response in which a hyperproliferative disease or disorder is no longer detectable in a subject for a certain amount of time after completing a therapeutic regimen (such a sustained response may be equated with a favorable prognosis and may be equivalent to a cure); slowing or reducing any associated tissue damage (e.g., reducing or eliminating a tumor); reducing, alleviating, or abrogating symptoms associated with a hyperproliferative disease or disorder in a subject; or preventing symptoms associated with a hyperproliferative disease or disorder from worsening or progressing. To date, a therapeutic agent that adequately treats cancer and any associated disease or disorder without severe side-effects has remained elusive.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. This disclosure having been described, the following examples are intended to illustrate, and not limit, this disclosure.

### EXAMPLES

A number of non-nucleoside derivatives were prepared according to Scheme (I) (in solution; see, e.g., Examples 2-325) or Scheme (II) (in solid phase; see, e.g., Examples 325-403) from commercially available starting materials or starting materials prepared by conventional synthetic or biosynthetic methods. Alternative methods of preparation are further described in specific examples when necessary.

Sample preparation for purification by high-pressure liquid chromatography (HPLC) involved diluting a sample with 2 ml of 0.2% trifluoroacetic acid (TFA) in acetonitrile and 2 mL H₂O, and then filtering with Pall GHP Acrodisc® GF 25 mm Syringe Filter with a GF/0.45 um GHP Membrane. The HPLC system used was a BioCAD® Sprint™ Perfusion Chromatography®, with UV wavelengths of 220 nm and 280 nm used and a flow rate of 15 mL/min. Solvent A is 0.1% TFA in mQ water, and Solvent B is 0.1% TFA in Acetonitrile (HPLC Grade). The column used was a Waters C18 (or C8) Symmetry Prep 7 um, 19×150 mm. The general liquid chromatography (LC) method used was a gradient protocol: 80% solvent A to 30% solvent A over 9 column volumes. Fractions containing a desired compound were combined, organic solvents removed in vacuo, with the remaining aqueous layer being frozen and lyophilized to obtain an amphorous solid that was generally expected to be the TFA salt of a desired product.

### Example 1

### General Compound Synthesis in Solution

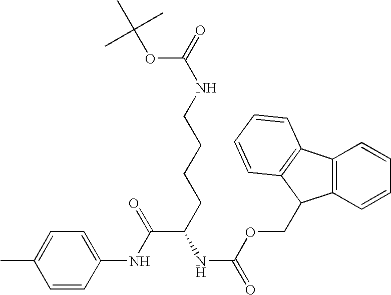

(a) Synthesis of (S)-(5-tert-Butoxycarbonylamino-1-p-tolylcarbamoyl-pentyl)-carbamic acid 9H-fluoren-9-ylmethyl ester (Intermediate #1): A solution of (S)-6-tert-Butoxycarbonylamino-2-(9H-fluoren-9-ylmethoxycarbonylamino)-hexanoic acid (3.0 g, 6.4 mmol), p-tolylamine (0.75 g, 7.1 mmol), and 2.7 g (8.3 mmol) of 2-(1H-benzotriazole-1-yl)-1,1,3,3-tetramethyluronium tetrafluoroborate in 150 mL of dimethylformamide was stirred at room temperature under inert atmosphere (nitrogen). Diisopropylethylamine (4.5 mL, 25.6 mmol) was added to the mixture and the reaction was stirred until deemed complete by thin layer chromatography (approximately 3 h).

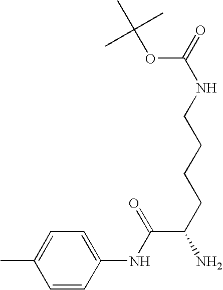

(b) Deprotection of 9H-fluoren-9-ylmethyl Ester (Fmoc) Protecting Group to Produce (S)-(5-Amino-5-p-tolylcarbamoyl-pentyl)-carbamic Acid tert-butyl Ester (Intermediate #2):

Fmoc deprotection of Intermediate #1 was accomplished by charging the crude reaction mixture with excess piperidine (20 mL, typically 5 equivalents or more), then allowing the mixture to stir for 30 minutes. Upon completion, the mixture was concentrated in vacuo then purified by flash chromatography using a gradient solvent system of 25%, 50%, then 75% ethyl acetate in hexanes to afford the pure free amine in 93% yield for the two steps: [M+H]⁺ calcd for C₁₈H₃₀N₃O₃, 336; found 336.

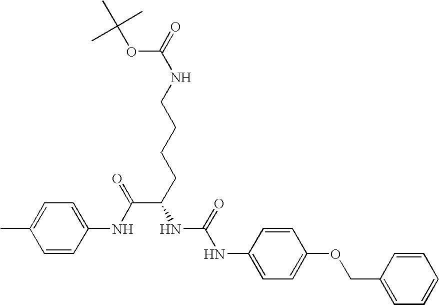

(c) Synthesis of (S)-{5-[3-(4-Benzyloxy-phenyl)-ureido]-5-p-tolylcarbamoyl-pentyl}-carbamic acid tert-butyl ester (Intermediate #3): To a solution of Intermediate #2 (40 mg, 0.12 mmol) in 2 mL of dichloromethane was added to 1-benzyloxy-4-isocyanato-benzene (32 mg, 0.14 mmol), and then stirred at room temperature under inert atmosphere (nitrogen) until deemed complete by TLC. Concentration in vacuo produced the crude Intermediate #3.

### Scheme (I) Compounds

### Example 2

### (S)-6-AMINO-2-[3-(4-BENZYLOXY-PHENYL)-UREIDO]-HEXANOIC ACID P-TOLYLAMIDE

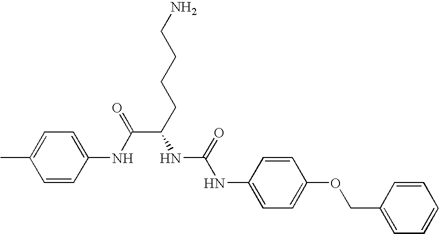

A solution of Intermediate #3 of Example 1 (0.12 mmol) in tetrahydrofuran (0.1M solution) was charged with trifluoroacetic acid (0.05M), and the mixture was stirred for 30 minutes. Upon completion, based on TLC, the mixture was reconcentrated in vacuo to produce a crude oily substance that was purified by HPLC to produce the title compound (16 mg, 28% yield for the two steps): 1H NMR (400 MHz, DMSO-d₆) δ 10.07 (s, 1H), 8.55 (s, 1H), 7.67 (br s, 3H), 7.49 (d, J=8.4 Hz, 2H), 7.42 (d, J=7.2 Hz, 2H), 7.38 (t, J=7.5 Hz, 2H), 7.31 (t, J=7.2 Hz, 1H), 7.28 (d, J=9.0 Hz, 2H), 7.11 (d, J=8.5 Hz, 2H), 6.89 (d, J=9.0 Hz, 2H), 6.44 (d, J=8.3 Hz, 1H), 5.03 (s, 2H), 4.37 (dt, J=5.7, 8.1 Hz, 1H), 2.81-2.75 (m, 2H), 2.25 (s, 3H), 1.76-1.69 (m, 1H), 1.63-1.52 (m, 3H), 1.44-1.3 (m, 2H); Low resolution mass spectrum (ES) m/e 461 [(M+1)⁺, calcd for C₂₇H₃₃N₄O₃: 461]; 99.7% purity based on HPLC.

### Example 3

### N-[1-(4-{[CYCLOHEXYL-(FURAN-2-CARBONYL)-AMINO]-METHYL}-PHENYLCARBAMOYL)-1-METHYL-ETHYL]-TEREPHTHALAMIC ACID METHYL ESTER

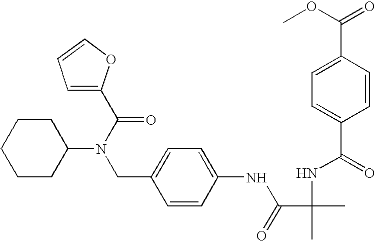

Furan-2-carboxylic acid (4-amino-benzyl)-cyclohexyl-amide, and 2-(9H-Fluoren-9-ylmethoxycarbonylamino)-2-methyl-propionic acid were coupled using the method described for Intermediates #1 and #2 of Example 1. Coupling with terephthalic acid monomethyl ester was achieved as described for Intermediate #1 of Example 1. Purification by HPLC produced the title compound. 1H NMR (400 MHz, DMSO-d₆) δ 9.42 (s, 1H), 8.55 (s, 1H), 8.43 (s, 4H), 7.77 (br s, 1H), 7.49 (d, J=8 Hz, 2H), 7.14 (d, J=9 Hz, 2H), 6.86 (br s, 1H), 6.56 (br s, 1H), 4.62 (br s, 2H), 4.10 (br s, 1H), 3.87 (s, 3H), 2.68 (s, 4H), 1.69-1.42 (m, 13H), 1.24-0.96 (m, 3H); Low resolution mass spectrum (ES) m/e 546 [(M+H)⁺, calcd for C₃₁H₃₆N₃O₆: 546]; 98% purity based on HPLC.

### Example 4

### N-[1-(4-{[CYCLOHEXYL-(FURAN-2-CARBONYL)-AMINO]-METHYL}-PHENYLCARBAMOYL)-1-METHYL-ETHYL]-TEREPHTHALAMIC ACID

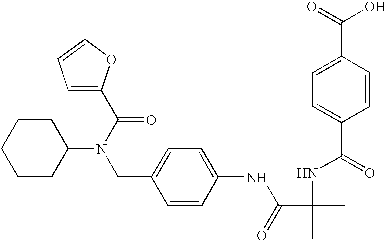

Standard hydrolysis (0.1N sodium hydroxide in methanol for 12 hours) of the compound from Example 3, and purification by HPLC produced the title compound. 1H NMR (400 MHz, DMSO-d₆) δ 13.18 (s, 1H), 9.41 (s, 1H), 8.51 (s, 1H), 8.03-7.98 (m, 4H), 7.77 (br s, 1H), 7.49 (d, J=8 Hz, 2H), 7.14 (d, J=8 Hz, 2H), 6.86 (br s, 1H), 6.55 (br s, 1H), 4.62 (s, 2H), 4.10 (br s, 1H), 1.7-1.42 (m, 7H), 1.52 (m, 6H), 1.27-0.96 (m, 3H); Low resolution mass spectrum (ES) m/e 532 [(M+H)⁺, calcd for C₃₀H₃₄N₃O₆: 532]; 90.4% purity based on HPLC.

### Example 5

### (S)-[5-AMINO-5-(4-{[CYCLOHEXYL-(FURAN-2-CARBONYL)-AMINO]-METHYL}-PHENYLCARBAMOYL)-PENTYL]-CARBAMIC ACID ALLYL ESTER

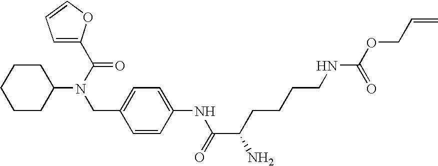

Furan-2-carboxylic acid (4-amino-benzyl)-cyclohexyl-amide was coupled to (S)-6-Allyloxycarbonylamino-2-(9H-fluoren-9-ylmethoxycarbonylamino)-hexanoic acid as described in the method for Intermediates #1 and #2 of Example 1. Purification by HPLC produced the title compound: 1H NMR (400 MHz, DMSO-d₆) δ 10.4 (s, 1H), 8.2 (d, J=3 Hz, 1H), 7.79 (br s, 1H), 7.51 (d, J=8 Hz, 2H), 7.24 (d, J=8 Hz, 2H), 7.15 (t, J=5 Hz, 1H), 6.89 (br s, 1H), 6.58 (br s, 1H), 5.86 (ddd, J=5, 11, 22 Hz, 1H), 5.23 (dd, J=2, 17 Hz, 1H), 5.13 (dd, J=1.4, 10 Hz, 1H), 4.64 (br s, 2H), 4.41 (d, J=5 Hz, 2H), 4.11 (t, J=11 Hz, 1H), 3.87 (m, 2H), 2.96 (q, J=6 Hz, 2H), 1.81-1.63 (m, 6H), 1.55-1.17 (m, 9H), 1.08-0.98 (m, 1H); Low resolution mass spectrum (ES) m/e 511 [(M+H)⁺, calcd for C₂₈H₃₉N₄O₅: 511]; 100% purity based on HPLC.

### Example 6

### (S)-[5-AMINO-1-(4-{[CYCLOHEXYL-(FURAN-2-CARBONYL)-AMINO]-METHYL}-PHENYLCARBAMOYL)-PENTYL]-CARBAMIC ACID 9H-FLUOREN-9-YL METHYL ESTER

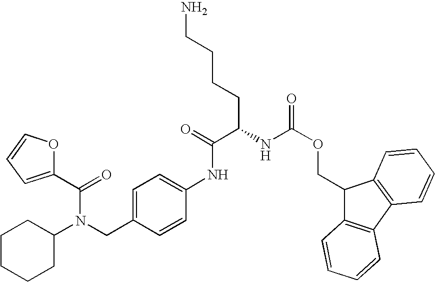

Furan-2-carboxylic acid (4-amino-benzyl)-cyclohexyl-amide was coupled to (S)-6-tert-butoxycarbonylamino-2-(9H-fluoren-9-ylmethoxycarbonylamino)-hexanoic acid as described in the method for Intermediate #1 of Example 1. Deprotection using the method described for Example 2 and purification by HPLC produced the title compound. 1H NMR (400 MHz, DMSO-d₆) δ 9.99 (s, 1H), 7.88 (d, J=8 Hz, 2H), 7.78 (br s, 1H), 7.72 (t, J=8 Hz, 2H), 7.64-7.59 (m, 4H), 7.52 (d, J=8 Hz, 2H), 7.40 (dt, J=3, 8 Hz, 2H), 7.31 (dd, J=7, 13 Hz, 2H), 7.19 (d, J=8 Hz, 2H), 6.88 (br s, 1H), 6.57 (br s, 1H), 4.63 (br s, 2H), 4.33-4.19 (m, 3H), 4.11 (dd, J=8, 14 Hz, 2H), 2.77 (qd, J=6, 12 Hz, 2H), 1.70-1.16 (m, 15H), 1.07-0.98 (m, 1H); Low resolution mass spectrum (ES) m/e 649 [(M+H)⁺, calcd for C₃₉H₄₅N₄O₅: 649]; 98.8% purity based on HPLC.

### Example 7

### (S)-FURAN-2-CARBOXYLIC ACID CYCLOHEXYL-[4-(2,6-DIAMINO-HEXANOYLAMINO)-BENZYL]-AMIDE

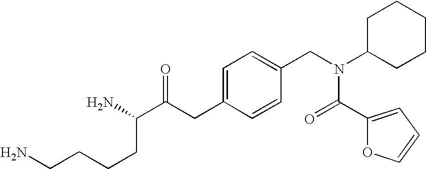

(S)-[5-Amino-1-(4-{[cyclohexyl-(furan-2-carbonyl)-amino]-methyl}-phenylcarbamoyl)-pentyl]-carbamic acid 9H-fluoren-9-ylmethyl ester from Example 6 was deprotected as described in the method for Intermediate #2 of Example 1. Purification by HPLC produced the title compound. 1H NMR (400 MHz, DMSO-d₆) δ 10.45 (s, 1H), 8.27 (d, J=3 Hz, 3H), 7.79 (s, 1H), 7.74 (s, 3H), 7.53 (d, J=8.5 Hz, 2H), 7.24 (d, J=8.5 Hz, 2H), 6.89 (s, 1H), 6.58 (s, 1H), 4.64 (s, 2H), 4.11 (br s, 1H), 3.91 (m, 1H), 2.76 (dd, J=6.5, 14 Hz, 1H), 1.81-1.76 (m, 2H), 1.71-1.63 (m, 4H), 1.58-1.45 (m, 5H), 1.40-1.33 (m, 2H), 1.27-1.17 (m, 2H), 1.08-0.98 (m, 1H); Low resolution mass spectrum (ES) m/e 427 [(M+H)⁺, calcd for C₂₄H₃₅N₄O₃: 427]; 99.1% purity based on HPLC.

### Example 8

### (S)-FURAN-2-CARBOXYLIC ACID {4-[2-AMINO-3-(4-TERT-BUTOXY-PHENYL)-PROPIONYLAMINO]-BENZYL}-CYCLOHEXYL-AMIDE

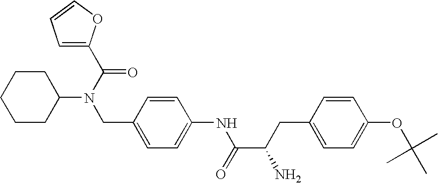

Furan-2-carboxylic acid (4-amino-benzyl)-cyclohexyl-amide was coupled to (S)-3-(4-tert-Butoxy-phenyl)-2-(9H-fluoren-9-ylmethoxycarbonylamino)-propionic acid as described in the method of Example 5. Purification by HPLC produced the title compound. 1H NMR (400 MHz, DMSO-d₆) δ 10.22 (s, 1H), 8.28 (br s, 3H), 7.79 (br s, 2H), 7.38 (d, J=8 Hz, 2H), 7.20 (d, J=8 Hz, 2H), 7.14 (d, J=8 Hz, 2H), 6.88 (d, J=8 Hz, 3H), 6.58 (br s, 1H), 4.63 (br s, 2H), 4.13-4.06 (m, 2H), 3.10-3.00 (m, 2H), 1.71-1.62 (m, 4H), 1.55-1.44 (m, 3H), 1.27-1.17 (m, 2H), 1.22 (s, 9H), 1.09-0.98 (m, 1H); Low resolution mass spectrum (ES) m/e 518 [(M+H)⁺, calcd for C₃₁H₄₀N₃O₄: 518]; 98.6% purity based on HPLC.

### Example 9

### (S)-FURAN-2-CARBOXYLIC ACID {4-[2-AMINO-3-(4-METHOXY-PHENYL)-PROPIONYLAMINO]-BENZYL}-CYCLOHEXYL-AMIDE

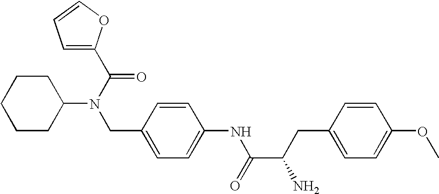

Furan-2-carboxylic acid (4-amino-benzyl)-cyclohexyl-amide was coupled to (S)-3-(4-methoxy-phenyl)-2-(9H-fluoren-9-ylmethoxycarbonylamino)-propionic acid as described in the method of Example 5. Purification by HPLC produced the title compound. 1H NMR (400 MHz, DMSO-d₆) δ 10.34 (s, 1H), 8.23 (br s, 3H), 7.79 (m, 1H), 7.45 (d, J=8 Hz, 2H), 7.23 (d, J=9 Hz, 2H), 7.16 (d, J=9 Hz, 2H), 6.88 (d, J=9 Hz, 3H), 6.58 (m, 1H), 4.64 (br s, 2H), 4.14-4.06 (m, 2H), 3.71 (s, 3H), 3.10 (dd, J=6, 14 Hz, 1H), 2.99 (dd, J=7, 14 Hz, 1H), 1.71-1.63 (m, 4H), 1.55-1.44 (m, 3H), 1.29-1.17 (m, 2H), 1.08-0.98 (m, 1H); Low resolution mass spectrum (ES) m/e 476 [(M+H)⁺, calcd for C₂₈H₃₄N₃O₄: 476]; 95.4% purity based on HPLC.

### Example 10

### (S)-FURAN-2-CARBOXYLIC ACID [4-(2-AMINO-3-PHENYL-PROPIONYLAMINO)-BENZYL]-CYCLOHEXYL-AMIDE

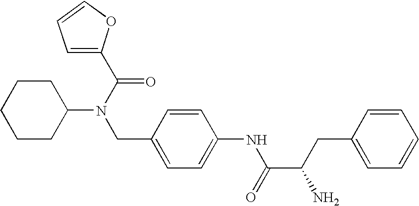

Furan-2-carboxylic acid (4-amino-benzyl)-cyclohexyl-amide was coupled to (S)-3-phenyl-2-(9H-fluoren-9-ylmethoxycarbonylamino)-propionic acid as described in the method of Example 5. Purification by HPLC produced the title compound. 1H NMR (400 MHz, DMSO-d₆) δ 10.32 (s, 1H), 8.29 (br s, 3H), 7.79 (br s, 1H), 7.42 (d, J=8 Hz, 2H), 7.34-7.21 (m, 7H), 6.89 (br s, 1H), 6.58 (br s, 1H), 4.63 (br s, 2H), 4.11 (m, 2H), 3.15 (dd, J=7, 14 Hz, 1H), 3.06 (dd, J=7, 14 Hz, 1H), 1.71-1.63 (m, 4H), 1.55-1.44 (m, 3H), 1.27-1.17 (m, 2H), 1.08-0.98 (m, 1H); Low resolution mass spectrum (ES) m/e 446 [(M+H)⁺, calcd for C₂₇H₃₂N₃O₃: 446]; 100% purity based on HPLC.

### Example 11

### (R)-FURAN-2-CARBOXYLIC ACID [4-(2-AMINO-3-PHENYL-PROPIONYLAMINO)-BENZYL]-CYCLOHEXYL-AMIDE

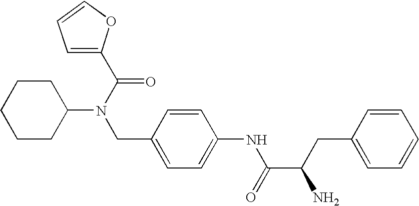

Furan-2-carboxylic acid (4-amino-benzyl)-cyclohexyl-amide was coupled to (R)-3-phenyl-2-(9H-fluoren-9-ylmethoxycarbonylamino)-propionic acid as described in the method of Example 5. Purification by HPLC produced the title compound. 1H NMR (400 MHz, DMSO-d₆) δ 10.33 (s, 1H), 8.30 (br s, 3H), 7.79 (br s, 1H), 7.43 (d, J=8 Hz, 2H), 7.34-7.21 (m, 7H), 6.89 (br s, 1H), 6.58 (br s, 1H), 4.63 (br s, 2H), 4.11 (br m, 2H), 3.15 (dd, J=7, 14 Hz, 1H), 3.06 (dd, J=7, 14 Hz, 1H), 1.71-1.63 (m, 4H), 1.55-1.44 (m, 3H), 1.27-1.17 (m, 2H), 1.09-0.98 (m, 1H); Low resolution mass spectrum (ES) m/e 446 [(M+H)⁺, calcd for C₂₇H₃₂N₃O₃: 446]; 100% purity based on HPLC.

### Example 12

### (S)-FURAN-2-CARBOXYLIC ACID [4-(2-AMINO-PROPIONYLAMINO)-BENZYL]-CYCLOHEXYL-AMIDE

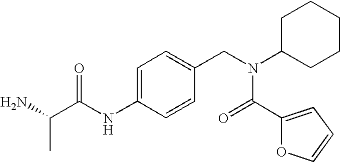

Furan-2-carboxylic acid (4-amino-benzyl)-cyclohexyl-amide coupled to (S)-2-(9H-Fluoren-9-ylmethoxycarbonylamino)-propionic acid as described in the method of Example 5. Purification by HPLC produced the title compound. 1H NMR (400 MHz, DMSO-d₆) δ 10.38 (s, 1H), 8.17 (br s, 3H), 7.79 (br s, 1H), 7.52 (d, J=8 Hz, 2H), 7.23 (d, J=8 Hz, 2H), 6.89 (br s, 1H), 6.58 (br s, 1H), 4.64 (br s, 2H), 4.11 (t, J=11 Hz, 1H), 3.97 (br s, 1H), 1.71-1.63 (m, 4H), 1.55-1.47 (m, 3H), 1.43 (d, J=7 Hz, 3H), 1.26-1.17 (m, 2H), 1.08-0.98 (m, 1H); Low resolution mass spectrum (ES) m/e 370 [(M+H)⁺, calcd for C₂₁H₂₈N₃O₃: 370]; 99% purity based on HPLC.

### Example 13

### (R)-FURAN-2-CARBOXYLIC ACID [4-(2-AMINO-PROPIONYLAMINO)-BENZYL]-CYCLOHEXYL-AMIDE

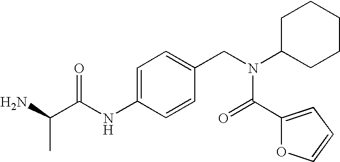

Furan-2-carboxylic acid (4-amino-benzyl)-cyclohexyl-amide was coupled to (R)-2-(9H-Fluoren-9-ylmethoxycarbonylamino)-propionic acid as described in the method of Example 5. Purification by HPLC produced the title compound. 1H NMR (400 MHz, DMSO-d₆) δ 10.36 (s, 1H), 8.16 (br s, 3H), 7.79 (br s, 1H), 7.52 (d, J=9 Hz, 2H), 7.23 (d, J=8 Hz, 2H), 6.89 (br s, 1H), 6.58 (br s, 1H), 4.64 (br s, 2H), 4.11 (t, J=11 Hz, 1H), 3.97 (br s, 1H), 1.71-1.63 (m, 4H), 1.55-1.47 (m, 3H), 1.43 (d, J=7 Hz, 3H), 1.26-1.17 (m, 2H), 1.08-0.98 (m, 1H); Low resolution mass spectrum (ES) m/e 370 [(M+H)⁺, calcd for C₂₁H₂₈N₃O₃: 370]; 99.3% purity based on HPLC.

### Example 14

### (S)-[3-AMINO-3-(4-{[CYCLOHEXYL-(FURAN-2-CARBONYL)-AMINO]-METHYL}-PHENYLCARBAMOYL)-PROPYL]-CARBAMIC ACID 9H-FLUOREN-9-YLMETHYL ESTER

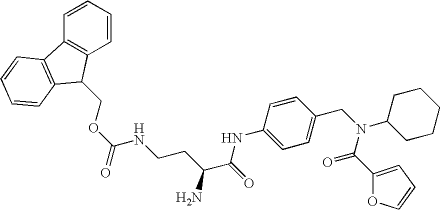

Furan-2-carboxylic acid (4-amino-benzyl)-cyclohexyl-amide was coupled to (S)-2-tert-Butoxycarbonylamino-6-(9H-fluoren-9-ylmethoxycarbonylamino)-hexanoic acid as described in the method of Example 6. Purification by HPLC produced the title compound. 1H NMR (400 MHz, DMSO-d₆) δ 10.40 (s, 1H), 8.23 (d, J=2 Hz, 3H), 7.88 (d, J=8 Hz, 2H), 7.78 (br s, 1H), 7.65 (d, J=7 Hz, 2H), 7.51 (d, J=8 Hz, 2H), 7.42-7.29 (m, 5H), 7.23 (d, J=8 Hz, 2H), 6.88 (br s, 1H), 6.58 (br s, 1H), 4.63 (br s, 2H), 4.32 (m, 2H), 4.19 (t, J=7 Hz, 1H), 4.10 (br s, 1H), 3.88 (m, 1H), 3.10 (dd, J=7, 13 Hz, 2H), 1.93 (d, J=7 Hz, 2H), 1.69-1.62 (m, 4H), 1.54-1.43 (m, 3H), 1.25-1.16 (m, 2H), 1.06-0.97 (m, 1H); Low resolution mass spectrum (ES) m/e 621 [(M+H)⁺, calcd for C₃₇H₄₁N₄O₅: 621]; 96.6% purity based on HPLC.

### Example 15

### (S)-[4-AMINO-1-(4-{[CYCLOHEXYL-(FURAN-2-CARBONYL)-AMINO]-METHYL}-PHENYLCARBAMOYL)-BUTYL]-CARBAMIC ACID 9H-FLUOREN-9-YLMETHYL ESTER

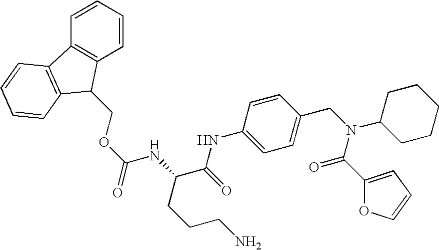

Furan-2-carboxylic acid (4-amino-benzyl)-cyclohexyl-amide was coupled to (S)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.06 (s, 1H), 7.89 (d, J=7.5 Hz, 2H), 7.79 (m, 1H), 7.74-7.65 (m, 6H), 7.53 (d, J=8.4 Hz, 2H), 7.40 (dt, J=3, 7.4 Hz, 2H), 7.31 (dd, J=6.7, 13.5 Hz, 2H), 7.20 (d, J=8.4 Hz, 2H), 6.88 (br s, 1H), 6.58 (br s, 1H), 4.63 (br s, 2H), 4.35-4.12 (m, 5H), 2.81-2.71 (m, 2H), 1.71-1.41 (m, 11H), 1.26-1.17 (m, 2H); Low resolution mass spectrum (ES) m/e 635 [(M+H)⁺, calcd for C₃₈H₄₃N₄O₅: 635]; 97.7% purity based on HPLC.

### Example 16

### (R)-[4-AMINO-1-(4-{[CYCLOHEXYL-(FURAN-2-CARBONYL)-AMINO]-METHYL}-PHENYLCARBAMOYL)-BUTYL]-CARBAMIC ACID 9H-FLUOREN-9-YLMETHYL ESTER

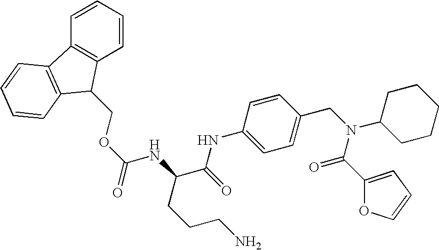

Furan-2-carboxylic acid (4-amino-benzyl)-cyclohexyl-amide was coupled to (R)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.06 (s, 1H), 7.89 (d, J=7.5 Hz, 2H), 7.79 (m, 1H), 7.74-7.66 (m, 6H), 7.53 (d, J=8.4 Hz, 2H), 7.40 (dt, J=2.5, 7.3 Hz, 2H), 7.31 (dd, J=6.7, 13.5 Hz, 2H), 7.20 (d, J=8.4 Hz, 2H), 6.88 (br s, 1H), 6.57 (br s, 1H), 4.63 (br s, 2H), 4.35-4.12 (m, 5H), 2.81-2.77 (m, 2H), 1.70-1.44 (m, 11H), 1.26-1.17 (m, 2H); Low resolution mass spectrum (ES) m/e 635 [(M+H)⁺, calcd for C₃₈H₄₃N₄O₅: 635]; 97.7% purity based on HPLC.

### Example 17

### (S)-[2-AMINO-2-(4-{[CYCLOHEXYL-(FURAN-2-CARBONYL)-AMINO]-METHYL}-PHENYLCARBAMOYL)-ETHYL]-CARBAMIC ACID 9H-FLUOREN-9-YLMETHYL ESTER

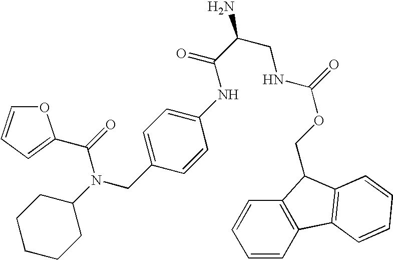

Furan-2-carboxylic acid (4-amino-benzyl)-cyclohexyl-amide was coupled to (S)-2-tert-Butoxycarbonylamino-3-(9H-fluoren-9-ylmethoxycarbonylamino)-propionic acid as described in the method of Example 6. Purification by HPLC produced the title compound. 1H NMR (400 MHz, DMSO-d₆) δ 10.42 (s, 1H), 8.26 (br s, 3H), 7.87 (d, J=7.5 Hz, 2H), 7.79 (br s, 1H), 7.63 (d, J=7.5 Hz, 2H), 7.58 (t, J=6 Hz, 1H), 7.48 (d, J=8.4 Hz, 2H), 7.39 (t, J=7.5 Hz, 2H), 7.27 (t, J=7.5 Hz, 2H), 7.20 (d, J=8.4 Hz, 2H), 6.89 (br s, 1H), 6.58 (br s, 1H), 4.63 (br s, 2H), 4.32-4.23 (m, 2H), 4.18 (t, J=6.7 Hz, 2H), 4.10 (m, 1H), 3.96 (m, 1H), 3.58-3.41 (m, 2H), 1.68-1.42 (m, 7H), 1.24-1.16 (m, 2H), 1.06-0.96 (m, 1H); Low resolution mass spectrum (ES) m/e 607 [(M+H)⁺, calcd for C₃₆H₃₉N₄O₅: 607]; 97.1% purity based on HPLC.

### Example 18

### [(4-{[CYCLOHEXYL-(FURAN-2-CARBONYL)-AMINO]-METHYL}-PHENYLCARBAMOYL)-METHYL]-CARBAMIC ACID 9H-FLUOREN-9-YLMETHYL ESTER

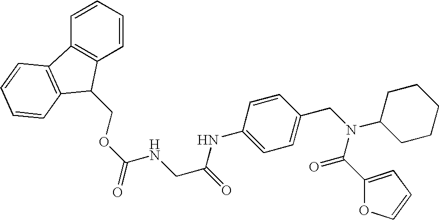

Furan-2-carboxylic acid (4-amino-benzyl)-cyclohexyl-amide was coupled to (9H-Fluoren-9-ylmethoxycarbonylamino)-acetic acid as described in the method for Intermediate #1 of Example 1. Purification by HPLC produced the title compound. 1H NMR (400 MHz, DMSO-d₆) δ 9.93 (s, 1H), 7.89 (d, J=7.5 Hz, 2H), 7.82 (m, 1H), 7.72 (d, J=7.4 Hz, 2H), 7.63-7.58 (m, 1H), 7.50 (t, J=8.4 Hz, 2H), 7.41 (t, J=7.4 Hz, 2H), 7.32 (t, J=7.4 Hz, 2H), 7.18 (d, J=8.4 Hz, 2H), 6.88 (br s, 1H), 6.57 (br s, 1H), 4.63 (br s, 2H), 4.30-4.18 (m, 3H), 4.10 (m, 1H), 3.77 (d, J=6 Hz, 2H), 1.70-1.62 (m, 4H), 1.51-1.44 (m, 3H), 1.25 (m, 2H), 1.07-0.96 (m, 1H); Low resolution mass spectrum (ES) m/e 578 [(M+H)⁺, calcd for C₃₅H₃₆N₃O₅: 578]; 98.4% purity based on HPLC.

### Example 19

### (S)-(5-AMINO-1-PHENYLCARBAMOYL-PENTYL)-CARBAMIC ACID 9H-FLUOREN-9-YLMETHYL ESTER

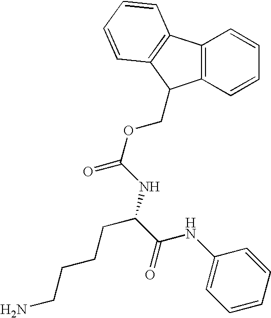

Aniline was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 10.03 (s, 1H), 7.89 (d, J=7.5 Hz, 2H), 7.73 (t, J=7.6 Hz, 2H), 7.64 (m, 4H), 7.60 (d, J=7.9 Hz, 2H), 7.41 (t, J=7.2 Hz, 2H), 7.34-7.28 (m, 4H), 7.04 (t, J=7.4 Hz, 1H), 4.33-4.20 (m, 3H), 4.15-4.10 (m, 1H), 2.77 (m, 2H), 1.74-1.27 (m, 6H); Low resolution mass spectrum (ES) m/e 444 [(M+H)⁺, calcd for C₂₇H₃₀N₃O₃: 444]; 95% purity based on HPLC.

### Example 20

### (S)-(5-AMINO-1-P-TOLYLCARBAMOYL-PENTYL)-CARBAMIC ACID 9H-FLUOREN-9-YLMETHYL ESTER

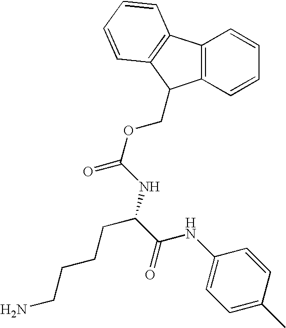

p-Tolylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.93 (s, 1H), 7.89 (d, J=7.5 Hz, 2H), 7.72 (t, J=7.6 Hz, 2H), 7.61 (m, 4H), 7.47 (d, J=8.3 Hz, 2H), 7.41 (t, J=7.3 Hz, 2H), 7.34-7.29 (m, 2H), 7.10 (d, J=8.3 Hz, 2H), 4.33-4.20 (m, 3H), 4.13-4.08 (m, 1H), 2.76 (m, 2H), 2.24 (s, 3H), 1.73-1.28 (m, 6H); Low resolution mass spectrum (ES) m/e 458 [(M+H)⁺, calcd for C₂₈H₃₂N₃O₃: 458]; 95% purity based on HPLC.

### Example 21

### (S)-(5-AMINO-1-BENZYLCARBAMOYL-PENTYL)-CARBAMIC ACID 9H-FLUOREN-9-YLMETHYL ESTER

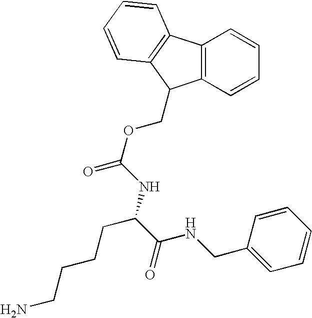

Benzylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.41 (t, J=5.9 Hz, 1H), 7.89 (d, J=7.5 Hz, 2H), 7.72 (t, J=6.7 Hz, 2H), 7.65 (br s, 3H), 7.51 (d, J=8.2 Hz, 1H), 7.41 (d, J=7.4 Hz, 2H), 7.33-7.20 (m, 7H), 4.32-4.19 (m, 5H), 4.02-3.97 (m, 1H), 2.75 (m, 2H), 1.70-1.22 (m, 6H); Low resolution mass spectrum (ES) m/e 458 [(M+H)⁺, calcd for C₂₈H₃₂N₃O₃: 458]; 95% purity based on HPLC.

### Example 22

### (S)-(5-AMINO-1-CYCLOHEXYLCARBAMOYL-PENTYL)-CARBAMIC ACID 9H-FLUOREN-9-YLMETHYL ESTER

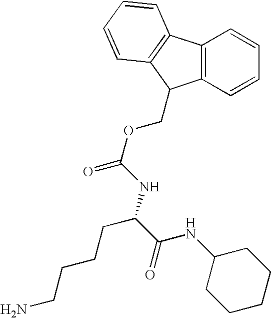

Cyclohexylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 7.89 (d, J=7.5 Hz, 2H), 7.73-7.65 (m, 6H), 7.40 (dd, J=8.1, 15.7 Hz, 3H), 7.31 (t, J=7 Hz, 2H), 4.30-4.18 (m, 3H), 3.92 (dd, J=8.5, 14.1 Hz, 1H), 3.53-3.46 (m, 1H), 2.75 (m, 2H), 1.70-1.46 (m, 9H), 1.35-1.07 (m, 7H); Low resolution mass spectrum (ES) m/e 450 [(M+H)⁺, calcd for C₂₇H₃₆N₃O₃: 450]; 96% purity based on HPLC.

### Example 23

### (S)-FURAN-2-CARBOXYLIC ACID (4-{6-AMINO-2-[2-(S)-AMINO-3-(1H-INDOL-3-YL)-PROPIONYLAMINO]-HEXANOYLAMINO}-BENZYL)-CYCLOHEXYL-AMIDE

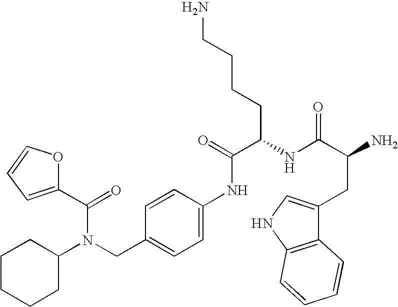

Furan-2-carboxylic acid (4-amino-benzyl)-cyclohexyl-amide was coupled to (S)-6-tert-butoxycarbonylamino-2-(9H-fluoren-9-ylmethoxycarbonylamino)-hexanoic acid as described in the method of Example 5, followed by coupling to (S)-2-tert-Butoxycarbonylamino-3-(1H-indol-3-yl)-propionic acid as described in the method of Example 6. Purification by HPLC produced the title compound. 1H NMR (400 MHz, DMSO-d₆) δ 11.00 (d, J=1.8 Hz, 1H), 10.19 (s, 1H), 8.91 (d, J=7.9 Hz, 1H), 8.02 (m, 3H), 7.80 (m, 1H), 7.71 (br s, 2.4H), 7.66 (d, J=7.9 Hz, 1H), 7.55 (d, J=8.5 Hz, 2H), 7.32 (d, J=8.14 Hz, 1H), 7.23 (m, 3H), 7.02 (t, J=7.5 Hz, 1H), 6.89 (t, J=7.4 Hz, 2H), 6.56 (m, 1H), 4.65 (br s, 2H), 4.48 (dd, J=7.8, 13.8 Hz, 1H), 4.09 (m, 2H), 3.25 (dd, J=4.9, 15 Hz, 1H), 3.06 (dd, J=8.2, 15 Hz, 1H), 2.76 (m, 2H), 1.76-1.18 (m, 15H), 1.09-0.99 (m, 1H); Low resolution mass spectrum (ES) m/e 612 [(M+H)⁺, calcd for C₃₅H₄₅N₆O₄: 612]; 96% purity based on HPLC.

### Example 24

### (S)-FURAN-2-CARBOXYLIC ACID (4-{6-AMINO-2-[(NAPHTHALENE-1-CARBONYL)-AMINO]-HEXANOYLAMINO}-BENZYL)-CYCLOHEXYL-AMIDE

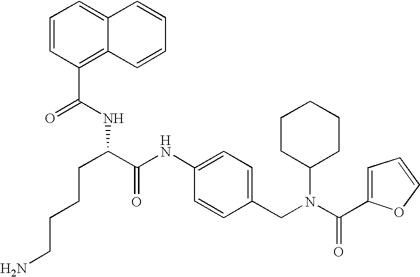

The title compound was prepared by coupling furan-2-carboxylic acid (4-amino-benzyl)-cyclohexyl-amide to (S)-6-tert-Butoxycarbonylamino-2-(9H-fluoren-9-ylmethoxycarbonylamino)-hexanoic acid, and then to naphthalene-1-carboxylic acid as described for the method of Example 23. Purification by HPLC produced the title compound. 1H NMR (400 MHz, DMSO-d₆) δ 10.17 (s, 1H), 8.74 (d, J=7.5 Hz, 1H), 8.23 (dd, J=3.5, 6.3 Hz, 1H), 8.02 (d, J=8.2 Hz, 1H), 7.97 (dd, J=3.3, 6.2 Hz, 1H), 7.79 (br s, 1H), 7.66 (m, 1H), 7.60-7.53 (m, 5H), 7.22 (d, J=8.5 Hz, 2H), 6.89 (br s, 1H), 6.58 (br s, 1H), 4.65-4.59 (m, 3H), 4.12 (m, 11H), 2.80 (p, J=6.1 Hz, 2H), 1.83-1.44 (m, 13H), 1.27-1.17 (m, 2H), 1.08-0.99 (m, 1H); Low resolution mass spectrum (ES) m/e 581 [(M+H)⁺, calcd for C₃₅H₄₁N₄O₄: 581]; 96.7% purity based on HPLC.

### Example 25

### (S)-FURAN-2-CARBOXYLIC ACID {4-[6-AMINO-2-(2-NAPHTHALEN-1-YL-ACETYLAMINO)-HEXANOYLAMINO]-BENZYL}-CYCLOHEXYL-AMIDE

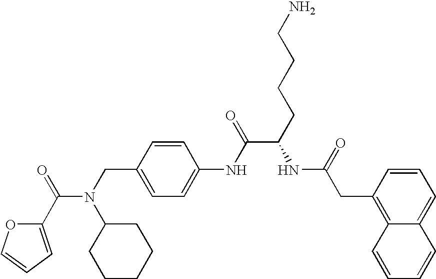

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then with naphthalen-1-yl-acetic acid as described in the method of Example 23. Purification by HPLC produced the title compound. 1H NMR (400 MHz, DMSO-d₆) δ 10.04 (s, 1H), 8.52 (d, J=8 Hz, 1H), 8.09 (m, 1H), 7.90 (m, 1H), 7.81 (m, 2H), 7.65 (m, 3H), 7.52-7.42 (m, 6H), 7.18 (d, J=8.5 Hz, 2H), 6.88 (br s, 1H), 6.57 (br s, 1H), 4.63 (m, 2H), 4.40 (m, 1H), 4.10 (m, 1H), 4.01 (d, J=15 Hz, 1H), 3.95 (d, J=15 Hz, 1H), 2.72 (m, 2H), 1.76-1.16 (m, 15H), 1.07-0.99 (m, 1H); Low resolution mass spectrum (ES) m/e 595 [(M+H)⁺, calcd for C₃₆H₄₃N₄O₄: 595]; 93.0% purity based on HPLC.

### Example 26

### (S)-FURAN-2-CARBOXYLIC ACID {4-[6-AMINO-2-(3-BIPHENYL-4-YL-UREIDO)-HEXANOYLAMINO]-BENZYL}-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid as described in the method of Example 5. Coupling to 4-isocyanato-biphenyl was done as described in the method for Intermediate #3 of Example 1, and then final deprotection as described in the method Example 2. Purification by HPLC produced the title compound. 1H NMR (400 MHz, DMSO-d₆) δ 10.17 (s, 1H), 8.85 (s, 1H), 7.78 (br s, 1H), 7.66 (br s, 3H), 7.60 (d, J=7.3 Hz, 2H), 7.54 (d, J=8.8 Hz, 4H), 7.48 (d, J=8.8 Hz, 2H), 7.41 (t, J=7.7 Hz, 2H), 7.28 (t, J=7.3 Hz, 1H), 7.20 (d, J=8.5 Hz, 2H), 6.88 (br s, 1H), 6.60 (m, 2H), 4.63 (br s, 2H), 4.40 (dd, J=7.9, 13.4 Hz, 1H), 4.11 (m, 1H), 2.82-2.74 (m, 2H), 1.79-1.31 (m, 13H), 1.26-1.16 (m, 2H), 1.07-0.98 (m, 1H); Low resolution mass spectrum (ES) m/e 622 [(M+H)⁺, calcd for C₃₇H₄₄N₅O₄: 622]; 98.0% purity based on HPLC.

### Example 27

### (S)-FURAN-2-CARBOXYLIC ACID [4-(6-AMINO-2-BENZOYLAMINO-HEXANOYLAMINO)-BENZYL]-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then benzoic acid as described in the method of Example 23, and purification by HPLC produced the title compound. 1H NMR (400 MHz, DMSO-d₆) δ 10.09 (s, 1H), 8.57 (d, J=7.7 Hz, 1H), 7.90 (d, J=8.6 Hz, 2H), 7.78 (br s, 1H), 7.64 (br s, 3H), 7.55 (m, 3H), 7.47 (t, J=7.4 Hz, 2H), 7.19 (d, J=8.5 Hz, 2H), 6.88 (br s, 1H), 6.57 (br s, 1H), 4.63 (br s, 2H), 4.55 (dd, J=7.4, 14.8 Hz, 1H), 4.10 (m, 1H), 2.81-2.76 (m, 2H), 1.81 (dd, J=7.6, 14.8 Hz, 2H), 1.0-1.35 (m, 11H), 1.26-1.16 (m, 2H), 1.07-0.97 (m, 1H); Low resolution mass spectrum (ES) m/e 531 [(M+H)⁺, calcd for C₃₁H₃₉N₄O₄: 531]; 99.0% purity based on HPLC.

### Example 28

### (S)-[1-(4-{[CYCLOHEXYL-(FURAN-2-CARBONYL)-AMINO]-METHYL}-PHENYLCARBAMOYL)-PROPYL]-CARBAMIC ACID 9H-FLUOREN-9-YLMETHYL ESTER

Furan-2-carboxylic acid (4-amino-benzyl)-cyclohexyl-amide was coupled to (S)-2-(9H-Fluoren-9-ylmethoxycarbonylamino)-butyric acid as described in the method for Intermediate #1 of Example 1. Purification by HPLC produced the title compound. ¹H NMR (400 MHz, DMSO-d₆) δ 9.99 (s, 1H), 7.88 (d, J=7.5 Hz, 2H), 7.78 (br m, 1H), 7.73 (t, J=7.3 Hz, 2H), 7.62 (d, J=7.9 Hz, 1H), 7.52 (d, J=8.4 Hz, 2H), 7.40 (dt, J=2.8, 7.4 Hz, 2H), 7.31 (dt, J=4.3, 7.3 Hz, 2H), 6.87 (br s, 1H), 6.57 (br s, 1H), 4.3 (br s, 2H), 4.27-4.02 (m, 5H), 1.75-1.43 (m, 9H), 1.25-1.16 (m, 2H), 1.07-0.95 (m, 1H); Low resolution mass spectrum (ES-m/e 606 [(M+H)⁺, calcd for C₃₇H₄₀N₃O₅: 606]; 97.7% purity based on HPLC.

### Example 29

### (S)-[1-(4-{[CYCLOHEXYL-(FURAN-2-CARBONYL)-AMINO]-METHYL}-PHENYL CARBAMOYL)-2-PHENYL-ETHYL]-CARBAMIC ACID 9H-FLUOREN-9-YLMETHYL ESTER

Furan-2-carboxylic acid (4-amino-benzyl)-cyclohexyl-amide was coupled to (S)-2-(9H-fluoren-9-ylmethoxycarbonylamino)-3-phenyl-propionic acid as described in the method for Intermediate #1 of Example 1. Purification by HPLC produced the title compound. 1H NMR (400 MHz, DMSO-d₆) δ 10.10 (s, 1H), 7.86 (d, J=7.5 Hz, 2H), 7.80 (m, 2H), 7.65 (t, J=7.7 Hz, 2H), 7.52 (d, J=8.4 Hz, 2H), 7.41-7.17 (m, 11H), 6.88 (br s, 1H), 6.57 (br s, 1H), 4.64 (br s, 2H), 4.42-4.36 (m, 1H), 4.18-4.09 (m, 4H), 3.02 (dd, J=4.4, 13.6 Hz, 1H), 2.88 (dd, J=10.4, 13.5 Hz, 1H), 1.70-1.44 (m, 7H), 1.25-1.16 (m, 2H), 1.07-0.98 (m, 1H); Low resolution mass spectrum (ES) m/e 668 [(M+H)⁺, calcd for C₄₂H₄₂N₃O₅: 668]; 96.7% purity based on HPLC.

### Example 30

### (S)-2-[6-TERT-BUTOXYCARBONYLAMINO-2-(S)-((9H-FLUOREN-9-YLMETHOXYCARBONYL)AMINO)-HEXANOYL-AMINO]-3-(1H-INDOL-3-YL)-PROPIONIC ACID METHYL ESTER

(S)-2-amino-3-(1H-indol-3-yl)-propionic acid methyl ester was coupled to (S)-6-tert-Butoxycarbonylamino-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 10.84 (s, 0.5H), 8.27 (d, J=7.2 Hz, 1H), 7.86 (d, J=7.5 Hz, 2H), 7.69 (dd, J=5.7, 6.8 Hz, 2H), 7.45 (d, J=7.8 Hz, 1H), 7.39 (t, J=7.4 Hz, 2H), 7.34-7.26 (m, 2H), 7.14 (s, 1H), 7.04 (t, J=7.4 Hz, 1H), 6.96 (t, J=7.5 Hz, 1H), 4.48 (dd, J=7.3, 13.7 Hz, 1H), 4.31-4.12 (m, 3H), 4.04-3.94 (m, 1H), 3.52 (s, 1H), 3.54-3.44 (m, 2H); Low resolution mass spectrum (ES) m/e 669 [(M+H)⁺, calcd for C₃₈H₄₅N₄O₇: 669]; 90% purity based on NMR.

### Example 31

### (S)-FURAN-2-CARBOXYLIC ACID {4-[6-AMINO-2-(3-NAPHTHALEN-1-YL-UREIDO)-HEXANOYLAMINO]-BENZYL}-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid then to 1-isocyanato-naphthalene as described in the method of Example 26. Purification by HPLC produced the title compound. 1H NMR (400 MHz, DMSO-d₆) δ 10.20 (s, 1H), 8.76 (s, 1H), 8.51 (s, 1H), 8.12 (d, J=8.2 Hz, 1H), 8.03 (d, J=6.9 Hz, 1H), 7.89 (m, 2H), 7.78 (br s, 1H), 7.70 (d, J=8.1 Hz, 1H), 7.65 (br s, 2.5H), 7.57-7.37 (m, 9H), 7.20 (d, J=8.5 Hz, 2H), 7.09 (d, J=8.2 Hz, 1H), 6.88 (br s, 1H), 6.58 (br s, 1H), 4.64 (br s, 2H), 4.46 (dd, J=7.8, 13.6 Hz, 1H), 4.11 (m, 1H), 3.47 (m, 2H), 2.83-2.75 (m, 2H), 1.81-1.40 (m, 15H), 1.26-1.17 (m, 2H), 1.07-0.98 (m, 1H); Low resolution mass spectrum (ES) m/e 596 [(M+H)⁺, calcd for C₃₅H₄₂N₅O₄: 596]; 83% purity based on HPLC.

### Example 32

### (R)-FURAN-2-CARBOXYLIC ACID {4-[6-AMINO-2-(3-NAPHTHALEN-1-YL-UREIDO)-HEXANOYLAMINO]-BENZYL}-CYCLOHEXYL-AMIDE

Furan-2-carboxylic acid (4-amino-benzyl)-cyclohexyl-amide was coupled to (R)-2-(9H-Fluoren-9-ylmethoxycarbonylamino)-butyric acid, and then to 1-isocyanato-naphthalene as described in the method of Example 26. Purification by HPLC produced the title compound. 1H NMR (400 MHz, DMSO-d₆) δ 9.99 (s, 1H), 7.88 (d, J=8.2 Hz, 1H), 8.03 (d, J=7.59 Hz, 2H), 7.78 (m, 1H), 7.73 (t, J=7.3 Hz, 2H), 7.62 (d, J=7.9 Hz, 1H), 7.52 (d, J=8.4 Hz, 2H), 7.40 (dt, J=7.4, 7.8 Hz, 2H), 7.31 (dt, J=4.3, 7.3 Hz, 2H), 7.18 (d, J=8.4 Hz, 2H), 6.87 (br s, 1H), 6.57 (br s, 1H), 4.63 (br s, 2H), 4.27-4.02 (m, 5H), 1.75-1.59 (m, 6H), 1.53-1.43 (m, 3H), 1.25-1.16 (m, 2H), 1.07-0.98 (m, 1H), 0.91 (t, J=7.3 Hz, 3H); Low resolution mass spectrum (ES) m/e 606 [(M+H)⁺, calcd for C₃₇H₄₀N₃O₅: 606]; 95% purity based on HPLC.

### Example 33

### (S)-[5-AMINO-1-(BENZYL-CYCLOHEXYL-CARBAMOYL)-PENTYL]-CARBAMIC ACID 9H-FLUOREN-9-YLMETHYL ESTER

Benzyl-cyclohexyl-amine was coupled to (S)-6-tert-Butoxycarbonylamino-2-(9H-fluoren-9-ylmethoxycarbonylamino)-hexanoic acid as described in the method of Example 23. Purification by HPLC produced the title compound. 1H NMR (400 MHz, DMSO-d₆) δ 7.88 (d, J=7.6 Hz, 2H), 7.7 (d, J=7.4 Hz, 2H), 7.4 (t, J=7.4 Hz, 2H), 7.35-7.18 (m, 5H), 7.18-7.10 (m, 2H), 4.66-4.36 (m, 3H), 4.33-4.05 (m, 4H), 3.8-3.67 (m, 1H), 2.76 (t, J=7.5 Hz, 1H), 2.62 (t, J=7.5 Hz, 1H), 1.72-0.89 (m, 16H); Low resolution mass spectrum (ES) m/e 540 [(M+H)⁺, calcd for C₃₄H₄₂N₃O₃: 540]; 95% purity based on HPLC.

### Example 34

### (S)-[5-AMINO-5-(BENZYL-CYCLOHEXYL-CARBAMOYL)-PENTYL]-CARBAMIC ACID TERT-BUTYL ESTER

Benzyl-cyclohexyl-amine was coupled to (S)-6-tert-Butoxycarbonylamino-2-(9H-fluoren-9-ylmethoxycarbonylamino)-hexanoic acid as described in the method of Example 5. Purification by HPLC produced the title compound. 1H NMR (400 MHz, DMSO-d₆) δ 7.39-7.23 (m, 3H), 7.22-7.14 (m, 2H), 4.66-4.39 (m, 3H), 4.15-4.01 (m, 0.5H), 3.96-3.84 (m, 0.5H), 3.67-3.46 (m, 2H), 2.94-2.64 (m, 2H), 1.82-0.91 (m, 25H); Low resolution mass spectrum (ES) m/e 418 [(M+H)⁺, calcd for C₂₄H₄₀N₃O₃: 418]; 90% purity based on HPLC.

### Example 35

### (S)-[5-[2-(S)-AMINO-3-(1H-INDOL-3-YL)-PROPIONYLAMINO]-1-(4-{[CYCLOHEXYL-(FURAN-2-CARBONYL)-AMINO]-METHYL}-PHENYLCARBAMOYL)-PENTYL]-CARBAMIC ACID 9H-FLUOREN-9-YLMETHYL ESTER

(S)-[4-Amino-1-(4-{[cyclohexyl-(furan-2-carbonyl)-amino]-methyl}-phenylcarbamoyl)-butyl]-carbamic acid 9H-fluoren-9-ylmethyl ester from Example 15 was coupled to (S)-2-tert-Butoxycarbonylamino-3-(1H-indol-3-yl)-propionic acid as described in the method of Example 6. Purification by HPLC produced the title compound. 1H NMR (400 MHz, DMSO-d₆) δ 11 (s, 1H), 10 (s, 1H), 8.43 (t, J=5.2 Hz, 1H), 8.03 (app d, J=3.5 Hz, 2H), 7.88 (app d, J=7.5 Hz, 2H), 7.78 (br s, 1H), 7.70 (dd, J=5.2, 6.8 Hz, 2H), 7.62 (d, J=7.7 Hz, 2H), 7.52 (d, J=8.3 Hz, 2H), 7.44-7.25 (m, 5H), 7.23-7.13 (m, 3H), 7.07 (t, J=7.4 Hz, 1H), 6.99 (t, J=7.4 Hz, 1H), 6.87 (br s, 1H), 6.57 (br s, 1H), 4.62 (br s, 2H), 4.3-3.7 (m, 4H), 3.23-3 (m, 5H), 1.78-0.98 (m, 16H); Low resolution mass spectrum (ES) m/e 835 [(M)⁺, calcd for C₅₀H₅₄N₆O₆: 835]; 95% purity based on HPLC.

### Example 36

### (R)-(5-AMINO-1-P-TOLYLCARBAMOYL-PENTYL)-CARBAMIC ACID 9H-FLUOREN-9-YLMETHYL ESTER

p-Tolylamine was coupled to (R)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.94 (s, 1H), 7.90 (d, J=7.6 Hz, 2H), 7.74 (t, J=7.6 Hz, 2H), 7.66 (br s, 4H), 7.49 (d, J=8.4 Hz, 2H), 7.42 (t, J=7.4 Hz, 2H), 7.33 (m, 2H), 7.11 (d, J=8.3 Hz, 2H), 4.34-4.09 (m, 4H), 2.78 (m, 2H), 2.25 (s, 3H), 1.74-1.49 (m, 4H), 1.43-1.29 (m, 2H); Low resolution mass spectrum (ES) m/e 458 [(M)⁺, calcd for C₂₈H₃₁N₃O₃: 458]; 90% purity based on HPLC.

### Example 37

### (S)-[1-(4-{[CYCLOHEXYL-(FURAN-2-CARBONYL)-AMINO]-METHYL}-PHENYLCARBAMOYL)-PENTYL]-CARBAMIC ACID 9H-FLUOREN-9-YLMETHYL ESTER

Furan-2-carboxylic acid (4-amino-benzyl)-cyclohexyl-amide was coupled to (S)-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 10.0 (s, 1H), 7.89 (d, J=7.5 Hz, 2H), 7.79 (br s, 1H), 6.74 (t, J=7.0 Hz, 2H), 7.64 (d, J=8.0 Hz, 1H), 7.54 (d, J=8.4 Hz, 2H), 7.41 (dt, J=3.4, 7.4 Hz, 2H), 7.31 (dt, J=3.5, 7.4 Hz, 2H), 7.19 (d, J=8.4 Hz, 2H), 6.88 (br s, 1H), 6.58 (br s, 1H), 4.64 (br s, 2H), 4.3-4.08 (m, 5H), 1.71-1.44 (m, 9H), 1.39-1.17 (m, 6H), 1.08-0.98 (m, 1H), 0.87 (t, J=6.8 Hz, 3H); Low resolution mass spectrum (ES) m/e 634 [(M+1)⁺, calcd for C₃₉H₄₄N₃O₅: 634]; 95% purity based on HPLC.

### Example 38

### (S)-[5-AMINO-1-(2-METHYL-1H-INDOL-5-YLCARBAMOYL)-PENTYL]-CARBAMIC ACID 9H-FLUOREN-9-YL METHYL ESTER

2-Methyl-1H-indol-5-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 10.6 (s, 1H), 9.72 (s, 1H), 7.89 (d, J=7.5 Hz, 2H), 7.79-7.53 (m, 6H), 7.41 (ddd, J=2.6, 7.3, 7.4 Hz, 2H), 7.37-7.27 (m, 2H), 7.22-7.04 (m, 2H), 6.05 (s, 1H), 4.35-4.18 (m, 3H), 4.13 (dt, J=5.4, 8.3 Hz, 1H), 2.78 (dq, J=5.9, 12 Hz, 2H), 2.34 (s, 3H), 1.78-1.27 (m, 6H); Low resolution mass spectrum (ES) m/e 497 [(M+1)⁺, calcd for C₃₀H₃₃N₄O₃: 497]; 96.4% purity based on HPLC.

### Example 39

### 3-{4-[2-((9H-FLUOREN-9-YLMETHOXYCARBONYL)AMINO)-2-METHYL-PROPIONYLAMINO]-PHENYL}-ACRYLIC ACID

3-(4-Amino-phenyl)-acrylic acid methyl ester was coupled to 2-(9H-Fluoren-9-ylmethoxycarbonylamino)-2-methyl-propionic acid as described in the method for Intermediate #1 of Example 1. Ester hydrolysis (0.1 N sodium hydroxide in methanol for 12 hours) was performed, and then purification by HPLC produced the title compound. 1H NMR (400 MHz, DMSO-d₆) δ 9.61 (s, 1H), 7.87 (d, J=7.5 Hz, 2H), 7.75-7.65 (m, 4H), 7.58 (d, J=8.5 Hz, 2H), 7.5 (d, J=16 Hz, 1H), 7.39 (t, J=7.3 Hz, 2H), 7.3 (t, J=6.8 Hz, 2H), 6.39 (d, J=16 Hz, 1H), 4.27 (app q, J=6.9 Hz, 2H), 4.19 (t, J=6.4 Hz, 1H), 1.41 (s, 6H); Low resolution mass spectrum (ES) m/e 471 [(M+1)⁺, calcd for C₂₈H₂₇N₂O₅: 471]; 86% purity based on HPLC.

### Example 40

### (S)-6-AMINO-2-(3-BIPHENYL-4-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 4-isocyanato-biphenyl as described in the method of Example 26. Purification by HPLC produced the title compound. 1H NMR (400 MHz, DMSO-d₆) δ 10.10 (s, 1H), 8.85 (s, 1H), 7.67 (br s, 3H), 7.61 (d, J=7.3 Hz, 2H), 7.56 (d, J=8.7 Hz, 2H), 7.49 (dd, J=8.6, 10.4 Hz, 4H), 7.42 (t, J=7.4 Hz, 2H), 7.30 (t, J=7.3 Hz, 1H), 7.12 (d, J=8.4 Hz, 2H), 6.59 (d, J=8.2 Hz, 1H), 4.42 (dd, J=8, 13.5 Hz, 1H), 2.79 (dd, J=6.8, 12.7 Hz, 2H), 2.25 (s, 3H), 1.8-1.71 (m, 1H), 1.67-1.52 (m, 3H), 1.42-1.32 (m, 2H); Low resolution mass spectrum (ES) m/e 431 [(M+1)⁺, calcd for C₂₆H₃₁N₄O₂: 431]; 99.9% purity based on HPLC.

### Example 41

### (S)-6-AMINO-2-[3-(4-PHENOXY-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-isocyanato-4-phenoxy-benzene as described in the method of Example 26. Purification by HPLC produced the title compound. 1H NMR (400 MHz, DMSO-d₆) δ 10.08 (s, 1H), 8.76 (s, 1H), 7.67 (br s, 3H), 7.50 (d, J=8.4 Hz, 2H), 7.40 (d, J=9.0 Hz, 2H), 7.33 (dd, J=7.5, 8.5 Hz, 2H), 7.11 (d, J=8.4 Hz, 2H), 7.06 (t, J=7.4 Hz, 1H), 6.92 (t, J=8.3 Hz, 4H), 6.52 (d, J=8.3 Hz, 1H), 4.38 (dd, J=8, 13.5 Hz, 1H), 2.82-2.74 (m, 2H), 2.24 (s, 3H), 1.77-1.69 (m, 1H), 1.64-1.52 (m, 3H), 1.45-1.30 (m, 2H); Low resolution mass spectrum (ES) m/e 447 [(M+1)⁺, calcd for C₂₆H₃₁N₄O₃: 447]; 99.7% purity based on HPLC.

### Example 42

### (S)-6-AMINO-2-[3-(4-TRIFLUOROMETHYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-isocyanato-4-trifluoromethyl-benzene as described in the method of Example 26. Purification by HPLC produced the title compound. 1H NMR (400 MHz, DMSO-d₆) δ 10.10 (s, 1H), 9.20 (s, 1H), 7.67 (br s, 3H), 7.58 (s, 4H), 7.49 (d, J=8.4 Hz, 2H), 7.12 (dd, J=8.4 Hz, 2H), 6.74 (d, J=8.1 Hz, 1H), 4.40 (dd, J=7.9, 13.4 Hz, 1H), 2.78 (br s, 2H), 2.25 (s, 3H), 1.8-1.71 (m, 1H), 1.67-1.52 (m, 3H), 1.46-1.32 (m, 2H); Low resolution mass spectrum (ES) m/e 423 [(M+1)⁺, calcd for C₂₁H₂₆F₃N₄O₂: 423]; 99.2% purity based on HPLC.

### Example 43

### (S)-6-AMINO-2-[3-(4-FLUORO-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-fluoro-4-isocyanato-benzene as described in the method of Example 26. Purification by HPLC produced the title compound. 1H NMR (400 MHz, DMSO-d₆) δ 10.08 (s, 1H), 8.76 (s, 1H), 7.65 (br s, 3H), 7.49 (d, J=8.4 Hz, 2H), 7.39 (m, 2H), 7.12 (d, J=8.4 Hz, 2H), 7.06 (t, J=8.9 Hz, 2H), 6.51 (d, J=8.3 Hz, 1H), 4.38 (dd, J=8.0, 13.5 Hz, 1H), 2.81-2.75 (m, 2H), 2.25 (s, 3H), 1.78-1.72 (m, 7H); Low resolution mass spectrum (ES) m/e 373 [(M+1)⁺, calcd for C₂₀H₂₆FN₄O₂: 373]; 90% purity based on HPLC.

### Example 44

### (S)-[5-AMINO-1-(4-FLUORO-PHENYLCARBAMOYL)-PENTYL]-CARBAMIC ACID 9H-FLUOREN-9-YLMETHYL ESTER

p-Fluorophenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d6) δ 10.1 (s, 1H); 7.89 (d, J=7.55, 2H); 7.72 (t, J=7.61 Hz, 2H); 7.69-7.58 (m, 5H); 7.41 (t, J=7.40 Hz, 2H); 7.31 (ddd, J=4.31, 7.29, 7.32 Hz, 2H); 7.14 (t, J=8.87 Hz, 2H); 4.35-4.18 (m, 3H); 4.10 (ddd, J=5.93, 8.46, 8.38, 1H); 2.77 (dddd, J=5.95, 5.95, 6.10, 12.56 Hz, 2H); 1.75-1.47 (m, 4H); 1.47-1.24 (m, 2H); Low resolution mass spectrum (ES) m/e 462 [(M+H)⁺, calcd for C₂₇H₂₉FN₃O₃: 462]; 100% purity based on HPLC.

### Example 45

### (S)-(5-AMINO-1-O-TOLYLCARBAMOYL-PENTYL)-CARBAMIC ACID 9H-FLUOREN-9-YLMETHYL ESTER

o-Tolylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d6) δ 9.32 (s, 1H); 7.89 (d, J=7.52 Hz, 2H); 7.73 (t, J=6.67 Hz, 2H); 7.69-7.57 (m, 4H); 7.41 (t, J=7.42 Hz, 2H); 7.36 (d, J=7.70 Hz, 1H); 7.31 (ddd, J=1.88, 7.39, 7.40 Hz, 2H); 7.20 (d, J=7.39 Hz, 1H); 7.16 (t, J=7.54 Hz, 1H); 7.08 (ddd, J=0.82, 7.51, 7.66 Hz, 1H); 4.37-4.13 (m, 4H); 2.79 (dddd, J=7.51, 7.51, 6.76, 12.82 Hz, 2H); 2.16 (s, 3H); 1.84-1.50 (m, 4H); 1.50-1.31 (m, 2H); Low resolution mass spectrum (ES) m/e 458 [(M+H)⁺, calcd for C₂₈H₃₂N₃O₃: 458]; 99% purity based on HPLC.

### Example 46

### (S)-FURAN-2-CARBOXYLIC ACID (4-{6-AMINO-2-[2-(6-BENZOYLAMINO-PURIN-9-YL)-ACETYLAMINO]-HEXANOYLAMINO}-BENZYL)-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then to (6-Benzoylamino-purin-9-yl)-acetic acid as described in the method of Example 23. Purification by HPLC produced the title compound. 1H NMR (400 MHz, DMSO-d₆) δ 11.16 (br s, 1H), 10.14 (s, 1H), 8.82 (d, J=8.0 Hz, 1H), 8.7 (s, 1H), 8.43 (s, 1H), 8.05 (d, J=7.3 Hz, 2H), 7.79 (br s, 1H), 7.65 (m, 4H), 7.51 (m, 4H), 7.21 (d, J=8.5 Hz, 2H), 6.89 (br s, 1H), 6.59 (br s, 1H), 5.59 (s, 2H), 4.64 (br s, 2H), 4.47 (dd, J=8.1, 13.8 Hz, 1H), 4.12 (m, 1H), 2.79 (m, 2H), 1.80-1.31 (m, 13H), 1.27-1.17 (m, 2H), 1.02 (m, 1H); Low resolution mass spectrum (ES) m/e 706 [(M+1)⁺, calcd for C₃₈H₄₄N₉O₅: 706]; 98.7% purity based on HPLC.

### Example 47

### (S)-(5-AMINO-1-M-TOLYLCARBAMOYL-PENTYL)-CARBAMIC ACID 9H-FLUOREN-9-YLMETHYL ESTER

m-Tolylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.94 (s, 1H), 7.89 (d, J=7.5 Hz, 2H), 7.86 (d, J=7.5 Hz, 1H), 7.73 (app t, J=7.3 Hz, 2H), 7.68-7.57 (m, 3H), 7.46-7.28 (m, 6H), 7.17 (t, J=7.8 Hz, 1H), 4.34-4.18 (m, 3H), 4.11 (ddd, J=5.7, 8.4, 8.5 Hz, 1H), 2.77 (dddd, J=6.4, 6.4, 6.2, 12.5 Hz, 2H), 2.26 (s, 3H), 1.77-1.23 (m, 6H); Low resolution mass spectrum (ES) m/e 458 [(M+1)⁺, calcd for C₂₈H₃₂N₃O₃: 458]; 100% purity based on HPLC.

### Example 48

### (S)-6-AMINO-2-(2-NAPHTHALEN-2-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to naphthalen-2-yl-acetic acid as described in the method of Example 23. Purification by HPLC produced the title compound. 1H NMR (400 MHz, DMSO-d₆) δ 9.97 (s, 1H), 8.46 (d, J=8.0 Hz, 1H), 7.87-7.82 (m, 3H), 7.76 (s, 1H), 7.65 (br s, 3H), 7.5-7.43 (m, 5H), 7.09 (d, J=8.3 Hz, 2H), 4.4 (dt, J=5.8, 8.3 Hz, 1H), 3.67 (s, 2H), 2.77-2.69 (m, 2H), 2.23 (s, 3H), 1.77-1.48 (m, 4H), 1.42-1.22 (m, 2H); Low resolution mass spectrum (ES) m/e 404 [(M+1)⁺, calcd for C₂₅H₃₀N₃O₂: 404]; 95.9% purity based on HPLC.

### Example 49

### (S)-[1-(BENZYL-CYCLOHEXYL-CARBAMOYL)-2-(1H-INDOL-3-YL)-ETHYL]-CARBAMIC ACID 9H-FLUOREN-9-YLMETHYL ESTER

Benzyl-cyclohexyl-amine was coupled to (S)-2-(9H-Fluoren-9-ylmethoxycarbonylamino)-3-(1H-indol-3-yl)-propionic acid as described in the method for Intermediate #1 of Example 1. Purification by HPLC produced the title compound. 1H NMR (400 MHz, DMSO-d₆) δ 7.87 (d, J=7.6 Hz, 2H), 7.71 (d, J=7.4 Hz, 1H), 7.61 (d, J=7.7 Hz, 1H), 7.44-6.94 (m, 14H), 4.93 (dd, J=7.2, 7.7 Hz, 1H), 4.46 (d, J=16.2 Hz, 1H), 4.53-4.07 (m, 5H), 3.17 (dd, J=8.5, 14.0 Hz, 1H), 3.05 (dd, J=6.3, 14.1 Hz, 1H), 1.7-0.67 (m, 10H); Low resolution mass spectrum (ES) m/e 598 [(M+1)⁺, calcd for C₃₉H₄₀N₃O₃: 598]; 85% purity based on HPLC.

### Example 50

### (S)-6-AMINO-2-(2-BIPHENYL-4-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to biphenyl-4-yl-acetic acid as described in the method of Example 23. Purification by HPLC produced the title compound. 1H NMR (400 MHz, DMSO-d₆) δ 9.98 (s, 1H), 8.43 (d, J=8.0 Hz, 1H), 7.671 (br s, 3H), 7.64 (d, J=7.2 Hz, 2H), 7.59 (d, J=8.2 Hz, 2H), 7.49-7.43 (m, 4H), 7.37-7.33 (m, 3H), 7.10 (d, J=8.3 Hz, 2H), 4.41 (dd, J=8.3, 13.8 Hz, 1H), 3.55 (s, 2H), 2.78-2.73 (m, 2H), 2.24 (s, 3H), 1.78-1.5 (m, 4H), 1.43-1.23 (m, 2H); Low resolution mass spectrum (ES) m/e 430 [(M+1)⁺, calcd for C₂₇H₃₂N₃O₂: 430]; 98.6% purity based on HPLC.

### Example 51

### (S)-NAPHTHALENE-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to naphthalene-2-carboxylic acid as described in the method of Example 23. Purification by HPLC produced the title compound. 1H NMR (400 MHz, DMSO-d₆) δ 10.07 (s, 1H), 8.75 (d, J=7.8 Hz, 1H), 8.55 (s, 1H), 8.05-7.98 (m, 4H), 7.68 (br s, 3H), 7.65-7.59 (m, 2H), 7.52 (d, J=8.4 Hz, 2H), 7.12 (d, J=8.4 Hz, 2H), 4.64 (q, J=7.5 Hz, 1H), 2.81 (m 2H), 2.25 (s, 3H), 1.87 (dd, J=7.6, 14.8 Hz, 2H), 1.65-1.39 (m, 4H); Low resolution mass spectrum (ES) m/e 390 [(M+1)⁺, calcd for C₂₄H₂₈N₃O₂: 390]; 96.9% purity based on HPLC.

### Example 52

### (S)-9H-FLUORENE-9-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 9H-fluorene-9-carboxylic acid as described in the method of Example 23. Purification by HPLC produced the title compound. 1H NMR (400 MHz, DMSO-d₆) δ 10.04 (s, 1H), 8.84 (d, J=8.1 Hz, 1H), 7.88 (dd, J=5.0, 7.4 Hz, 2H), 7.71 (br s, 3H), 7.53 (t, J=7.4 Hz, 2H), 7.49 (d, J=8.4 Hz, 2H), 7.41 (dd, J=7.8, 16.6 Hz, 2H), 7.32 (dtd, J=0.9, 7.4, 11.3 Hz, 2H), 7.12 (d, J=8.4 Hz, 2H), 4.99 (s, 1H), 4.48 (dt, J=5.7, 8.4 Hz, 1H), 2.81 (m 2H), 2.25 (s, 3H); Low resolution mass spectrum (ES) m/e 429 [(M+1)⁺, calcd for C₂₇H₃₀N₃O₂: 429]; 98.6% purity based on HPLC.

### Example 53

### (S)-6-AMINO-2-(2-9H-FLUOREN-9-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (9H-Fluoren-9-yl)-acetic acid as described in the method of Example 23. Purification by HPLC produced the title compound. 1H NMR (400 MHz, DMSO-d₆) δ 10.09 (s, 1H), 8.32 (d, J=7.8 Hz, 1H), 7.87 (dd, J=6.0, 6.9 Hz, 2H), 7.70 (br s, 3H), 7.55 (m, 4H), 7.37 (dt, J=7.5, 10.1 Hz, 2H), 7.29 (dt, J=1.0, 7.5 Hz, 1H), 7.20 (dt, J=0.9, 7.5 Hz, 1H), 7.14 (d, J=8.3 Hz, 2H), 4.56 (dt, J=5.8, 8.2 Hz, 1H), 4.36 (t, J=7.5 Hz, 1H), 2.78 (m 2H), 2.62 (d, J=7.6 Hz, 2H); Low resolution mass spectrum (ES) m/e 443 [(M+1)⁺, calcd for C₂₈H₃₂N₃O₂: 443]; 97.4% purity based on HPLC.

### Example 54

### (S)-BIPHENYL-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to biphenyl-2-carboxylic acid as described in the method of Example 23. Purification by HPLC produced the title compound. 1H NMR (400 MHz, DMSO-d₆) δ 9.89 (s, 1H), 8.39 (d, J=7.7 Hz, 1H), 7.66 (br s, 3H), 7.53-7.4 (m, 8H), 7.33-7.24 (m, 3H), 7.11 (d, J=8.3 Hz, 2H), 4.32 (dt, J=5.5, 8.4 Hz, 1H), 2.71 (m, 2H), 2.25 (s, 3H), 1.65-1.39 (m, 4H), 1.20-1.08 (m, 2H); Low resolution mass spectrum (ES) m/e 417 [(M+1)⁺, calcd for C₂₆H₃₀N₃O₂: 417]; 100% purity based on HPLC.

### Example 55

### (+/−)-2-[6-AMINO-2-(S)-((9H-FLUOREN-9-YLMETHOXYCARBONYL)AMINO)-HEXANOYL-AMINO]-3-(1H-INDOL-3-YL)-PROPIONIC ACID METHYL ESTER

(S)-2-amino-3-(1H-indol-3-yl)-propionic acid methyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.26 (d, J=7.2 Hz, 0.6H), 7.86 (d, J=7.6 Hz, 2H), 7.67 (dd, J₁=7.9, 8.27 Hz, 2H), 7.44 (d, J=7.9 Hz, 1H), 7.39 (t, J=7.4 Hz, 2H), 7.34-7.25 (m, 3H), 7.12 (s, 1H), 7.05 (dd, J=7.5, 7.7 Hz, 1H), 6.96 (dd, J=7.1, 7.4 Hz, 1H), 4.53-4.43 (m, 1H), 4.26 (d, J=6.9 Hz, 2H), 4.21-4.14 (m, 1H), 3.99 (dd, J=5.1, 9.1 Hz, 1H), 3.52 (s, 3H), 3.12 (dd, J=6.0, 14.7 Hz, 1H), 3.05 (dd, J=7.6, 14.7 Hz, 1H), 2.70 (t, J=7.1 Hz, 2H), 1.63-1.40 (m, 4H), 1.33-1.14 (m, 2H); Low resolution mass spectrum (ES) m/e 570 [(M+1)⁺, calcd for C₃₃H₃₇N₄O₅: 570]; 100% purity based on HPLC.

### Example 56

### (S)-{5-((9H-FLUOREN-9-YLMETHOXYCARBONYL)AMINO)-5-[2-(1H-INDOL-3-YL)-ETHYLCARBAMOYL]-PENTYL}-CARBAMIC ACID TERT-BUTYL ESTER

2-(1H-Indol-3-yl)-ethylamine was coupled to (S)-6-tert-Butoxycarbonylamino-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 7.87 (d, J=7.5 Hz, 2H), 7.71 (d, J=7.1 Hz, 2H), 7.51 (d, J=8.1 Hz, 1H), 7.4 (t, J=7.4 Hz, 2H), 7.34-7.27 (m, 3H), 7.10 (s, 1H), 7.04 (dd, J=7.1, 7.5 Hz, 1H), 6.95 (dd, J=7.2, 7.5 Hz, 1H), 4.31-4.16 (m, 3H), 3.88 (dd, J=5.0, 9.0 Hz, 1H), 3.39-3.22 (m, 2H), 2.91-2.81 (m, 2H), 2.78 (t, J=7.4 Hz, 2H), 1.60-1.39 (m, 3H), 1.33 (s, 9H), 1.31-1.09 (m, 3H); Low resolution mass spectrum (ES) m/e 611 [(M+1)⁺, calcd for C₃₆H₄₃N₄O₅: 611]; 92% purity based on HPLC.

### Example 57

### (S)-{5-AMINO-1-[2-(1H-INDOL-3-YL)-ETHYLCARBAMOYL]-PENTYL}-CARBAMIC ACID 9H-FLUOREN-9-YLMETHYL ESTER

(S)-{5-((9H-Fluoren-9-ylmethoxycarbonyl)amino)-5-[2-(1H-indol-3-yl)-ethylcarbamoyl]-pentyl}-carbamic acid tert-butyl ester from Example 56 was deprotected as described in the method for Intermediate #2 of Example 1. Purification by HPLC produced the title compound. 1H NMR (400 MHz, DMSO-d₆) δ 7.88 (d, J=7.6 Hz, 2H), 7.71 (t, J=6.5 Hz, 2H), 7.52 (d, J=7.9 Hz, 1H), 7.41 (t, J=7.3 Hz, 2H), 7.35-7.27 (m, 3H), 7.11 (s, 1H), 7.05 (dd, J=7.3, 7.6 Hz, 1H), 6.96 (dd, J=7.1, 7.7 Hz, 1H), 4.28 (d, J=7.1 Hz, 2H), 4.24-4.16 (m, 1H), 3.90 (dd, J=5.1, 9.0 Hz, 1H), 3.37-3.24 (m, 2H), 2.79 (dd, J=7.3, 7.5 Hz, 2H), 2.75-2.65 (m, 2H), 1.65-1.39 (m, 4H), 1.37-1.12 (m, 2H); Low resolution mass spectrum (ES) m/e 512 [(M+1)⁺, calcd for C₃₁H₃₅N₄O₃: 512]; 100% purity based on HPLC.

### Example 58

### (S)-2-[6-AMINO-2-((9H-FLUOREN-9-YLMETHOXYCARBONYL)AMINO)-HEXANOYL-(R)-AMINO]-3-(1H-INDOL-3-YL)-PROPIONIC ACID METHYL ESTER

(R)-2-amino-3-(1H-indol-3-yl)-propionic acid methyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.28 (d, J=7.9 Hz, 0.5H), 7.87 (d, J=7.5 Hz, 2H), 7.70 (d, J=7.4 Hz, 2H), 7.47 (d, J=8.2 Hz, 1H), 7.40 (t, J=7.3 Hz, 2H), 7.34-7.26 (m, 3H), 7.10 (s, 1H), 7.05 (dd, J=7.4, 7.5 Hz, 1H), 6.97 (dd, J=7.3, 7.5 Hz, 1H), 4.52 (dd, J=5.7, 8.3 Hz, 1H), 4.26 (d, J=7.1 Hz, 2H), 4.22-4.14 (m, 1H), 4.00 (t, J=5.0 Hz, 1H), 3.58 (d, J=8.9 Hz, 3H), 3.15 (m, 1H), 3.02 (dd, J=5.5, 14.4 Hz, 1H), 2.71-2.57 (m, 2H), 1.49-1.25 (m, 4H), 1.19-1.10 (m, 2H); Low resolution mass spectrum (ES) m/e 570 [(M+1)⁺, calcd for C₃₃H₃₇N₄O₅: 570]; 100% purity based on HPLC.

### Example 59

### (S)-3-{4-[6-AMINO-2-((9H-FLUOREN-9-YLMETHOXYCARBONYL)AMINO)-HEXANOYLAMINO]-PHENYL}-ACRYLIC ACID ETHYL ESTER

3-(4-Amino-phenyl)-acrylic acid methyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 7.87 (d, J=7.5 Hz, 2H), 7.83-7.51 (m, 7H), 7.40 (dd, J=6.3, 7.3 Hz, 2H), 7.31 (dd, J=7.3, 12.0 Hz, 2H), 6.49 (d, J=15.8 Hz, 1H), 4.34-4.04 (m, 6H), 2.75 (t, J=7.4 Hz, 2H), 1.75-1.25 (m, 6H), 1.22 (t, J=7.0 Hz, 3H); Low resolution mass spectrum (ES) m/e 543 [(M+1)⁺, calcd for C₃₂H₃₆N₃O₅: 543]; 100% purity based on HPLC.

### Example 60

### (S,S)-6-AMINO-2-(2-AMINO-3-NAPHTHALEN-1-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (S)-2-tert-Butoxycarbonylamino-3-naphthalen-1-yl-propionic acid as described in the method of Example 23. Purification by HPLC produced the title compound. 1H NMR (400 MHz, DMSO-d₆) δ 10.06 (s, 1H), 8.75 (d, J=8.0 Hz, 1H), 8.27 (br s, 3H), 8.20 (d, J=8.3 Hz, 1H), 7.92 (d, J=8.0 Hz, 1H), 7.78 (d, J=8.1 Hz, 1H), 7.84-7.69 (br s, 3H), 7.55 (m, 2H), 7.47 (d, J=8.5 Hz, 2H), 7.36 (d, J=6.9 Hz, 1H), 7.28 (app t, J=7.5 Hz, 1H), 7.154 (d, J=8.2 Hz, 2H), 4.55 (dd, J=7.7, 14.2 Hz, 1H), 4.19 (t, J=7.1 Hz, 1H), 3.50 (dd, J=7.3, 14.0 Hz, 1H), 3.41 (dd, J=7.1, 14.1 Hz, 1H), 2.75 (t, J=7.5 Hz, 2H), 2.28 (s, 3H), 1.74-1.65 (m, 1H), 1.61-1.5 (m, 3H), 1.42-1.24 (m, 2H); Low resolution mass spectrum (ES) m/e 433 [(M+1)⁺, calcd for C₂₈H₃₃N₄O₂: 433]; 98.8% purity based on HPLC.

### Example 61

### (S,R)-6-AMINO-2-(2-AMINO-3-NAPHTHALEN-1-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (R)-2-tert-Butoxycarbonylamino-3-naphthalen-2-yl-propionic acid as described in the method of Example 23. Purification by HPLC produced the title compound. 1H NMR (400 MHz, DMSO-d₆) δ 10.08 (s, 1H), 8.79 (d, J=8.0 Hz, 1H), 8.26 (br s, 3H), 7.92-7.85 (m, 2H), 7.77 (s, 1H), 7.70 (br s, 3H), 7.55-7.45 (m, 5H), 7.11 (d, J=8.4 Hz, 2H), 4.38 (dd, J=7.9, 13.9 Hz, 1H), 4.24 (br m, 1H), 3.24 (d, J=6.8, 13.7 Hz, 1H), 3.17 (dd, J=7.7, 13.7 Hz, 1H), 2.55 (m, 2H), 2.24 (s, 1H), 1.56-1.29 (m, 4H), 1.08-0.91 (m, 2H); Low resolution mass spectrum (ES) m/e 433 [(M+1)⁺, calcd for C₂₆H₃₃N₄O₂: 433]; 95.6% purity based on HPLC.

### Example 62

### (S)-4-METHYL-NAPHTHALENE-1-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 4-methyl-naphthalene-1-carboxylic acid as described in the method of Example 23. Purification by HPLC produced the title compound. ¹H NMR (400 MHz, DMSO-d₆) δ 10.09 (s, 1H), 8.66 (d, J=7.6 Hz, 1H), 8.28 (d, J=7.9 Hz, 1H), 8.08 (d, J=8.3 Hz, 1H), 7.69 (br s, 3H), 7.62-7.53 (m, 5H), 7.41 (d, J=7.2 Hz, 1H), 7.14 (d, J=8.4 Hz, 2H), 4.62 (dd, J=8.1, 13.8 Hz, 1H), 2.85-2.77 (m, 2H), 2.69 (s, 3H), 2.27 (s, 3H), 1.87-1.74 (m, 2H), 1.67-1.41 (m, 4H); Low resolution mass spectrum (ES) m/e 404 [(M+1)⁺, calcd for C₂₅H₃₀N₃O₂: 404]; 100% purity based on HPLC.

### Example 63

### (S)-2-METHYL-NAPHTHALENE-1-CARBOXYLIC ACID (5-AMINO-1-P-TOLYLCARBAMOYL-PENTYL)-AMIDE

The title compound was prepared by coupling p-Tolylamine to (S)-6-tert-Butoxycarbonylamino-2-(9H-fluoren-9-ylmethoxycarbonylamino)-hexanoic acid, and then to 2-methyl-naphthalene-1-carboxylic acid as described in the method of Example 23. Purification by HPLC produced the title compound. ¹H NMR (400 MHz, DMSO-d₆) δ 10.10 (s, 1H), 8.78 (d, J=7.1 Hz, 1H), 7.92-7.86 (m, 3H), 7.69 (br s, 3H), 7.55 (d, J=8.4 Hz, 2H), 7.53-7.45 (m, 2H), 7.40 (d, J=8.5 Hz, 1H), 7.15 (d, J=8.3 Hz, 2H), 4.64 (dd, J 7.9, 13.5 Hz, 1H), 2.83-2.75 (m, 2H), 2.43 (s, 3H), 2.27 (s, 3H), 1.85-1.69 (m, 2H), 1.66-1.41 (m, 4H); Low resolution mass spectrum (ES) m/e 404 [(M+1)⁺, calcd for C₂₅H₃₀N₃O₂: 404]; 98.9% purity based on HPLC.

### Example 64

### (S)-4-FLUORO-NAPHTHALENE-1-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 4-fluoro-naphthalene-1-carboxylic acid as described in the method of Example 23. Purification by HPLC produced the title compound. 1H NMR (400 MHz, DMSO-d₆) δ 10.10 (s, 1H), 8.77 (d, J=7.5 Hz, 1H), 8.32 (m, 1H), 8.11 (m, 1H), 7.71-7.65 (m, 6H), 7.53 (d, J=8.4 Hz, 2H), 7.40 (dd, J=8.0, 10.6 Hz, 1H), 7.13 (d, J=8.3 Hz, 2H), 4.61 (dd, J=8.4, 13.5 Hz, 1H), 2.84-2.76 (m, 2H), 2.26 (s, 3H), 1.85-1.72 (m, 2H), 1.66-1.38 (m, 4H); Low resolution mass spectrum (ES) m/e 408 [(M+1)⁺, calcd for C₂₄H₂₇FN₃O₂: 408]; 96.3% purity based on HPLC.

### Example 65

### (S)-3-METHOXY-NAPHTHALENE-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 3-methoxy-naphthalene-2-carboxylic acid as described in the method of Example 23. Purification by HPLC produced the title compound. ¹H NMR (400 MHz, DMSO-d₆) δ 10.10 (s, 1H), 8.67 (d, J=7.6 Hz, 1H), 8.38 (s, 1H), 7.97 (d, J=8.1 Hz, 1H), 7.89 (d, J=8.2 Hz, 1H), 7.64 (br s, 3H), 7.58-7.5 (m, 4H), 7.42 (ddd, J=0.9, 6.8, 8.0 Hz, 1H), 7.14 (d, J=8.4 Hz, 2H), 4.73 (dt, J=5.4, 7.8 Hz, 1H), 4.03 (s, 3H), 2.84-2.76 (m, 2H), 2.26 (s, 3H), 1.93-1.74 (m, 2H), 1.62-1.55 (m, 2H), 1.49-1.38 (m, 2H); Low resolution mass spectrum (ES) m/e 420 [(M+1)⁺, calcd for C₂₅H₃₀N₃O₃: 420]; 98.8% purity based on HPLC.

### Example 66

### (S)-6-METHOXY-NAPHTHALENE-2-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to 6-methoxy-naphthalene-2-carboxylic acid as described in the method of Example 23. Purification by HPLC produced the title compound. 1H NMR (400 MHz, DMSO-d₆) δ 10.05 (s, 1H), 8.64 (d, J=7.7 Hz, 1H), 8.48 (s, 1H), 7.94 (m, 2H), 7.889 (d, J=8.6 Hz, 1H), 7.67 (br s, 3H), 7.50 (d, J=8.4 Hz, 2H), 7.41 (d, J=8.4 Hz, 2H), 7.38 (d, J=2.3 Hz, 1H), 7.22 (dd, J=2.3, 9.0 Hz, 1H), 4.62 (dd, J=7.6, 14.8 Hz, 1H), 3.90 (s, 3H), 2.83-2.78 (m, 2H), 2.24 (s, 3H), 1.87-1.82 (m, 2H), 1.63-1.37 (m, 4H); Low resolution mass spectrum (ES) m/e 420 [(M+1)⁺, calcd for C₂₅H₃₀N₃O₃: 420]; 99.3% purity based on HPLC.

### Example 67

### (S)-ACENAPHTHENE-5-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid, and then to acenaphthene-5-carboxylic acid as described in the method of Example 23. Purification by HPLC produced the title compound. 1H NMR (400 MHz, DMSO-d₆) δ 10.07 (s, 1H), 8.55 (d, J=7.7 Hz, 1H), 8.10 (d, J=8.4 Hz, 1H), 7.99 (d, J=7.1 Hz, 1H), 7.68 (br s, 3H), 7.54-7.49 (m, 3H), 7.36 (d, J=7.7 Hz, 2H), 7.13 (d, J=8.3 Hz, 2H), 4.64 (dd, J=7.7, 14.4 Hz, 1H), 3.34 (obscured, 4H), 2.84-2.79 (m, 2H), 2.26 (s, 3H), 1.85-1.80 (m, 2H), 1.65-1.41 (m, 4H); Low resolution mass spectrum (ES) m/e 416 [(M+1)⁺, calcd for C₂₆H₃₀N₃O₂: 416]; 99.5% purity based on HPLC.

### Example 68

### (S)-6-AMINO-2-(NAPHTHALENE-1-SULFONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to naphthalene-1-sulfonyl chloride as described in the method of Example 26. Purification by HPLC produced the title compound. 1H NMR (400 MHz, DMSO-d₆) δ 9.70 (s, 1H), 8.69 (d, J=8.5 Hz, 1H), 8.45 (d, J=9.1 Hz, 1H), 8.14 (dd, J=0.8, 7.3 Hz, 1H), 8.11 (d, J=8.2 Hz, 1H), 8.01 (d, J=8.1 Hz, 1H), 7.69 (ddd, J=1.2, 6.9, 8.5 Hz, 1H), 7.63 (t, J=7.9 Hz, 1H), 7.61 (br s, 3H), 7.53 (t, J=7.8 Hz, 1H), 7.10 (d, J=8.4 Hz, 2H), 7.00 (d, J=8.5 Hz, 2H), 3.83 (dt, J=5.7, 8.8 Hz, 1H), 2.59-2.5 (m, 2H), 2.21 (s, 3H), 1.57-1.45 (m, 2H), 1.37-1.15 (m, 3H), 1.09-0.99 (m, 1H); Low resolution mass spectrum (ES) m/e 426 [(M+1)⁺, calcd for C₂₃H₂₈N₃O₃S: 426]; 100% purity based on HPLC.

### Example 69

### (S)-6-AMINO-2-(NAPHTHALENE-2-SULFONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to naphthalene-2-sulfonyl chloride as described in the method of Example 26. Purification by HPLC produced the title compound. 1H NMR (400 MHz, DMSO-d₆) δ 9.98 (s, 1H), 8.37 (d, J=1.2 Hz, 1H), 8.21 (d, J=9.0 Hz, 1H), 7.98 (t, J=7.5 Hz, 2H), 7.93 (d, J=8.2 Hz, 1H), 7.79 (dd, J=1.8, 8.7 Hz, 1H), 7.63 (m, 4H), 7.57 (t, J=7.2 Hz, 1H), 7.03 (d, J=8.4 Hz, 2H), 6.89 (d, J=8.4 Hz, 2H), 3.92 (dt, J=6.0, 8.7 Hz, 1H), 2.72-2.62 (m, 2H), 2.17 (s, 3H), 1.64-1.43 (m, 4H), 1.39-1.3 (m, 1H), 1.25-1.06 (m, 1H); Low resolution mass spectrum (ES) m/e 426 [(M+1)⁺, calcd for C₂₃H₂₈N₃O₃S: 426]; 99.7% purity based on HPLC.

### Example 70

### (S)-6-AMINO-2-(BIPHENYL-4-SULFONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to biphenyl-4-sulfonyl chloride as described in the method of Example 26. Purification by HPLC produced the title compound. 1H NMR (400 MHz, DMSO-d₆) δ 9.82 (s, 1H), 8.13 (d, J=9.3 Hz, 1H), 7.81 (d, J=8.4 Hz, 2H), 7.67 (m, 5H), 7.50-7.39 (m, 5H), 7.19 (d, J=8.4 Hz, 2H), 6.97 (d, J=8.4 Hz, 2H), 3.89 (dt, J=6.1, 8.8 Hz, 1H), 2.76-2.72 (m, 2H), 2.18 (s, 3H), 1.65-1.46 (m, 4H), 1.42-1.34 (m, 1H), 1.29-1.20 (m, 1H); Low resolution mass spectrum (ES) m/e 452 [(M+1)⁺, calcd for C₂₅H₃₀N₃O₃S: 452]; 99.7% purity based on HPLC.

### Example 71

### (S)-6-AMINO-2-(3-NAPHTHALEN-1-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-isocyanato-naphthalene as described in the method of Example 26. Purification by HPLC produced the title compound. ¹H NMR (400 MHz, DMSO-d₆) δ 10.14 (s, 1H), 8.77 (s, 1H), 8.14 (d, J=8.3 Hz, 1H), 8.04 (d, J=7.6 Hz, 1H), 7.90 (d, J=8.1 Hz, 1H), 7.66 (br s, 3H), 7.56-7.51 (m, 4.3H), 7.41 (t, J=7.9 Hz, 1H), 7.12 (m, 2.7H), 4.47 (dd, J=7.4, 13.8 Hz, 1H), 2.81 (m, 2H), 2.26 (s, 3H), 1.82-1.75 (m, 1H), 1.69-1.56 (m, 3H), 1.45-1.37 (m, 2H); Low resolution mass spectrum (ES) m/e 405 [(M+1)⁺, calcd for C₂₄H₂₉N₄O₂: 405]; 99.8% purity based on HPLC.

### Example 72

### (S)-6-AMINO-2-(3-NAPHTHALEN-2-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-isocyanato-naphthalene as described in the method of Example 26. Purification by HPLC produced the title compound. ¹H NMR (400 MHz, DMSO-d₆) δ 10.12 (s, 1H), 8.97 (s, 1H), 8.03 (s, 1H), 7.78 (dd, J=4.3, 8.4 Hz, 2H), 7.72 (d, J=8.3 Hz, 1H), 7.68 (br s, 3H), 7.51 (d, J=8.3 Hz, 2H), 7.41 (m, 2H), 7.32 (t, J=7.5 Hz, 1H), 7.12 (d, J=8.2 Hz, 2H), 6.66 (d, J=8.4 Hz, 1H), 4.43 (dd, J=7.8, 13.8 Hz, 1H), 2.81 (m, 2H), 2.25 (s, 3H), 1.81-1.74 (m, 1H), 1.68-1.53 (m, 3H), 1.47-1.35 (m, 2H); Low resolution mass spectrum (ES) m/e 405 [(M+1)⁺, calcd for C₂₄H₂₉N₄O₂: 405]; 99.0% purity based on HPLC.

### Example 73

### (S)-6-AMINO-2-(3-BIPHENYL-2-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-Isocyanato-biphenyl as described in the method of Example 26. Purification by HPLC produced the title compound. 1H NMR (400 MHz, DMSO-d₆) δ 10.04 (s, 1H), 7.88 (d, J=8.2 Hz, 1H), 7.67 (br s, 3H), 7.62 (s, 1H), 7.46 (m, 4H), 7.38 (t, J=7.2 Hz, 1H), 7.33 (d, J=7.7 Hz, 2H), 7.24 (t, J=7.7 Hz, 1H), 7.13 (d, J=7.5 Hz, 1H), 7.09 (d, J=8.1 Hz, 2H), 7.03 (m, 2H), 4.43 (dd, J=7.7, 14.0 Hz, 1H), 2.75 (m, 2H), 2.23 (s, 3H), 1.68-1.63 (m, 1H), 1.57-1.47 (m, 3H), 1.37-1.21 (m, 2H); Low resolution mass spectrum (ES) m/e 431 [(M+1)⁺, calcd for C₂₆H₃₁N₄O₂: 431]; 99.6% purity based on HPLC.

### Example 74

### (S)-6-AMINO-2-[3-(4-BENZYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-benzyl-4-isocyanato-benzene as described in the method of Example 26. Purification by HPLC produced the title compound. 1H NMR (400 MHz, DMSO-d₆) δ 10.07 (s, 1H), 8.66 (s, 1H), 7.68 (br s, 3H), 7.49 (d, J=8.4 Hz, 2H), 7.27 (m, 4H), 7.17 (m, 3H), 7.11 (d, J=8.5 Hz, 2H), 7.07 (d, J=8.5 Hz, 2H), 6.51 (d, J=8.3 Hz, 1H), 4.37 (dt, J=5.7, 8.1 Hz, 1H), 3.84 (s, 2H), 2.81-2.74 (m, 2H), 2.25 (s, 3H), 1.76-1.69 (m, 1H), 1.63-1.51 (m, 3H), 1.43-1.30 (m, 2H); Low resolution mass spectrum (ES) m/e 445 [(M+1)⁺, calcd for C₂₇H₃₃N₄O₂: 445]; 99.8% purity based on HPLC.

### Example 75

### (S)-6-AMINO-2-[3-(4-BENZOYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (4-isocyanato-phenyl)-phenyl-methanone as described in the method of Example 26. Purification by HPLC produced the title compound. 1H NMR (400 MHz, DMSO-d₆) δ 10.12 (s, 1H), 9.28 (s, 1H), 7.70-7.63 (m, 7H), 7.55 (m, 4H), 7.50 (d, J=8.4 Hz, 2H), 7.12 (d, J=8.5 Hz, 2H), 6.79 (d, J=8.2 Hz, 1H), 4.41 (dt, J=5.6, 8.0 Hz, 1H), 2.82-2.76 (m, 2H), 2.25 (s, 3H), 1.80-1.73 (m, 1H), 1.67-1.52 (m, 3H), 1.45-1.32 (m, 2H); Low resolution mass spectrum (ES) m/e 459 [(M+1)⁺, calcd for C₂₇H₃₁N₄O₃: 449]; 99.7% purity based on HPLC.

### Example 76

### (S)-6-AMINO-2-[3-(2-BIPHENYL-4-YL-ETHYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 4-(2-Isocyanato-ethyl)-biphenyl as described in the method of Example 26. Purification by HPLC produced the title compound. 1H NMR (400 MHz, DMSO-d₆) δ 9.98 (s, 1H), 7.68 (br s, 3H), 7.62 (d, J=7.5 Hz, 2H), 7.51 (d, J=8.1 Hz, 2H), 7.49 (d, J=8.4 Hz, 2H), 7.45 (t, J=7.7 Hz, 2H), 7.34 (t, J=7.4 Hz, 1H), 7.29 (d, J=8.1 Hz, 2H), 7.10 (d, J=8.1 Hz, 2H), 6.27 (d, J=8.5 Hz, 1H), 6.14 (t, J=5.7 Hz, 1H), 4.29 (dt, J=5.6, 8.2 Hz, 1H), 3.34-3.22 (m, 2H), 2.80-2.75 (m, 1H), 2.72 (t, J=7.1 Hz, 2H), 2.25 (s, 3H), 1.68-1.48 (m, 4H), 1.40-1.23 (m, 2H); Low resolution mass spectrum (ES) m/e 459 [(M+1)⁺, calcd for C₂₈H₃₅N₄O₂: 459]; 99.6% purity based on HPLC.

### Example 77

### (S)-6-AMINO-2-(2-(S)-AMINO-3-NAPHTHALEN-1-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to (R)-2-tert-Butoxycarbonylamino-3-naphthalen-1-yl-propionic acid as described in the method of Example 23. Purification by HPLC produced the title compound. ¹H NMR (400 MHz, DMSO-d₆) δ 10.03 (s, 1H), 8.66 (d, J=8.0 Hz, 1H), 8.34 (br s, 3H), 8.20 (d, J=8.3 Hz, 1H), 7.97 (d, J=7.8 Hz, 1H), 7.88 (d, J=8.2 Hz, 1H), 7.68 (br s, 3H), 7.64-7.57 (m, 2H), 7.46 (m, 3H), 7.40 (d, J=6.9 Hz, 1H), 7.10 (d, J=8.4 Hz, 1H), 4.32 (dd, J=7.4, 14.1 Hz, 1H), 4.23 (br s, 1H), 3.50 (dd, J=8.5, 13.7 Hz, 1H), 3.44 (dd, J=6.8, 13.8 Hz, 1H), 2.63 (m, 2H), 2.23 (s, 3H), 1.40-1.33 (m, 3H), 1.26-1.2 (m, 1H), 0.94-0.78 (m, 2H); Low resolution mass spectrum (ES) m/e 433 [(M+1)⁺, calcd for C₂₆H₃₃N₄O₂: 433]; 97.0% purity based on HPLC.

### Example 78

### (S)-6-AMINO-2-[3-(5,6,7,8-TETRAHYDRO-NAPHTHALEN-1-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to 5-Isocyanato-1,2,3,4-tetrahydro-naphthalene as described in the method of Example 26. Purification by HPLC produced the title compound. 1H NMR (400 MHz, DMSO-d₆) δ 10.08 (s, 1H), 7.75 (s, 1H), 7.66 (br s, 3H), 7.63 (d, J=7.9 Hz, 1H), 7.50 (d, J=8.4 Hz, 2H), 7.12 (d, J=8.4 Hz, 2H), 7.01 (d, J=8.3 Hz, 1H), 6.96 (t, J=7.8 Hz, 1H), 6.69 (d, J=7.5 Hz, 1H), 4.38 (dd, J=7.8, 13.4 Hz, 1H), 2.78 (m, 2H), 2.69 (t, J=5.9 Hz, 2H), 2.25 (s, 3H), 1.79-1.3 (m, 11H); Low resolution mass spectrum (ES) m/e 409 [(M+1)⁺, calcd for C₂₄H₃₃N₄O₂: 409]; 97.8% purity based on HPLC.

### Example 79

### NAPHTHALENE-1-CARBOXYLIC ACID (5-AMINO-1-P-TOLYLCARBAMOYL-PENTYL)-AMIDE

p-Tolylamine was coupled to (S)-6-tert-Butoxycarbonylamino-2-(9H-fluoren-9-ylmethoxycarbonylamino)-hexanoic acid then to Naphthalene-1-carboxylic acid as described in the method of Example 23. Purification by HPLC produced the title compound. 1H NMR (400 MHz, DMSO-d₆) δ 10.10 (s, 1H), 8.74 (d, J=7.5 Hz, 1H), 8.24 (dq, J=3.3, 6.8 Hz, 1H), 8.03 (d, J=8.3 Hz, 1H), 7.98 (dq, J=3.4, 6.9 Hz, 1H), 7.70 (br s, 3H), 7.67 (dd, J=1.1, 7.0 Hz, 1H), 7.58-7.54 (m, 5H), 7.14 (d, J=8.4 Hz, 2H), 4.63 (ddd, J=5.4, 7.9, 8.9 Hz, 1H), 2.84-2.78 (m, 2H), 2.27 (s, 3H), 1.86-1.75 (m, 2H), 1.6-1.42 (m, 4H); Low resolution mass spectrum (ES) m/e 390 [(M+1)⁺, calcd for C₂₄H₂₈N₃O₂: 460]; 99.9% purity based on HPLC.

### Example 80

### (S)-[5-AMINO-1-(3-FLUORO-PHENYLCARBAMOYL)-PENTYL]-CARBAMIC ACID 9H-FLUOREN-9-YLMETHYL ESTER

m-Fluoro phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 10.3 (s, 1H), 7.89 (d, J=7.5 Hz, 2H), 7.72 (t, J=7.4 Hz, 2H), 7.69-7.56 (m, 4H), 7.41 (t, J=7.4 Hz, 2H), 7.38-7.27 (m, 4H), 6.88 (dd, J=7.6, 9.3 Hz, 1H), 4.35-4.16 (m, 3H), 4.11 (ddd, J=5.8, 8.4, 8.4 Hz, 1H), 2.77 (dt, J=5.9, 8.1 Hz, 2H), 1.76-1.23 (m, 6H); Low resolution mass spectrum (ES) m/e 462 [(M+1)⁺, calcd for C₂₇H₂₉FN₃O₃: 462]; 100% purity based on HPLC.

### Example 81

### (S)-[5-AMINO-1-(INDAN-5-YLCARBAMOYL)-PENTYL]-CARBAMIC ACID 9H-FLUOREN-9-YLMETHYL ESTER

Indan-5-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.88 (s, 1H), 7.89 (d, J=7.5 Hz, 2H), 7.73 (t, J=7.5 Hz, 2H), 7.6 (d, J=8.1 Hz, 2H), 7.51 (s, 1H), 7.41 (t, J=7.5 Hz, 1H), 7.33 (dd, J=3.4, 7.4 Hz, 2H), 7.28 (d, J=8.1 Hz, 1H), 7.13 (d, J=8.1 Hz, 1H), 4.36-4.17 (m, 1H), 4.25 (AB q, J=7.32, 21.9 Hz, 2H), 4.11 (dt, J=5.9, 8.4 Hz, 1H), 2.87-2.7 (m, 6H), 1.98 (p, J=7.4 Hz, 2H), 1.74-1.25 (m, 6H); Low resolution mass spectrum (ES) m/e 484 [(M+1)⁺, calcd for C₃₀H₃₄N₃O₃: 484]; 98.5% purity based on HPLC.

### Example 82

### (S)-[5-AMINO-1-(1H-INDAZOL-5-YLCARBAMOYL)-PENTYL]-CARBAMIC ACID 9H-FLUOREN-9-YLMETHYL ESTER

1H-Indazol-5-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 8.15 (s, 1H), 7.99-7.84 (m, 2H), 7.79-7.57 (m, 5H), 7.51-7.28 (m, 4H), 4.48-4.08 (m, 4H), 2.78 (d, J=6.2 Hz, 2H), 1.92-1.18 (m, 6H); Low resolution mass spectrum (ES) m/e 484 [(M+1)⁺, calcd for C₂₈H₃₀N₅O₃: 484]; 85.2% purity based on HPLC.

### Example 83

### (S)-[5-AMINO-1-(4-METHOXY-PHENYLCARBAMOYL)-PENTYL]-CARBAMIC ACID 9H-FLUOREN-9-YLMETHYL ESTER

p-Methoxy phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.88 (s, 1H), 7.89 (d, J=7.5 Hz, 2H), 7.73 (t, J=7.5 Hz, 2H), 7.6 (br s, 2H), 7.61 (d, J=8.2 Hz, 2H), 7.5 (d, J=9.0 Hz, 2H), 7.41 (t, J=7.4 Hz, 2H), 7.31 (ddd, J=4.1, 7.4, 7.4 Hz, 2H), 6.87 (d, J=6.9 Hz, 2H), 4.35-4.17 (m, 3H), 4.09 (ddd, J=5.9, 8.5, 8.5 Hz, 1H), 3.71 (s, 3H), 2.77 (dd, J=5.7, 10.6 Hz, 2H), 1.79-1.23 (m, 6H); Low resolution mass spectrum (ES) m/e 474 [(M+1)⁺, calcd for C₂₈H₃₂N₃O₄: 484]; 98.3% purity based on HPLC.

### Example 84

### (R)-(2-AMINO-2-P-TOLYLCARBAMOYL-ETHYL)-CARBAMIC ACID 9H-FLUOREN-9-YLMETHYL ESTER

p-Tolylamine was coupled to (R)-2-tert-Butoxycarbonylamino-3-(9H-fluoren-9-ylmethoxycarbonylamino)-propionic acid as described in the method of Example 6. Purification by HPLC produced the title compound. 1H NMR (400 MHz, DMSO-d₆) δ 7.86 (d, J=7.6 Hz, 2H), 7.64-7.57 (m, 2H), 7.4 (t, J=8.2 Hz, 4H), 7.28 (t, J=7.4 Hz, 2H), 7.11 (d, J=8.3 Hz, 2H), 4.32-4.22 (m, 2H), 4.16 (t, J=6.6 Hz, 1H), 3.93 (t, J=5.5 Hz, 1H), 3.53 (m, 1H), 3.44 (dd, J=6.0, 14.4 Hz, 1H), 2.22 (s, 3H); Low resolution mass spectrum (ES) m/e 416 [(M+1)⁺, calcd for C₂₅H₂₆N₃O₃: 416]; 99% purity based on HPLC.

### Example 85

### (S)-2-P-TOLYLCARBAMOYL-PYRROLIDINE-1-CARBOXYLIC ACID 9H-FLUOREN-9-YLMETHYL ESTER

p-Tolylamine was coupled to (S)-Pyrrolidine-1,2-dicarboxylic acid 1-(9H-fluoren-9-ylmethyl) ester as described in the method for intermediate #1 of Example 1. Purification by HPLC produced the title compound. 1H NMR (400 MHz, DMSO-d₆) δ 10.12 (s, 0.3H), 9.94 (s, 0.3H), 7.87 (d, J=7.4 Hz, 1H), 7.79 (d, J=7.6 Hz, 1H), 7.64 (t, J=7.6 Hz, 1H), 7.59-7.47 (m, 2H), 7.45-7.36 (m, 2H), 7.36-7.26 (m, 2H), 7.13-6.98 (m, 3H), 4.46 (dd, J=3.2, 8.7 Hz, 0.5H), 4.29-4.13 (m, 2.5H), 4.09-3.99 (m, 1H), 3.54-3.29 (m, 2H), 2.35-2.09 (m, 4H), 2.00-1.75 (m, 3H); Low resolution mass spectrum (ES) m/e 427 [(M+1)⁺, calcd for C₂₇H₂₇N₂O₃: 427]; 98% purity based on HPLC.

### Example 86

### (R)-2-P-TOLYLCARBAMOYL-PYRROLIDINE-1-CARBOXYLIC ACID 9H-FLUOREN-9-YLMETHYL ESTER

p-Tolylamine was coupled to (R)-Pyrrolidine-1,2-dicarboxylic acid 1-(9H-fluoren-9-ylmethyl) ester as described in the method for Intermediate #1 of Example 1. Purification by HPLC produced the title compound. 1H NMR (400 MHz, DMSO-d₆) δ 10.12 (s, 0.3H), 9.94 (s, 0.3H), 7.87 (d, J=7.4 Hz, 1H), 7.79 (d, J=7.6 Hz, 1H), 7.64 (t, J=7.6 Hz, 1H), 7.59-7.47 (m, 2H), 7.45-7.36 (m, 2H), 7.36-7.26 (m, 2H), 7.13-6.98 (m, 3H), 4.46 (dd, J=3.2, 8.7 Hz, 0.5H), 4.29-4.13 (m, 2.5H), 4.09-3.99 (m, 1H), 3.54-3.29 (m, 2H), 2.35-2.09 (m, 4H), 2.00-1.75 (m, 3H); Low resolution mass spectrum (ES) m/e 427 [(M+1)⁺, calcd for C₂₇H₂₇N₂O₃: 427]; 98% purity based on HPLC.

### Example 87

### (4R,S)-4-HYDROXY-2-P-TOLYLCARBAMOYL-PYRROLIDINE-1-CARBOXYLIC ACID 9H-FLUOREN-9-YLMETHYL ESTER

p-Tolylamine was coupled to (4R,S)-4-Hydroxy-pyrrolidine-1,2-dicarboxylic acid 1-(9H-fluoren-9-ylmethyl) ester as described in the method for Intermediate #1 of Example 1. Purification by HPLC produced the title compound. 1H NMR (400 MHz, DMSO-d₆) δ 10.17 (s, 0.15H), 10.01 (s, 0.15H), 7.87 (d, J=7.5 Hz, 1H), 7.78 (d, J=7.6 Hz, 1H), 7.64 (dd, J=5.8, 6.7 Hz, 1H), 7.61-7.52 (m, 1H), 7.51 (d, J=8.4 Hz, 1H), 7.46-7.36 (m, 2H), 7.35-7.27 (m, 2H), 7.12-7.02 (m, 3H), 4.56 (t, J=7.9 Hz, 0.5H), 4.41-4.28 (m, 1.5H), 4.27-4.15 (m, 2H), 4.06-3.98 (m, 1H), 3.57-3.34 (m, 2H); Low resolution mass spectrum (ES) m/e 443 [(M+1)⁺, calcd for C₂₇H₂₇N₂O₄: 443]; 98% purity based on HPLC.

### Example 88

### (S)-N-(9-{[5-AMINO-1-(BENZYL-CYCLOHEXYL-CARBAMOYL)-PENTYLCARBAMOYL]-METHYL}-9H-PURIN-6-YL)-BENZAMIDE

Benzyl-cyclohexyl-amine was coupled to (S)-6-tert-Butoxycarbonylamino-2-(9H-fluoren-9-ylmethoxycarbonylamino)-hexanoic acid, and then to (6-Benzoylamino-purin-9-yl)-acetic acid as described in the method of Example 23. Purification by HPLC produced the title compound. ¹H NMR (400 MHz, DMSO-d₆) δ 8.68 (d, J=6.9 Hz, 1H), 8.4 (s, 1H), 8.02 (d, J=7.3 Hz, 2H), 7.64 (dd, J=7.2, 7.5 Hz, 1H), 7.55 (dd, J=7.4, 7.8 Hz, 2H), 7.32-7.10 (m, 5H), 5.05 (s, 0.85H), 4.99 (s, 0.65H), 4.84 (d, J=5.0, 9.0 Hz, 0.5H), 4.6 (d, J=19.5 Hz, 0.7H), 4.51 (d, J=18.1 Hz, 0.7H), 4.46-4.35 (m, 0.9H), 4.24-4.13 (m, 0.8H), 3.8-3.65 (m, 1H), 2.83-2.7 (m, 1H); Low resolution mass spectrum (ES) m/e 597 [(M+1)⁺, calcd for C₃₃H₄₁N₈O₃: 597]; 98% purity based on HPLC.

### Example 89

### (S)-6-AMINO-2-(3-1H-INDOL-3-YL-PROPIONYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 3-(1H-Indol-3-yl)-propionic acid as described in the method of Example 23. Purification by HPLC produced the title compound. ¹H NMR (400 MHz, DMSO-d₆) δ 10.77 (s, 1H), 9.96 (s, 1H), 8.17 (d, J=8.0 Hz, 1H), 7.68 (br s, 3H), 7.53 (d, J=7.9 Hz, 1H), 7.48 (d, J=8.4 Hz, 2H), 7.32 (d, J=8.1 Hz, 1H), 7.11 (m, 3H), 7.05 (t, J=7.7 Hz, 1H), 6.96 (d, J=7.0 Hz, 1H), 4.41 (dt, J=5.6, 8.4 Hz, 1H), 2.92 (t, J=7.7 Hz, 2H), 2.77-2.71 (m, 2H), 2.54 (m, 2H), 2.25 (s, 3H), 1.72-1.65 (m, 1H), 1.61-1.47 (m, 3H), 1.37-1.21 (m, 2H); Low resolution mass spectrum (ES) m/e 407 [(M+1)⁺, calcd for C₂₄H₃₁N₄O₂: 407]; 96.6% purity based on HPLC.

### Example 90

### (S)-6-AMINO-2-[3-(1H-INDOL-3-YL)-2-(S)-METHYLAMINO-PROPIONYLAMINO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (S)-3-(1H-Indol-3-yl)-2-methylamino-propionic acid as described in the method of Example 23. Purification by HPLC produced the title compound. 1H NMR (400 MHz, DMSO-d₆) δ 11.03 (d, J=2.1 Hz, 1H), 10.14 (s, 1H), 9.04 (d, J=8.1 Hz, 1H), 8.88 (br s, 1H), 8.71 (br s, 1H), 7.75 (br s, 3H), 7.58 (d, J=8.0 Hz, 1H), 7.50 (d, J=8.4 Hz, 2H), 7.32 (d, J=8.1 Hz, 1H), 7.21 (d, J=2.4 Hz, 1H), 7.14 (d, J=8.4 Hz, 2H), 7.02 (t, J=7.3 Hz, 1H), 6.88 (t, J=7.5 Hz, 1H), 4.51 (dt, J=5.9, 8.1 Hz, 1H), 4.11 (m, 1H), 3.28 (dd, J=6.0, 15.0 Hz, 1H), 3.2 (t, J=6.5, 15.1 Hz, 1H), 2.78-2.74 (m, 2H), 2.47 (t, J=4.9 Hz, 3H), 2.27 (s, 3H), 1.79-1.52 (m, 3H), 1.42-1.23 (m, 3H); Low resolution mass spectrum (ES) m/e 436 [(M+1)⁺, calcd for C₂₅H₃₄N₅O₂: 436]; 96.5% purity based on HPLC.

### Example 91

### (S)-6-AMINO-2-(2-NAPHTHALEN-1-YL-ACETYLAMINO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to Naphthalen-1-yl-acetic acid as described in the method of Example 23. Purification by HPLC produced the title compound. 1H NMR (400 MHz, DMSO-d₆) δ 9.98 (s, 1H), 8.53 (d, J=8.1 Hz, 1H), 8.11 (m, 1H), 7.92 (m, 1H), 7.81 (m, 1H), 7.68 (br s, 3H), 7.50 (m, 3H), 7.47 (d, J=8.4 Hz, 2H), 7.45 (m, 2H), 7.10 (d, J=8.3 Hz, 2H), 4.41 (dt, J=5.5, 8.4 Hz, 1H), 4.00 (q, J=15.0 Hz, 2H), 2.73 (m, 2H), 2.24 (s, 3H), 1.75 (m, 1H), 1.68-1.60 (m, 1H), 1.54 (m, 2H), 1.42-1.26 (m, 2H); Low resolution mass spectrum (ES) m/e 404 [(M+1)⁺, calcd for C₂₅H₃₀N₃O₂: 404]; 99.9% purity based on HPLC.

### Example 92

### (S)-[5-AMINO-1-(METHYL-P-TOLYL-CARBAMOYL)-PENTYL]-CARBAMIC ACID 9H-FLUOREN-9-YLMETHYL ESTER

p-Tolylmethylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 7.90 (d, J=7.5 Hz, 2H), 7.73 (dd, J=5.0, 7.3 Hz, 2H), 7.58 (br s, 3H), 7.56 (d, J=8.1 Hz, 1H), 7.42 (t, J=7.5 Hz, 2H), 7.33 (dt, J=3.6, 7.3 Hz, 2H), 7.26 (m, 4H), 4.25-4.18 (m, 3H), 4.04 (dd, J=8.3, 13.4 Hz, 1H), 3.13 (s, 3H), 2.63 (m, 2H), 2.32 (s, 3H), 1.49-1.43 (m, 2H), 1.29-1.16 (m, 3H), 1.04-0.96 (m, 1H); Low resolution mass spectrum (ES) m/e 472 [(M+1)⁺, calcd for C₂₉H₃₄N₃O₃: 472]; 88% purity based on HPLC.

### Example 93

### (S)-6-AMINO-2-[3-(4-METHOXY-BIPHENYL-3-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 3-Isocyanato-4-methoxy-biphenyl as described in the method of Example 26. Purification by HPLC produced the title compound. 1H NMR (400 MHz, DMSO-d₆) δ 10.04 (s, 1H), 8.43 (d, J=2.3 Hz, 1H), 8.27 (s, 1H), 7.66 (br s, 3H), 7.50 (m, 4H), 7.39 (t, J=7.8 Hz, 2H), 7.35 (d, J=8.1 Hz, 1H), 7.27 (t, J=7.3 Hz, 1H), 7.16 (dd, J=2.3, 8.4 Hz, 1H), 7.09 (d, J=8.3 Hz, 2H), 7.04 (d, J=8.5 Hz, 1H), 4.34 (dt, J=5.5, 8.2 Hz, 1H), 3.87 (s, 3H), 2.77 (m, 2H), 2.23 (s, 3H), 1.76-1.69 (m, 1H), 1.62-1.51 (m, 3H), 1.45-1.30 (m, 2H); Low resolution mass spectrum (ES) m/e 461 [(M+1)⁺, calcd for C₂₇H₃₃N₄O₃: 461]; 99.8% purity based on HPLC.

### Example 94

### (S)-6-AMINO-2-(3-BIPHENYL-4-YL-UREIDO)-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d₆) δ 10.84 (s, 1H), 9.92 (s, 1H), 8.89 (s, 1H), 7.72 (d, J=1.6 Hz, 1H), 7.69 (br s, 3H), 7.61 (d, J=7.3 Hz, 2H), 7.56 (d, J=8.8 Hz, 2H), 7.49 (d, J=8.8 Hz, 2H), 7.42 (t, J=7.7 Hz, 2H), 7.29 (t, J=7.4 Hz, 1H), 7.18 (d, J=8.6 Hz, 1H), 7.13 (dd, J=1.9, 8.7 Hz, 1H), 6.59 (d, J=8.2 Hz, 1H), 6.06 (s, 1H), 4.04 (dd, J=7.8, 13.6 Hz, 1H), 2.84-2.76 (m, 2H), 2.35 (s, 3H), 1.82-1.73 (m, 1H), 1.69-1.55 (m, 3H), 1.47-1.34 (m, 2H); Low resolution mass spectrum (ES) m/e 470 [(M+1)⁺, calcd for C₂₈H₃₂N₅O₂: 470]; 99.9% purity based on HPLC.

### Example 95

### (S)-6-AMINO-2-[3-(3,5-BIS-TRIFLUOROMETHYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-3,5-bis-trifluoromethyl-benzene as described in the method of Example 26. Purification by HPLC produced the title compound. 1H NMR (400 MHz, DMSO-d₆) δ 10.09 (s, 1H), 9.65 (s, 1H), 8.06 (s, 1H), 7.70 (br s, 3H), 7.57 (s, 1H), 7.50 (d, J=8.4 Hz, 2H), 7.12 (d, J=8.4 Hz, 2H), 6.98 (d, J=7.6 Hz, 1H), 4.38 (dd, J=7.9, 13.2 Hz, 1H), 2.82-2.75 (m, 2H), 2.25 (s, 3H), 1.82-1.51 (m, 4H), 1.46-1.3 (m, 2H); Low resolution mass spectrum (ES) m/e 491 [(M+1)⁺, calcd for C₂₂H₂₅F₆N₄O₂: 491]; 99.4% purity based on HPLC.

### Example 96

### (R,S)-6-AMINO-2-[3-(1-NAPHTHALEN-1-YL-ETHYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (R)-1-(1-Isocyanato-ethyl)-naphthalene as described in the method of Example 26. Purification by HPLC produced the title compound. 1H NMR (400 MHz, DMSO-d₆) δ 9.95 (s, 1H), 8.10 (m, 1H), 7.92 (m, 1H), 7.80 (d, J=7.7 Hz, 1H), 7.67 (br s, 3H), 7.52-7.47 (m, 4H), 7.44 (d, J=8.3 Hz, 2H), 7.09 (d, J=8.4 Hz, 2H), 6.77 (d, J=8.0 Hz, 1H), 6.22 (d, J=8.5 Hz, 1H), 5.53 (p, J=5.9 Hz, 1H), 4.29 (dd, J=7.9, 13.8 Hz, 1H), 2.78 (br s, 2H), 2.24 (s, 3H), 1.71-1.63 (m, 1H), 1.91-1.5 (m, 3H), 1.46 (d, J=6.8 Hz, 3H), 1.40-1.26 (m, 2H); Low resolution mass spectrum (ES) m/e 433 [(M+1)⁺, calcd for C₂₆H₃₃N₄O₂: 433]; 99.2% purity based on HPLC.

### Example 97

### (S,S)-6-AMINO-2-[3-(1-NAPHTHALEN-1-YL-ETHYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (S)-1-(1-Isocyanato-ethyl)-naphthalene as described in the method of Example 26. Purification by HPLC produced the title compound. 1H NMR (400 MHz, DMSO-d₆) δ 10.01 (s, 1H), 8.13 (d, J=8.0 Hz, 1H), 7.95 (m, 1H), 7.82 (dd, J=2.9, 6.0 Hz, 1H), 7.65 (br s, 3H), 7.58-7.48 (m, 6H), 7.12 (d, J=8.3 Hz, 2H), 6.78 (d, J=8.1 Hz, 1H), 6.22 (d, J=8.5 Hz, 1H), 5.55 (p, J=6.9 Hz, 1H), 4.30 (dd, J=8.0, 13.9 Hz, 1H), 2.75-2.67 (m, 2H), 2.25 (s, 3H), 1.69-1.60 (m, 1H), 1.54-1.46 (m, 2H), 1.45 (d, J=6.9 Hz, 3H), 1.35-1.21 (m, 2H); Low resolution mass spectrum (ES) m/e 433 [(M+1)⁺, calcd for C₂₆H₃₃N₄O₂: 433]; 99.8% purity based on HPLC.

### Example 98

### (S)-6-AMINO-2-[3-(3-PHENOXY-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-3-phenoxy-benzene as described in the method of Example 26. Purification by HPLC produced the title compound. 1H NMR (400 MHz, DMSO-d₆) δ 10.06 (s, 1H), 8.89 (s, 1H), 7.68 (br s, 3H), 7.48 (d, J=8.4 Hz, 2H), 7.38 (dd, J=7.6, 8.3 Hz, 2H), 7.22 (m, 2H), 7.13 (m, 3H), 7.00 (m, 3H), 6.57-6.53 (m, 2H), 4.33 (dd, J=8.0, 13.5 Hz, 1H), 2.8-2.75 (m, 2H), 2.25 (s, 3H), 1.76-1.68 (m, 1H), 1.63-1.51 (m, 3H), 1.43-1.28 (m, 2H); Low resolution mass spectrum (ES) m/e 447 [(M+1)⁺, calcd for C₂₆H₃₁N₄O₃: 447 99.7% purity based on HPLC.

### Example 99

### (S)-6-AMINO-2-[PHENYLSULFONAMIDE-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to Benzenesulfonyl isocyanate as described in the method of Example 23. Purification by HPLC produced the title compound. 1H NMR (400 MHz, DMSO-d₆) δ 10.77 (s, 1H), 10.04 (s, 1H), 7.90 (dd, J=1.2, 7.6 Hz, 2H), 7.7-7.58 (m, 6H), 7.43 (d, J=8.8 Hz, 2H), 7.10 (d, J=8.1 Hz, 2H), 6.92 (d, J=8.0 Hz, 1H), 4.21 (dd, J=7.7, 13.4 Hz, 1H), 2.73-2.68 (m, 2H), 2.24 (s, 3H), 1.7-1.62 (m, 1H), 1.58-1.4 (m, 3H), 1.27-1.16 (m, 2H); Low resolution mass spectrum (ES) m/e 419 [(M+1)⁺, calcd for C₂₀H₂₇N₄O₂S: 419]; 95.2% purity based on HPLC.

### Example 100

### (S,S)-6-AMINO-2-[2-AMINO-3-(1H-INDOL-3-YL)-PROPIONYLAMINO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-tert-Butoxycarbonylamino-3-(1H-indol-3-yl)-propionic acid as described in the method of Example 23. Purification by HPLC produced the title compound. 1H NMR (400 MHz, DMSO-d₆) δ 11.02 (d, J=1.3 Hz, 1H), 10.14 (s, 1H), 8.93 (d, J=7.8 Hz, 1H), 8.04 (br s, 3H), 7.79 (br s, 3H), 7.68 (d, J=7.9 Hz, 1H), 7.51 (d, J=8.4 Hz, 2H), 7.34 (d, J=8.1 Hz, 1H), 7.21 (d, J=2.3 Hz, 1H), 7.14 (d, J=8.3 Hz, 2H), 7.05 (m, 1H), 6.92 (t, J=7.4 Hz, 1H), 4.48 (dd, J=8.0, 13.8 Hz, 1H), 4.10 (m, 1H), 3.27 (dd, J=4.9, 14.9 Hz, 1H), 3.08 (dd, J=8.2, 14.9 Hz, 1H), 2.8-2.75 (m, 2H), 2.26 (s, 3H), 1.81-1.53 (m, 4H), 1.47-1.29 (m, 2H); Low resolution mass spectrum (ES) m/e 422 [(M+1)⁺, calcd for C₂₄H₃₂N₅O₂: 422]; 96.1% purity based on HPLC.

### Example 101

### (S)-(4-AMINO-1-P-TOLYLCARBAMOYL-BUTYL)-CARBAMIC ACID 9H-FLUOREN-9-YLMETHYL ESTER

p-Tolylamine was coupled to (S)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.00 (s, 1H), 7.9 (d, J=7.5 Hz, 2H), 7.76-7.71 (m, 6H), 7.49 (d, J=8.3 Hz, 2H), 7.42 (t, J=7.4 Hz, 2H), 7.33 (dd, J=7.2, 12.5 Hz, 2H), 7.12 (d, J=8.3 Hz, 2H), 4.34-4.2 (m, 3H), 4.16 (dd, J=8.0, 13.5 Hz, 1H), 2.79 (m, 2H), 2.25 (s, 3H), 1.78-1.55 (m, 4H); Low resolution mass spectrum (ES) m/e 444 [(M+1)⁺, calcd for C₂₇H₃₀N₃O₃: 444]; 99.9% purity based on HPLC.

### Example 102

### (R)-(4-AMINO-1-P-TOLYLCARBAMOYL-BUTYL)-CARBAMIC ACID 9H-FLUOREN-9-YLMETHYL ESTER

p-Tolylamine was coupled to (R)-5-tert-Butoxycarbonylamino-2-(9H-fluoren-9-ylmethoxycarbonylamino)-pentanoic acid as described in the method of Example 6. Purification by HPLC produced the title compound. 1H NMR (400 MHz, DMSO-d₆) δ 10.00 (s, 1H), 7.9 (d, J=7.5 Hz, 2H), 7.76-7.71 (m, 6H), 7.49 (d, J=8.4 Hz, 2H), 7.42 (t, J=7.4 Hz, 2H), 7.33 (dd, J=7.2, 12.5 Hz, 2H), 7.12 (d, J=8.3 Hz, 2H), 4.34-4.2 (m, 3H), 4.16 (dd, J=8.0, 13.4 Hz, 1H), 2.79 (m, 2H), 2.25 (s, 3H), 1.78-1.56 (m, 4H); Low resolution mass spectrum (ES) m/e 444 [(M+1)⁺, calcd for C₂₇H₃₀N₃O₃: 444]; 99.9% purity based on HPLC.

### Example 103

### (S)-(2-AMINO-1-P-TOLYLCARBAMOYL-ETHYL)-CARBAMIC ACID 9H-FLUOREN-9-YLMETHYL ESTER

p-Tolylamine was coupled to (S)-3-tert-Butoxycarbonylamino-2-(9H-fluoren-9-ylmethoxycarbonylamino)-propionic acid as described in the method of Example 6. Purification by HPLC produced the title compound. 1H NMR (400 MHz, DMSO-d₆) δ 10.14 (s, 1H), 7.97 (br s, 3H), 7.90 (d, J=7.5 Hz, 2H), 7.83 (d, J=8.2 Hz, 1H), 7.73 (dd, J=1.9, 7.2 Hz, 2H), 7.50 (d, J=8.3 Hz, 2H), 7.42 (t, J=7.4 Hz, 2H), 7.33 (t, J=7.4 Hz, 2H), 7.14 (d, J=8.3 Hz, 2H), 4.47-4.33 (m, 3H), 4.26 (t, J=6.7 Hz, 1H), 3.24 (m, 1H), 3.05 (m, 1H), 2.26 (s, 3H); Low resolution mass spectrum (ES) m/e 416 [(M+1)⁺, calcd for C₂₅H₂₆N₃O₃: 416]; 98.6% purity based on HPLC.

### Example 104

### (S)-(6-AMINO-1-P-TOLYLCARBAMOYL-HEXYL)-CARBAMIC ACID 9H-FLUOREN-9-YLMETHYL ESTER

p-Tolylamine was coupled to (S)-8-tert-Butoxycarbonylamino-2-(9H-fluoren-9-ylmethoxycarbonylamino)-octanoic acid as described in the method of Example 6. Purification by HPLC produced the title compound. 1H NMR (400 MHz, DMSO-d₆) δ 9.94 (s, 1H), 7.89 (d, J=7.5 Hz, 2H), 7.76-7.65 (m, 6H), 7.49 (d, J=8.4 Hz, 2H), 7.42 (t, J=7.4 Hz, 2H), 7.32 (dt, J=3.1, 7.3 Hz, 2H), 7.11 (d, J=8.3 Hz, 2H), 4.29-4.2 (m, 3H), 4.12 (dd, J=8.2, 14.0 Hz, 1H), 2.79-2.74 (m, 2H), 2.25 (s, 3H), 1.71-1.61 (m, 2H), 1.55-1.48 (m, 2H), 1.39-1.31 (m, 4H); Low resolution mass spectrum (ES) m/e 473 [(M+1)⁺, calcd for C₂₉H₃₄N₃O₃: 473]; 98.3% purity based on HPLC.

### Example 105

### (S)-[5-AMINO-1-(4-TERT-BUTYL-PHENYLCARBAMOYL)-PENTYL]-CARBAMIC ACID 9H-FLUOREN-9-YLMETHYL ESTER

p-t-Butylphenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d₆) δ 9.96 (s, 1H), 7.89 (d, J=7.5 Hz, 2H), 7.73 (t, J=7.6 Hz, 2H), 7.69-7.59 (m, 4H), 7.50 (d, J=8.7 Hz, 2H), 7.41 (t, J=6.8 Hz, 2H), 7.36-7.28 (m, 4H), 4.35-4.17 (m, 3H), 4.12 (ddd, J=5.7, 8.3, 8.3 Hz, 1H), 2.77 (dddd, J=6.4, 6.4, 6.6, 12.8 Hz, 2H), 1.77-1.28 (m, 6H), 1.24 (s, 9H); Low resolution mass spectrum (ES) m/e 500 [(M+1)⁺, calcd for C₃₁H₃₈N₃O₃: 500]; 92.4% purity based on HPLC.

### Example 106

### (S)-[5-AMINO-1-(4-DIETHYLAMINO-PHENYLCARBAMOYL)-PENTYL]-CARBAMIC ACID 9H-FLUOREN-9-YLMETHYL ESTER

N,N-Diethyl-benzene-1,4-diamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d₆) δ 11.22 (br s, 1H), 10.36 (br s, 1H), 9.67 (br s, 1H), 7.89 (d, J=7.5 Hz, 2H), 7.72 (t, J=7.3 Hz, 4H), 7.65 (br s, 2H), 7.41 (ddd, J=2.3, 7.3, 7.4 Hz, 2H), 7.31 (ddd, J=5.3, 7.3, 7.3 Hz, 2H), 6.63 (br s, 1H), 4.34-4.26 (m, 2H), 4.22 (dd, J=6.8, 13.4 Hz, 1H), 4.1 (dd, J=8.2, 13.6 Hz, 2H), 3.42 (br s, 4H), 2.77 (dd, J=6.2, 12.3 Hz, 2H), 1.77-1.24 (m, 6H), 1.00 (t, J=5.9 Hz, 6H); Low resolution mass spectrum (ES) m/e 515 [(M+1)⁺, calcd for C₃₁H₃₉N₄O₃: 515]; 92.4% purity based on HPLC.

### Example 107

### (S)-[5-AMINO-1-(4-CYCLOHEXYL-PHENYLCARBAMOYL)-PENTYL]-CARBAMIC ACID 9H-FLUOREN-9-YLMETHYL ESTER

p-Cyclohexylphenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. 1H NMR (400 MHz, DMSO-d₆) δ 9.93 (br s, 1H), 7.89 (d, J=7.5 Hz, 2H), 7.72 (t, J=7.7 Hz, 2H), 7.69-7.57 (m, 4H), 7.49 (d, J=8.4 Hz, 2H), 7.41 (t, J=6.8 Hz, 2H), 7.31 (dd, J=4.7, 7.3, 7.3 Hz, 2H), 7.14 (d, J=8.4 Hz, 2H), 4.34-4.18 (m, 3H), 4.11 (ddd, J=6.2, 8.3, 8.3 Hz, 1H), 2.7 (dd, J=6.4, 12.7 Hz, 2H), 2.42 (br s, 1H), 1.86-1.59 (m, 7H), 1.59-1.47 (m, 2H), 1.45-1.26 (m, 6H), 1.26-1.14 (m, 1H); Low resolution mass spectrum (ES) m/e 526 [(M+1)⁺, calcd for C₃₃H₄₀N₃O₃: 526]; 95.9% purity based on HPLC.

### Example 108

### (S)-(3-AMINO-1-P-TOLYLCARBAMOYL-PROPYL)-CARBAMIC ACID 9H-FLUOREN-9-YLMETHYL ESTER

p-Tolylamine was coupled to (S)-4-tert-Butoxycarbonylamino-2-(9H-fluoren-9-ylmethoxycarbonylamino)-butanoic acid as described in the method of Example 6. Purification by HPLC produced the title compound. 1H NMR (400 MHz, DMSO-d₆) δ 9.99 (s, 1H), 7.89 (d, J=7.5 Hz, 2H), 7.84 (d, J=7.9 Hz, 1H), 7.76-7.66 (m, 4H), 7.47 (d, J=8.3 Hz, 2H), 7.41 (t, J=7.4 Hz, 2H), 7.32 (ddd, J=3.8, 7.3, 7.3 Hz, 2H), 7.12 (d, J=8.2 Hz, 2H), 4.36-4.18 (m, 4H), 2.83 (dd, J=6.1, 11.8 Hz, 2H), 2.25 (s, 3H), 2.05-1.85 (m, 2H); Low resolution mass spectrum (ES) m/e 430 [(M+1)⁺, calcd for C₂₆H₂₈N₃O₃: 430]; 98.2% purity based on HPLC.

### Example 109

### (3-P-TOLYLCARBAMOYL-PIPERIDIN-3-YL)-CARBAMIC ACID 9H-FLUOREN-9-YLMETHYL ESTER

p-Tolylamine was coupled to 3-(9H-Fluoren-9-ylmethoxycarbonylamino)-piperidine-1,3-dicarboxylic acid 1-tert-butyl ester as described in the method of Example 6. Purification by HPLC produced the title compound. 1H NMR (400 MHz, DMSO-d₆) δ 9.76 (s, 1H), 8.74 (br s, 1H), 8.26 (br s, 1H), 7.88 (d, J=7.3 Hz, 2H), 7.81 (br s, 1H), 7.73 (d, J=4.5 Hz, 2H), 7.46-7.22 (m, 6H), 7.11 (d, J=8.3 Hz, 2H), 4.37 (dd, J=7.1, 10.4 Hz, 1H), 4.29 (dd, J=6.7, 10.4 Hz, 2H), 4.2 (t, J=6.7 Hz, 1H), 3.19-2.92 (m, 4H), 2.24 (s, 2H), 1.89 (br s, 2H), 1.56 (br s, 2H); Low resolution mass spectrum (ES) m/e 456 [(M+1)⁺, calcd for C₂₈H₃₀N₃O₃: 456]; 98.2% purity based on HPLC.

### Example 110

### (S)-[5-((9H-FLUOREN-9-YLMETHOXYCARBONYL)AMINO)-5-P-TOLYLCARBAMOYL-PENTYL]-TRIMETHYL-AMMONIUM

p-Tolylamine was coupled to (S)-[5-Carboxy-5-(9H-fluoren-9-ylmethoxycarbonylamino)-pentyl]-trimethyl-ammonium chloride as described in the method for Intermediate #1 of Example 1. Purification by HPLC produced the title compound. 1H NMR (400 MHz, DMSO-d₆) δ 9.97 (s, 1H), 7.90 (d, J=7.5 Hz, 2H), 7.74 (t, J=8.1 Hz, 2H), 7.69 (d, J=8.0 Hz, 1H), 7.49 (d, J=8.3 Hz, 2H), 7.42 (t, J=7.3 Hz, 2H), 7.33 (dt, J=4.0, 7.4 Hz, 2H), 7.11 (d, J=8.3 Hz, 2H), 4.34-4.20 (m, 3H), 4.15 (dd, J=8.5, 13.9 Hz, 1H), 3.28-3.24 (m, 2H), 3.02 (s, 9H), 2.25 (s, 2H), 1.79-1.62 (m, 4H), 1.43-1.24 (m, 2H); Low resolution mass spectrum (ES) m/e 500 [(M)⁺, calcd for C₃₁H₃₈N₃O₃: 500]; 96.4% purity based on HPLC.

### Example 111

### (S)-[4-(3,3-DIMETHYL-GUANIDINO)-1-P-TOLYLCARBAMOYL-BUTYL]-CARBAMIC ACID 9H-FLUOREN-9-YLMETHYL ESTER

p-Tolylamine was coupled to (S)-6-(3,3-Dimethyl-guanidino)-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 9.96 (s, 1H), 7.90 (d, J=7.5 Hz, 2H), 7.73 (d, J=7.8 Hz, 2H), 7.67 (d, J=7.9 Hz, 1H), 7.49 (d, J=8.3 Hz, 2H), 7.43-7.30 (m, 7H), 7.11 (d, J=8.3 Hz, 2H), 4.34-4.21 (m, 3H), 4.15 (dd, J=8.0, 13.6 Hz, 1H), 3.17 (m, 2H), 2.93 (s, 6H), 2.25 (s, 3H), 1.76-1.46 (m, 4H); Low resolution mass spectrum (ES) m/e 514 [(M+1)⁺, calcd for C₃₀H₃₆N₅O₃: 514]; 94.5% purity based on HPLC.

### Example 112

### (S)-(3-METHYLSULFANYL-1-P-TOLYLCARBAMOYL-PROPYL)-CARBAMIC ACID 9H-FLUOREN-9-YLMETHYL ESTER

p-Tolylamine was coupled to (S)-2-(9H-Fluoren-9-ylmethoxycarbonylamino)-4-methylsulfanyl-butyric acid as described in the method for Intermediate #1 of Example 1. Purification by HPLC produced the title compound. 1H NMR (400 MHz, DMSO-d₆) δ 9.95 (s, 1H), 7.89 (d, J=7.5 Hz, 2H), 7.73 (dd, J=7.2, 13.3 Hz, 3H), 7.48 (d, J=8.3 Hz, 2H), 7.42 (t, J=7.4 Hz, 2H), 7.32 (dt, J=3.1, 7.4 Hz, 2H), 7.11 (d, J=8.3 Hz, 2H), 4.3-4.2 (m, 4H), 2.58-2.43 (m, 2H), 2.25 (s, 3H), 2.06 (s, 3H), 1.99-1.85 (m, 2H); Low resolution mass spectrum (ES) m/e 461 [(M+1)⁺, calcd for C₂₇H₂₉N₂O₃S: 461]; 90.4% purity based on HPLC.

### Example 113

### (S)-(1-P-TOLYLCARBAMOYL-4-UREIDO-BUTYL)-CARBAMIC ACID 9H-FLUOREN-9-YLMETHYL ESTER

p-Tolylamine was coupled to (S)-2-(9H-Fluoren-9-ylmethoxycarbonylamino)-6-ureido-hexanoic acid as described in the method for Intermediate #1 of Example 1. Purification by HPLC produced the title compound. 1H 1H NMR (400 MHz, DMSO-d₆) δ 9.93 (s, 1H), 7.89 (d, J=7.5 Hz, 2H), 7.74 (dd, J=4.7, 7.2 Hz, 2H), 7.66 (d, J=8.0 Hz, 1H), 7.49 (d, J=8.4 Hz, 2H), 7.41 (d, J=7.4 Hz, 2H), 7.33 (dt, J=4.5, 7.1 Hz, 2H), 7.10 (d, J=8.3 Hz, 2H), 5.90 (t, J=5.5 Hz, 1H), 5.42 (s, 2H), 4.28-4.2 (m, 2H), 4.15 (dd, J=8.5, 13.6 Hz, 1H), 3.07-2.91 (m, 2H), 2.25 (s, 3H), 1.72-1.34 (m, 4H); Low resolution mass spectrum (ES) m/e 487 [(M+1)⁺, calcd for C₂₈H₃₁N₄O₄: 487]; 97.5% purity based on HPLC.

### Example 114

### (S)-2,6-DIAMINO-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid as described in the method of Example 5 and further deprotected as described in the method of Example 2. Purification by HPLC produced the title compound. ¹H NMR (400 MHz, DMSO-d₆) δ 10.47 (s, 1H), 8.29 (br s, 3H), 7.78 (br s, 3H), 7.49 (d, J=8.4 Hz, 2H), 7.16 (d, J=8.3 Hz, 2H), 3.92 (br s, 1H), 2.76 (br s, 2H), 2.27 (s, 3H), 1.85-1.75 (m, 2H), 1.59-1.51 (m, 2H), 1.41-1.35 (m, 2H); Low resolution mass spectrum (ES) m/e 236 [(M+1)⁺, calcd for C₁₃H₂₁N₃O: 236]; 100% purity based on HPLC.

### Example 115

### (S)-[2-(2-AMINO-ACETYLAMINO)-1-P-TOLYLCARBAMOYL-ETHYL]-CARBAMIC ACID 9H-FLUOREN-9-YLMETHYL ESTER

tert-Butoxycarbonylamino-acetic acid was coupled to the compound of Example 103 as described in the method of Example 6. Purification by HPLC produced the title compound. 1H NMR (400 MHz, DMSO-d₆) δ 10.01 (s, 1H), 8.43 (br s, 1H), 7.98 (br s, 3H), 7.90 (d, J=7.5 Hz, 2H), 7.73 (dd, J=3.6, 7.2 Hz, 2H), 7.64 (d, J=8.1 Hz, 1H), 7.49 (d, J=8.3 Hz, 2H), 7.42 (t, J=7.4 Hz, 2H), 7.33 (dd, J=6.9, 13.3 Hz, 2H), 7.12 (d, J=8.3 Hz, 2H), 4.38-4.22 (m, 4H), 3.53-3.46 (m, 4H), 2.25 (s, 3H); Low resolution mass spectrum (ES) m/e 473 [(M+1)⁺, calcd for C₂₇H₂₉N₄O₄: 473]; 99.2% purity based on HPLC.

### Example 116

### (S)-[6-AMINO-1-(4-CYCLOHEXYL-PHENYLCARBAMOYL)-HEXYL]-CARBAMIC ACID 9H-FLUOREN-9-YLMETHYL ESTER

p-Cyclohexyphenylamine was coupled to (S)-8-tert-Butoxycarbonylamino-2-(9H-fluoren-9-ylmethoxycarbonylamino)-octanoic acid as described in the method of Example 6. Purification by HPLC produced the title compound. 1H NMR (400 MHz, DMSO-d₆) δ 9.92 (s, 1H), 7.89 (d, J=7.5 Hz, 2H), 7.73 (t, J=7.8 Hz, 2H), 7.67-7.54 (m, 3H), 7.48 (d, J=8.5 Hz, 2H), 7.41 (t, J=7.3 Hz, 3H), 7.31 (ddd, J=3.1, 7.3, 7.4 Hz, 2H), 7.13 (d, J=8.5 Hz, 2H), 4.31-4.18 (m, 3H), 4.11 (dd, J=8.1, 14.1 Hz, 1H), 2.75 (dd, J=6.7, 13.4 Hz, 2H), 2.42 (br s, 1H), 1.82-1.44 (m, 9H), 1.44-1.12 (m, 9H); Low resolution mass spectrum (ES) m/e 540 [(M+1)⁺, calcd for C₃₄H₄₂N₃O₃: 540]; 98% purity based on HPLC.

### Example 117

### (S)-6-AMINO-2-[3-(4-BENZYLOXY-PHENYL)-UREIDO]-HEXANOIC ACID (4-CYCLOHEXYL-PHENYL)-AMIDE

p-Cyclohexyphenylamine was coupled to (S)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. 1H NMR (400 MHz, DMSO-d₆) δ 10.07 (s, 1H), 8.54 (s, 1H), 7.66 (br s, 3H), 7.51 (d, J=8.6 Hz, 2H), 7.44-7.36 (m, 4H), 7.32 (d, J=7.1 Hz, 1H), 7.28 (d, J=9.1 Hz, 2H), 7.15 (d, J=8.6 Hz, 2H), 6.89 (d, J=9.1 Hz, 2H), 6.43 (d, J=8.3 Hz, 1H), 5.03 (s, 2H), 4.38 (dd, J=7.9, 13.7 Hz, 1H), 2.8-2.75 (m, 2H), 2.43 (m, 1H), 1.78-1.68 (m, 6H), 1.64-1.19 (m, 10H); Low resolution mass spectrum (ES) m/e 529 [(M+1)⁺, calcd for C₃₂H₄₁N₄O₃: 529]; 90.1% purity based on HPLC.

### Example 118

### (S)-6-AMINO-2-[3-(3-BROMO-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Bromo-3-isocyanato-benzene as described in the method of Example 26. Purification by HPLC produced the title compound. ¹H NMR (400 MHz, DMSO-d₆) δ 10.09 (s, 1H), 8.97 (m, 1H), 7.82 (s, 1H), 7.66 (br s, 3H), 7.49 (d, J=8.4 Hz, 2H), 7.19 (d, J=5.3 Hz, 2H), 7.12 (d, J=8.3 Hz, 2H), 7.07 (m, 1H), 6.65 (m, 1H), 4.38 (dd, J=7.9, 13.4 Hz, 1H), 2.78 (br s, 2H), 2.25 (s, 3H), 1.79-1.7 (m, 1H), 1.66-1.5 (m, 3H), 1.45-1.3 (m, 2H); Low resolution mass spectrum (ES) m/e 433 [(M)⁺, calcd for C₂₀H₂₆BrN₄O₂: 433]; 100% purity based on HPLC.

### Example 119

### (S)-6-AMINO-2-(3-BIPHENYL-4-YL-UREIDO)-HEXANOIC ACID (4-CYCLOHEXYL-PHENYL)-AMIDE

p-Cyclohexylphenylamine was coupled to (S)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d₆) δ 10.11 (s, 1H), 8.88 (m, 1H), 7.70 (br s, 3H), 7.61 (d, J=7.3 Hz, 2H), 7.57-7.47 (m, 6H), 7.42 (t, J=7.7 Hz, 2H), 7.29 (t, J=7.3 Hz, 1H), 7.16 (d, J=8.5 Hz, 2H), 6.63 (m, 1H), 4.41 (dd, J=7.8, 13.5 Hz, 1H), 2.83-2.75 (m, 2H), 2.44 (m, 1H), 1.76-1.5 (m, 9H), 1.43-1.30 (m, 6H), 1.26-1.19 (m, 1H); Low resolution mass spectrum (ES) m/e 499 [(M+1)⁺, calcd for C₃₁H₃₉N₄O₂: 499]; 98.1% purity based on HPLC.

### Example 120

### (S)-7-AMINO-2-(3-BENZYL-UREIDO)-HEPTANOIC ACID (4-CYCLOHEXYL-PHENYL)-AMIDE

p-Cyclohexyphenylamine was coupled to (S)-8-tert-Butoxycarbonylamino-2-(9H-fluoren-9-ylmethoxycarbonylamino)-octanoic acid, and then to benzyl isocyanate as described in the method of Example 26. Purification by HPLC produced the title compound. 1H NMR (400 MHz, DMSO-d₆) δ 9.97 (s, 1H), 7.61 (br s, 3H), 7.48 (d, J=8.5 Hz, 2H), 7.34-7.26 (m, 2H), 7.26-7.17 (m, 3H), 7.14 (d, J=8.5 Hz, 2H), 6.54 (t, J=6.0 Hz, 1H), 6.29 (d, J=8.5 Hz, 1H), 4.43 (dd, J=7.6, 14.1 Hz, 2H), 2.79-2.68 (m, 2H), 2.42 (br s, 1H), 1.86-1.58 (m, 6H), 1.57-1.43 (m, 3H), 1.42-1.12 (m, 9H); Low resolution mass spectrum (ES) m/e 451 [(M+1)⁺, calcd for C₂₇H₃₉N₄O₂: 450]; 100% purity based on HPLC.

### Example 121

### (S)-7-AMINO-2-[3-(4-BENZYLOXY-PHENYL)-UREIDO]-HEPTANOIC ACID (4-CYCLOHEXYL-PHENYL)-AMIDE

p-Cyclohexyphenylamine was coupled to (S)-8-tert-Butoxycarbonylamino-2-(9H-fluoren-9-ylmethoxycarbonylamino)-octanoic acid, and then to 1-Benzyloxy-4-isocyanato-benzene as described in the method of Example 26. Purification by HPLC produced the title compound. 1H NMR (400 MHz, DMSO-d₆) δ 10.00 (s, 1H), 8.49 (s, 1H), 7.59 (br s, 3H), 7.49 (d, J=8.5 Hz, 2H), 7.45-7.21 (m, 7H), 7.14 (d, J=8.5 Hz, 2H), 6.88 (d, J=9.0 Hz, 2H), 6.40 (d, J=8.3 Hz, 1H), 5.01 (s, 2H), 4.37 (dd, J=7.4, 13.8 Hz, 1H), 2.79-2.69 (m, 2H), 2.43 (br s, 1H), 1.86-1.62 (m, 6H), 1.62-1.42 (m, 3H), 1.42-1.12 (m, 9H); Low resolution mass spectrum (ES) m/e 543 [(M+1)⁺, calcd for C₃₃H₄₃N₄O₃: 543]; 97% purity based on HPLC.

### Example 122

### (S)-2-P-TOLYLCARBAMOYL-PIPERIDINE-1-CARBOXYLIC ACID 9H-FLUOREN-9-YLMETHYL ESTER

p-Tolylamine was coupled to (S)-Piperidine-1,2-dicarboxylic acid 1-(9H-fluoren-9-ylmethyl) ester as described in the method for Intermediate #1 of Example 1. Purification by HPLC produced the title compound. 1H NMR (400 MHz, DMSO-d₆) δ 7.92-7.75 (m, 2H), 7.68-7.25 (m, 7H), 7.16-6.96 (m, 3H), 4.79 (br s, 0.5H), 4.68 (br s, 0.5H), 4.41-4.11 (m, 3H), 3.92 (d, J=11.6 Hz, 0.5H), 3.77 (d, J=11.2 Hz, 0.5H), 3.37-3.16 (m, 1H), 2.28-2.00 (m, 4H), 1.78-1.50 (m, 3H), 1.40-1.16 (m, 2H); Low resolution mass spectrum (ES) m/e 441 [(M+1)⁺, calcd for C₂₈H₂₉N₂O₃: 441]; 95% purity based on HPLC.

### Example 123

### (R)-2-P-TOLYLCARBAMOYL-PIPERIDINE-1-CARBOXYLIC ACID 9H-FLUOREN-9-YLMETHYL ESTER

p-Tolylamine was coupled to (R)-Piperidine-1,2-dicarboxylic acid 1-(9H-fluoren-9-ylmethyl) ester as described in the method for Intermediate #1 of Example 1. Purification by HPLC produced the title compound. 1H NMR (400 MHz, DMSO-d₆) δ 7.92-7.75 (m, 2H), 7.68-7.25 (m, 7H), 7.16-6.96 (m, 3H), 4.79 (br s, 0.5H), 4.68 (br s, 0.5H), 4.41-4.11 (m, 3H), 3.92 (d, J=11.6 Hz, 0.5H), 3.77 (d, J=11.2 Hz, 0.5H), 3.37-3.16 (m, 1H), 2.28-2.00 (m, 4H), 1.78-1.50 (m, 3H), 1.40-1.16 (m, 2H); Low resolution mass spectrum (ES) m/e 441 [(M+1)⁺, calcd for C₂₈H₂₉N₂O₃: 441]; 95% purity based on HPLC.

### Example 124

### (S)-(5-DIMETHYLAMINO-1-P-TOLYLCARBAMOYL-PENTYL)-CARBAMIC ACID 9H-FLUOREN-9-YLMETHYL ESTER

p-Tolylamine was coupled to (S)-6-Dimethylamino-2-(9H-fluoren-9-ylmethoxycarbonylamino)-hexanoic acid as described in the method for Intermediate #1 of Example 1. Purification by HPLC produced the title compound. 1H NMR (400 MHz, DMSO-d₆) δ 7.86 (d, J=7.5 Hz, 2H), 7.70 (t, J=7.9 Hz, 2H), 7.47-7.36 (m, 4H), 7.32 (d, J=6.9 Hz, 1H), 7.29 (d, J=7.2 Hz, 1H), 7.10 (d, J=8.3 Hz, 2H), 4.34-4.03 (m, 4H), 2.99 (t, J=7.7 Hz, 2H), 2.72 (s, 6H), 2.22 (s, 3H), 1.75-1.52 (m, 4H), 1.44.1.18 (m, 2H); Low resolution mass spectrum (ES) m/e 486 [(M+1)⁺, calcd for C₃₀H₃₆N₃O₃: 486]; 85% purity based on HPLC.

### Example 125

### (S)-6-AMINO-2-[3-(9H-FLUOREN-2-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. 1H NMR (400 MHz, DMSO-d₆) δ 10.10 (s, 1H), 8.86 (s, 1H), 7.74 (m, 3H), 7.69 (br s, 3H), 7.51 (m, 3H), 7.32 (m, 2H), 7.22 (dt, J=1.1, 7.4 Hz, 1H), 7.12 (dt, J=8.3 Hz, 2H), 6.61 (t, J=8.2 Hz, 1H), 4.41 (dt, J=5.5, 8.1 Hz, 1H), 3.85 (s, 2H), 2.83-2.76 (m, 2H), 2.25 (s, 3H), 1.79-1.72 (m, 1H), 1.66-1.53 (m, 3H), 1.45-1.33 (m, 2H); Low resolution mass spectrum (ES) m/e 443 [(M+1)⁺, calcd for C₂₇H₃₁N₄O₂: 443]; 99.7% purity based on HPLC.

### Example 126

### (S)-6-AMINO-2-[3-(9H-FLUOREN-9-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to 9-Isocyanato-9H-fluorene as described in the method of Example 26. Purification by HPLC produced the title compound. 1H NMR (400 MHz, DMSO-d₆) δ 10.09 (s, 1H), 7.84 (dd, J=2.7, 7.5 Hz, 2H), 7.70 (br s, 3H), 7.52 (m, 4H), 7.41 (dd, J=6.9, 14.0 Hz, 2H), 7.31 (ddt, J=0.9, 7.5, 8.4 Hz, 2H), 7.13 (d, J=8.5 Hz, 2H), 6.64 (d, J=8.7 Hz, 1H), 6.57 (s, 0.4H), 6.25 (d, J=8.5 Hz, 1H), 5.82 (d, J=8.6 Hz, 1H), 4.47 (dt, J=5.9, 8.2 Hz, 1H), 2.82-2.78 (m, 2H), 2.26 (s, 3H), 1.77-1.7 (m, 1H), 1.63-1.55 (m, 3H), 1.45-1.34 (m, 2H); Low resolution mass spectrum (ES) m/e 443 [(M+1)⁺, calcd for C₂₇H₃₁N₄O₂: 443]; 98% purity based on HPLC.

### Example 127

### (S)-6-AMINO-2-(3-BIPHENYL-3-YL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to 3-Isocyanato-biphenyl as described in the method of Example 26. Purification by HPLC produced the title compound. 1H NMR (400 MHz, DMSO-d₆) δ 10.09 (s, 1H), 8.87 (s, 1H), 7.73 (m, 1H), 7.68 (br s, 3H), 7.58 (dd, J=1.2, 8.3 Hz, 2H), 7.50 (d, J=8.5 Hz, 2H), 7.46 (t, J=7.7 Hz, 2H), 7.35 (m, 3H), 7.19 (m, 1H), 7.12 (dd, J=8.3 Hz, 2H), 6.62 (dd, J=8.2 Hz, 1H), 4.40 (dt, J=5.6, 8.1 Hz, 1H), 2.79 (dd, J=7.3, 13.2 Hz, 2H), 2.25 (s, 3H), 1.78-1.72 (m, 1H), 1.66-1.54 (m, 3H), 1.44-1.34 (m, 2H); Low resolution mass spectrum (ES) m/e 431 [(M+1)⁺, calcd for C₂₆H₃₁N₄O₂: 431]; 95.9% purity based on HPLC.

### Example 128

### (R)-[5-AMINO-1-(4-CYCLOHEXYL-PHENYLCARBAMOYL)-PENTYL]-CARBAMIC ACID 9H-FLUOREN-9-YLMETHYL ESTER

p-Cyclohexylphenylamine was coupled to (R)-6-tert-Butoxycarbonylamino-2-(9H-fluoren-9-ylmethoxycarbonylamino)-hexanoic acid as described in the method of Example 6 and purified by HPLC to produce the title compound. 1H NMR (400 MHz, DMSO-d₆) δ 9.95 (s, 1H), 7.89 (d, J=7.5 Hz, 2H), 7.72 (t, J=7.7 Hz, 2H), 7.68 (br s, 3H), 7.62 (d, J=8.0 Hz, 1H), 7.5 (d, J=8.4 Hz, 2H), 7.42 (t, J=6.9 Hz, 2H), 7.32 (dd, J=7.2, 12.0 Hz, 2H), 7.14 (d, J=8.4 Hz, 2H), 4.33-4.20 (m, 3H), 4.12 (dd, J=8.2, 13.8 Hz, 1H), 2.8-2.75 (m, 2H), 2.43 (br s, 1H), 1.78-1.19 (m, 16H); Low resolution mass spectrum (ES) m/e 526 [(M+1)⁺, calcd for C₃₃H₄₀N₃O₃: 526]; 95.8% purity based on HPLC.

### Example 129

### (S)-[2-(4-AMINOMETHYL-PHENYL)-1-P-TOLYLCARBAMOYL-ETHYL]-CARBAMIC ACID 9H-FLUOREN-9-YLMETHYL ESTER

p-Tolylamine was coupled to (S)-3-[4-(tert-Butoxycarbonylamino-methyl)-phenyl]-2-(9H-fluoren-9-ylmethoxycarbonylamino)-propionic acid as described in the method of Example 6 and purified by HPLC to produce the title compound. 1H NMR (400 MHz, DMSO-d₆) δ 7.85 (d, J=7.0 Hz, 2H), 7.67-7.60 (br, 2H), 7.48-7.24 (m, 10H), 7.11 (d, J=8.3 Hz, 2H), 4.39-4.09 (m, 4H), 3.02 (dd, J=4.2, 14.0 Hz, 2H), 2.87 (t, J=11.2, 13.2 Hz, 2H), 2.23 (s, 3H); Low resolution mass spectrum (ES) m/e 506.2 [(M+H)⁺, calcd for C₃₂H₃₂N₃O₃: 506.6]; 90% purity based on NMR.

### Example 130

### (S)-(4-(R)-(3-AMINO-PROPIONYLOXY)-2-P-TOLYLCARBAMOYL-PYRROLIDINE-1-CARBOXYLIC ACID 9H-FLUOREN-9-YLMETHYL ESTER)

(4R,S)-4-Hydroxy-2-p-tolylcarbamoyl-pyrrolidine-1-carboxylic acid 9H-fluoren-9-ylmethyl ester from Example 87 was coupled to 3-tert-Butoxycarbonylamino-propionic acid as described in the method of Example 6 and purified by HPLC to produce the title compound. 1H NMR (400 MHz, DMSO-d₆) δ 7.88 (dd, J=3.1, 7.5 Hz, 1H), 7.80 (d, J=7.7 Hz, 1H), 7.66-7.48 (m, 3H), 7.47-7.38 (m, 2H), 7.36-7.28 (m, 2H), 7.14-7.03 (m, 3H), 5.31 (br s, 1H), 4.57 (t, J=7.7, 7.2 Hz, 1H), 4.38 (t, J=8.3, 7.7 Hz, 1H), 4.33-4.22 (m, 2H), 4.10-4.00 (m, 2H), 3.75 (dd, J=5.0, 12.1 Hz, 1H), 3.65 (d, J=12.5 Hz, 1H), 3.03 (t, J=6.6 Hz, 2H), 2.71-2.62 (m, 2H), 2.22 (s, 3H); Low resolution mass spectrum (ES) m/e 514.2 [(M+H)⁺, calcd for C₃₀H₃₂N₃O₅: 514.6]; 90% purity based on NMR.

### Example 131

### (S)-[4-(R)-(2-AMINO-ACETOXY)-2-P-TOLYLCARBAMOYL-PYRROLIDINE-1-CARBOXYLIC ACID 9H-FLUOREN-9-YLMETHYL ESTER]

(4R,S)-4-Hydroxy-2-p-tolylcarbamoyl-pyrrolidine-1-carboxylic acid 9H-fluoren-9-ylmethyl ester of Example 87 was coupled to tert-Butoxycarbonylamino-acetic acid as described in the method for M-2024 and purified by HPLC to produce the title compound. 1H NMR (400 MHz, DMSO-d6) δ 7.88 (d, J=7.7 Hz, 1H), 7.79 (d, J=7.7 Hz, 1H), 7.62 (t, J=7.0, 7.2 Hz, 1H), 7.59-7.47 (m, 2H), 7.47-7.37 (m, 2H), 7.37-7.27 (m, 2H), 7.14-7.03 (m, 3H), 5.40 (br s, 1H), 4.58 (t, J=7.7, 7.2 Hz, 1H), 4.40 (t, J=8.2, 7.7 Hz, 1H), 4.34-4.20 (m, 2H), 4.09-3.98 (m, 2H), 3.87-3.74 (m, 3H), 3.55 (d, J=11.8 Hz, 1H), 2.22 (s, 3H); Low resolution mass spectrum (ES) m/e 500.2 [(M+H)⁺, calcd for C₂₉H₃₀N₃O₅: 500.6]; 90% purity based on NMR.

### Example 132

### (R)-4-[2-(S)-(2-AMINO-ACETYLAMINO)-ACETOXY]-2-P-TOLYLCARBAMOYL-PYRROLIDINE-1-CARBOXYLIC ACID 9H-FLUOREN-9-YLMETHYL ESTER

(4R,S)-4-Hydroxy-2-p-tolylcarbamoyl-pyrrolidine-1-carboxylic acid 9H-fluoren-9-ylmethyl ester from Example 87 was coupled to (2-tert-Butoxycarbonylamino-acetylamino)-acetic acid as described in the method of Example 6 and purified by HPLC to produce the title compound. 1H NMR (400 MHz, DMSO-d6) δ 7.89 (d, J=7.7 Hz, 1H), 7.80 (d, J=8.1 Hz, 1H), 7.66-7.49 (m, 3H), 7.47-7.38 (m, 2H), 7.38-7.28 (m, 2H), 7.15-7.02 (m, 3H), 5.31 (br s, 1H), 4.59 (t, J=8.3, 7.7 Hz, 1H), 4.39 (t, J=7.7, 7.5 Hz, 1H), 4.33-4.21 (m, 2H), 4.12-4.01 (m, 4H), 3.99 (s, 2H), 3.76 (dd, J=5.3, 12.3 Hz, 1H), 3.66-3.59 (m, 3H), 2.22 (s, 3H); Low resolution mass spectrum (ES) m/e 557.2 [(M+H)⁺, calcd for C₃₁H₃₃N₄O₆: 557.7]; 90% purity based on NMR.

### Example 133

### (S)-6-AMINO-2-(3-BENZYL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid then to benzyl isocyanate as described in the method of Example 26. Purification by HPLC produced the title compound. 1H NMR (400 MHz, DMSO-d₆) δ 9.99 (s, 1H), 7.72 (br s, 3H), 7.49 (d, J=8.4 Hz, 2H), 7.32-7.29 (m, 2H), 7.25-7.20 (m, 3H), 7.11 (d, J=8.3 Hz, 2H), 6.60 (dt, J=2.5, 5.8 Hz, 1H), 6.33 (dd, J=2.1, 8.4 Hz, 1H), 4.32 (dd, J=8.1, 13.9 Hz, 1H), 4.22 (d, J=5.9 Hz, 2H), 2.77 (t, J=7.4 Hz, 2H), 2.25 (s, 3H), 1.72-1.63 (m, 1H), 1.59-1.50 (m, 3H), 1.43-1.27 (m, 2H); Low resolution mass spectrum (ES) m/e 369 [(M+1)⁺, calcd for C₂₁H₂₉N₄O₂: 369]; 99.4% purity based on HPLC.

### Example 134

### (S)-6-AMINO-2-(3-PHENETHYL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to (2-Isocyanato-ethyl)-benzene as described in the method of Example 26. Purification by HPLC produced the title compound. 1H NMR (400 MHz, DMSO-d₆) δ 9.97 (s, 1H), 7.68 (br s, 3H), 7.48 (d, J=8.4 Hz, 2H), 7.30-7.27 (m, 2H), 7.21-7.17 (m, 3H), 7.10 (d, J=8.3 Hz, 2H), 6.25 (d, J=8.4 Hz, 1H), 6.11 (t, J=5.4 Hz, 1H), 4.28 (dd, J=8.1, 13.8 Hz, 1H), 3.23 (ddd, J=2.4, 7.2, 12.6 Hz, 2H), 2.80-2.72 (m, 2H), 2.67 (t, J=7.2 Hz, 2H), 2.25 (s, 3H), 1.69-1.6 (m, 1H), 1.57-1.46 (m, 3H), 1.40-1.24 (m, 2H); Low resolution mass spectrum (ES) m/e 383 [(M+1)⁺, calcd for C₂₂H₃₁N₄O₂: 383]; 100% purity based on HPLC.

### Example 135

### (S)-6-AMINO-2-(3-P-TOLYL-UREIDO)-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to p-tolyl isocyanate as described in the method of Example 26. Purification by HPLC produced the title compound. 1H NMR (400 MHz, DMSO-d₆) δ 10.07 (s, 1H), 8.65-8.62 (m, 1H), 7.71 (br s, 3H), 7.50 (d, J=8.3 Hz, 2H), 7.26 (d, J=8.1 Hz, 2H), 7.11 (d, J=8.3 Hz, 2H), 7.02 (d, J=8.3 Hz, 2H), 6.54-6.49 (m, 1H), 4.37 (dd, J=7.9, 13.5 Hz, 1H), 2.79 (m, 2H), 2.25 (s, 3H), 2.21 (s, 3H), 1.77-1.68 (m, 1H), 1.64-1.51 (m, 3H), 1.45-1.31 (m, 2H); Low resolution mass spectrum (ES) m/e 369 [(M+1)⁺, calcd for C₂₁H₂₉N₄O₂: 369]; 100% purity based on HPLC.

### Example 136

### (S)-6-AMINO-2-[3-(4-METHYL-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanatomethyl-4-methyl-benzene as described in the method of Example 26. Purification by HPLC produced the title compound. 1H NMR (400 MHz, DMSO-d₆) δ 9.98 (s, 1H), 7.69 (br s, 3H), 7.48 (d, J=8.4 Hz, 2H), 7.12 (m, 6H), 6.53 (t, J=5.8 Hz, 1H), 6.29 (d, J=8.4 Hz, 1H), 4.31 (dd, J=8.1, 13.9 Hz, 1H), 4.16 (d, J=5.7 Hz, 2H), 2.81-2.73 (m, 2H), 2.26 (s, 3H), 2.25 (s, 3H), 1.71-1.63 (m, 1H), 1.60-1.49 (m, 3H), 1.42-1.24 (m, 2H); Low resolution mass spectrum (ES) m/e 383 [(M+1)⁺, calcd for C₂₂H₃₁N₄O₂: 383]; 99.6% purity based on HPLC.

### Example 137

### (R)-6-AMINO-2-[3-(9H-FLUOREN-2-YL)-UREIDO]-HEXANOIC ACID (4-CYCLOHEXYL-PHENYL)-AMIDE

p-Cyclohexylphenyl amine was coupled to (R)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. 1H NMR (400 MHz, DMSO-d₆) δ 10.11 (s, 1H), 8.84 (s, 1H), 7.74 (m, 3H), 7.66 (br s, 3H), 7.52 (m, 3H), 7.32 (m, 2H), 7.22 (dt, J=1.0, 7.5 Hz, 1H), 7.16 (d, J=8.6 Hz, 2H), 6.59 (d, J=8.2 Hz, 1H), 4.42 (dd, J=7.8, 13.5 Hz, 1H), 3.85 (s, 2H), 2.83-2.75 (m, 2H), 2.47-2.41 (m, 1H), 1.78-1.19 (m, 16H); Low resolution mass spectrum (ES) m/e 511 [(M+1)⁺, calcd for C₃₂H₃₉N₄O₂: 511]; 91.1% purity based on HPLC.

### Example 138

### (4-P-TOLYLCARBAMOYL-PIPERIDIN-4-YL)-CARBAMIC ACID 9H-FLUOREN-9-YLMETHYL ESTER

p-Tolylamine was coupled to 4-(9H-Fluoren-9-ylmethoxycarbonylamino)-piperidine-1,4-dicarboxylic acid mono-tert-butyl ester as described in the method of Example 6 and purified by HPLC to produce the title compound. 1H NMR (400 MHz, DMSO-d₆) δ 9.48 (s, 1H), 8.48 (br s, 1H), 8.30 (br s, 3H), 7.89 (d, J=7.5 Hz, 2H), 7.78-7.62 (m, 3H), 7.38 (t, J=7.5 Hz, 4H), 7.26 (t, J=7.0 Hz, 2H), 7.06 (d, J=8.4 Hz, 2H), 4.33 (d, J=6.8 Hz, 2H), 4.18 (t, J=5.9 Hz, 1H), 3.23-2.97 (m, 4H), 2.21 (s, 3H), 2.12 (br s, 4H); Low resolution mass spectrum (ES) m/e 456 [(M+1)⁺, calcd for C₂₈H₃₀N₃O₃: 456]; 98% purity based on HPLC.

### Example 139

### (S)-6-AMINO-2-[3-(4-METHOXY-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanatomethyl-4-methoxy-benzene as described in the method of Example 26. Purification by HPLC produced the title compound. 1H NMR (400 MHz, DMSO-d₆) δ 9.48 (s, 1H), 9.98 (s, 1H), 7.70 (br s, 3H), 7.48 (d, J=8.4 Hz, 2H), 7.16 (d, J=8.6 Hz, 2H), 7.11 (d, J=8.3 Hz, 2H), 6.86 (d, J=8.6 Hz, 2H), 6.50 (t, J=5.9 Hz, 1H), 6.27 (d, J=8.5 Hz, 1H), 4.31 (dd, J=8.1, 13.8 Hz, 1H), 4.14 (dd, J=2.3, 5.5 Hz, 2H), 3.72 (s, 3H), 2.81-2.73 (m, 2H), 2.25 (s, 3H), 1.71-1.49 (m, 4H), 1.42-1.24 (m, 2H); Low resolution mass spectrum (ES) m/e 399 [(M+1)⁺, calcd for C₂₂H₃₁N₄O₃: 399]; 99.3% purity based on HPLC.

### Example 140

### (S)-6-AMINO-2-[3-(3,4-DICHLORO-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1,2-Dichloro-4-isocyanatomethyl-benzene as described in the method of Example 26. Purification by HPLC produced the title compound. 1H NMR (400 MHz, DMSO-d₆) δ 9.98 (s, 1H), 7.71 (br s, 3H), 7.56 (d, J=8.3 Hz, 1H), 7.49 (m, 3H), 7.23 (dd, J=1.9, 8.3 Hz, 1H), 7.10 (d, J=8.3 Hz, 2H), 6.70 (m, 1H), 6.42 (m, 1H), 4.29 (dd, J=8.2, 13.9 Hz, 1H), 4.21 (d, J=6.1 Hz, 2H), 2.81-2.73 (m, 2H), 2.25 (s, 3H), 1.73-1.64 (m, 1H), 1.58-1.51 (m, 3H), 1.43-1.27 (m, 2H); Low resolution mass spectrum (ES) m/e 437 [(M)⁺, calcd for C₂₁H₂₇Cl₂N₄O₂: 438]; 99.3% purity based on HPLC.

### Example 141

### (R)-6-AMINO-2-(3-BENZYL-UREIDO)-HEXANOIC ACID (4-CYCLOHEXYL-PHENYL)-AMIDE

p-Cyclohexylphenyl amine was coupled to (R)-6-tert-Butoxycarbonylamino-2-(9H-fluoren-9-ylmethoxycarbonylamino)-hexanoic acid then to benzyl isocyanate as described in the method of Example 26. Purification by HPLC produced the title compound. 1H NMR (400 MHz, DMSO-d₆) δ 10.00 (s, 1H), 7.68 (br s, 3H), 7.50 (d, J=8.5 Hz, 2H), 7.32-7.28 (m, 2H), 7.25-7.20 (m, 3H), 7.15 (d, J=8.5 Hz, 2H), 6.59 (t, J=6.0 Hz, 1H), 6.32 (d, J=8.5 Hz, 1H), 4.32 (dd, J=8.0, 13.9 Hz, 1H), 4.22 (dd, J=2.6, 5.7 Hz, 2H), 2.79-2.74 (m, 2H), 2.43 (br s, 1H), 1.78-1.51 (m, 9H), 1.42-1.17 (m, 7H); Low resolution mass spectrum (ES) m/e 437 [(M+1)⁺, calcd for C₂₆H₃₇N₄O₂: 438]; 97.4% purity based on HPLC.

### Example 142

### (R)-6-AMINO-2-(3-BIPHENYL-4-YL-UREIDO)-HEXANOIC ACID (4-CYCLOHEXYL-PHENYL)-AMIDE

p-Cyclohexylphenyl amine was coupled to (R)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d₆) δ 10.09 (s, 1H), 8.85 (s, 1H), 7.66 (br s, 3H), 7.61 (d, J=7.2 Hz, 2H), 7.57-7.47 (m, 6H), 7.42 (t, J=7.7 Hz, 2H), 7.30 (t, J=7.3 Hz, 1H), 7.16 (d, J=8.5 Hz, 2H), 6.59 (d, J=8.2 Hz, 1H), 4.42 (dd, J=7.8, 13.5 Hz, 1H), 2.83-2.75 (m, 2H), 2.44 (m, 1H), 1.76-1.50 (m, 9H), 1.41-1.12 (m, 7H); Low resolution mass spectrum (ES) m/e 499 [(M+1)⁺, calcd for C₃₁H₃₉N₄O₂: 499]; 97% purity based on HPLC.

### Example 143

### (S)-[5-AMINO-1-(BIPHENYL-4-YLCARBAMOYL)-PENTYL]-CARBAMIC ACID 9H-FLUOREN-9-YLMETHYL ESTER

Biphenyl-4-ylamine was coupled to (S)-6-tert-Butoxycarbonylamino-2-(9H-fluoren-9-ylmethoxycarbonylamino)-hexanoic acid as described in the method of Example 6, and then purified by HPLC to produce the title compound. 1H NMR (400 MHz, DMSO-d₆) δ 10.15 (s, 1H), 7.90 (d, J=7.5 Hz, 2H), 7.76-7.63 (m, 12H), 7.46-7.40 (m, 4H), 7.35-7.31 (m, 3H), 4.36-4.22 (m, 3H), 4.17 (dd, J=8.3, 13.7 Hz, 1H), 2.82-2.77 (m, 2H), 1.78-1.30 (m, 6H); Low resolution mass spectrum (ES) m/e 520 [(M+1)⁺, calcd for C₃₃H₃₄N₃O₃: 520]; 98% purity based on HPLC.

### Example 144

### (R)-[5-AMINO-1-(BIPHENYL-4-YLCARBAMOYL)-PENTYL]-CARBAMIC ACID 9H-FLUOREN-9-YLMETHYL ESTER

Biphenyl-4-ylamine was coupled to (R)-6-tert-Butoxycarbonylamino-2-(9H-fluoren-9-ylmethoxycarbonylamino)-hexanoic acid as described in the method of Example 6 and then purified by HPLC to produce the title compound. 1H NMR (400 MHz, DMSO-d₆) δ 10.16 (s, 1H), 7.90 (d, J=7.5 Hz, 2H), 7.76-7.63 (m, 12H), 7.46-7.40 (m, 4H), 7.35-7.31 (m, 3H), 4.35-4.22 (m, 3H), 4.17 (dd, J=8.4, 13.7 Hz, 1H), 2.82-2.77 (m, 2H), 1.78-1.32 (m, 6H); Low resolution mass spectrum (ES) m/e 520 [(M+1)⁺, calcd for C₃₃H₃₄N₃O₃: 520]; 98.7% purity based on HPLC.

### Example 145

### (S)-3-(4-AMINOMETHYL-PHENYL)-2-(3-BIPHENYL-4-YL-UREIDO)-N-P-TOLYL-PROPIONAMIDE

p-Tolylamine was coupled to (S)-3-[4-(tert-Butoxycarbonylamino-methyl)-phenyl]-2-(9H-fluoren-9-ylmethoxycarbonylamino)-propionic acid, and then to 4-Isocyanato-biphenyl as described in the method of Example 26. Purification by HPLC produced the title compound. 1H NMR (400 MHz, DMSO-d₆) δ 10.15 (s, 1H), 8.84 (s, 1H), 8.13 (br s, 3H), 7.59 (d, J=7.3 Hz, 2H), 7.53 (d, J=8.7 Hz, 2H), 7.49-7.27 (m, 11H), 7.12 (d, J=8.4 Hz, 2H), 6.61 (d, J=8.3 Hz, 1H), 4.68 (dt, J=5.3, 8.4 Hz, 1H), 4.01-3.97 (m, 2H), 3.12 (dd, J=4.9, 13.8 Hz, 1H), 2.91 (dd, J=8.7, 13.8 Hz, 1H), 2.26 (s, 3H); Low resolution mass spectrum (ES) m/e 479 [(M+1)⁺, calcd for C₃₀H₃₁N₄O₂: 479]; 94.5% purity based on HPLC.

### Example 146

### (S)-3-(4-AMINOMETHYL-PHENYL)-2-[3-(4-BENZYLOXY-PHENYL)-UREIDO]-N-P-TOLYL-PROPIONAMIDE

p-Tolylamine was coupled to (S)-3-[4-(tert-Butoxycarbonylamino-methyl)-phenyl]-2-(9H-fluoren-9-ylmethoxycarbonylamino)-propionic acid, and then to 1-Benzyloxy-4-isocyanato-benzene as described in the method of Example 26. Purification by HPLC produced the title compound. 1H NMR (400 MHz, DMSO-d₆) δ 10.11 (s, 1H), 8.53 (s, 1H), 8.14 (s, 3H), 7.47 (d, J=8.4 Hz, 2H), 7.42-7.30 (m, 9H), 7.24 (d, J=9.0 Hz, 2H), 7.12 (d, J=8.4 Hz, 2H), 6.87 (d, J=9.1 Hz, 2H), 6.46 (d, J=8.4 Hz, 1H), 5.02 (s, 2H), 4.64 (dt, J=5.3, 8.5 Hz, 1H), 4.01-3.97 (m, 2H), 3.09 (dd, J=5.0, 13.8 Hz, 1H), 2.88 (dd, J=8.7, 13.7 Hz, 1H), 2.25 (s, 3H); Low resolution mass spectrum (ES) m/e 509 [(M+1)⁺, calcd for C₃₁H₃₃N₄O₃: 509]; 95.9% purity based on HPLC.

### Example 147

### (S)-3-(4-AMINOMETHYL-PHENYL)-2-[3-(9H-FLUOREN-2-YL)-UREIDO]-N-P-TOLYL-PROPIONAMIDE

p-Tolylamine was coupled to (S)-3-[4-(tert-Butoxycarbonylamino-methyl)-phenyl]-2-(9H-fluoren-9-ylmethoxycarbonylamino)-propionic acid, and then to 2-Isocyanato-9H-fluorene as described in the method of Example 26. Purification by HPLC produced the title compound. 1H NMR (400 MHz, DMSO-d₆) δ 10.15 (s, 1H), 8.82 (s, 1H), 8.12 (br s, 3H), 7.75-7.68 (m, 3H), 7.50 (m, 3H), 7.39-7.28 (m, 6H), 7.22 (t, J=7.4 Hz, 1H), 7.12 (d, J=8.3 Hz, 2H), 6.60 (d, J=8.3 Hz, 1H), 4.68 (dt, J=5.4, 8.4 Hz, 1H), 4.00 (m, 2H), 3.84 (s, 2H), 3.13 (dd, J=5.0, 13.8 Hz, 1H), 2.92 (dd, J=8.8, 13.9 Hz, 1H), 2.26 (s, 3H); Low resolution mass spectrum (ES) m/e 491 [(M+1)⁺, calcd for C₃₁H₃₁N₄O₂: 491]; 99.0% purity based on HPLC.

### Example 148

### (S)-3-(4-AMINOMETHYL-PHENYL)-2-(3-BENZYL-UREIDO)-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to benzyl isocyanate as described in the method of Example 26. Purification by HPLC produced the title compound. 1H NMR (400 MHz, DMSO-d6) δ 10.08 (s, 1H), 8.15 (br s, 3H), 7.54 (d, J=8 Hz, 1H), 7.49 (d, J=9 Hz, 2H), 7.36-7.27 (m, 8H), 7.23-7.19 (m, 3H), 6.58 (t, J=6 Hz, 1H), 6.36 (d, J=8.5 Hz, 1H), 4.59 (dt, J=5.76, 8.39 Hz, 1H), 4.17 (dd, J=2.79, 5.53 Hz, 2H), 4.00-3.99 (m, 2H), 3.04 (dd, J=−5.25, 13.64 Hz, 1H), 2.84 (dd, J=−8.54, 13.60 Hz, 1H), 1.26 (s, 9H), Low resolution mass spectrum (ES) m/e 459 [(M+H)⁺, calcd for C₂₈H₃₅N₄O₂: 548]; 99.7% purity based on HPLC.

### Example 149

### (S)-3-(4-AMINOMETHYL-PHENYL)-N-(4-TERT-BUTYL-PHENYL)-2-[3-(3,4-DICHLORO-BENZYL)-UREIDO]-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to 1,2-Dichloro-4-isocyanatomethyl-benzene as described in the method of Example 26. Purification by HPLC produced the title compound. 1H NMR (400 MHz, DMSO-d6) d 10.07 (s, 1H), 8.15 (br s, 3H), 7.54 (d, J=8 Hz, 1H), 7.49 (d, J=9 Hz, 2H), 7.45 (d, J=2 Hz, 1H), 7.36-7.28 (m, 6H), 7.18 (dd, J=2 and 8 Hz, 1H), 6.69-6.66 (m, 1H), 6.48-6.45 (m, 1H), 4.56 (dt, J=6 and 8 Hz, 1H), 4.17 (dd, J=4 and 5 Hz, 2H), 3.99 (d, J=5 Hz, 2H), 3.04 (dd, J=5 and 14 Hz, 2H), 2.84 (dd, J=9 and 14 Hz, 1H), 1.26 (s, 9H), Low resolution mass spectrum (ES) m/e 527 [(M+H)⁺, calcd for C₂₈H₃₃Cl₂N₄O₂: 527]; 99.6% purity based on HPLC.

### Example 150

### (S)-3-(4-AMINOMETHYL-PHENYL)-2-(3-BIPHENYL-4-YL-UREIDO)-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to 4-Isocyanatomethyl-biphenyl as described in the method of Example 26. Purification by HPLC produced the title compound. 1H NMR (400 MHz, DMSO-d6) δ 10.20 (s, 1H), 8.86 (s, 1H), 8.13 (br s, 3H), 7.59 (d, J=7.45 Hz, 2H), 7.53 (t, J=9.00 Hz, 4H), 7.46-7.27 (m, 11H), 6.62 (d, J=8.27 Hz, 1H), 4.69 (dt, J=5.71, 8.30 Hz, 1H), 3.99 (d, J=5.18 Hz, 2H), 3.12 (dd, J=4.98, 13.76 Hz, 1H), 2.91 (dd, J=8.56, 13.74 Hz, 1H), 1.26 (s, 9H); Low resolution mass spectrum (ES) m/e 521 [(M+H)⁺, calcd for C₃₃H₃₇N₄O₂: 521]; 100% purity based on HPLC.

### Example 151

### (S)-3-(4-AMINOMETHYL-PHENYL)-2-(3-BENZHYDRYL-UREIDO)-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to (Phenyl-isocyanato-methyl)-benzene as described in the method of Example 26. Purification by HPLC produced the title compound. ¹H NMR (400 MHz, DMSO-d6) δ 10.08 (s, 1H), 8.13 (br s, 3H), 7.47 (d, J=8.65 Hz, 1H), 7.35-7.18 (m, 17H), 6.35 (d, J=8.44 Hz, 1H), 5.85 (d, J=8.52 Hz, 1H), 4.58 (dd, J=7.95, 13.72 Hz, 1H), 3.98 (d, J=5.45 Hz, 2H), 3.05 (dd, J=5.28, 13.62 Hz, 1H), 2.84 (dd, J=7.94, 13.67 Hz, 1H), 1.25 (s, 9H); Low resolution mass spectrum (ES) m/e 535 [(M+H)⁺, calcd for C₃₄H₃₉N₄O₂: 535]; 100% purity based on HPLC.

### Example 152

### (S)-3-(4-AMINOMETHYL-PHENYL)-N-(4-TERT-BUTYL-PHENYL)-2-[3-(9H-FLUOREN-2-YL)-UREIDO]-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid then to 2-Isocyanato-9H-fluorene as described in the method of Example 26. Purification by HPLC produced the title compound. ¹H NMR (400 MHz, DMSO-d6) δ 10.20 (s, 1H), 8.86 (s, 1H), 8.15 (br s, 3H), 7.73 (dd, J=8.00, 14.41 Hz, 2H), 7.69 (s, 1H), 7.53-7.50 (m, 3H), 7.39-7.28 (m, 8H), 7.22 (t, J=7.41, 7.41 Hz, 1H), 6.62 (d, J=8.29 Hz, 1H), 4.68 (dd, J=8.29, 13.52 Hz, 1H), 3.99 (d, J=5.52 Hz, 2H), 3.84 (s, 2H), 3.13 (dd, J=4.94, 13.75 Hz, 1H), 2.92 (dd, J=8.50, 13.74 Hz, 1H), 1.26 (s, 9H); Low resolution mass spectrum (ES) m/e 533 [(M+H)⁺, calcd for C₃₄H₃₇N₄O₂: 533]; 96.1% purity based on HPLC.

### Example 153

### (S)-FURAN-2-CARBOXYLIC ACID {4-[6-AMINO-2-(3-BENZYL-UREIDO)-HEXANOYLAMINO]-BENZYL}-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then to benzyl isocyanate as described in the method of Example 26. Purification by HPLC produced the title compound. ¹H NMR (400 MHz, DMSO-d6) δ 10.05 (s, 1H), 7.79 (br s, 1H), 7.66 (br s, 3H), 7.53 (d, J=8.40 Hz, 2H), 7.32-7.28 (m, 2H), 7.24-7.19 (m, 5H), 6.89 (br s, 1H), 6.58-6.56 (m, 2H), 6.30 (d, J=8.43 Hz, 1H), 4.64 (s, 2H), 4.32 (dd, J=7.79, 13.75 Hz, 1H), 4.22 (d, J=4.82 Hz, 2H), 4.11 (m, 1H), 2.80-2.74 (m, 2H), 1.71-1.18 (m, 15H), 1.08-0.99 (m, 1H); Low resolution mass spectrum (ES) m/e 560 [(M+H)⁺, calcd for C₃₂H₄₂N₅O₄: 560]; 100% purity based on HPLC.

### Example 154

### (S)-FURAN-2-CARBOXYLIC ACID (4-{6-AMINO-2-[3-(3,4-DICHLORO-BENZYL)-UREIDO]-HEXANOYLAMINO}-BENZYL)-CYCLOHEXYL-AMIDE

Furan-2-carboxylic acid (4-amino-benzyl)-cyclohexyl-amide was coupled to (S)-6-tert-Butoxycarbonylamino-2-(9H-fluoren-9-ylmethoxycarbonylamino)-hexanoic acid, and then to 1,2-Dichloro-4-isocyanatomethyl-benzene as described in the method of Example 26. Purification by HPLC produced the title compound. ¹H NMR (400 MHz, DMSO-d6) δ 10.06 (s, 1H), 7.80 (br s, 1H), 7.67 (br s, 3H), 7.57-7.52 (m, 3H), 7.47 (s, 1H), 7.24-7.18 (m, 3H), 6.89 (br s, 1H), 6.68 (t, J=5.97, 5.97 Hz, 1H), 6.58 (br s, 1H), 6.42 (d, J=8.38 Hz, 1H), 4.64 (br s, 2H), 4.29 (dd, J=7.88, 13.82 Hz, 1H), 4.21 (d, J=5.91 Hz, 2H), 4.11 (br m, 1H), 2.79-2.74 (m, 2H), 1.71-1.17 (m, 15H), 1.08-0.98 (m, 1H); Low resolution mass spectrum (ES) m/z: 628 and 630 [(M+H)⁺, calcd for C32H40N5O4: 628]; 100% purity based on HPLC.

### Example 155

### 2-[(4-CYCLOHEXYL-PHENYLCARBAMOYL)-METHYL]-PIPERAZINE-1-CARBOXYLIC ACID 9H-FLUOREN-9-YLMETHYL ESTER

p-Cyclohexylaniline was coupled to (+/−)-2-Carboxymethyl-piperazine-1,4-dicarboxylic acid 4-tert-butyl ester 1-(9H-fluoren-9-ylmethyl) ester as described in the method of Example 6. Purification by HPLC produced the title compound. ¹H NMR (400 MHz, DMSO-d6) d 9.99 (s, 1H), 9.20 (d, J=8.11 Hz, 1H), 8.74 (d, J=8.78 Hz, 1H), 7.86 (d, J=7.45 Hz, 2H), 7.61 (d, J=7.43 Hz, 1H), 7.47 (d, J=7.78 Hz, 3H), 7.39 (t, J=7.35, 7.35 Hz, 2H), 7.30 (t, J=7.45, 7.45 Hz, 1H), 7.25 (br s, 1H), 7.12 (d, J=8.04 Hz, 2H), 4.71-4.69 (m, 1H), 4.22 (s, 2H), 4.14 (s, 1H), 3.99 (d, J=13.77 Hz, 1H), 3.41 (d, J=12.64 Hz, 1H), 3.28-3.19 (m, 3H), 2.93-2.85 (m, 2H), 2.60 (dd, J=6.79, 14.93 Hz, 1H), 2.41 (br m, 1H), 1.77-1.67 (m, 5H), 1.38-1.18 (m, 5H); Low resolution mass spectrum (ES) m/z 524 [(M+H)⁺, calcd for C₃₃H₃₈N₃O₃: 524]; 100% purity based on HPLC.

### Example 156

### (+/−)-2-(4-CYCLOHEXYL-PHENYLCARBAMOYL)-PIPERAZINE-1-CARBOXYLIC ACID 9H-FLUOREN-9-YLMETHYL ESTER

p-Cyclohexylaniline was coupled to (+/−)-piperazine-1,2,4-tricarboxylic acid 4-tert-butyl ester 1-(9H-fluoren-9-ylmethyl) ester as described in the method of Example 6. Purification by HPLC produced the title compound. ¹H NMR (400 MHz, DMSO-d6) δ 10.15 (d, J=28.52 Hz, 1H), 9.29 (br s, 1H), 8.65 (br m, 1H), 7.88 (d, J=18.64 Hz, 2H), 7.67-7.09 (m, 10H), 4.90 (s, 1H), 4.40-4.25 (m, 3H), 4.10-3.29 (m, under D2O), 2.98 (s, 1H), 1.77-1.68 (m, 5H), 1.36-1.21 (m, 5H); Low resolution mass spectrum (ES) m/e 510 [(M+H)⁺, calcd for C₃₂H₃₆N₃O₃: 510]; 100% purity based on HPLC.

### Example 157

### 3-(4-CYCLOHEXYL-PHENYLCARBAMOYL)-PIPERAZINE-1-CARBOXYLIC ACID 9H-FLUOREN-9-YLMETHYL ESTER

p-Cyclohexylaniline was coupled to (+/−)-piperazine-1,2,4-tricarboxylic acid 1-tert-butyl ester 4-(9H-fluoren-9-ylmethyl) ester as described in the method of Example 6. Purification by HPLC produced the title compound. 1H NMR (400 MHz, DMSO-d6) δ 10.67 (s, 1H), 9.43 (br s, 2H), 7.89 (d, J=6.82 Hz, 2H), 7.63 (d, J=7.25 Hz, 2H), 7.50 (d, J=8.38 Hz, 2H), 7.44-7.40 (m, 2H), 7.33 (br, 2H), 7.22 (d, J=8.38 Hz, 2H), 4.43-4.29 (m, 4H), 4.10 (m, 1H), 3.88 (d, J=13.76 Hz, 1H), 3.37-3.25 (m, 3H under D2O), 3.09 (br s, 1H), 2.46 (m, 1H under DMSO-D6), 1.83-1.68 (m, 5H), 1.42-1.20 (m, 5H); Low resolution mass spectrum (ES) m/z 510 [(M+H)⁺, calcd for C₃₂H₃₆N₃O₃: 510]; 100% purity based on HPLC.

### Example 158

### 2-[(4-CYCLOHEXYL-PHENYLCARBAMOYL)-METHYL]-PIPERAZINE-1-CARBOXYLIC ACID BENZYLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to benzyl isocyanate as described in the method of Example 26. Purification by HPLC produced the title compound. 1H NMR (400 MHz, DMSO-d6) δ 9.99 (s, 1H), 9.15 (d, J=9.59 Hz, 1H), 8.76-8.74 (m, 1H), 7.47 (d, J=8.48 Hz, 2H), 7.31 (t, J=−5.72, 5.72 Hz, 1H), 7.26-7.17 (m, 5H), 7.14 (d, J=8.49 Hz, 2H), 4.70 (br s, 1H), 4.24 (d, J=5.59 Hz, 2H), 4.03 (d, J=13.88 Hz, 1H), 3.46 (d, J=12.58 Hz, 1H), 3.26 (d, J=11.80 Hz, 1H), 3.18-3.07 (m, 2H), 2.98-2.88 (m, 2H), 2.60 (dd, J=5.61, 15.44 Hz, 1H), 2.45-2.41 (m, 1H), 1.78-1.68 (m, 5H), 1.42-1.17 (m, 5H); Low resolution mass spectrum (ES) m/e 435 [(M+H)⁺, calcd for C₂₆H₃₅N₄O₂: 435]; 98.9% purity based on HPLC.

### Example 159

### 2-[(4-CYCLOHEXYL-PHENYLCARBAMOYL)-METHYL]-PIPERAZINE-1-CARBOXYLIC ACID 4-CHLORO-BENZYLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 1-Chloro-4-isocyanatomethyl-benzene as described in the method of Example 26. Purification by HPLC produced the title compound. 1H NMR (400 MHz, DMSO-d6) δ 9.99 (s, 1H), 9.21 (d, J=9.23 Hz, 1H), 8.80-8.77 (br m, 1H), 7.46 (d, J=8.48 Hz, 2H), 7.33 (t, J=5.65, 5.65 Hz, 1H), 7.24 (s, 4H), 7.14 (d, J=−8.54 Hz, 2H), 4.72-4.71 (br m, 1H), 4.21 (d, J=5.57 Hz, 2H), 4.02 (d, J=13.60 Hz, 1H), 3.44 (d, J=12.58 Hz, 1H), 3.26 (d, J=11.80 Hz, 1H), 3.16-3.09 (m, 2H), 2.91 (dd, J=8.29, 15.36 Hz, 2H), 2.62 (dd, J=6.13, 15.29 Hz, 1H), 2.46-2.41 (m, 1H), 1.78-1.67 (m, 5H), 1.41-1.16 (m, 5H); Low resolution mass spectrum (ES) m/e 469, 471 [(M+H)⁺, calcd for C₂₆H₃₄ClN₄O₂: 469]; 99.7% purity based on HPLC.

### Example 160

### 2-[(4-CYCLOHEXYL-PHENYLCARBAMOYL)-METHYL]-PIPERAZINE-1-CARBOXYLIC ACID 3,4-DICHLORO-BENZYLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 1,2-Dichloro-4-isocyanatomethyl-benzene as described in the method of Example 26. Purification by HPLC produced the title compound. 1H NMR (400 MHz, DMSO-d6) δ 9.97 (s, 1H), 9.14 (br s, 1H), 8.74-8.73 (br m, 1H), 7.50 (d, J=1.89 Hz, 1H), 7.44 (d, J=8.38 Hz, 3H), 7.38 (t, J=5.71, 5.71 Hz, 1H), 7.21 (dd, J=1.94, 8.29 Hz, 1H), 7.13 (d, J=−8.54 Hz, 2H), 4.70-4.69 (br m, 1H), 4.21 (d, J=5.55 Hz, 2H), 4.01 (d, J=13.84 Hz, 1H), 3.44 (d, J=12.53 Hz, 1H), 3.26 (d, J=11.69 Hz, 1H), 3.18-3.07 (m, 2H), 2.94-2.88 (m, 2H), 2.61 (dd, J=6.12, 15.35 Hz, 1H), 2.45-2.40 (m, 1H), 1.78-1.68 (m, 5H), 1.41-1.16 (m, 5H); Low resolution mass spectrum (ES) m/e 503, 505 [(M+H)⁺, calcd for C₂₆H₃₃Cl₂N₄O₂: 503]; 99.3% purity based on HPLC.

### Example 161

### 2-[(4-CYCLOHEXYL-PHENYLCARBAMOYL)-METHYL]-PIPERAZINE-1-CARBOXYLIC ACID BIPHENYL-4-YLAMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 4-Isocyanato-biphenyl as described in the method of Example 26. Purification by HPLC produced the title compound. 1H NMR (400 MHz, DMSO-d6) δ 10.05 (s, 1H), 9.18 (br d, J=8.83 Hz, 1H), 8.95, (s, 1H), 8.81-8.78 (br m, 1H), 7.62 (d, J=7.25 Hz, 2H), 7.56 (d, J=8.77 Hz, 2H), 7.50-7.41 (m, 6H), 7.31 (t, J=7.34, 7.34 Hz, 1H), 7.12 (d, J=8.54 Hz, 1H), 4.87-4.86 (br m, 1H), 4.17 (d, J=13.84 Hz, 1H), 3.49 (d, J=12.60 Hz, 1H), 3.34-3.18 (m, 3H), 3.05-32.94 (m, 3H), 2.74 (dd, J=6.27, 15.47 Hz, 1H), 2.42 (br m, 1H), 1.77-1.67 (m, 5H), 1.39-1.15 (m, 5H); Low resolution mass spectrum (ES) m/e 497 [(M+H)⁺, calcd for C₃₁H₃₇N₄O₂: 497]; 99.1% purity based on HPLC.

### Example 162

### 2-[(4-CYCLOHEXYL-PHENYLCARBAMOYL)-METHYL]-PIPERAZINE-1-CARBOXYLIC ACID (9H-FLUOREN-2-YL)-AMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 2-Isocyanato-9H-fluorene as described in the method of Example 26. Purification by HPLC produced the title compound. 1H NMR (400 MHz, DMSO-d6) δ 10.05 (s, 1H), 9.18 (d, J=9.54 Hz, 1H), 8.94 (s, 1H), 8.81-8.79 (m, 1H), 7.76 (dd, J=7.91, 12.57 Hz, 2H), 7.67 (d, J=1.03 Hz, 1H), 7.52 (d, J=7.42 Hz, 1H), 7.46 (d, J=−8.54 Hz, 2H), 7.37-7.32 (m, 2H), 7.24 (dt, J=0.99, 7.43, 7.44 Hz, 1H), 7.12 (d, J=8.55 Hz, 2H), 4.87-4.86 (m, 1H), 4.18 (d, J=13.89 Hz, 1H), 3.83 (s, 2H), 3.49 (d, J=12.43 Hz, 1H), 3.34-3.19 (m, 3H), 3.05-2.93 (m, 2H), 2.75 (dd, J=6.37, 15.42 Hz, 1H), 2.41 (br m, 1H), 1.77-1.66 (m, 5H), 1.39-1.14 (m, 5H); Low resolution mass spectrum (ES) m/e 509 [(M+H)⁺, calcd for C₃₂H₃₇N₄O₂: 509]; 99.2% purity based on HPLC.

### Example 163

### 2-[(4-CYCLOHEXYL-PHENYLCARBAMOYL)-METHYL]-PIPERAZINE-1-CARBOXYLIC ACID (4-BENZYLOXY-PHENYL)-AMIDE

p-Cyclohexylaniline was coupled to (+/−)-2-Carboxymethyl-piperazine-1,4-dicarboxylic acid 4-tert-butyl ester 1-(9H-fluoren-9-ylmethyl) ester, and then to 1-Benzyloxy-4-isocyanato-benzene as described in the method of Example 26. Purification by HPLC produced the title compound. 1H NMR (400 MHz, DMSO-d6) δ 10.03 (s, 1H), 9.19 (d, J=8.78 Hz, 1H), 8.80-8.78 (br m, 1H), 8.68 (s, 1H), 7.47-7.36 (m, 6H), 7.32 (td, J=2.12, 2.12, 5.29 Hz, 1H), 7.27 (d, J=9.07 Hz, 2H), 7.13 (d, J=8.54 Hz, 2H), 6.90 (d, J=9.07 Hz, 2H), 5.04 (s, 2H), 4.81-4.80 (br m, 1H), 4.13 (d, J=13.82 Hz, 1H), 3.48 (d, J=12.45 Hz, 1H), 3.31-3.14 (m, 3H), 3.00-2.94 (m, 2H), 2.68 (dd, J=5.91, 15.47 Hz, 1H), 2.43 (br m, 1H), 1.78-1.67 (m, 5H), 1.41-1.16 (m, 5H); Low resolution mass spectrum (ES) m/e 527 [(M+H)⁺, calcd for C₃₂H₃₉N₄O₃: 527]; 99.2% purity based on HPLC.

### Example 164

### (R)-6-AMINO-2-[3-(4-CHLORO-BENZYL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 1-chloro-4-isocyanatomethyl-benzene as described in the method of Example 26. Purification by HPLC produced the title compound. 1H NMR (400 MHz, DMSO-d6) δ 10.00 (s, 1H), 7.67 (br s, 3H), 7.49 (d, J=8.53 Hz, 2H), 7.36 (d, J=8.43 Hz, 2H), 7.25 (d, J=8.43 Hz, 2H), 7.15 (d, J=8.54 Hz, 2H), 6.63 (t, J=6.07, 6.07 Hz, 1H), 6.35 (d, J=8.46 Hz, 1H), 4.31 (dd, J=7.95, 13.92 Hz, 1H), 4.20 (d, J=6.08 Hz, 2H), 2.79-2.74 (m, 2H), 2.43 (br m, 1H), 1.78-1.50 (m, 9H), 1.41-1.19 (m, 7H); Low resolution mass spectrum (ES) m/e 471, 473 [(M+H)⁺, calcd for C₂₆H₃₆ClN₄O₂: 471]; 99.1% purity based on HPLC.

### Example 165

### (R)-6-AMINO-2-[3-(3,4-DICHLORO-BENZYL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 1,2-Dichloro-4-isocyanatomethyl-benzene as described in the method of Example 26. Purification by HPLC produced the title compound. 1H NMR (400 MHz, DMSO-d6) δ 9.99 (s, 1H), 7.66 (br s, 3H), 7.56 (d, J=8.25 Hz, 1H), 7.51-7.47 (m, 3H), 7.23 (dd, J=1.97, 8.28 Hz, 1H), 7.14 (d, J=8.55 Hz, 2H), 6.68 (t, J=−6.14, 6.14 Hz, 1H), 6.42 (d, J=−8.46 Hz, 1H), 4.30 (dd, J=−8.08, 13.97 Hz, 1H), 4.21 (d, J=6.12 Hz, 2H), 2.79-2.74 (m, 2H), 2.43 (br m, 1H), 1.78-1.50 (m, 9H), 1.41-1.16 (m, 7H); Low resolution mass spectrum (ES) m/e 505, 507 [(M+H)⁺, calcd for C₂₆H₃₅ClN₄O₂: 505]; 99.1% purity based on HPLC.

### Example 166

### (R)-6-AMINO-2-[3-(4-BENZYLOXY-PHENYL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. 1H NMR (400 MHz, DMSO-d6) δ 10.08 (s, 1H), 8.54 (s, 1H), 7.65 (br s, 3H), 7.51 (d, J=8.54 Hz, 2H), 7.44-7.36 (m, 5H), 7.32 (d, J=7.22 Hz, 1H), 7.28 (d, J=9.03 Hz, 2H), 7.15 (d, J=8.56 Hz, 2H), 6.89 (d, J=9.05 Hz, 2H), 6.43 (d, J=8.27 Hz, 1H), 5.03 (s, 2H), 4.38 (dd, J=7.86, 13.64 Hz, 1H), 2.82-2.74 (m, 2H), 2.43 (br m, 1H), 1.78-1.19 (m, 16H); Low resolution mass spectrum (ES) m/e 529 [(M+H)⁺, calcd for C₃₂H₄₁N₄O₃: 529]; 92.3% purity based on HPLC.

### Example 167

### (S)-6-AMINO-2-[3-(4-CHLORO-BENZYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-chloro-4-isocyanatomethyl-benzene as described in the method of Example 26. Purification by HPLC produced the title compound. 1H NMR (400 MHz, DMSO-d6) δ 10.08 (s, 1H), 8.54 (s, 1H), 7.65 (br s, 3H), 7.51 (d, J=8.54 Hz, 2H), 7.44-7.36 (m, 5H), 7.32 (d, J=7.22 Hz, 1H), 7.28 (d, J=9.03 Hz, 2H), 7.15 (d, J=8.56 Hz, 2H), 6.89 (d, J=−9.05 Hz, 2H), 6.43 (d, J=−8.27 Hz, 1H), 5.03 (s, 2H), 4.38 (dd, J=7.86, 13.64 Hz, 1H), 2.82-2.74 (m, 2H), 2.43 (br m, 1H), 1.78-1.19 (m, 16H); Low resolution mass spectrum (ES) m/e 403 [(M+H)⁺, calcd for C₂₁H₂₈ClN₄O₂: 403]; 92.3% purity based on HPLC.

### Example 168

### (R)-6-AMINO-2-(3-BENZYL-UREIDO)-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to benzyl isocyanate as described in the method of Example 26. Purification by HPLC produced the title compound. 1H NMR (400 MHz, DMSO-d6) δ 10.00 (s, 1H), 7.72 (br s, 3H), 7.49 (d, J=8.41 Hz, 2H), 7.32-7.20 (m, 5H), 7.11 (d, J=8.35 Hz, 2H), 6.60 (t, J=5.97, 5.97 Hz, 1H), 6.33 (d, J=8.46 Hz, 1H), 4.32 (dd, J=8.28, 13.97 Hz, 1H), 4.22 (d, J=−5.80 Hz, 2H), 2.81-2.73 (m, 2H), 2.25 (s, 3H), 1.72-1.50 (m, 4H), 1.43-1.24 (m, 2H); Low resolution mass spectrum (ES) m/e 369 [(M+H)⁺, calcd for C₂₁H₂₉N₄O₂: 369]; 96.8% purity based on HPLC.

### Example 169

### (R)-6-AMINO-2-[3-(4-CHLORO-BENZYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1-chloro-4-isocyanatomethyl-benzene as described in the method of Example 26. Purification by HPLC produced the title compound. 1H NMR (400 MHz, DMSO-d6) δ 9.99 (s, 1H), 7.70 (br s, 3H), 7.48 (d, J=8.39 Hz, 2H), 7.36 (d, J=8.42 Hz, 2H), 7.25 (d, J=−8.45 Hz, 2H), 7.11 (d, J=−8.30 Hz, 2H), 6.64 (t, J=−6.05, 6.05 Hz, 1H), 6.36 (d, J=8.45 Hz, 1H), 4.30 (dd, J=8.09, 13.82 Hz, 1H), 4.20 (d, J=5.97 Hz, 2H), 2.79-2.73 (m, 2H), 2.25 (s, 3H), 1.72-1.63 (m, 1H), 1.58-1.51 (m, 3H), 1.42-1.24 (m, 2H); Low resolution mass spectrum (ES) m/e 403, 405 [(M+H)⁺, calcd for C₂₁H₂₈ClN₄O₂: 403]; 95.7% purity based on HPLC.

### Example 170

### (R)-6-AMINO-2-[3-(3,4-DICHLORO-BENZYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1,2-dichloro-4-isocyanatomethyl-benzene as described in the method of Example 26. Purification by HPLC produced the title compound. 1H NMR (400 MHz, DMSO-d6) δ 9.98 (s, 1H), 7.71 (br s, 3H), 7.56 (d, J=8.25 Hz, 1H), 7.50-7.48 (m, 3H), 7.23 (dd, J=1.95, 8.27 Hz, 1H), 7.10 (d, J=8.35 Hz, 2H), 6.70 (t, J=5.44, 5.44 Hz, 1H), 6.43 (d, J=8.33 Hz, 1H), 0.29 (dd, J=8.21, 13.84 Hz, 1H), 4.21 (d, J=6.06 Hz, 2H), 2.81-2.73 (m, 2H), 2.25 (s, 3H), 1.73-1.51 (m, 4H), 1.43-1.24 (m, 2H); Low resolution mass spectrum (ES) m/e 437, 439 [(M+H)⁺, calcd for C₂₁H₂₇Cl₂N₄O₂: 437]; 99.8% purity based on HPLC.

### Example 171

### (R)-6-AMINO-2-(3-BIPHENYL-4-YL-UREIDO)-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 4-Isocyanato-biphenyl as described in the method of Example 26. Purification by HPLC produced the title compound. 1H NMR (400 MHz, DMSO-d6) δ 10.10 (s, 1H), 8.93 (s, 1H), 7.74 (br s, 3H), 7.61 (d, J=7.31 Hz, 2H), 7.55 (d, J=8.75 Hz, 2H), 7.50 (t, J=8.84, 8.84 Hz, 4H), 7.42 (t, J=7.72, 7.72 Hz, 2H), 7.29 (t, J=7.33, 7.33 Hz, 1H), 7.12 (d, J=8.39 Hz, 2H), 6.67 (d, J=7.87 Hz, 1H), 4.40 (dd, J=7.98, 13.42 Hz, 1H), 2.83-2.76 (m, 2H), 2.25 (s, 3H), 1.80-1.71 (m, 1H), 1.67-1.52 (m, 3H), 1.49-1.31 (m, 2H); Low resolution mass spectrum (ES) m/e 431 [(M+H)⁺, calcd for C₂₆H₃₁N₄O₂: 431]; 99.9% purity based on HPLC.

### Example 172

### (R)-6-AMINO-2-[3-(4-BENZYLOXY-PHENYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. 1H NMR (400 MHz, DMSO-d6) δ 10.07 (s, 1H), 8.58 (s, 1H), 7.71 (br s, 3H), 7.50 (d, J=8.42 Hz, 2H), 7.43-7.27 (m, 7H), 7.11 (d, J=8.40 Hz, 2H), 6.89 (d, J=9.05 Hz, 2H), 6.47 (d, J=8.26 Hz, 1H), 5.03 (s, 2H), 4.37 (dd, J=8.02, 13.53 Hz, 1H), 2.82-2.74 (m, 2H), 2.25 (s, 3H), 1.77-1.68 (m, 1H), 1.64-1.52 (m, 3H), 1.44-1.28 (m, 2H); Low resolution mass spectrum (ES) m/e 461 [(M+H)⁺, calcd for C₂₇H₃₃N₄O₃: 461]; 99.7% purity based on HPLC.

### Example 173

### (R)-6-AMINO-2-[3-(9H-FLUOREN-2-YL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. 1H NMR (400 MHz, DMSO-d6) δ 10.11 (s, 1H), 8.86 (s, 1H), 7.76-7.69 (m, 6H), 7.52-7.50 (m, 3H), 7.34-7.31 (m, 2H), 7.22 (dt, J=0.98, 7.45, 7.47 Hz, 1H), 7.12 (d, J=8.39 Hz, 2H), 6.61 (d, J=8.07 Hz, 1H), 4.41 (dd, J=7.98, 13.44 Hz, 1H), 3.85 (s, 2H), 2.82-2.76 (m, 2H), 2.25 (s, 3H), 1.80-1.72 (m, 1H), 1.67-1.52 (m, 3H), 1.47-1.33 (m, 2H); Low resolution mass spectrum (ES) m/e 443 [(M+H)⁺, calcd for C₂₇H₃₁N₄O₂: 443]; 99.5% purity based on HPLC.

### Example 174

### (S)-3-(4-AMINOMETHYL-PHENYL)-2-[3-(4-BENZYLOXY-PHENYL)-UREIDO]-N-(4-TERT-BUTYL-PHENYL)-PROPIONAMIDE

p-t-Butyl-phenylamine was coupled to (S)-3-[4-(tert-Butoxycarbonylamino-methyl)-phenyl]-2-(9H-fluoren-9-ylmethoxycarbonylamino)-propionic acid, and then to 1-Benzyloxy-4-isocyanato-benzene as described in the method of Example 26. Purification by HPLC produced the title compound. 1H NMR (400 MHz, DMSO-d6) δ 10.16 (s, 1H), 8.54 (s, 1H), 8.13 (br s, 3H), 7.50 (d, J=8.74 Hz, 2H), 7.43-7.29 (m, 11H), 7.25 (d, J=6.97 Hz, 2H), 6.88 (d, J=7.03 Hz, 2H), 6.46 (d, J=8.41 Hz, 1H), 5.02 (s, 2H), 4.64 (dt, J=5.35, 8.42, 8.46 Hz, 1H), 4.00-3.98 (m, 2H), 3.09 (dd, J=5.02, 13.74 Hz, 1H), 2.88 (dd, J=8.60, 13.74 Hz, 1H), 1.26 (s, 9H); Low resolution mass spectrum (ES) m/e 551 [(M+H)⁺, calcd for C₃₄H₃₉N₄O₃: 551]; 99.8% purity based on HPLC.

### Example 175

### (R)-[2-(4-AMINOMETHYL-PHENYL)-1-P-TOLYLCARBAMOYL-ETHYL]-CARBAMIC ACID 9H-FLUOREN-9-YLMETHYL ESTER

p-Tolylamine to (R)-3-[4-(tert-Butoxycarbonylamino-methyl)-phenyl]-2-(9H-fluoren-9-ylmethoxycarbonylamino)-propionic acid as described in the method of Example 6. Purification by HPLC produced the title compound. 1H NMR (400 MHz, DMSO-d6) δ 7.85 (d, J=7.5 Hz, 2H), 7.64 (br d, J=6.6 Hz, 2H), 7.44 (d, J=8.6 Hz, 2H), 7.42-7.24 (m, 8H), 7.11 (d, J=8.1 Hz, 2H), 4.35 (dd, J=4.4, 10.1 Hz, 1H), 4.27-4.09 (m, 3H), 3.94 (s, 2H), 3.02 (dd, J=4.4, 13.8 Hz, 1H), 2.86 (dd, J=10.5, 13.6 Hz, 1H), 2.23 (s, 3H), 1.42 (s, 1H); Low resolution mass spectrum (ES) m/e 506 [(M+H)⁺, calcd for C₃₂H₃₂N₃O₃: 506]; 95% purity based on HPLC.

### Example 176

### (S)-[5-(2-AMINO-ACETYLAMINO)-1-P-TOLYLCARBAMOYL-PENTYL]-CARBAMIC ACID 9H-FLUOREN-9-YLMETHYL ESTER

(S)-6-Amino-2-(2-9H-fluoren-9-yl-acetylamino)-hexanoic acid p-tolylamide of Example 53 was coupled to tert-Butoxycarbonylamino-acetic acid as described in the method of Example 6. Purification by HPLC produced the title compound. 1H NMR (400 MHz, DMSO-d6) δ 7.87 (d, J=7.5 Hz, 2H), 7.70 (t, J=7.7 Hz, 2H), 7.44 (d, J=8.4 Hz, 2H), 7.40 (ddd, J=2.1, 7.5, 8.1 Hz, 2H), 7.30 (dd, J=8.4, 12.7 Hz, 2H), 7.09 (d, J=8.3 Hz, 2H), 4.33-4.16 (m, 3H), 4.07 (dd, J=5.3, 9.0 Hz, 1H), 3.46 (s, 1H), 3.13-3.06 (m, 1H), 2.74 (t, J=7.7 Hz, 2H), 2.22 (s, 3H), 1.22-1.72 (m, 6H+A22); Low resolution mass spectrum (ES) m/e 515 [(M+H)⁺, calcd for C₃₀H₃₅N₄O₄: 515]; 85% purity based on HPLC.

### Example 177

### (S)-[4-(2-AMINO-ACETYLAMINO)-1-P-TOLYLCARBAMOYL-BUTYL]-CARBAMIC ACID 9H-FLUOREN-9-YLMETHYL ESTER

(S)-(4-Amino-1-p-tolylcarbamoyl-butyl)-carbamic acid 9H-fluoren-9-ylmethyl ester of Example 101 was coupled to tert-Butoxycarbonylamino-acetic acid as described in the method of Example 6. Purification by HPLC produced the title compound. 1H NMR (400 MHz, DMSO-d6) δ 7.86 (d, J=7.5 Hz, 2H), 7.69 (t, J=7.3 Hz, 2H), 7.44 (d, J=8.3 Hz, 2H). 7.40 (ddd, J=2.1, 7.2, 8.5 Hz, 2H), 7.30 (dd, J=7.0, 13.2 Hz, 2H), 7.09 (d, J=8.3 Hz, 2H), 4.32-4.16 (m, 3H), 4.08 (dd, J=5.3, 8.8 Hz, 1H), 3.47 (s, 2H). 3.18-3.07 (m, 2H), 2.22 (s, 3H), 1.77-1.33 (m, 4H); Low resolution mass spectrum (ES) m/e 501 [(M+H)⁺, calcd for C₂₉H₃₃N₄O₄: 501]; 85% purity based on HPLC.

### Example 178

### (S)-[2-(3-AMINO-PROPIONYLAMINO)-1-P-TOLYLCARBAMOYL-ETHYL]-CARBAMIC ACID 9H-FLUOREN-9-YLMETHYL ESTER

(S)-(2-Amino-1-p-tolylcarbamoyl-ethyl)-carbamic acid 9H-fluoren-9-ylmethyl ester of Example 103 was coupled to 3-tert-Butoxycarbonylamino-propionic acid as described in the method of Example 6. Purification by HPLC produced the title compound. 1H NMR (400 MHz, DMSO-d6) δ 7.87 (d, J=7.5 Hz, 2H), 7.69 (t, J=6.4, 5.9 Hz, 2H), 7.44 (d, J=8.4 Hz, 2H), 7.40 (t, J=7.5 Hz, 2H), 7.31 (dd, J=6.8, 14.1 Hz, 2H), 7.10 (d, J=8.3 Hz, 2H), 4.35-4.17 (m, 4H), 3.43 (dd, J=6.6, 13.5 Hz, 1H), 3.35 (dd, J=6.2, 13.4 Hz, 1H), 2.93 (t, J=6.9 Hz, 2H), 2.40 (t, J=6.8 Hz, 2H), 2.22 (s, 3H); Low resolution mass spectrum (ES) m/e 487 [(M+H)⁺, calcd for C₂₈H₃₁N₄O₄: 487]; 98% purity based on HPLC.

### Example 179

### (S)-[3-(3-AMINO-PROPIONYLAMINO)-1-P-TOLYLCARBAMOYL-PROPYL]-CARBAMIC ACID 9H-FLUOREN-9-YLMETHYL ESTER

(S)-(3-Amino-1-p-tolylcarbamoyl-propyl)-carbamic acid 9H-fluoren-9-ylmethyl ester of Example 108 was coupled to 3-tert-Butoxycarbonylamino-propionic acid as described in the method of Example 6. Purification by HPLC produced the title compound. 1H NMR (400 MHz, DMSO-d6) δ 7.87 (d, J=7.5 Hz, 2H), 7.71 (t, J=6.5 Hz, 2H), 7.44 (d, J=8.3 Hz, 2H), 7.40 (ddd, J=1.5, 7.7, 8.1 Hz, 2H), 7.31 (dd, J=7.2, 14.0 Hz, 2H), 7.10 (d, J=8.3 Hz, 2H), 4.31-4.17 (m, 3H), 4.12 (dd, J=5.9, 8.6 Hz, 1H), 3.17-3.08 (m, 2H), 2.96 (t, J=7.0, 6.6 Hz, 2H), 2.42 (t, J=6.8 Hz, 2H), 2.23 (s, 3H), 1.91-1.69 (m, 2H); Low resolution mass spectrum (ES) m/e 501 [(M+H)⁺, calcd for C₂₉H₃₃N₄O₄: 501]; 100% purity based on HPLC.

### Example 180

### (S)-[2-(4-AMINO-PHENYL)-1-P-TOLYLCARBAMOYL-ETHYL]-CARBAMIC ACID 9H-FLUOREN-9-YLMETHYL ESTER

p-Tolylamine was coupled to (S)-3-(4-tert-Butoxycarbonylamino-phenyl)-2-(9H-fluoren-9-ylmethoxycarbonylamino)-propionic acid as described in the method of Example 6. Purification by HPLC produced the title compound. 1H NMR (400 MHz, DMSO-d6) δ 7.86 (d, J=7.5 Hz, 2H), 7.64 (dd, J=4.4, 7.5 Hz, 2H), 7.43 (d, J=8.1 Hz, 2H), 7.39 (t, J=7.7 Hz, 2H), 7.32-7.21 (m, 4H), 7.10 (d, J=8.3 Hz, 2H), 6.98-6.87 (br, 2H), 4.30 (dd, J=5.3, 9.9 Hz, 1H), 4.21-4.07 (m, 3H), 2.95 (dd, J=4.6, 13.6 Hz, 1H), 2.80 (dd, J=10.3, 13.6 Hz, 1H), 2.23 (s, 3H); Low resolution mass spectrum (ES) m/e 492 [(M+H)⁺, calcd for C₃₁H₃₀N₃O₃: 492]; 91% purity based on HPLC.

### Example 181

### (S)-(5-ACETYLAMINO-1-P-TOLYLCARBAMOYL-PENTYL)-CARBAMIC ACID 9H-FLUOREN-9-YLMETHYL ESTER

(S)-6-Amino-2-(2-9H-fluoren-9-yl-acetylamino)-hexanoic acid p-tolylamide of Example 53 was coupled to acetyl chloride as described in the method for Intermediate #3 of Example 1. Purification by HPLC produced the title compound. 1H NMR (400 MHz, DMSO-d6) δ 9.90 (s, 1H), 7.86 (d, J=7.7 Hz, 2H), 7.70 (t, J=6.6 Hz, 2H), 7.43 (d, J=8.3 Hz, 2H), 7.40 (ddd, J=1.8, 7.2, 8.1 Hz, 2H), 7.30 (dd, J=7.2, 12.3 Hz, 2H), 7.08 (d, J=8.3 Hz, 2H), 4.29-4.16 (m, 3H), 4.05 (dd, J=5.5, 9.0, 1H), 3.04-2.93 (m, 2H), 2.22 (s, 3H), 1.75 (s, 3H), 1.68-1.52 (m, 3H), 1.46-1.22 (m, 3H); Low resolution mass spectrum (ES) m/e 500 [(M+H)⁺, calcd for C₃₀H₃₄N₃O₄: 500]; 97% purity based on HPLC.

### Example 182

### (+/−)-4-(2-AMINO-ACETYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and final coupling to tert-Butoxycarbonylamino-acetic acid was as described in the method of Example 6. Purification by HPLC produced the title compound. 1H NMR (400 MHz, DMSO-d6) δ 10.01 (s, 0.5H), 9.77 (br s, 0.5H), 8.07-8.02 (m, 3H), 7.88 (s, 2H), 7.63-7.29 (m, 8H), 7.09 (s, 2H), 4.56 (d, J=30.72 Hz, 1H), 4.32-4.01 (m, 5H), 3.89-3.78 (m, 3H) under D2O, 3.32-3.30 (m, 1H) under D20, 3.21-2.99 (m, 3H), 2.58-2.55 (m, 0.5H), 2.45-2.32 (m, 1.5H), 2.23 (d, J=5.36 Hz, 3H); Low resolution mass spectrum (ES) m/e 513 [(M+H)⁺, calcd for C₃₀H₃₃N₄O₄: 513]; 98% purity based on HPLC.

### Example 183

### 4-(3-AMINO-PROPIONYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to 3-tert-Butoxycarbonylamino-propionic acid as described in the method of Example 182. Purification by HPLC produced the title compound. 1H NMR (400 MHz, DMSO-d6) δ 9.96 (s, 0.5H), 9.78 (s, 0.5H), 7.87 (d, J=6.18 Hz, 2H), 7.64-7.29 (m, 11H), 7.08 (s, 2H), 4.56 (d, J=20.79 Hz, 1H), 4.31-4.22 (m, 5H), 3.31 (d, J=12.11 Hz, 1H), 3.12-2.92 (m, 4H), 2.82-2.64 (m, 3H), 2.45-2.32 (m, 1H), 2.23 (d, J=3.09 Hz, 3H); Low resolution mass spectrum (ES) m/e 527 [(M+H)⁺, calcd for C₃₁H₃₅N₄O₄: 527]; 96.6% purity based on HPLC.

### Example 184

### 4-((S)-2-AMINO-PROPIONYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to (S)-2-tert-Butoxycarbonylamino-propionic acid as described in the method of Example 182. Purification by HPLC produced the title compound. 1H NMR (400 MHz, DMSO-d6) δ 10.16-9.59 (m, 1H), 8.11 (s, 3H), 7.87 (d, J=6.73 Hz, 2H), 7.64-7.28 (m, 8H), 7.12-7.09 (m, 2H), 4.65-4.11 (m, 6.5H), 3.98-3.84 (m, 1.5H), 3.29-2.87 (m, 2.5H), 2.77-2.65 (m, 1H), 2.42-2.31 (m, 0.5H), 2.23 (s, 3H), 1.41 (d, J=6.82 Hz, 0.6H), 1.32-1.28 (m, 2.2H); Low resolution mass spectrum (ES) m/e 527 [(M+H)⁺, calcd for C₃₁H₃₅N₄O₄: 527]; 97.7% purity based on HPLC.

### Example 185

### 4-(R-2-AMINO-PROPIONYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to (R)-2-tert-Butoxycarbonylamino-propionic acid as described in the method of Example 182. Purification by HPLC produced the title compound. 1H NMR (400 MHz, DMSO-d6) δ 10.16-9.60 (m, 1H), 8.12 (s, 3H), 7.87 (d, J=6.62 Hz, 2H), 7.73-7.28 (m, 8H), 7.12-7.09 (m, 2H), 4.65-4.11 (m, 5H), 3.98-3.34 (m, XH under D2O), 3.25-2.65 (m, 4H), 2.59-2.42 (m, nH under DMSO), 2.23 (s, 3H), 1.42-1.28 (m, 3H); Low resolution mass spectrum (ES) m/e 527 [(M+H)⁺, calcd for C₃₁H₃₅N₄O₄: 527]; 99.2% purity based on HPLC.

### Example 186

### 4-(2-ACETYLAMINO-ACETYL)-2-(P-TOLYLCARBAMOYL-METHYL)-PIPERAZINE-1-CARBOXYLIC ACID 9H-FLUOREN-9-YLMETHYL ESTER

(+/−)-4-(2-Amino-acetyl)-2-(p-tolylcarbamoyl-methyl)-piperazine-1-carboxylic acid 9H-fluoren-9-ylmethyl ester of example 182 was coupled to acetyl chloride as described in the method for Intermediate #3 of Example 1. Purification by HPLC produced the title compound. 1H NMR (400 MHz, DMSO-d6) δ 9.94 (s, 0.5H), 9.75 (s, 0.5H), 8.05-8.01 (m, 1H), 7.87 (d, J=5.91 Hz, 2H), 7.64-7.29 (m, 8H), 7.08 (m, 2H), 4.28-4.05 (m, 4H), 3.97-3.82 (m, 3H), 3.28-2.88 (m, 3H), 2.71 (br, 0.5H), 2.40-2.32 (m, 0.5), 2.23 (s, 3H), 1.87 (d, J=8.34 Hz, 3H); Low resolution mass spectrum (ES) m/e 555 [(M+H)⁺, calcd for C₃₂H₃₅N₄O₅: 555]; 97.7% purity based on HPLC.

### Example 187

### 4-(2-ETHYLAMINO-ACETYL)-2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester, and then to Ethylamino-acetic acid as described in the method of Example 182. Purification by HPLC produced the title compound. 1H NMR (400 MHz, DMSO-d6) δ 10.00 (s, 0.5H), 9.80 (s, 0.5H), 8.76 (br m, 2H), 8.87 (brm, 2H), 7.63-7.29 (m, 8H), 7.08 (m, 2H), 4.60-4.56 (m, 1H), 4.33-4.07 (m, 5H), 3.21-2.67 (m, 5H), 2.44-2.36 (m, 0.6), 2.23-2.22 (d, 3H), 1.45 (s, 2.6H), 1.21 (t, J=7.25, 1.8H), 1.16 (t, J=7.26, 1.2H); Low resolution mass spectrum (ES) m/e 541 [(M+H)⁺, calcd for C₃₂H₃₇N₄O₄: 541]; 93.9% purity based on HPLC.

### Example 188

### (S)-5-AMINO-2-(3-BIPHENYL-4-YL-UREIDO)-PENTANOIC ACID P-TOLYLAMIDE

p-Tolylamine was coupled to (S)-5-tert-Butoxycarbonylamino-2-(9H-fluoren-9-ylmethoxycarbonylamino)-pentanoic acid, and then to 4-Isocyanato-biphenyl as described in the method of Example 26. Purification by HPLC produced the title compound. 1H NMR (400 MHz, DMSO-d6) δ 10.16 (s, 1H), 8.93 (br s, 3H), 7.61 (d, J=7.23 Hz, 2H), 7.56 (d, J=8.77 Hz, 2H), 7.49 (t, J=8.80, 8.80 Hz, 4H), 7.41 (d, J=7.92 Hz, 2H), 7.30 (t, J=7.86, 7.86 Hz, 1H), 7.13 (d, J=8.37 Hz, 2H), 6.66 (d, J=8.28 Hz, 1H), 4.48 (dd, J=6.28, 13.97 Hz, 1H), 2.85-2.83 (m, 2H), 2.26 (s, 3H), 1.84-1.56 (m, 4H); Low resolution mass spectrum (ES) m/e 417 [(M+H)⁺, calcd for C₂₅H₂₉N₄O₂: 417]; 99.2% purity based on HPLC.

### Example 189

### (S)-5-AMINO-2-[3-(4-BENZYLOXY-PHENYL)-UREIDO]-PENTANOIC ACID P-TOLYLAMIDE

p-Tolylamine was coupled to (S)-5-tert-Butoxycarbonylamino-2-(9H-fluoren-9-ylmethoxycarbonylamino)-pentanoic acid, and then to 1-Benzyloxy-4-isocyanato-benzene as described in the method of Example 26. Purification by HPLC produced the title compound. 1H NMR (400 MHz, DMSO-d6) δ 10.13 (s, 1H), 8.62 (s, 1H), 7.71 (br s, 3H), 7.50 (d, J=8.42 Hz, 2H), 7.44-7.36 (m, 4H), 7.33-7.27 (m, 3H), 7.12 (d, J=8.39 Hz, 2H), 6.90 (d, J=9.04 Hz, 2H), 6.50 (d, J=8.33 Hz, 1H), 5.03 (s, 2H), 4.43 (dd, J=6.09, 13.66 Hz, 1H), 2.83-2.82 (m, 2H), 2.25 (s, 3H), 1.79-1.71 (m, 1H), 1.67-1.56 (m, 3H); Low resolution mass spectrum (ES) m/e 447 [(M+H)⁺, calcd for C₂₆H₃₁N₄O₃: 447]; 98.2% purity based on HPLC.

### Example 190

### (S)-5-AMINO-2-[3-(9H-FLUOREN-2-YL)-UREIDO]-PENTANOIC ACID P-TOLYLAMIDE

p-Tolylamine was coupled to (S)-5-tert-Butoxycarbonylamino-2-(9H-fluoren-9-ylmethoxycarbonylamino)-pentanoic acid, and then to 2-Isocyanato-9H-fluorene as described in the method of Example 26. Purification by HPLC produced the title compound. ¹H NMR (400 MHz, DMSO-d6) δ (S)-5-Amino-2-[3-(9H-fluoren-2-yl)-ureido]-pentanoic acid p-tolylamide: 1H NMR (400 MHz, DMSO-d6) δ 10.16 (s, 1H), 8.93 (s, 1H), 7.77-7.72 (br m, 6H), 7.52-7.50 (m, 3H), 7.34-7.31 (m, 2H), 7.22 (t, J=7.40, 7.40 Hz, 1H), 7.13 (d, J=8.40 Hz, 2H), 6.67 (d, J=8.23 Hz, 1H), 4.48 (dd, J=6.07, 13.59 Hz, 1H), 3.86 (s, 2H), 2.85-2.84 (m, 2H), 2.26 (s, 3H), 1.84-1.62 (m, 4H); Low resolution mass spectrum (ES) m/e 429 [(M+H)⁺, calcd for C₂₆H₂₉N₄O₂: 429]; 99.6% purity based on HPLC.

### Example 191

### (R)-5-AMINO-2-(3-BIPHENYL-4-YL-UREIDO)-PENTANOIC ACID P-TOLYLAMIDE

p-Tolylamine was coupled to (R)-5-tert-Butoxycarbonylamino-2-(9H-fluoren-9-ylmethoxycarbonylamino)-pentanoic acid, and then to 4-Isocyanato-biphenyl as described in the method of Example 26. Purification by HPLC produced the title compound. ¹H NMR (400 MHz, DMSO-d6) δ 10.16 (s, 1H), 8.92 (s, 1H), 7.70 (br s, 3H), 7.62-7.60 (m, 2H), 7.56 (d, J=8.73 Hz, 2H), 7.49 (t, J=8.85, 8.85 Hz, 4H), 7.42 (t, J=7.73, 7.73 Hz, 2H), 7.30 (t, J=7.35, 7.35 Hz, 1H), 7.13 (d, J=8.37 Hz, 2H), 6.66 (d, J=8.28 Hz, 1H), 4.48 (dd, J=6.19, 13.83 Hz, 1H), 2.85-2.83 (m, 2H), 2.26 (s, 3H), 1.84-1.61 (m, 4H); Low resolution mass spectrum (ES) m/e 417 [(M+H)⁺, calcd for C₂₅H₂₉N₄O₂: 417]; 93.9% purity based on HPLC.

### Example 192

### (R)-5-AMINO-2-[3-(4-BENZYLOXY-PHENYL)-UREIDO]-PENTANOIC ACID P-TOLYLAMIDE

p-Tolylamine was coupled to (R)-5-tert-Butoxycarbonylamino-2-(9H-fluoren-9-ylmethoxycarbonylamino)-pentanoic acid, and then to 1-Benzyloxy-4-isocyanato-benzene as described in the method of Example 26. Purification by HPLC produced the title compound. ¹H NMR (400 MHz, DMSO-d6) δ 10.13 (s, 1H), 8.63 (s, 1H), 7.73 (br s, 3H), 7.44-7.36 (m, 4H), 7.33-7.27 (m, 3H), 7.12 (d, J=8.40 Hz, 2H), 6.90 (d, J=9.07 Hz, 2H), 6.52 (d, J=8.31 Hz, 1H), 5.03 (s, 2H), 4.43 (dd, J=6.35, 13.88 Hz, 1H), 2.83-2.82 (m, 2H), 2.25 (s, 3H), 1.78-1.58 (m, 4H); Low resolution mass spectrum (ES) m/e 447 [(M+H)⁺, calcd for C₂₆H₃₁N₄O₃: 447]; 94.7% purity based on HPLC.

### Example 193

### (R)-5-AMINO-2-[3-(9H-FLUOREN-2-YL)-UREIDO]-PENTANOIC ACID P-TOLYLAMIDE

p-Tolylamine was coupled to (R)-5-tert-Butoxycarbonylamino-2-(9H-fluoren-9-ylmethoxycarbonylamino)-pentanoic acid then to 2-Isocyanato-9H-fluorene as described in the method of Example 26. Purification by HPLC produced the title compound. ¹H NMR (400 MHz, DMSO-d6) δ 10.16 (s, 1H), 8.92 (s, 1H), 7.81-7.72 (m, 6H), 7.52-7.50 (m, 3H), 7.34-7.31 (m, 2H), 7.24-7.12 (m, 3H), 6.66 (d, J=8.24 Hz, 1H), 4.48 (dd, J=6.11, 13.66 Hz, 1H), 3.86 (s, 2H), 2.85-2.83 (m, 2H), 2.26 (s, 3H), 1.84-1.62 (m, 4H); Low resolution mass spectrum (ES) m/e 429 [(M+H)⁺, calcd for C₂₆H₂₉N₄O₂: 429]; 93.6% purity based on HPLC.

### Example 194

### (S)-6-AMINO-2-[3-(4-HYDROXY-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Hydrogenation overnight using 10% palladium on carbon in ethanol under a hydrogen atmosphere followed by purification by HPLC produced the title compound. ¹H NMR (400 MHz, DMSO-d6) δ 10.05 (s, 1H), 8.97 (s, 1H), 8.39 (s, 1H), 7.68 (br s, 3H), 7.49 (d, J=8.42 Hz, 2H), 7.14 (d, J=8.87 Hz, 2H), 7.11 (d, J=8.48 Hz, 2H), 6.63 (d, J=8.86 Hz, 2H), 6.56 (br s, 0.5H), 6.38 (d, J=8.28 Hz, 1H), 4.36 (dd, J=8.01, 13.54 Hz, 1H), 2.80-2.76 (m, 2H), 2.25 (s, 3H), 1.76-1.67 (m, 1H), 1.63-1.52 (m, 3H), 1.43-1.31 (m, 2H); Low resolution mass spectrum (ES) m/e 371 [(M+H)⁺, calcd for C₂₀H₂₇N₄O₃: 371]; 86.6% purity based on HPLC.

### Example 195

### (R)-6-AMINO-2-[3-(9H-FLUOREN-9-YL)-UREIDO]-HEXANOIC ACID (4-CYCLOHEXYL-PHENYL)-AMIDE

4-Cyclohexyl-phenylamine was coupled to (R)-6-tert-Butoxycarbonylamino-2-(9H-fluoren-9-ylmethoxycarbonylamino)-hexanoic acid, and then to 9-Isocyanato-9H-fluorene as described in the method of Example 26. Purification by HPLC produced the title compound. ¹H NMR (400 MHz, DMSO-d6) δ 10.09 (s, 1H), 7.84 (d, J=6.42 Hz, 2H), 7.67 (br, 3H), 7.55-7.48 (m, 3H), 7.44-7.37 (m, 2H), 7.31 (td, J=7.48, 7.48, 10.62 Hz, 2H), 7.17 (d, J=8.54 Hz, 2H), 6.63 (d, J=8.65 Hz, 1H), 6.26 (d, J=8.47 Hz, 1H), 5.82 (d, J=8.59 Hz, 1H), 4.47 (dd, J=7.90, 13.97 Hz, 1H), 2.82-2.77 (m, 2H), 2.44 (br m, 1H), 1.79-1.69 (m, 6H), 1.64-1.53 (m, 3H), 1.46-1.31 (m, 6H), 1.26-1.20 (m, 1H); Low resolution mass spectrum (ES) m/e 511 [(M+H)⁺, calcd for C₃₂H₃₉N₄O₂: 511]; 90.5% purity based on HPLC.

### Example 196

### (S)-6-AMINO-2-{3-[4-(4-NITRO-BENZYLOXY)-PHENYL]-UREIDO}-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-(4-nitrophenyl)methyloxy-4-isocyanato-benzene as described in the method of Example 26. Purification by HPLC produced the title compound. ¹H NMR (400 MHz, DMSO-d6) δ 10.06 (s, 1H), 8.56 (s, 1H), 8.25 (d, J=8.77 Hz, 2H), 7.71-7.65 (m, 5H), 7.49 (d, J=8.40 Hz, 2H), 7.29 (d, J=7.05 Hz, 2H), 7.11 (d, J=8.30 Hz, 2H), 6.92 (d, J=9.06 Hz, 2H), 6.44 (d, J=8.29 Hz, 1H), 5.21 (s, 2H), 4.37 (dd, J=8.02, 13.64 Hz, 1H), 2.80-2.75 (m, 2H), 2.25 (s, 3H), 1.77-1.68 (m, 1H), 1.63-1.50 (m, 3H), 1.43-1.30 (m, 2H); Low resolution mass spectrum (ES) m/e 506 [(M+H)⁺, calcd for C₂₇H₃₂N₅O₅: 506]; 97.5% purity based on HPLC.

### Example 197

### 2-(P-TOLYLCARBAMOYL-METHYL)-PIPERAZINE-1-CARBOXYLIC ACID 9H-FLUOREN-9-YLMETHYL ESTER

p-Tolylamine was coupled to (+/−)-2-Carboxymethyl-piperazine-1,4-dicarboxylic acid 4-tert-butyl ester 1-(9H-fluoren-9-ylmethyl) ester as described in the method of Example 6. Purification by HPLC produced the title compound. ¹H NMR (400 MHz, DMSO-d6) δ 9.99 (s, 1H), 9.23 (s, 1H), 8.75 (s, 1H), 7.86 (d, J=7.30 Hz, 2H), 7.62 (d, J=7.41 Hz, 1H), 7.48-7.46 (br m, 3H), 7.39 (t, J=7.40, 7.40 Hz, 2H), 1.32-1.26 (m, 2H), 7.10 (d, J=8.07 Hz, 2H), 4.68-4.67 (m, 1H), 4.25 (br s, 2H), 4.17 (br s, 1H), 3.98 (d, J=12.60 Hz, 1H), 3.41 (d, J=12.72 Hz, 1H), 3.28-3.17 (m, 3H), 2.90 (dd, J=8.30, 15.06 Hz, 2H), 2.56 (dd, J=6.27, 15.03 Hz, 1H), 2.23 (s, 3H); Low resolution mass spectrum (ES) m/e 456 [(M+H)⁺, calcd for C₂₈H₃₀N₃O₃: 456]; 99.7% purity based on HPLC.

### Example 198

### (R)-[4-AMINO-1-(4-CYCLOHEXYL-PHENYLCARBAMOYL)-BUTYL]-CARBAMIC ACID 9H-FLUOREN-9-YLMETHYL ESTER

4-Cyclohexyl-phenylamine was coupled to (R)-5-tert-Butoxycarbonylamino-2-(9H-fluoren-9-ylmethoxycarbonylamino)-pentanoic acid as described in the method of Example 6. Purification by HPLC produced the title compound. ¹H NMR (400 MHz, DMSO-d6) δ 10.02 (s, 1H), 7.89 (d, J=7.51 Hz, 2H), 7.76-7.71 (m, 6H), 7.50 (d, J=8.29 Hz, 2H), 7.42 (t, J=7.30, 7.30 Hz, 2H), 7.32 (dd, J=6.81, 12.89 Hz, 2H), 7.15 (d, J=8.46 Hz, 2H), 4.34-4.14 (m, 4H), 2.81-2.80 (m, 2H), 2.43 (br, 1H), 1.78-1.60 (m, 9H), 1.41-1.19 (m, 5H); Low resolution mass spectrum (ES) m/e 512 [(M+H)⁺, calcd for C₃₂H₃₈N₃O₃: 512]; 99.5% purity based on HPLC.

### Example 199

### (S)-4-{4-[3-(5-AMINO-1-P-TOLYLCARBAMOYL-PENTYL)-UREIDO]-PHENOXYMETHYL}-BENZOIC ACID METHYL ESTER

p-Tolylamine was coupled to (S)-6-tert-Butoxycarbonylamino-2-(9H-fluoren-9-ylmethoxycarbonylamino)-hexanoic acid, and then to 4-(4-Isocyanato-phenoxymethyl)-benzoic acid methyl ester as described in the method of Example 26. Purification by HPLC produced the title compound. ¹H NMR (400 MHz, DMSO-d6) δ 10.07 (s, 1H), 8.54 (s, 1H), 7.97 (d, J=8.27 Hz, 2H), 7.64 (br s, 3H), 7.57 (d, J=8.29 Hz, 2H), 7.49 (d, J=8.39 Hz, 2H), 7.28 (d, J=9.04 Hz, 2H), 7.11 (d, J=8.35 Hz, 2H), 6.90 (d, J=9.05 Hz, 2H) 6.43 (d, J=8.28 Hz, 1H), 5.14 (s, 2H), 4.37 (dd, J=7.97, 13.62 Hz, 1H), 3.85 (s, 3H), 2.82-2.74 (m, 2H), 2.25 (s, 3H), 1.77-1.68 (m, 1H), 1.63-1.52 (m, 3H), 1.44-1.29 (m, 2H0; Low resolution mass spectrum (ES) m/e 519 [(M+H)⁺, calcd for C₂₉H₃₅N₄O₅: 519]; 93.0% purity based on HPLC.

### Example 200

### (S)-[4-AMINO-1-(4-CYCLOHEXYL-PHENYLCARBAMOYL)-BUTYL]-CARBAMIC ACID 9H-FLUOREN-9-YLMETHYL ESTER

4-Cyclohexyl-phenylamine was coupled to (S)-5-tert-Butoxycarbonylamino-2-(9H-fluoren-9-ylmethoxycarbonylamino)-pentanoic acid as described in the method of Example 6. Purification by HPLC produced the title compound. ¹H NMR (400 MHz, DMSO-d6) δ 10.01 (s, 1H), 7.90 (d, J=7.51 Hz, 2H), 7.75-7.71 (m, 5H), 7.50 (d, J=8.42 Hz, 2H), 7.42 (t, J=7.34, 7.34 Hz, 2H), 7.32 (dd, J=6.91, 12.74 Hz, 2H), 7.15 (d, J=8.44 Hz, 2H), 4.34-4.13 (m, 4H), 2.81-2.79 (m, 2H), 2.43 _br, 1H), 1.78-1.56 (m, 9H), 1.41-1.19 (m, 5H); Low resolution mass spectrum (ES) m/e 512 [(M+H)⁺, calcd for C₃₂H₃₈N₃O₃: 512]; 99.5% purity based on HPLC.

### Example 201

### (R)-5-AMINO-2-(3-BIPHENYL-4-YL-UREIDO)-PENTANOIC ACID (4-CYCLOHEXYL-PHENYL)-AMIDE

4-Cyclohexyl-phenylamine was coupled to (R)-5-tert-Butoxycarbonylamino-2-(9H-fluoren-9-ylmethoxycarbonylamino)-pentanoic acid, and then to 4-Isocyanato-biphenyl as described in the method of Example 26. Purification by HPLC produced the title compound. ¹H NMR (400 MHz, DMSO-d6) δ 10.17 (s, 1H), 8.91 (s, 1H), 7.67 (br s, 3H), 7.61 (d, J=7.28 Hz, 2H), 7.57-7.47 (m, 6H), 7.42 (t, J=7.69, 7.69 Hz, 2H), 7.30 (t, J=7.34, 7.34 Hz, 1H), 7.17 (d, J=8.53 Hz, 2H), 6.64 (d, J=8.25 Hz, 1H), 4.48 (dd, J=6.47, 13.77 Hz, 1H), 2.84-2.82 (m, 2H), 2.44 (br m, 1H), 1.78-1.57 (m, 8H), 1.42-1.20 (m, 4H); Low resolution mass spectrum (ES) m/e 485 [(M+H)⁺, calcd for C₃₀H₃₇N₄O₂: 485]; 99.5% purity based on HPLC.

### Example 202

### (R)-5-AMINO-2-[3-(4-BENZYLOXY-PHENYL)-UREIDO]-PENTANOIC ACID (4-CYCLOHEXYL-PHENYL)-AMIDE

4-Cyclohexyl-phenylamine was coupled to (R)-5-tert-Butoxycarbonylamino-2-(9H-fluoren-9-ylmethoxycarbonylamino)-pentanoic acid, and then to 1-Benzyloxy-4-isocyanato-benzene as described in the method of Example 26. Purification by HPLC produced the title compound. ¹H NMR (400 MHz, DMSO-d6) δ 10.14 (s, 1H), 8.60 (s, 1H), 7.67 (br s, 3H), 7.51 (d, J=8.53 Hz, 2H), 7.43-7.36 (m, 4H), 7.32 (d, J=8.24 Hz, 1H), 7.28 (d, J=9.05 Hz, 2H), 7.16 (d, J=8.54 Hz, 2H), 6.90 (d, J=9.03 Hz, 2H), 6.48 (d, J=8.31 Hz, 1H), 5.03 (s, 2H), 4.44 (dd, J=6.22, 13.70 Hz, 1H), 2.83-2.81 (m, 2H), 2.44 (br m, 1H), 1.78-1.60 1.42-1.19 (m, 4H); Low resolution mass spectrum (ES) m/e 515 [(M+H)⁺, calcd for C₃₁H₃₉N₄O₃: 515]; 99.0% purity based on HPLC.

### Example 203

### (R)-5-AMINO-2-[3-(9H-FLUOREN-2-YL)-UREIDO]-PENTANOIC ACID (4-CYCLOHEXYL-PHENYL)-AMIDE

4-Cyclohexyl-phenylamine was coupled to (R)-5-tert-Butoxycarbonylamino-2-(9H-fluoren-9-ylmethoxycarbonylamino)-pentanoic acid then to 2-Isocyanato-9H-fluorene as described in the method of Example 26. Purification by HPLC produced the title compound. ¹H NMR (400 MHz, DMSO-d6) δ 10.17 (s, 1H), 8.90 (s, 1H), 7.77-7.68 (m, 5H), 7.53-7.51 (m, 3H), 7.35-7.31 (m, 3H), 7.22 (dt, J=0.98, 7.47, 7.51 Hz, 1H), 7.17 (d, J=8.56 Hz, 2H), 6.64 (d, J=8.21 Hz, 1H), 4.48 (dd, J=6.47, 13.83 Hz, 1H), 3.86 (s, 2H), 2.84-2.83 (m, 2H), 2.44 (br m, 1H), 1.82-1.62 (m, 8H), 1.42-1.19 (m, 4H); Low resolution mass spectrum (ES) m/e 497 [(M+H)⁺, calcd for C₃₁H₃₇N₄O₂: 497]; 99.0% purity based on HPLC.

### Example 204

### (S)-6-AMINO-2-[3-(3-BENZYLOXY-PHENYL)-UREIDO]-HEXANOIC ACID P-TOLYLAMIDE

(S)-(5-Amino-5-p-tolylcarbamoyl-pentyl)-carbamic acid tert-butyl ester from Intermediate #2 of Example 1 was coupled to 1-Benzyloxy-3-isocyanato-benzene as described in the method of Example 26. Purification by HPLC produced the title compound. ¹H NMR (400 MHz, DMSO-d6) δ 10.08 (s, 1H), 8.78 (s, 1H), 7.71 (br s, 3H), 7.50 (d, J=8.40 Hz, 2H), 7.44-7.36 (m, 4H), 7.34-7.30 (m, 1H), 7.21 (s, 1H), 7.14-7.10 (m, 3H), 6.86 (d, J=8.17 Hz, 1H), 6.61-6.55 (m, 2H), 5.04 (s, 2H), 4.37 (dd, J=7.90, 13.46 Hz, 1H), 2.82-2.74 (m, 2H), 2.25 (s, 3H), 1.78-1.69 (m, 1H), 1.65-1.53 (m, 3H), 1.45-1.30 (m, 2H); Low resolution mass spectrum (ES) m/e 461 [(M+H)⁺, calcd for C₂₇H₃₃N₄O₃: 461]; 99.6% purity based on HPLC.

### Example 205

### (R)-6-AMINO-2-[3-(3-BENZYLOXY-PHENYL)-UREIDO]-HEXANOIC ACID P-TOLYLAMIDE

p-Tolylamine was coupled to (R)-6-tert-Butoxycarbonylamino-2-(9H-fluoren-9-ylmethoxycarbonylamino)-hexanoic acid, and then to 1-Benzyloxy-3-isocyanato-benzene as described in the method of Example 26. Purification by HPLC produced the title compound. ¹H NMR (400 MHz, DMSO-d6) δ 10.08 (s, 1H), 8.78 (s, 1H), 7.72 (br s, 3H), 7.50 (d, J=8.42 Hz, 2H), 7.44-7.36 (m, 4H), 7.32 (t, J=7.11, 7.11 Hz, 1H), 7.21 (t, J=2.15, 2.15 Hz, 1H), 7.14-7.10 (m, 3H), 6.86 (dd, J=1.17, 8.09 Hz, 1H), 6.61-6.55 (m, 2H), 5.04 (s, 2H), 4.37 (dd, J=8.00, 13.47 Hz, 1H), 2.81-2.75 (m, 2H), 2.25 (s, 3H), 1.78-1.69 (m, 1H), 1.65-1.53 (m, 3H), 1.46-1.31 (m, 2H); Low resolution mass spectrum (ES) m/e 461 [(M+H)⁺, calcd for C₂₇H₃₃N₄O₃: 461; 99.6% purity based on HPLC.

### Example 206

### (S)-6-AMINO-2-[3-(4-BENZYLOXY-PHENYL)-UREIDO]-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid, and then to 1-Benzyloxy-4-isocyanato-benzene as described in the method of Example 26. Purification by HPLC produced the title compound. ¹H NMR (400 MHz, DMSO-d6) δ 9.42 (s, 1H), 8.55 (s, 1H), 8.43 (s, 4H), 7.77 (br s, 1H), 7.49 (d, J=8 Hz, 2H), 7.14 (d, J=9 Hz, 2H), 6.86 (br s, 1H), 6.56 (br s, 1H), 4.62 (br s, 2H), 4.10 (br s, 1H), 3.87 (s, 3H), 2.68 (s, 4H), 1.69-1.42 (m, 13H), 1.24-0.96 (m, 3H); Low resolution mass spectrum (ES) m/e 500 [(M+H)⁺, calcd for C₂₉H₃₄N₅O₃: 500]; 99.9% purity based on HPLC.

### Example 207

### (S)-6-AMINO-2-[3-(9H-FLUOREN-2-YL)-UREIDO]-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid then to 2-Isocyanato-9H-fluorene as described in the method of Example 26. Purification by HPLC produced the title compound. ¹H NMR (400 MHz, DMSO-d6) δ 10.84 (s, 1H), 9.92 (s, 1H), 8.89 (s, 1H), 7.76-7.72 (br m, 7H), 7.51 (d, J=7.43 Hz, 1H), 7.32 (t, J=7.23, 7.23 Hz, 2H), 7.22 (dt, J=1.00, 7.59, 7.73 Hz, 1H), 7.18 (d, J=8.64 Hz, 1H), 7.14 (dd, J=1.86, 8.65 Hz, 1H), 6.60 (d, J=8.20 Hz, 1H), 6.06 (s, 1H), 4.44 (dd, J=7.80, 13.51 Hz, 1H), 3.86 (s, 2H), 2.84-2.76 (m, 2H), 2.35 (s, 3H), 1.82-1.74 (m, 1H), 1.69-1.54 (m, 3H), 1.49-1.35 (m, 2H); Low resolution mass spectrum (ES) m/e 482 [(M+H)⁺, calcd for C₂₉H₃₂N₅O₂: 482]; 99.5% purity based on HPLC.

### Example 208

### (S)-[5-AMINO-1-(1H-INDOL-4-YLCARBAMOYL)-PENTYL]-CARBAMIC ACID 9H-FLUOREN-9-YLMETHYL ESTER

1H-Indol-4-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 11.15 (s, 1H); 9.60 (s, 1H); 7.75 (t, J=6.58 Hz, 2H); 7.71-7.61 (m, 4H); 7.57 (d, J=7.63 Hz, 1H); 7.42 (t, J=7.45 Hz, 2H); 7.36-7.26 (m, 3H); 7.16 (d, 8.07 Hz, 1H); 7.02 (t, J=7.88 Hz, 1H); 6.65 (br s, 1H); 4.42-4.19 (m, 4H); 2.79 (dddd, J=6.7; 6.7; 6.1; 11.6 Hz, 2H); 1.84-1.63 (m, 2H); 1.63-1.52 (m, 2H); 1.52-1.32 (m, 2H); Low resolution mass spectrum (ES) m/e 483 [(M+H)⁺, calcd for C₂₉H₃₁N₄O₃: 483]; 91.5% purity based on HPLC.

### Example 209

### (S)-[5-AMINO-1-(4-METHYLAMINO-PHENYLCARBAMOYL)-PENTYL]-CARBAMIC ACID 9H-FLUOREN-9-YLMETHYL ESTER

N-Methyl-benzene-1,4-diamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.15 (s, 1H); 8.1 (br s, 3H); 7.90 (d, J=7.49 Hz, 2H); 7.77-7.60 (m, 8H); 7.46-7.29 (m, 6H); 4.30 (d, J=6.71, 2H); 4.27-4.19 (m, 1H); 4.16-4.08 (m, 1H); 3.98 (br s, 1H); 2.78 (br s, 2H); 1.77-1.28 (m, 6H); Low resolution mass spectrum (ES) m/e 473 [(M+H)⁺, calcd for C₂₈H₃₃N₄O₃: 473]; 99% purity based on HPLC.

### Example 210

### (S)-[5-AMINO-1-(4-BENZYLOXY-3-CHLORO-PHENYLCARBAMOYL)-PENTYL]-CARBAMIC ACID 9H-FLUOREN-9-YLMETHYL ESTER

4-Benzyloxy-3-chloro-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.1 (s, 1H); 7.90 (d, J=7.54 Hz, 2H); 7.82 (d, J=2.38 Hz, 1H); 7.73 (t, J=7.18 Hz, 2H); 7.69-7.58 (m, 4H); 7.49-7.37 (m, 7H); 7.36-7.29 (m, 3H); 7.2 (d, J=9 Hz, 1H); 5.17 (s, 2H); 4.35-4.19 (m, 3H); 4.07 (dd, J=8.29; 13.9 Hz; 1H); 2.78 (dddd, J=6.04; 6.04; 6.27; 12.67 Hz; 2H); 1.76-1.59 (m, 2H); 1.59-1.47 (m, 2H); 1.47-1.25 (m, 2H); Low resolution mass spectrum (ES) m/e 584 [(M+H)⁺, calcd for C₃₄H₃₅ClN₃O₄: 584]; 98% purity based on HPLC.

### Example 211

### (S)-[5-AMINO-1-(3-BENZYLOXY-PHENYLCARBAMOYL)-PENTYL]-CARBAMIC ACID 9H-FLUOREN-9-YLMETHYL ESTER

3-Benzyloxy-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ; 10.0 (s, 1H); 7.90 (d, J=7.56 Hz, 2H); 7.74 (t, J=7.41 Hz, 2H); 7.70-7.56 (m, 4H); 7.46-7.26 (m, 10H); 7.21 (d, J=8.1 Hz, 1H); 7.14 (d, J=8.39; 1H); 6.72 (dd, J=1.83; 8.09 Hz; 1H); 5.07 (s, 2H); 4.35-4.19 (m, 3H); 4.12 (ddd, J=5.66; 8.42; 8.28 Hz; 1H); 2.78 (dddd, J=6.04; 6.04; 6.27; 12.67 Hz; 2H); 1.75-1.60 (m, 2H); 1.60-1.48 (m, 2H); 1.48-1.25 (m, 2H). Low resolution mass spectrum (ES) m/e 550 [(M+H)⁺, calcd for C₃₁H₃₇N₃O₆: 550]; 97% purity based on HPLC.

### Example 212

### (S)-6-AMINO-2-[3-(3-BENZYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Benzyl-3-isocyanato-benzene as described in the method of Example 26. Purification by HPLC produced the title compound. ¹H NMR (400 MHz, DMSO-d6) δ 10.06 (s, 1H), 8.69 (s, 1H), 7.70 (br s, 3H), 7.49 (d, J=8.42 Hz, 2H), 7.30-7.10 (m, 10H), 6.78 (d, J=7.51 Hz, 1H), 6.52 (d, J=8.19 Hz, 1H), 4.35 (dd, J=8.01, 13.48 Hz, 1H), 3.86 (s, 2H), 2.80-2.75 (m, 2H), 2.25 (s, 3H), 1.77-1.68 (m, 1H), 1.63-1.51 (m, 3H), 1.43-1.27 (m, 2H); Low resolution mass spectrum (ES) m/e 445 [(M+H)⁺, calcd for C₂₇H₃₃N₄O₂: 445]; 99.4% purity based on HPLC.

### Example 213

### (S)-6-AMINO-2-[3-(2,3-DIHYDRO-BENZO[1,4]DIOXIN-6-YL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 6-Isocyanato-2,3-dihydro-benzo[1,4]dioxine as described in the method of Example 26. Purification by HPLC produced the title compound. ¹H NMR (400 MHz, DMSO-d6) δ 10.07 (s, 1H), 8.53 (s, 1H), 7.67 (br s, 3H), 7.49 (d, J=8.42 Hz, 2H), 7.11 (d, J=8.37 Hz, 2H), 7.04 (d, J=1.07 Hz, 1H), 6.70-6.69 (m, 2H), 6.43 (d, J=8.28 Hz, 1H), 4.36 (dd, J=8.03, 13.59 Hz, 1H), 4.17 (dd, J=5.10, 11.93 Hz, 4H), 2.82-2.74 (m, 2H), 2.25 (s, 3H), 1.76-1.52 (m, 4H), 1.44-1.30 (m, 2H); Low resolution mass spectrum (ES) m/e 413 [(M+H)⁺, calcd for C₂₂H₂₉N₄O₄: 413]; 99.9% purity based on HPLC.

### Example 214

### (S)-6-AMINO-2-{3-[4-(6-METHYL-BENZOTHIAZOL-2-YL)-PHENYL]-UREIDO}-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 2-(4-Isocyanato-phenyl)-benzothiazole as described in the method of Example 26. Purification by HPLC produced the title compound. ¹H NMR (400 MHz, DMSO-d6) δ 10.12 (s, 1H), 9.18 (s, 1H), 7.94 (d, J=8.81 Hz, 2H), 7.88-7.86 (m, 2H), 7.69 (br m, 3H), 7.58 (d, J=8.80 Hz, 2H), 7.51 (d, J=8.42 Hz, 2H), 7.32 (dd, J=1.13, 8.35 Hz, 1H), 7.12 (d, J=8.40 Hz, 2H), 6.75 (d, J=8.12 Hz, 1H), 4.42 (dd, J=7.95, 13.37 Hz, 1H), 2.83-2.75 (m, 2H), 2.44 (s, 3H), 2.25 (s, 3H), 1.81-1.73 (m, 1H), 1.68-1.52 (m, 3H), 1.47-1.32 (m, 2H); Low resolution mass spectrum (ES) m/e 502 [(M+H)⁺, calcd for C₂₈H₃₂N₅O₂S: 502]; 99.8% purity based on HPLC.

### Example 215

### (S)-[2-(4-AMINOMETHYL-PHENYL)-1-(2-METHYL-1H-INDOL-5-YLCARBAMOYL)-ETHYL]-CARBAMIC ACID 9H-FLUOREN-9-YLMETHYL ESTER

2-Methyl-1H-indol-5-ylamine was coupled to (S)-3-[4-(tert-Butoxycarbonylamino-methyl)-phenyl]-2-(9H-fluoren-9-ylmethoxycarbonylamino)-propionic acid as described in the method of Example 6. Purification by HPLC produced the title compound. ¹H NMR (400 MHz, DMSO-d6) δ 10.84 (s, 1H), 9.89 (s, 1H), 8.14 (br s, 3H), 7.89 (d, J=7.54 Hz, 2H), 7.78 (d, J=8.55 Hz, 1H), 7.73-7.68 (m, 3H), 7.43-7.36 (m, 6H), 7.31 (t, J=7.39, 7.39 Hz, 2H), 7.19 (d, J=8.55 Hz, 1H), 7.11 (dd, J=1.74, 8.65 Hz, 1H), 6.07 (s, 1H), 4.42 (dt, J=4.65, 9.63, 9.68 Hz, 1H), 4.26-4.13 (m, 3H), 4.01-3.97 (m, 2H), 3.07 (dd, J=4.39, 13.59 Hz, 1H), 2.92 (dd, J=10.43, 13.57 Hz, 1H), 2.36 (s, 3H); Low resolution mass spectrum (ES) m/e 545 [(M+H)⁺, calcd for C₃₄H₃₃N₄O₃: 545]; 100% purity based on HPLC.

### Example 216

### (S)-[5-AMINO-1-(3,4-DIMETHYL-PHENYLCARBAMOYL)-PENTYL]-CARBAMIC ACID 9H-FLUOREN-9-YLMETHYL ESTER

3,4-Dimethyl-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 9.85 (s, 1H); 7.90 (d, J=7.52 Hz, 1H); 7.74 (t, J=7.26 Hz, 2H); 7.69-7.55 (m, 4H); 7.42 (t, J=7.43 Hz, 2H); 7.38-7.28 (m, 4H); 7.05 (d, J=8.19 Hz, 1H); 4.36-4.19 (m, 3H); 4.11 (ddd, J=5.8; 8.4; 8.4 Hz, 1H); 2.78 (d, J=5.96 Hz, 2H); 2.18 (s, 3H); 2.16 (s, 3H); 1.75-1.46 (m, 4H); 1.44-1.25 (m, 2H). Low resolution mass spectrum (ES) m/e 472 [(M+H)⁺, calcd for C₂₉H₃₄N₃O₃: 472]; 99% purity based on HPLC.

### Example 217

### (S)-[1-(3,4-DICHLORO-PHENYLCARBAMOYL)-5-FORMYLAMINO-PENTYL]-CARBAMIC ACID 9H-FLUOREN-9-YLMETHYL ESTER

3,4-Dichloro-phenylamine was coupled to (S)-6-tert-Butoxycarbonylamino-2-(9H-fluoren-9-ylmethoxycarbonylamino)-hexanoic acid as described in the method of Example 6. Purification by HPLC produced unexpectedly the title compound. ¹H NMR (400 MHz, DMSO-d6) δ 7.95 (t, J=5.6 Hz, 1H); 7.89 (d, J=7.49 Hz, 2H); 7.72 (dd, J=4.56; 7.24 Hz, 2H); 7.64 (br s, 3H); 7.52-7.45 (m, 2H); 7.42 (t, J=7.50 Hz, 3H); 7.33 (t, J=7.44 Hz, 2H); 7.20 (dd, J=1.83; 8.24 Hz, 1H); 4.33-4.14 (m, 3H); 3.87 (ddd, J=5.5; 8.7; 8.7 Hz, 1H); 2.7 (t, J=6.60 Hz, 1H); 1.59-1.39 (m, 4H); 1.36-1.14 (m, 2H). Low resolution mass spectrum (ES) m/e 540 [(M+H)⁺, calcd for C₂₈H₂₈N₃O₄: 540]; 99% purity based on HPLC.

### Example 218

### (S)-[5-AMINO-1-(3-TRIFLUOROMETHYL-PHENYLCARBAMOYL)-PENTYL]-CARBAMIC ACID 9H-FLUOREN-9-YLMETHYL ESTER

3-trifluoromethyl-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ; 10.4 (s, 1H); 8.11 (s, 1H); 7.89 (d, J=7.52 Hz, 2H); 7.79 (d, J=8.37 Hz, 1H); 7.71 (dd, J=7.04; 13.1 Hz, 3H); 7.66-7.59 (m, 2H); 7.56 (d, J=7.99 Hz, 1H); 7.46-7.37 (m, 3H); 7.54-7.27 (m, 2H); 4.35-4.15 (m, 3); 4.11 (dd, J=8.30; 13.55 Hz, 1H); 2.78 (dt, J=6.14; 18.9 Hz, 2H); 1.81-1.45 (m, 4H); 1.43-1.25 (m, 2H). Low resolution mass spectrum (ES) m/e 512 [(M+H)⁺, calcd for C₂₈H₂₉N₃O₃: 512]; 95% purity based on HPLC.

### Example 219

### (S)-[1-(3-ACETYL-PHENYLCARBAMOYL)-5-AMINO-PENTYL]-CARBAMIC ACID 9H-FLUOREN-9-YLMETHYL ESTER

3-acetyl-phenylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.26 (s, 1H); 8.18 (s, 1H); 7.87 (dd, 1H, J=4.3; 11.2 Hz 3H); 7.73 (t, J=7.15 Hz, 2H); 7.69-7.58 (m, 4H); 7.47 (t, J=7.91 Hz, 1H); 7.41 (ddd, J=1.4; 7.4; 7.4 Hz; 7.32 (ddd, J=2.8; 6.7; 7.4 Hz, 2H); 4.35-4.18 (m, 3H); 4.12 (dd, J=8.4; 13.7, 1H); 2.78 (dt, J=6.13; 18.6, 2H); 2.55 (s, 3H); 1.79-1.47 (m, 4H); 1.47-1.26 (m, 2H). Low resolution mass spectrum (ES) m/e 486 [(M+H)⁺, calcd for C₂₉H₃₂N₃O₄: 486]; 94% purity based on HPLC.

### Example 220

### (S)-{4-[R-(PYRROLIDINE-2-CARBONYL)-AMINO]-1-P-TOLYLCARBAMOYL-BUTYL}-CARBAMIC ACID 9H-FLUOREN-9-YLMETHYL ESTER

(S)-(4-Amino-1-p-tolylcarbamoyl-butyl)-carbamic acid 9H-fluoren-9-ylmethyl ester from Example 101 was coupled to (R)-Pyrrolidine-1,2-dicarboxylic acid 1-tert-butyl ester as described in the method of Example 6. Purification by HPLC produced the title compound. ¹H NMR (400 MHz, DMSO-d6) 7.87 (d, J=8 Hz, 2H), 7.70 (t, J=9 and 8 Hz, 2H), 7.45 (d, 8 Hz, 2H), 7.40 (ddd, J=2, 7, 8 Hz, 2H), 7.35-7.26 (m, 2H), 7.10 (d, J=8 Hz, 2H), 4.36-4.07 (m, 5H), 3.15-3.05 (m, 3H), 2.22 (s, 3H), 1.81-1.44 (m, 5H), 1.38-1.08 (m, 3H); Low resolution mass spectrum (ES) m/e 542 [(M+H)⁺, calcd for C₃₂H₃₇N₄O₄: 542]; 89% purity based on HPLC.

### Example 221

### (S)-[3-(2-AMINO-ACETYLAMINO)-1-P-TOLYLCARBAMOYL-PROPYL]-CARBAMIC ACID 9H-FLUOREN-9-YLMETHYL ESTER

(S)-(3-Amino-1-p-tolylcarbamoyl-propyl)-carbamic acid 9H-fluoren-9-ylmethyl ester of Example 108 was coupled to tert-Butoxycarbonylamino-acetic acid as described in the method of Example 6. Purification by HPLC produced the title compound. ¹H NMR (400 MHz, DMSO-d6) 7.86 (d, J=8 Hz, 2H), 7.70 (t, J=7 Hz, 2H), 7.46-7.36 (m, 4H), 7.31 (dd, J=7, 14 Hz, 2H), 7.10 (d, J=8 Hz, 2H), 4.32-4.17 (m, 3H), 4.12 (dd, J=5, 9 Hz, 1H), 3.49 (s, 2H), 3.28-3.06 (m, 2H), 2.22 (s, 3H), 1.93-1.70 (m, 2H); Low resolution mass spectrum (ES) m/e 487 [(M+H)⁺, calcd for C₂₈H₃₁N₄O₄: 487]; 99% purity based on HPLC.

### Example 222

### (S)-{2-[2-(2-AMINO-ACETYLAMINO)-ACETYLAMINO]-1-P-TOLYLCARBAMOYL-ETHYL}-CARBAMIC ACID 9H-FLUOREN-9-YLMETHYL ESTER

(S)-(2-Amino-1-p-tolylcarbamoyl-ethyl)-carbamic acid 9H-fluoren-9-ylmethyl ester of Example 103 was coupled to (2-tert-Butoxycarbonylamino-acetylamino)-acetic acid as described in the method of Example 6. Purification by HPLC produced the title compound. ¹H NMR (400 MHz, DMSO-d6) 7.87 (d, J=8 Hz, 2H), 7.69 (dd, J=4, 7 Hz, 2H), 7.48-7.36 (m, 4H), 7.31 (dd, J=7, 15 Hz, 2H), 7.09 (d, J=8.0 Hz, 2H), 4.35-4.14 (m, 6H), 3.76 (s, 2H), 3.40 (d, J=6 Hz, 2H), 2.23 (s, 3H); Low resolution mass spectrum (ES) m/e 530 [(M+H)⁺, calcd for C₂₉H₃₂N₅O₅: 530]; 95% purity based on HPLC.

### Example 223

### (S)-[4-(2-METHYLAMINO-ACETYLAMINO)-1-P-TOLYLCARBAMOYL-BUTYL]-CARBAMIC ACID 9H-FLUOREN-9-YLMETHYL ESTER

(S)-(4-Amino-1-p-tolylcarbamoyl-butyl)-carbamic acid 9H-fluoren-9-ylmethyl ester from Example 101 was coupled to Methylamino-acetic acid as described in the method of Example 6. Purification by HPLC produced the title compound. ¹H NMR (400 MHz, DMSO-d6) 7.86 (d, J=7 Hz, 2H), 7.69 (t, J=7 Hz, 2H), 7.44 (d, J=8 Hz, 2H), 7.38 (ddd, J=2, 8, 8 Hz, 2H), 7.30 (dd, J=7, 13 Hz, 2H), 7.09 (d, J=8 Hz, 2H), 4.33-4.04 (m, 4H), 3.18-3.04 (m, 2H), 2.51 (s, 2H), 2.22 (s, 3H), 1.77-1.43 (m, 4H), 1.42 (s, 3H); Low resolution mass spectrum (ES) m/e 515 [(M+H)⁺, calcd for C₃₀H₃₅N₄O₄: 515]; 95% purity based on HPLC.

### Example 224

### (S)-6-AMINO-2-[3-(4-BENZYLAMINO-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-4-nitro-benzene as described in the method of Example 26. Hydrogenation for 4 hours using 10% palladium on carbon in ethanol under a hydrogen atmosphere followed by mono-alkylation with benzyl bromide and sodium hydride in THF. Deprotection was as described in the method of Example 2 and purification by HPLC produced the title compound. ¹H NMR (400 MHz, DMSO-d6) δ 10.05 (s, 1H), 8.45 (s, 1H), 7.67 (br s, 3H), 7.49 (d, J=8.42 Hz, 2H), 7.39-7.16 (m, 9H), 7.11 (d, J=8.37 Hz, 2H), 6.72 (br, 3H), 6.42 (d, J=7.09 Hz, 1H), 4.37-4.29 (m, 3.5H), 3.36 (t, J=5.23 1H), 2.81-2.74 (m, 2H), 2.25 (s, 3H), 1.75-1.67 (m, 1H), 1.62-1.28 (m, 5H); Low resolution mass spectrum (ES) m/e 461 [(M+H)⁺, calcd for C₂₇H₃₄N₅O₂: 461]; 83% purity based on HPLC.

### Example 225

### (S)-6-AMINO-2-{3-[4-(4-FLUORO-BENZYLAMINO)-PHENYL]-UREIDO}-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-4-nitro-benzene as described in the method of Example 26. Hydrogenation for 4 hours using 10% palladium on carbon in ethanol under a hydrogen atmosphere was followed by mono-alkylation with 4-fluorobenzyl bromide and sodium hydride in THF. Deprotection was as described in the method of Example 2 and purification by HPLC produced the title compound. ¹H NMR (400 MHz, DMSO-d6) δ 10.05 (s, 1H), 8.39 (s, 1H), 7.67 (br s, 3H), 7.49 (d, J=8.43 Hz, 2H), 7.40-7.37 (m, 3H), 7.18-7.10 (m, 7H0, 6.39 (d, J=7.86 Hz, 1H), 4.34 (dd, J=7.90, 13.55 Hz, 1H), 4.25 (s, 2H), 3.37-3.34 (m, 1H), 2.81-2.73 (m, 2H), 2.25 (s, 3H), 1.75-1.66 (m, 1H), 1.62-1.29 (m, 5H); Low resolution mass spectrum (ES) m/e 478 [(M+H)⁺, calcd for C₂₇H₃₃FN₅O₂: 478]; 85% purity based on HPLC.

### Example 226

### (S)-6-AMINO-2-[3-(9H-FLUOREN-2-YL)-UREIDO]-HEXANOIC ACID (4-CYCLOHEXYL-PHENYL)-AMIDE

4-Cyclohexyl-phenylamine was coupled to (S)-6-tert-Butoxycarbonylamino-2-(9H-fluoren-9-ylmethoxycarbonylamino)-hexanoic acid, and then to 2-Isocyanato-9H-fluorene as described in the method of Example 26. Purification by HPLC produced the title compound. ¹H NMR (400 MHz, DMSO-d6) δ 10.11 (s, 1H), 8.86 (s, 1H), 7.76-7.72 (m, 3H), 7.68 (br s, 3H), 7.53-7.51 (m, 3H), 7.34-7.31 (m, 2H), 7.22 (t, J=7.86, 7.86 Hz, 1H), 7.16 (d, J=8.53 Hz, 2H), 6.60 (d, J=8.16 Hz, 1H), 4.42 (dd, J=7.79, 13.48 Hz, 1H), 3.85 (s, 2H), 2.83-2.75 (m, 2H), 2.44 (m, 1H), 1.78-1.54 (m, 9H), 1.42-1.19 (m, 7H); Low resolution mass spectrum (ES) m/e 511 [(M+H)⁺, calcd for C₃₂H₃₉FN₄O₂: 511]; 99.0% purity based on HPLC.

### Example 227

### (S)-3-(4-AMINOMETHYL-PHENYL)-2-[3-(4-BENZYLOXY-PHENYL)-UREIDO]-N-(2-METHYL-1H-INDOL-5-YL)-PROPIONAMIDE

2-Methyl-1H-indol-5-ylamine was coupled to (S)-3-[4-(tert-Butoxycarbonylamino-methyl)-phenyl]-2-(9H-fluoren-9-ylmethoxycarbonylamino)-propionic acid, and then to 1-Benzyloxy-4-isocyanato-benzene as described in the method of Example 26. Purification by HPLC produced the title compound. ¹H NMR (400 MHz, DMSO-d6) δ 10.84 (s, 1H), 9.94 (s, 1H), 8.53 (s, 1H), 8.12 (br s, 3H), 7.69 (d, J=1.66 Hz, 1H), 7.43-7.29 (m, 9H), 7.25 (d, J=9.05 Hz, 2H), 7.18 (d, J=8.59 Hz, 1H), 7.10 (dd, J=1.91, 8.65 Hz, 1H), 6.88 (d, J=9.06 Hz, 2H), 6.43 (d, J=9.06 Hz, 1H), 6.06 (s, 1H), 5.02 (s, 2H), 4.66 (dd, J=8.35, 13.70 Hz, 1H), 3.99 (q, J=5.4 Hz, 2H), 3.11 (dd, J=5.15, 13.71 Hz, 1H), 2.90 (dd, J=8.46, 13.70 Hz, 1H), 2.35 (s, 3H); Low resolution mass spectrum (ES) m/e 548 [(M+H)⁺, calcd for C₃₃H₃₄N₅O₃: 548]; 99.6% purity based on HPLC.

### Example 228

### (S)-[5-AMINO-1-(4-AMINOMETHYL-PHENYLCARBAMOYL)-PENTYL]-CARBAMIC ACID 9H-FLUOREN-9-YLMETHYL ESTER

(4-Amino-benzyl)-carbamic acid tert-butyl ester was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.2 (s, 1H); 8.11 (br s, 3H); 7.90 (d, J=7.54 Hz, 2H); 7.78-7.60 (m, 7H); 7.47-7.28 (m, 5H); 4.29 (d, J=6.71 Hz, 1H); 4.26-4.20 (m, 1H); 4.13 (ddd, J=5.49, 8.61, 8.59 Hz, 1H); 3.98 (d, J=4.78 Hz, 2H); 2.85-2.70 (m, 2H); 1.78-1.48 (m, 4H); 1.48-1.27 (m, 2H); Low resolution mass spectrum (ES) m/e 473 [(M+H)⁺, calcd for C₂₈H₃₃N₄O₃: 473]; 98% purity based on HPLC.

### Example 229

### (S)-[5-AMINO-1-(6-METHYL-PYRIDIN-3-YLCARBAMOYL)-PENTYL]-CARBAMIC ACID 9H-FLUOREN-9-YLMETHYL ESTER

6-Methyl-pyridin-3-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 10.4 (s, 1H); 8.16 (s, 1H); 7.97 (d, J=8.41 Hz, 1H); 7.90 (d, J=7.48 Hz, 2H); 7.23 (t, J=7.0 Hz, 2H); 7.69-7.55 (m, 4H); 7.42 (ddd, J=1.60, 7.35, 7.28 Hz, 2H); 7.32 (dd, J=6.99, 13.43 Hz, 2H); 4.37-4.18 (m, 5H); 3.92 (td, J=4.72, 9.68 Hz, 1H); 2.78 (ddd. J=7.01, 12.47, 12.14 Hz, 2H); 2.25 (s, 3H); 1.76-1.46 (m, 4H); 1.47-1.28 (m, 2H); Low resolution mass spectrum (ES) m/e 459 [(M+H)⁺, calcd for C₂₇H₃₁N₄O₃: 459]; 94% purity based on HPLC.

### Example 230

### (S)-[1-(4-ACETYLAMINO-PHENYLCARBAMOYL)-5-AMINO-PENTYL]-CARBAMIC ACID 9H-FLUOREN-9-YLMETHYL ESTER

N-(4-Amino-phenyl)-acetamide was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 9.96 (s, 1H); 9.87 (s, 1H); 7.89 (d, J=7.57 Hz, 2H); 7.72 (t, J=7.57 Hz, 2H); 7.68-7.55 (m, 4H); 7.49 (s, 4H); 7.41 (t, J=7.44 Hz, 2H); 7.35-7.27 (m, 2H); 4.35-4.18 (m, 3H); 4.10 (ddd, J=5.65, 8.44, 8.39 Hz, 1H); 2.77 (dddd, J=6.49, 6.49, 6.15, 12.60 Hz, 2H); 2.00 (s, 3H); 1.77-1.46 (m, 4H); 1.46-1.24 (m, 2H); Low resolution mass spectrum (ES) m/e 501 [(M+H)⁺, calcd for C₂₉H₃₃N₄O₄: 501]; 99% purity based on HPLC.

### Example 231

### (S)-[1-(3-ACETYLAMINO-PHENYLCARBAMOYL)-5-AMINO-PENTYL]-CARBAMIC ACID 9H-FLUOREN-9-YLMETHYL ESTER

N-(3-Amino-phenyl)-acetamide was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) d 10.0 (s, 1H); 9.92 (s, 1H); 7.95 (s, 1H); 7.89 (J=7.57 Hz, 2H); 7.72 (t, J=8.18 Hz, 2H); 7.68-7.54 (m, 3H); 7.41 (ddd, J=2.27, 7.32, 7.35 Hz, 2H); 7.36-7.28 (m, 3H); 7.23-7.14 (m, 2H); 4.35-4.17 (m, 3H); 4.13 (ddd, J=5.61, 8.56, 8.61 Hz, 1H); 2.77 (dddd, J=5.53, 6.19, 6.13, 11.73 Hz, 2H); 1.74-1.46 (m, 2); 1.46-1.25 (m, 2H); Low resolution mass spectrum (ES) m/e 501 [(M+H)⁺, calcd for C₂₉H₃₃N₄O₄: 501]; 97% purity based on HPLC.

### Example 232

### (S)-[5-AMINO-1-(PYRIDIN-4-YLCARBAMOYL)-PENTYL]-CARBAMIC ACID 9H-FLUOREN-9-YLMETHYL ESTER

Pyridin-4-ylamine was coupled to (S)-6-tert-Butoxycarbonylamino-2-(9H-fluoren-9-ylmethoxycarbonylamino)-hexanoic acid as described in the method of Example 6. Purification by HPLC produced the title compound. ¹H NMR (400 MHz, DMSO-d6) δ 11.2 (s, 1H); 8.64 (d, J=6.34 Hz, 2H); 7.94 (d, J=6.54 Hz, 2H); 7.89 (d, J=7.54 Hz, 2H); 7.84 (d, J=7.04 Hz, 1H); 7.75-7.60 (m, 4H); 7.41 (t, J=7.46 Hz, 2H); 7.32 (t, J=7.43 Hz, 2H); 4.38-4.19 (m, 3H); 4.19-4.10 (m, 1H); 2.77 (td, J=5.71, 6.08, 6.06, 12.5 Hz, 2H); 1.81-1.27 (m, 6H); Low resolution mass spectrum (ES) m/e 445 [(M+H)⁺, calcd for C₂₆H₂₉N₄O₃: 445]; 100% purity based on HPLC.

### Example 233

### (S)-[5-(2-AMINO-ACETYLAMINO)-1-(2-METHYL-1H-INDOL-5-YLCARBAMOYL)-PENTYL]-CARBAMIC ACID 9H-FLUOREN-9-YLMETHYL ESTER

(S)-[5-Amino-1-(2-methyl-1H-indol-5-ylcarbamoyl)-pentyl]-carbamic acid 9H-fluoren-9-yl methyl ester of Example 38 was coupled to tert-Butoxycarbonylamino-acetic acid as described in the of Example 6. Purification by HPLC produced the title compound. ¹H NMR (400 MHz, DMSO-d6) 7.87 (d, J=7.9 Hz, 2H), 7.75-7.64 (m, 3H), 7.44-7.36 (m, 2H), 7.31 (dd, J=6.4 and 12.9 Hz, 2H), 7.16 (d, J=7.9 Hz, 1H), 7.08 (dd, J=1.8, 9.6 Hz, 1H), 6.04 (s, 1H), 4.33-4.15 (m, 3H), 4.10 (dd, J=5.3, 9.2 Hz, 1H), 3.47 (s, 2H), 3.15-3.05 (m, 2H), 2.33 (s, 3H), 1.77-1.54 (m, 3H), 1.40-1.22 (m, 3H); Low resolution mass spectrum (ES) m/e 554 [(M+H)⁺, calcd for C₃₂H₃₆N₅O₄: 554]; 78% purity based on HPLC.

### Example 234

### (S)-3-(4-AMINOMETHYL-PHENYL)-2-(3-BIPHENYL-4-YL-UREIDO)-N-(2-METHYL-1H-INDOL-5-YL)-PROPIONAMIDE

2-Methyl-1H-indol-5-ylamine coupled to (S)-3-[4-(tert-Butoxycarbonylamino-methyl)-phenyl]-2-(9H-fluoren-9-ylmethoxycarbonylamino)-propionic acid, and then to 4-Isocyanato-biphenyl as described in the method of Example 26. Purification by HPLC produced the title compound. ¹H NMR (400 MHz, DMSO-d6) δ 10.84 (s, 1H), 9.98 (s, 1H), 8.85 (s, 1H), 8.12 (br s, 3H), 7.70 (d, J=1.48 Hz, 1H), 7.60 (d, J=8.39 Hz, 2H), 7.54 (d, J=8.73 Hz, 2H), 7.46-7.27 (m, 9H), 7.19 (d, J=8.61 Hz, 1H), 7.11 (dd, J=1.87, 8.65 Hz, 1H), 6.59 (d, J=8.20 Hz, 1H), 6.07 (s, 1H), 4.70 (dd, J=8.23, 13.60 Hz, 1H), 3.99 (q, J=4.83, 4.83, 4.90 Hz, 2H), 3.14 (dd, J=5.08, 13.74 Hz, 1H), 2.93 (dd, J=8.41, 13.74 Hz, 1H), 2.36 (s, 3H); Low resolution mass spectrum (ES) m/e 518 [(M+H)⁺, calcd for C₃₂H₃₂N₅O₂: 518]; 99.3% purity based on HPLC.

### Example 235

### (S)-3-(4-AMINOMETHYL-PHENYL)-2-[3-(9H-FLUOREN-2-YL)-UREIDO]-N-(2-METHYL-1H-INDOL-5-YL)-PROPIONAMIDE

2-Methyl-1H-indol-5-ylamine was coupled to (S)-3-[4-(tert-Butoxycarbonylamino-methyl)-phenyl]-2-(9H-fluoren-9-ylmethoxycarbonylamino)-propionic acid, and then to 2-Isocyanato-9H-fluorene as described in the method of Example 26. Purification by HPLC produced the title compound. ¹H NMR (400 MHz, DMSO-d6) δ 10.83 (s, 1H), 9.96 (s, 1H), 8.81 (s, 1H), 8.09 (br s, 3H), 7.75-7.69 (m, 4H), 7.50 (d, J=7.43 Hz, 1H), 7.38-7.27 (m, 6H), 7.22 (d, J=7.43 Hz, 1H), 7.18 (d, J=9.10 Hz, 1H), 7.10 (dd, J=1.85, 8.65 Hz, 1H), 6.56 (d, J=8.36 Hz, 1H), 6.06 (s, 1H), 4.69 (dd, J=8.19, 13.57 Hz, 1H), 3.98 (d, J=5.51 Hz, 2H), 3.83 (s, 2H), 3.14 (dd, J=5.09, 13.71 Hz, 1H), 2.93 (dd, J=8.33, 13.76 Hz, 1H), 2.34 (s, 3H); Low resolution mass spectrum (ES) m/e 530 [(M+H)⁺, calcd for C₃₃H₃₂N₅O₂: 530]; 95.3% purity based on HPLC.

### Example 236

### (S)-6-AMINO-2-[3-(4-STYRYL-PHENYL)-UREIDO]-HEXANOIC ACID P-TOLYLAMIDE

p-Tolylamine was coupled to (S)-6-tert-Butoxycarbonylamino-2-(9H-fluoren-9-ylmethoxycarbonylamino)-hexanoic acid, and then to 1-Isocyanato-4-styryl-benzene as described in the method of Example 26. Purification by HPLC produced the title compound. ¹H NMR (400 MHz, DMSO-d6) δ 10.09 (s, 1H), 8.86 (s, 1H), 7.67 (br s, 3H), 7.55 (d, J=7.40 Hz, 2H), 7.51-7.47 (m, 4H), 7.40 (d, J=8.72 Hz, 2H), 7.35 (t, J=7.67 Hz, 2H), 7.23 (t, J=7.32 Hz, 1H), 7.14 (d, J=5.05 Hz, 2H), 7.11 (d, J=3.48 Hz, 2H), 6.60 (d, J=8.21 Hz, 1H), 4.40 (dd, J=7.99, 13.49 Hz, 1H), 2.79 (br m, 2H), 2.25 (s, 3H), 1.79-1.71 (m, 1H), 1.66-1.53 (m, 3H), 1.46-1.31 (m, 2H); Low resolution mass spectrum (ES) m/e 457 [(M+H)⁺, calcd for C₂₈H₃₃N₄O₂: 457]; 96.7% purity based on HPLC.

### Example 237

### (S)-[3-(2-AMINO-ACETYLAMINO)-1-(2-METHYL-1H-INDOL-5-YLCARBAMOYL)-PROPYL]-CARBAMIC ACID 9H-FLUOREN-9-YLMETHYL ESTER

2-Methyl-1H-indol-5-ylamine was coupled to (S)-4-tert-Butoxycarbonylamino-2-(9H-fluoren-9-ylmethoxycarbonylamino)-butyric acid, and then to tert-Butoxycarbonylamino-acetic acid as described in the method of Example 182. Purification by HPLC produced the title compound. ¹H NMR (400 MHz, DMSO-d6) δ 7.87 (d, J=7.7 Hz, 2H), 7.72 (t, J=7.0 Hz, 2H), 7.66 (d, J=1.3 Hz, 1H), 7.40 (ddd, J=2.9, 7.5, 7.2 Hz, 2H), 7.31 (dd, J=7.2, 14.3 Hz, 2H), 7.17 (d, J=8.6 Hz, 1H), 7.08 (dd, J=2.0, 8.8 Hz, 1H), 4.34-4.12 (m, 4H), 3.32-3.09 (m, 3H), 2.33 (s, 3H), 1.96-1.72 (m, 2H); Low resolution mass spectrum (ES) m/e 526 [(M+H)⁺, calcd for C₃₀H₃₂N₅O₄: 526]; 86% purity based on HPLC.

### Example 238

### (S)-[4-(2-AMINO-ACETYLAMINO)-1-(2-METHYL-1H-INDOL-5-YLCARBAMOYL)-BUTYL]-CARBAMIC ACID 9H-FLUOREN-9-YLMETHYL ESTER

2-Methyl-1H-indol-5-ylamine was coupled to (S)-5-tert-Butoxycarbonylamino-2-(9H-fluoren-9-ylmethoxycarbonylamino)-pentanoic acid, and then to tert-Butoxycarbonylamino-acetic acid as described in the method of Example 182. Purification by HPLC produced the title compound. ¹H NMR (400 MHz, DMSO-d6) δ 7.87 (d, J=7.7 Hz, 2H), 7.75-7.65 (m, 3H), 7.40 (ddd, J=3.5, 7.5, 7.0 Hz, 2H), 7.31 (dd, J=7.0, 12.9 Hz, 2H), 7.16 (d, J=8.8 Hz, 1H), 7.09 (dd, J=1.8, 9.4 Hz, 1H), 4.36-4.17 (m, 3H), 4.12 (dd, J=5.3, 9.0 Hz, 1H), 3.48 (s, 2H), 3.20-3.04 (m, 2H), 2.33 (s, 3H), 1.81-1.36 (m, 4H); Low resolution mass spectrum (ES) m/e 540 [(M+H)⁺, calcd for C₃₁H₃₄N₅O₄: 540]; 84% purity based on HPLC.

### Example 239

### (S)-6-(2-AMINO-ACETYLAMINO)-2-[3-(4-BENZYLOXY-PHENYL)-UREIDO]-HEXANOIC ACID (2-METHYL-1H-INDOL-5-YL)-AMIDE

2-Methyl-1H-indol-5-ylamine was coupled to (S)-6-tert-Butoxycarbonylamino-2-(9H-fluoren-9-ylmethoxycarbonylamino)-hexanoic acid as described in the method of Example 6, and then to tert-Butoxycarbonylamino-acetic acid as described in the method for Intermediate #1 of Example 1. Final coupling to 1-Benzyloxy-4-isocyanato-benzene as described in the method of Example 26 and purification by HPLC produced the title compound. ¹H NMR (400 MHz, DMSO-d6) δ 7.67 (d, J=1.8 Hz, 1H), 7.43-7.22 (m, 8H), 7.16 (d, J=8.6 Hz, 1H), 7.09 (dd, J=2.0, 8.6 Hz, 1H), 6.91-6.84 (m, 2H), 5.00 (s, 2H), 4.33 (dd, J=5.5, 8.1 Hz, 1H), 3.46 (s, 2H), 3.10 (t, J=8.1, 6.4 Hz, 2H), 2.33 (s, 3H), 1.80-1.23 (m, 6H); low resolution mass spectrum (ES) m/e 557 [(M+H)⁺, calcd for C₃₁H₃₇N₆O₄: 557]; 97% purity based on HPLC.

### Example 240

### (S)-4-(2-AMINO-ACETYLAMINO)-2-[3-(9H-FLUOREN-2-YL)-UREIDO]-N-(2-METHYL-1H-INDOL-5-YL)-BUTYRAMIDE

2-Methyl-1H-indol-5-ylamine was coupled to (S)-4-tert-Butoxycarbonylamino-2-(9H-fluoren-9-ylmethoxycarbonylamino)-butyric acid as described in the method of Example 6, and then to tert-Butoxycarbonylamino-acetic acid as described in the method for Intermediate #1 of Example 1. Final coupling to 2-isocyanato-9H-fluorene as described in the method of Example 26 and purification by HPLC produced the title compound. ¹H NMR (400 MHz, DMSO-d6) δ 7.77-7.64 (m, 4H), 7.50 (d, J=7.0 Hz, 1H), 7.36-7.27 (m, 3H), 7.22 (d, J=7.7 Hz, 1H), 7.18 (d, J=8.6 Hz, 1H), 7.09 (dd, J=1.5, 8.6 Hz, 1H), 4.42 (dd, J=5.5, 7.5 Hz, 1H), 3.83 (s, 2H), 3.50 (s, 2H), 3.33-3.10 (m, 2H), 2.33 (s, 3H), 2.03-1.72 (m, 2H); low resolution mass spectrum (ES) m/e 511 [(M+H)⁺, calcd for C₂₉H₃₁N₆O₃: 511]; 81% purity based on HPLC.

### Example 241

### (S)-4-(2-AMINO-ACETYLAMINO)-2-[3-(4-BENZYLOXY-PHENYL)-UREIDO]-N-(2-METHYL-1H-INDOL-5-YL)-BUTYRAMIDE

2-Methyl-1H-indol-5-ylamine was coupled to (S)-4-tert-Butoxycarbonylamino-2-(9H-fluoren-9-ylmethoxycarbonylamino)-butyric acid as described in the method of Example 6, and then to tert-Butoxycarbonylamino-acetic acid as described in the method for Intermediate #1 of Example 1. Final coupling to 1-Benzyloxy-4-isocyanato-benzene as described in the method of Example 26 and purification by HPLC produced the title compound. ¹H NMR (400 MHz, DMSO-d6) δ 7.65 (d, J=1.8 Hz, 1H), 7.45-7.22 (m, 9H), 7.17 (d, J=8.8 Hz, 1H), 7.08 (dd, J=1.8, 8.9 Hz, 1H), 7.03 (d, J=9.2 Hz, 1H), 6.95 (d, J=9.2 Hz, 1H), 6.88 (d, J=9.2 Hz, 2H), 5.06 (s, 1H), 5.00 (s, 2H), 4.38 (dd, J=5.5, 8.3 Hz, 1H), 3.49 (s, 2H), 3.32-3.08 (m, 2H), 2.32 (s, 3H), 1.97-1.68 (m, 2H); low resolution mass spectrum (ES) m/e 529 [(M+H)⁺, calcd for C₂₉H₃₃N₆O₄: 529]; 77% purity based on HPLC.

### Example 242

### (S)-[4-(2-ETHYLAMINO-ACETYLAMINO)-1-P-TOLYLCARBAMOYL-BUTYL]-CARBAMIC ACID 9H-FLUOREN-9-YLMETHYL ESTER

2-Methyl-1H-indol-5-ylamine was coupled to (S)-5-tert-Butoxycarbonylamino-2-(9H-fluoren-9-ylmethoxycarbonylamino)-pentanoic acid as described in the method of Example 6, and then to Ethylamino-acetic acid as described in the method for Intermediate #1 of Example 1. Purification by HPLC produced the title compound. ¹H NMR (400 MHz, DMSO-d6) δ 7.87 (d, J=7.7 Hz, 2H), 7.71 (t, J=7.7 Hz, 2H), 7.45 (d, J=8.3 Hz, 2H), 7.40 (ddd, J=2.0, 7.7, 7.7 Hz, 2H), 7.35-7.27 (m, 2H), 7.09 (d, J=8.3 Hz, 2H), 4.34-4.16 (m, 3H), 4.10 (dd, J=5.3, 9.0 Hz, 1H), 3.63 (s, 2H), 3.20-3.07 (m, 2H), 2.91 (dd, J=7.9, 13.6 Hz, 2H), 2.23 (s, 3H), 1.77-1.37 (m, 4H), 1.13 (t, J=7.0 Hz, 3H); low resolution mass spectrum (ES) m/e 529 [(M+H)⁺, calcd for C₃₁H₃₇N₄O₄: 529]; 86% purity based on HPLC.

### Example 243

### (S)-6-AMINO-2-[3-(4-PHENETHYL-PHENYL)-UREIDO]-HEXANOIC ACID P-TOLYLAMIDE

The title compound was prepared as described in Example 2 with a final coupling to 1-Isocyanato-4-phenethyl-benzene. ¹H NMR (400 MHz, DMSO-d6) δ 10.07 (s, 1H), 8.64 (s, 1H), 7.68 (br s, 3H), 7.50 (d, J=8.39 Hz, 2H), 7.28-7.24 (m, 4H), 7.20-7.10 (m, 5H), 7.06 (d, J=8.43 Hz, 2H), 6.51 (d, J=8.21 Hz, 1H), 4.38 (dd, J=7.98, 13.52 Hz, 1H), 2.85-2.77 (m, 6H), 2.25 (s, 3H), 1.77-1.69 (m, 1H), 1.64-1.51 (m, 3H), 1.45-1.30 (m, 2H); low resolution mass spectrum (ES) m/e 459 [(M+H)⁺, calcd for C₂₈H₃₅N₄O₂: 459]; 96.6% purity based on HPLC.

### Example 244

### (S)-6-AMINO-2-[3-(4-BUTOXY-PHENYL)-UREIDO]-HEXANOIC ACID P-TOLYLAMIDE

The title compound was prepared as described in Example 2 with a final coupling to 1-Butoxy-4-isocyanato-benzene. ¹H NMR (400 MHz, DMSO-d6) δ 10.06 (s, 1H), 8.57-8.55 (m, 1H), 7.73 (br s, 3H), 7.50 (d, J=8.39 Hz, 2H), 7.26 (d, J=8.97 Hz, 2H), 7.11 (d, J=8.33 Hz, 2H), 6.80 (d, J=9.05 Hz, 2H), 6.48-6.46 (m, 1H), 4.36 (dd, J=7.94, 13.51 Hz, 1H), 3.88 (t, J=6.48 Hz, 2H), 2.79-2.78 (br m, 2H), 2.25 (s, 3H), 1.77-1.53 (m, 6H), 1.46-1.30 (m, 4H), 0.92 (t, J=7.39 Hz, 3H); low resolution mass spectrum (ES) m/e 427 [(M+H)⁺, calcd for C₂₄H₃₅N₄O₃: 427]; 99.5% purity based on HPLC.

### Example 245

### (S)-4-[3-(5-AMINO-1-P-TOLYLCARBAMOYL-PENTYL)-UREIDO]-BENZOIC ACID ETHYL ESTER

The title compound was prepared as described in Example 2 with a final coupling to 4-Isocyanato-benzoic acid ethyl ester. ¹H NMR (400 MHz, DMSO-d6) δ 10.10 (s, 1H), 9.24-9.23 (m, 1H), 7.84 (d, J=8.79 Hz, 2H), 7.71 (br s, 3H), 7.53-7.49 (m, 4H), 7.12 (d, J=8.36 Hz, 2H), 6.79 (t, J=6.27 Hz, 1H), 4.39 (dd, J=7.94, 13.33 Hz, 1H), 4.26 (q, J=7.09 Hz, 2H), 2.82-2.75 (m, 2H), 2.25 (s, 3H), 1.80-1.71 (m, 1H), 1.67-1.51 (m, 3H), 1.45-1.33 (m, 2H), 1.29 (t, J=7.09 Hz, 3H); low resolution mass spectrum (ES) m/e 427 [(M+H)⁺, calcd for C₂₃H₃₁N₄O₄: 427]; 99.6% purity based on HPLC.

### Example 246

### (S)-7-AMINO-3-[3-(4-BENZYLOXY-PHENYL)-UREIDO]-HEPTANOIC ACID P-TOLYLAMIDE

The title compound was prepared as described in Example 2 with an initial coupling with (S)-7-tert-Butoxycarbonylamino-3-(9H-fluoren-9-ylmethoxycarbonylamino)-heptanoic acid. ¹H NMR (500 MHz, DMSO-d6) δ 9.87 (s, 1H), 8.42 (d, J=3.84 Hz, 1H), 7.67 (br s, 3H), 7.47 (d, J=−8.44 Hz, 2H), 7.42 (d, J=−6.94 Hz, 2H), 7.38 (t, J=−7.46, 7.46 Hz, 2H), 7.31 (t, J=7.20 Hz, 1H), 7.28 (d, J=9.07 Hz, 2H), 7.09 (d, J=8.47 Hz, 2H), 6.87 (d, J=9.03 Hz, 2H), 6.20 (dd, J=4.55, 8.47 Hz, 1H), 5.02 (s, 2H), 4.05-3.99 (m, 1H), 2.80-2.73 (m, 2H), 2.54-2.44 (m, 2H), 2.24 (s, 3H), 1.60-1.30 (m, 6H); low resolution mass spectrum (ES) m/e 475 [(M+H)⁺, calcd for C₂₈H₃₅N₄O₃: 475]; 98.7% purity based on HPLC.

### Example 247

### (S)-7-AMINO-3-[3-(9H-FLUOREN-2-YL)-UREIDO]-HEPTANOIC ACID P-TOLYLAMIDE

The title compound was prepared as described in Example 246 with a final coupling with 2-isocyanato-9H-fluorene. ¹H NMR (500 MHz, DMSO-d6) δ 9.90 (s, 1H), 8.72 (s, 1H), 7.75-7.74 (m, 2H), 7.71 (d, J=8.28 Hz, 1H), 7.67 (br s, 3H), 7.51 (d, J=7.47 Hz, 1H), 7.48 (d, J=−8.44 Hz, 2H), 7.32 (t, J=7.96 Hz, 2H), 7.22 (dt, J=−0.92, 7.47 Hz, 1H), 7.09 (d, J=8.44 Hz, 2H), 6.36 (d, J=8.71 Hz, 1H), 4.10-4.03 (m, 1H), 3.84 (s, 2H), 2.81-2.75 (m, 2H), 2.57-2.48 (m, 2H), 2.24 (s, 3H), 1.61-1.32 (m, 6H); low resolution mass spectrum (ES) m/e 457 [(M+H)⁺, calcd for C₂₈H₃₃N₄O₂: 457]; 99.8% purity based on HPLC.

### Example 248

### (S)-6-AMINO-2-{3-[4-(2-FLUORO-BENZYLOXY)-PHENYL]-UREIDO}-HEXANOIC ACID P-TOLYLAMIDE

Intermediate #3 (50 mg) of Example 1 was dissolved in 4 mL of ethanol and hydrogenated under atmospheric hydrogen at room temperature for 12 hours. Filtering off the insolubles and concentrating down in vacuo produced the crude phenol. Alkylation with 1-Bromomethyl-2-fluoro-benzene using standard alkylating conditions of sodium hydride (3 equivalents) in dimethylformamide (0.2M) for 4 hours and quenching with water produced crude material after isolation with ethyl acetate. Deprotection of the crude material was performed described in Example 2. Purification by HPLC produced the title compound. ¹H NMR (500 MHz, DMSO-d6) δ 10.07 (s, 1H), 8.59 (s, 1H), 7.70 (br s, 3H), 7.53 (dt, J=1.42, 7.64, 7.78 Hz, 1H), 7.50 (d, J=−8.42 Hz, 2H), 7.41 (dt, J=−1.74, 7.51, 7.57 Hz, 1H), 7.29 (d, J=9.06 Hz, 2H), 7.26-7.21 (m, 2H), 7.11 (d, J=8.46 Hz, 2H), 6.91 (d, J=9.03 Hz, 2H), 6.47 (d, J=8.33 Hz, 1H), 5.06 (s, 2H), 4.37 (dt, J=5.61, 8.11, 8.17 Hz, 1H), 2.79-2.78 (m, 2H), 2.25 (s, 3H), 1.76-1.69 (m, 1H), 1.63-1.52 (m, 3H), 1.44-1.31 (m, 2H); low resolution mass spectrum (ES) m/e 479 [(M+H)⁺, calcd for C₂₇H₃₂N₄O₃: 479]; 99.6% purity based on HPLC.

### Example 249

### (S)-6-AMINO-2-{3-[4-(3-FLUORO-BENZYLOXY)-PHENYL]-UREIDO}-HEXANOIC ACID P-TOLYLAMIDE

The title compound was prepared as described in Example 248 with a final coupling reaction with 1-Bromomethyl-3-fluoro-benzene. ¹H NMR (500 MHz, DMSO-d6) δ 10.07 (s, 1H), 8.57 (s, 1H), 7.69 (br s, 3H), 7.49 (d, J=8.43 Hz, 2H), 7.42 (dd, J=8.10, 14.04 Hz, 1H), 7.30-7.24 (m, 4H), 7.16-7.10 (m, 3H), 6.90 (d, J=8.97 Hz, 2H), 6.46 (d, J=8.24 Hz, 1H), 5.06 (s, 2H), 4.37 (dt, J=5.70, 8.08, Hz, 1H), 2.81-2.75 (m, 2H), 2.25 (s, 3H), 1.76-1.69 (m, 1H), 1.63-1.51 (m, 3H), 1.44-1.31 (m, 2H); low resolution mass spectrum (ES) m/e 479 [(M+H)⁺, calcd for C₂₇H₃₂N₄O₃: 479]; 99.0% purity based on HPLC.

### Example 250

### (S)-6-AMINO-2-{3-[4-(4-FLUORO-BENZYLOXY)-PHENYL]-UREIDO}-HEXANOIC ACID P-TOLYLAMIDE

The title compound was prepared as described in Example 248 with a final coupling reaction with 1-Bromomethyl-4-fluoro-benzene. ¹H NMR (500 MHz, DMSO-d6) δ 10.07 (s, 1H), 8.57 (s, 1H), 7.69 (br s, 3H), 7.50-7.46 (m, 4H), 7.28 (d, J=9.09 Hz, 2H), 7.20 (t, J=−8.88 Hz, 2H), 7.11 (d, J=−8.39 Hz, 2H), 6.89 (d, J=−9.06 Hz, 2H), 6.46 (d, J=−8.33 Hz, 1H), 5.01 (s, 2H), 4.37 (dt, J=5.67, 8.14 Hz, 1H), 2.81-2.75 (m, 2H), 2.25 (s, 3H), 1.75-1.69 (m, 1H), 1.63-1.51 (m, 3H), 1.44-1.31 (m, 2H); low resolution mass spectrum (ES) m/e 479 [(M+H)⁺, calcd for C₂₇H₃₂N₄O₃: 479]; 97.9% purity based on HPLC.

### Example 251

### (S)-6-AMINO-2-[3-(4-{[METHYL-(4-NITRO-PHENYL)-AMINO]-METHYL}-PHENYL)-UREIDO]-HEXANOIC ACID P-TOLYLAMIDE

The title compound was prepared as described in Example 2 with a final coupling reaction with (4-Isocyanato-benzyl)-methyl-(4-nitro-phenyl)-amine. (4-Isocyanato-benzyl)-methyl-(4-nitro-phenyl)-amine was prepared from the coupling of 1-Chloromethyl-4-isocyanato-benzene to Methyl-(4-nitro-phenyl)-amine in dimethylformamide at room temperature under an inert atmosphere and was used directly in the coupling reaction without further purification. ¹H NMR (500 MHz, DMSO-d6) δ 10.05 (s, 1H), 8.73 (s, 1H), 8.02 (d, J=9.45 Hz, 2H), 7.66 (br s, 3H), 7.49 (d, J=−8.43 Hz, 2H), 7.34 (d, J=8.56 Hz, 2H), 7.09 (dd, J=8.50, 13.03 Hz, 4H), 6.81 (d, J=9.52 Hz, 2H), 6.52 (d, J=7.95 Hz, 1H), 4.66 (s, 2H), 4.40-4.34 (m, 1H), 3.16 (s, 3H), 2.81-2.73 (m, 2H), 2.25 (s, 3H), 1.77-1.68 (m, 1H), 1.64-1.52 (m, 3H), 1.43-1.31 (m, 2H); low resolution mass spectrum (ES) m/e 519 [(M+H)⁺, calcd for C₂₈H₃₅N₆O₄: 519]; 81.5% purity based on HPLC.

### Example 252

### (S)-6-AMINO-2-(3-{4-[2-(4-METHOXY-PHENYL)-VINYL]-PHENYL}-UREIDO)-HEXANOIC ACID P-TOLYLAMIDE

The title compound was prepared as described in Example 2 with a final coupling reaction with 1-Isocyanato-4-(p-methoxy)styryl-benzene. ¹H NMR (500 MHz, DMSO-d6) δ 10.09 (s, 1H), 8.64 (s, 1H), 8.83 (d, J=2.39 Hz, 1H), 7.68 (br s, 3H), 7.50 (t, J=8.20 Hz, 4H), 7.43 (d, J=−8.70 Hz, 2H), 7.38 (d, J=−8.67 Hz, 2H), 7.12 (d, J=−8.45 Hz, 2H), 7.01 (d, J=−5.72 Hz, 2H), 6.93 (d, J=8.61 Hz, 2H), 6.59 (dd, J=2.34, 8.23 Hz, 1H), 4.40 (dd, J=8.10, 13.54 Hz, 1H), 3.76 (s, 3H), 2.82-2.76 (m, 2H), 2.25 (s, 3H), 1.78-1.71 (m, 1H), 1.65-1.53 (m, 3H), 1.45-1.32 (m, 2H); low resolution mass spectrum (ES) m/e 487 [(M+H)⁺, calcd for C₂₉H₃₅N₄O₃: 487]; 96.7% purity based on HPLC.

### Example 253

### (S)-6-AMINO-2-[3-(4-BENZYLOXY-3-CHLORO-PHENYL)-UREIDO]-HEXANOIC ACID P-TOLYLAMIDE

The title compound was prepared as described in Example 2 with a final coupling reaction with 1-Benzyloxy-2-chloro-4-isocyanato-benzene. ¹H NMR (500 MHz, DMSO-d6) δ 10.07 (s, 1H), 8.77 (d, J=1.16 Hz, 1H), 7.68 (br s, 3H), 7.65 (s, 1H), 7.49 (d, J=8.43 Hz, 2H), 7.45 (d, J=7.05 Hz, 2H), 7.39 (t, J=7.59 Hz, 2H), 7.33 (t, J=7.25 Hz, 1H), 7.11 (dd, J=3.60, 4.85 Hz, 4H), 6.56 (d, J=8.26 Hz, 1H), 5.12 (s, 2H), 4.36 (dd, J=8.06, 13.48 Hz, 1H), 2.81-2.75 (m, 2H), 2.25 (s, 3H), 1.76-1.70 (m, 1H), 1.64-1.51 (m, 3H), 1.43-1.31 (m, 2H); low resolution mass spectrum (ES) m/e 495 [(M+H)⁺, calcd for C₂₉H₃₂ClN₄O₃: 495]; 94.9% purity based on HPLC.

### Example 254

### (S)-6-AMINO-2-[3-(4-BENZYLOXY-PHENYL)-UREIDO]-HEXANOIC ACID (3,4-DIMETHYL-PHENYL)-AMIDE

The title compound was prepared as described in Example 2 with a final coupling reaction with 3,4-Dimethyl-phenylamine. ¹H NMR (400 MHz, DMSO-d6) δ 7.43-7.20 (m, 9H), 7.03 (d, J=8.3 Hz, 1H), 6.87 (d, J=9.0 Hz, 2H), 5.00 (s, 2H), 4.31 (dd, J=5.5, 8.3 Hz, 1H), 2.75 (t, J=7.5 Hz, 2H), 2.16 (s, 3H), 2.14 (s, 3H), 1.77-1.63 (m, 1H), 1.63-1.46 (m, 3H), 1.43-1.24 (m, 2H); low resolution mass spectrum (ES) m/e 475 [(M+H)⁺, calcd for C₂₈H₃₅N₄O₃: 475]; 99% purity based on HPLC.

### Example 255

### (S)-6-AMINO-2-[3-(4-BENZYLOXY-PHENYL)-UREIDO]-HEXANOIC ACID CYCLOPENTYLAMIDE

The title compound was prepared as described in Example 2 with an initial coupling reaction to cyclopentylamine. ¹H NMR (400 MHz, DMSO-d6) δ 7.43-7.26 (m, 5H), 7.22 (d, J=8.8 Hz, 2H), 6.87 (d, J=9.4 Hz, 2H), 5.00 (s, 2H), 4.12 (dd, J=5.7, 7.7 Hz, 1H), 3.96 (qu, J=6.4, 5.9. 13.4 Hz, 1H), 2.73 (t, J=7.7 Hz, 2H), 1.84-1.70 (m, 2H), 1.67-1.16 (m, 12H); low resolution mass spectrum (ES) m/e 439 [(M+H)⁺, calcd for C₂₅H₃₅N₄O₃: 439]; 100% purity based on HPLC.

### Example 256

### (S)-6-AMINO-2-{3-[4-(3,5-DIFLUORO-BENZYLOXY)-PHENYL]-UREIDO}-HEXANOIC ACID P-TOLYLAMIDE

The title compound was prepared as described in Example 248 with a final coupling reaction with 1-Bromomethyl-3,5-difluoro-benzene. ¹H NMR (500 MHz, DMSO-d6) δ 10.07 (s, 1H), 8.57 (s, 1H), 7.67 (br s, 3H), 7.49 (d, J=−8.43 Hz, 2H), 7.29 (d, J=−8.94 Hz, 2H), 7.21-7.14 (m, 3H), 7.11 (d, J=8.47 Hz, 2H), 7.06-7.02 (m, 1H), 6.90 (d, J=9.00 Hz, 2H), 6.46 (dd, J=1.83, 8.36 Hz, 1H), 5.07 (s, 2H), 4.37 (dt, J=5.81, 8.03 Hz, 1H), 2.81-2.75 (m, 2H), 2.25 (s, 3H), 1.76-1.69 (m, 1H), 1.63-1.51 (m, 3H), 1.44-1.29 (m, 2H); low resolution mass spectrum (ES) m/e 497 [(M+H)⁺, calcd for C₂₇H₃₁F₂N₄O₃: 497]; 98.8% purity based on HPLC.

### Example 257

### (S)-6-AMINO-2-[3-(3-BENZOOXAZOL-2-YL-PHENYL)-UREIDO]-HEXANOIC ACID P-TOLYLAMIDE

The title compound was prepared as described in Example 251 with a final coupling reaction with (4-Isocyanato-benzyl)-methyl-p-tolyl-amine. (4-Isocyanato-benzyl)-methyl-p-tolyl-amine was prepared from the coupling of 1-Chloromethyl-4-isocyanato-benzene to Methyl-(4-methylphenyl)-amine in dimethylformamide at room temperature under an inert atmosphere and was used directly in the coupling reaction without further purification. ¹H NMR (500 MHz, DMSO-d6) δ 10.49 (s, 1H), 9.30 (br, 2H), 8.13 (s, 1H), 7.71 (br s, 3H), 7.46 (dd, J=2.68, 8.57 Hz, 4H), 7.29 (d, J=8.56 Hz, 2H), 7.22-7.15 (m, 6H), 4.06 (br s, 2H), 3.83 (br s, 1H), 3.22 (s, 3H), 2.77-2.73 (m, 2H), 2.32 (s, 3H), 2.27 (s, 3H), 1.95-1.24 (m, 6H); low resolution mass spectrum (ES) m/e 488 [(M+H)⁺, calcd for C₂₉H₃₈N₅O₂: 488]; 91.7% purity based on HPLC.

### Example 258

### (S)-6-AMINO-2-[3-(4-BENZYLOXY-PHENYL)-UREIDO]-HEXANOIC ACID BUTYLAMIDE

The title compound was prepared as described in Example 2 with an initial coupling reaction with butylamine. ¹H NMR (400 MHz, DMSO-d6) δ 7.44-7.27 (m, 5H), 7.24 (d, J=8.9 Hz, 2H), 6.87 (d, J=8.9 Hz, 2H), 5.01 (s, 2H), 4.13 (t, J=8.1 Hz, 1H), 3.10-2.98 (m, 2H), 2.73 (t, J=7.7 Hz, 2H), 1.68-1.18 (m, 10H), 0.84 (t, J=7.3 Hz, 3H); low resolution mass spectrum (ES) m/e 427 [(M+H)⁺, calcd for C₂₄H₃₅N₄O₃: 427]; 100% purity based on HPLC.

### Example 259

### (S)-6-AMINO-2-[3-(4-BENZYLOXY-PHENYL)-UREIDO]-HEXANOIC ACID (2-AMINO-ETHYL)-ESTER

The title compound was prepared as described in Example 2 with an initial coupling reaction with 2-Amino-ethanol. Isolation and purification produced the title compound of this Example and of Example 260. ¹H NMR (400 MHz, DMSO-d6) δ 8.48 (s, 1H), 8.06 (t, J=5.6 Hz, 1H), 7.61 (br s, 3H), 7.45-7.28 (m, 5H), 7.25 (d, J=9.1 Hz, 2H), 6.88 (d, J=8.7 Hz, 2H), 6.27 (d, J=8.1 Hz, 1H), 5.02 (s, 2H), 4.67 (br, 1H), 4.19 (dd, J=7.5, 13.3 Hz, 1H), 3.43-3.35 (m, 2H), 3.13 (qu, 2H), 2.75 (t, J=7.7 Hz, 2H), 1.69-1.19 (m, 6H); low resolution mass spectrum (ES) m/e 415.1 [(M+H)⁺, calcd for C₂₂H₃₁N₄O₄: 414.5]; XX % purity based on HPLC.

### Example 260

### (S)-6-AMINO-2-[3-(4-BENZYLOXY-PHENYL)-UREIDO]-HEXANOIC ACID (2-HYDROXY-ETHYL)-AMIDE

The title compound was prepared as described in Example 2 with an initial coupling reaction with 2-Amino-ethanol. Isolation and purification produced the title compound of this Example and of Example 259. ¹H NMR (400 MHz, DMSO-d6) δ 8.49 (s, 1H), 8.06 (t, J=5.6 Hz, 1H), 7.64 (br s, 3H), 7.45-7.28 (m, 5H), 7.25 (d, J=9.1 Hz, 2H), 6.88 (d, J=8.7 Hz, 2H), 6.28 (d, J=8.3 Hz, 1H), 5.01 (s, 2H), 4.67 (br, 1H), 4.18 (dd, J=7.5, 13.3 Hz, 1H), 3.43-3.35 (m, 2H), 3.13 (qu, 2H), 2.75 (t, J=7.7 Hz, 2H), 1.69-1.19 (m, 6H); low resolution mass spectrum (ES) m/e 415 [(M+H)⁺, calcd for C₂₂H₃₁N₄O₄: 415]; 100% purity based on HPLC.

### Example 261

### (S)-6-AMINO-2-{3-[4-(PYRIDIN-3-YLMETHOXY)-PHENYL]-UREIDO}-HEXANOIC ACID P-TOLYLAMIDE

The title compound was prepared as described in Example 262 with a final coupling reaction with 3-Bromomethyl-pyridine. ¹H NMR (400 MHz, DMSO-d6) δ 10.06 (s, 1H), 7.69 (br s, 3H), 7.59 (dd, J=4.96, 7.71 Hz, 1H), 7.49 (d, J=8.41 Hz, 2H), 7.30 (d, J=9.16 Hz, 2H), 7.11 (d, J=8.31 Hz, 2H), 6.93 (d, J=9.05 Hz, 2H), 5.13 (s, 2H), 4.37 (dd, J=7.81, 13.39 Hz, 1H), 2.81-2.76 (m, 2H), 2.25 (s, 3H), 1.77-1.68 (m, 1H), 1.61-1.34 (m, 5H); low resolution mass spectrum (ES) m/e 462 [(M+H)⁺, calcd for C₂₈H₃₂N₅O₃: 462]; 80.1% purity based on HPLC.

### Example 262

### (S)-6-AMINO-2-[4-(3-PYRIDIN-4-YL-UREIDO)-BENZYLAMINO]-HEXANOIC ACID P-TOLYLAMIDE

The title compound was prepared as described in Example 2 with a final coupling reaction with 1-Chloromethyl-4-isocyanato-benzene and quenching with Pyridin-4-ylamine. Purification by HPLC produced the title compound. ¹H NMR (400 MHz, DMSO-d6) δ 10.07 (s, 1H), 8.94 (s, 1H), 8.23 (d, J=7.38 Hz, 2H), 8.13 (s, 2H), 7.73 (br s, 3H), 7.49 (d, J=8.43 Hz, 2H), 7.41 (d, J=8.62 Hz, 2H), 7.26 (d, J=8.63 Hz, 2H), 7.11 (d, J=8.38 Hz, 2H), 6.81 (d, J=7.47 Hz, 2H), 6.67 (d, J=−8.08 Hz, 1H), 5.23 (s, 2H), 4.35 (dd, J=−8.00, 13.54 Hz, 1H), 2.81-2.74 (m, 2H), 2.25 (s, 3H), 1.77-1.68 (m, 1H), 1.65-1.21 (m, 5H); low resolution mass spectrum (ES) m/e 461 [(M+H)⁺, calcd for C₂₆H₃₃N₆O₂: 461]; 93.8% purity based on HPLC.

### Example 263

### (S)—N-(6-AMINO-HEXYL)-2-[3-(4-BENZYLOXY-PHENYL)-UREIDO]-3-PHENYL-PROPIONAMIDE

((S)-2-(9H-Fluoren-9-ylmethoxycarbonylamino)-3-phenyl-propionic acid was coupled to (6-Amino-hexyl)-carbamic acid tert-butyl ester as described in Example 5. Coupling to 1-Benzyloxy-4-isocyanato-benzene as described in the preparation of Example 2 produced the titled compound. ¹H NMR (400 MHz, DMSO-d6) δ 7.42-7.15 (m, 12H), 6.85 (d, J=9.0 Hz, 2H), 4.99 (s, 2H), 4.36 (dd, J=5.7, 8.3 Hz, 1H), 3.09-2.86 (m, 3H), 2.82-2.69 (m, 3H), 1.47 (quintet, J=7.0, 8.1, 15.4 Hz, 2H), 1.37-1.10 (m, 6H); low resolution mass spectrum (ES) m/e 489 [(M+H)⁺, calcd for C₂₉H₃₇N₄O₃: 489]; 100% purity based on HPLC.

### Example 264

### (S)-6-AMINO-2-[3-(4-BENZYLOXY-PHENYL)-UREIDO]-HEXANOIC ACID (4-METHYL-CYCLOHEXYL)-AMIDE

The title compound was prepared as described in Example 2 with an initial coupling reaction with 4-Methyl-cyclohexylamine. ¹H NMR (400 MHz, DMSO-d6) δ 7.90 (d, J=7.9 Hz, 0.5H), 7.86 (d, J=7.5 Hz, 0.5H), 7.43-7.26 (m, 5H), 7.22 (d, J=9.0 Hz, 1H), 7.21 (d, J=9.2 Hz, 1H), 6.87 (d, J=8.8 Hz, 2H), 5.00 (s, 2H), 4.22 (dd, J=5.5, 7.5 Hz, 0.5H), 4.10 (dd, J=5.5, 7.6 Hz, 0.5H), 3.78-3.69 (br, 0.5H), 3.50-3.37 (br, 0.5H), 2.72 (t, J=7.5 Hz, 2H), 1.76-1.36 (m, 9H), 1.34-1.05 (m, 5H), 0.99-0.88 (m, 1H), 0.85 (d, J=8.8 Hz, 1.5H), 0.83 (d, J=8.6 Hz, 1.5H); low resolution mass spectrum (ES) m/e 467 [(M+H)⁺, calcd for C₂₇H₃₉N₄O₃: 467]; 99% purity based on HPLC.

### Example 265

### (S)-6-AMINO-2-[3-(4-BENZYLOXY-PHENYL)-UREIDO]-HEXANOIC ACID CYCLOHEXYLMETHYL-AMIDE

The title compound was prepared as described in Example 2 with an initial coupling reaction with Cyclohexyl-methylamine. ¹H NMR (400 MHz, DMSO-d6) 7.43-7.26 (m, 5H), 7.23 (d, J=9.0 Hz, 2H), 6.87 (d, J=9.0 Hz, 2H), 5.00 (s, 2H), 4.14 (dd, J=5.5, 7.7 Hz, 1H), 2.92 (dd, J=6.8, 13.4 Hz, 1H), 2.86 (dd, J=6.8, 13.2 Hz, 1H), 2.72 (t, J=7.9, 7.5 Hz, 2H), 1.68-1.01 (m, 15H), 0.83 (br, 2H); low resolution mass spectrum (ES) m/e 467 [(M+H)⁺, calcd for C₂₇H₃₉N₄O₃: 467]; 96% purity based on HPLC.

### Example 266

### (S)-6-AMINO-2-[3-(4-BENZYLOXY-PHENYL)-UREIDO]-HEXANOIC ACID CYCLOPROPYLAMIDE

The title compound was prepared as described in Example 2 with an initial coupling reaction with Cyclopropylamine. ¹H NMR (400 MHz, DMSO-d6) δ 7.43-7.26 (m, 5H), 7.22 (d, J=9.2 Hz, 2H), 6.87 (d, J=9.2 Hz, 2H), 5.00 (s, 2H), 4.06 (dd, J=5.7, 7.7 Hz, 1H), 2.73 (t, J=7.5, 7.7 Hz, 2H), 2.59 (septet, 1H), 1.64-1.37 (m, 4H), 1.35-1.16 (m, 2H), 0.65-0.57 (m, 2H), 0.42-0.33 (m, 2H); low resolution mass spectrum (ES) m/e 411 [(M+H)⁺, calcd for C₂₃H₃₁N₄O₃: 411]; 100% purity based on HPLC.

### Example 267

### (S)-[5-AMINO-1-(5-PHENYL-2H-PYRAZOL-3-YLCARBAMOYL)-PENTYL]-CARBAMIC ACID 9H-FLUOREN-9-YLMETHYL ESTER

The title compound was prepared as described in Example 6 with an initial coupling reaction with 5-Phenyl-2H-pyrazol-3-ylamine. ¹H NMR (400 MHz, DMSO-d6) δ H-NMR (400 MHz) 10.49 (s, 1H); 7.89 (d, 2H, J=7.5 Hz); 7.72 (dd, 4H, J=7.5 Hz, J=14.7 Hz); 7.57 (m, 3H); 7.42 (m, 4H); 7.33 (m, 3H); 6.85 (s, 1H); 4.34-4.14 (m, 4H); 2.78 (dddd, 2H, J=7.1 Hz, J=7.1 Hz, J=6.7 Hz, J=12.3 Hz); 1.75-1.28 (m, 6H); low resolution mass spectrum (ES) m/e 510 [(M+H)⁺, calcd for C₃₀H₃₂N₅O₃: 510]; 100% purity based on HPLC.

### Example 268

### (S)-3-(2-AMINO-ACETYLAMINO)-2-[3-(4-BENZYLOXY-PHENYL)-UREIDO]-N-P-TOLYL-PROPIONAMIDE

4-Methyl-phenylamine was coupled to (S)-3-tert-Butoxycarbonylamino-2-(9H-fluoren-9-ylmethoxycarbonylamino)-propionic acid, and then to tert-Butoxycarbonylamino-acetic acid as described in the method of Example 182. Coupling to 1-Benzyloxy-4-isocyanato-benzene as described in the method of Example 2. Purification by HPLC produced the title compound. ¹H NMR (400 MHz, DMSO-d6) δ 7.46 (d, J=8.6 Hz, 2H), 7.43-7.23 (m, 7H), 7.10 (d, J=8.1 Hz, 2H), 6.88 (d, J=9.2 Hz, 2H), 5.01 (s, 2H), 4.45 (t, J=6.4, 5.9 Hz, 1H), 3.51-3.44 (m, 4H), 2.23 (s, 3H); low resolution mass spectrum (ES) m/e 476 [(M+H)⁺, calcd for C₂₆H₃₀N₅O₄: 476]; 94% purity based on HPLC.

### Example 269

### (S)-6-AMINO-2-[3-(4-PIPERIDIN-1-YL-PHENYL)-UREIDO]-HEXANOIC ACID P-TOLYLAMIDE

The title compound was prepared as described in the method of Example 2 with a final coupling to 1-(4-Isocyanato-phenyl)-piperidine. ¹H NMR (500 MHz, DMSO-d6) δ 10.08 (s, 1H), 8.99 (br s, 1H), 7.70 (br s, 3H), 7.50-7.46 (br m, 6H), 7.11 (d, J=8.45 Hz, 2H), 6.68 (br s, 1H), 4.36 (dd, J=7.98, 13.51 Hz, 1H), 3.38 (br s, 3H), 2.81-2.75 (m, 2H), 2.25 (s, 3H), 1.82-1.31 (m, 13H); low resolution mass spectrum (ES) m/e 438 [(M+H)⁺, calcd for C₂₅H₃₆N₅O₂: 438]; 95.1% purity based on HPLC.

### Example 270

### (S)-6-AMINO-2-[3-(4-BENZOOXAZOL-2-YL-PHENYL)-UREIDO]-HEXANOIC ACID P-TOLYLAMIDE

The title compound was prepared as described in the method of Example 2 with a final coupling to 2-(4-Isocyanato-phenyl)-benzooxazole. ¹H NMR (500 MHz, DMSO-d6) δ 10.12 (s, 1H), 9.27 (s, 1H), 8.08 (d, J=8.62 Hz, 2H), 7.74-7.71 (br m, 4H), 7.63 (d, J=8.60 Hz, 2H), 7.51 (d, J=8.08 Hz, 2H), 7.39-7.37 (m, 2H), 7.12 (d, J=7.97 Hz, 2H), 6.81 (d, J=7.79 Hz, 1H), 4.42 (dd, J=6.88, 13.04 Hz, 1H), 2.80-2.79 (m, 2H), 2.25 (s, 3H), 1.79-1.76 (m, 1H), 1.65-1.57 (m, 3H), 1.45-1.39 (m, 2H); low resolution mass spectrum (ES) m/e 472 [(M+H)⁺, calcd for C₂₇H₃₀N₅O₃: 472]; 97.3% purity based on HPLC.

### Example 271

### (S)-6-AMINO-2-[3-(3-BENZOOXAZOL-2-YL-PHENYL)-UREIDO]-HEXANOIC ACID P-TOLYLAMIDE

The title compound was prepared as described in the method of Example 2 with a final coupling to 2-(3-Isocyanato-phenyl)-benzooxazole. ¹H NMR (500 MHz, DMSO-d6) δ 10.12 (s, 1H), 9.10 (s, 1H), 8.49 (s, 1H), 7.82-7.79 (m, 2H), 7.77-7.75 (m, 1H), 7.67 (br m, 3H), 7.51 (d, J=8.42 Hz, 2H), 7.48-7.40 (m, 4H), 7.13 (d, J=8.35 Hz, 2H), 6.67 (d, J=8.02 Hz, 1H), 4.42 (dt, J=5.95, 8.09 Hz, 1H), 2.83-2.77 (m, 2H), 2.25 (s, 3H), 1.82-1.75 (m, 1H), 1.69-1.55 (m, 3H), 1.46-1.34 (m, 2H); low resolution mass spectrum (ES) m/e 472 [(M+H)⁺, calcd for C₂₇H₃₀N₅O₃: 472]; 98.5% purity based on HPLC.

### Example 272

### (S)-6-AMINO-2-[3-(5-P-TOLYLOXYMETHYL-[1,3,4]THIADIAZOL-2-YL)-UREIDO]-HEXANOIC ACID P-TOLYLAMIDE

The title compound was prepared as described in the method of Example 2 with a final coupling to 2-Isocyanato-5-p-tolyloxymethyl-[1,3,4]thiadiazole. ¹H NMR (500 MHz, DMSO-d6) δ 10.95 (br s, 1H), 10.14 (s, 1H), 7.65 (br s, 3H), 7.48 (d, J=8.41 Hz, 2H), 7.13-7.09 (m, 5H), 6.93 (d, J=8.55 Hz, 2H), 5.38 (s, 2H), 4.42 (dt, J=5.48, 7.90 Hz, 1H), 2.80-2.74 (m, 2H), 2.25 (s, 3H), 2.23 (s, 3H), 1.81-1.74 (m, 1H), 1.68-1.51 (m, 3H), 1.40-1.29 (m, 2H); low resolution mass spectrum (ES) m/e 483 [(M+H)⁺, calcd for C₂₄H₃₁N₆O₃S: 483]; 90.4% purity based on HPLC.

### Example 273

### (S)-6-AMINO-2-{3-[4-(4-CHLORO-BENZYLOXY)-PHENYL]-UREIDO}-HEXANOIC ACID P-TOLYLAMIDE

The title compound was prepared as described in the method of Example 248 with a final coupling to 1-Bromomethyl-4-chloro-benzene. ¹H NMR (400 MHz, DMSO-d6) δ 10.05 (s, 1H), 8.56 (s, 1H), 7.68 (br s, 3H), 7.49 (d, J=8.42 Hz, 2H), 7.44 (s, 4H), 7.28 (d, J=9.04 Hz, 2H), 7.11 (d, J=−8.33 Hz, 2H), 6.89 (d, J=−9.06 Hz, 2H), 6.45 (d, J=−8.24 Hz, 1H), 5.03 (s, 2H), 4.37 (dd, J=7.99, 13.57 Hz, 1H), 2.79-2.76 (m, 2H), 2.25 (s, 3H), 1.77-1.67 (m, 1H), 1.64-1.52 (m, 3H), 1.45-1.30 (m, 2H); low resolution mass spectrum (ES) m/e 495 [(M+H)⁺, calcd for C₂₇H₃₂ClN₄O₃: 495]; 98.9% purity based on HPLC.

### Example 274

### (S)-6-AMINO-2-{3-[4-(2,4-DICHLORO-BENZYLOXY)-PHENYL]-UREIDO}-HEXANOIC ACID P-TOLYLAMIDE

The title compound was prepared as described in the method of Example 248 with a final coupling to 1-Bromomethyl-2,4-dichloro-benzene. ¹H NMR (400 MHz, DMSO-d6) d 10.05 (s, 1H), 8.59 (s, 1H), 7.69 (br s, 3H), 7.67 (d, J=2.06 Hz, 1H), 7.59 (d, J=8.33 Hz, 1H), 7.49 (d, J=8.43 Hz, 2H), 7.47 (dd, J=2.11, 8.34 Hz, 1H), 7.30 (d, J=9.06 Hz, 2H), 7.11 (d, J=8.39 Hz, 2H), 6.91 (d, J=9.05 Hz, 2H), 6.47 (d, J=7.32 Hz, 1H), 5.07 (s, 2H), 4.37 (dd, J=7.94, 13.54 Hz, 1H), 2.81-2.76 (m, 2H), 2.25 (s, 3H), 1.77-1.69 (m, 1H), 1.64-1.53 (m, 3H), 1.45-1.32 (m, 2H); low resolution mass spectrum (ES) m/e 529, 531 [(M+H)⁺, calcd for C₂₇H₃₁Cl₂N₄O₃: 529, 531]; 99.4% purity based on HPLC.

### Example 275

### (S)-6-AMINO-2-{3-[4-(3-NITRO-BENZYLOXY)-PHENYL]-UREIDO}-HEXANOIC ACID P-TOLYLAMIDE

The title compound was prepared as described in the method of Example 248 with a final coupling to 1-Bromomethyl-3-nitro-benzene. ¹H NMR (400 MHz, DMSO-d6) δ 10.04 (s, 1H), 8.57 (s, 1H), 8.27 (s, 1H), 8.17 (d, J=8.32 Hz, 1H), 7.88 (d, J=7.68 Hz, 1H), 7.68 (t, J=−7.91, Hz, 1H), 7.69 (br s, 3H), 7.48 (d, J=−8.43 Hz, 2H), 7.29 (d, J=−9.06 Hz, 2H), 7.10 (d, J=8.34 Hz, 2H), 6.92 (d, J=9.07 Hz, 2H), 6.46 (br m, 1H), 5.19 (s, 2H), 4.36 (q, J=7.77 Hz, 1H), 2.81-2.73 (m, 2H), 2.25 (s, 3H), 1.77-1.68 (m, 1H), 1.64-1.51 (m, 3H), 1.45-1.30 (m, 2H); low resolution mass spectrum (ES) m/e 506 [(M+H)⁺, calcd for C₂₇H₃₂N₅O₅: 506]; 95.1% purity based on HPLC.

### Example 276

### (S)-3-{4-[3-(5-AMINO-1-P-TOLYLCARBAMOYL-PENTYL)-UREIDO]-PHENOXYMETHYL}-BENZOIC ACID METHYL ESTER

The title compound was prepared as described in the method of Example 248 with a final coupling of 3-Bromomethyl-benzoic acid methyl ester. ¹H NMR (400 MHz, DMSO-d6) δ 10.07 (s, 1H), 8.57 (s, 1H), 8.02 (s, 1H), 7.91 (d, J=7.77 Hz, 1H), 7.71 (d, J=7.69 Hz, 1H), 7.69 (br, 3H), 7.54 (t, J=7.70 Hz, 1H), 7.49 (d, J=8.43 Hz, 2H), 7.29 (d, J=9.05 Hz, 2H), 7.11 (d, J=−8.40 Hz, 2H), 6.91 (d, J=−9.06 Hz, 2H), 6.46 (d, J=−6.12 Hz, 1H), 5.12 (s, 2H), 4.37 (dd, J=7.88, 13.55 Hz, 1H), 3.86 (s, 3H), 2.82-2.73 (m, 2H), 2.25 (s, 3H), 1.77-1.68 (m, 1H), 1.64-1.51 (m, 3H), 1.45-1.30 (m, 2H); low resolution mass spectrum (ES) m/e 519 [(M+H)⁺, calcd for C₂₉H₃₅N₄O₅: 519]; 94.8% purity based on HPLC.

### Example 277

### (2S)-6-AMINO-2-{3-[4-(1-PHENYL-ETHOXY)-PHENYL]-UREIDO}-HEXANOIC ACID P-TOLYLAMIDE

The title compound was prepared as described in the method of Example 248 with a final coupling of racemic (1-Bromo-ethyl)-benzene. ¹H NMR (400 MHz, DMSO-d6) δ 10.03 (s, 1H), 8.47 (s, 1H), 7.65 (br s, 3H), 7.48 (d, J=8.35 Hz, 2H), 7.37 (d, J=7.02 Hz, 2H), 7.32 (t, J=7.53 Hz, 2H), 7.23 (t, J=7.17 Hz, 1H), 7.18 (d, J=8.99 Hz, 2H), 7.10 (d, J=8.38 Hz, 2H), 6.77 (d, J=9.04 Hz, 2H), 6.40 (d, J=8.21 Hz, 1H), 5.38 (q, J=6.36 Hz, 1H), 4.35 (dd, J=8.00, 13.60 Hz, 1H), 2.79-2.74 (m, 2H), 2.24 (s, 3H), 1.75-1.66 (m, 1H), 1.61-1.51 (m, 3H), 1.51 (d, J=6.37 Hz, 3H), 1.42-1.28 (m, 2H); low resolution mass spectrum (ES) m/e 475 [(M+H)⁺, calcd for C₂₈H₃₅N₄O₃: 475]; 100% purity based on HPLC.

### Example 278

### 2-[1-(4-AMINO-BUTYL)-3-(4-BENZYLOXY-PHENYL)-UREIDO]-N-P-TOLYL-ACETAMIDE

[(4-tert-Butoxycarbonylamino-butyl)-(9H-fluoren-9-ylmethoxycarbonyl)-amino]-acetic acid was coupled to 4-methylphenylamine, and then to 1-Benzyloxy-4-isocyanato-benzene as described in the method of Example 2. Purification by HPLC produced the title compound. ¹H NMR (400 MHz, DMSO-d6) δ 9.91 (s, 1H), 8.26 (s, 1H), 7.65 (br s, 3H), 7.48 (d, J=8.36 Hz, 2H), 7.43 (d, J=6.94 Hz, 2H), 7.38 (t, J=7.22, 7.22 Hz, 2H), 7.34-7.30 (m, 3H), 7.11 (d, J=8.37 Hz, 2H), 6.90 (d, J=9.00 Hz, 2H), 5.04 (s, 2H), 4.11 (s, 2H), 3.38 (br, 2H), 2.81-2.80 (br m, 2H), 2.25 (s, 3H), 1.56 (br, 4H); low resolution mass spectrum (ES) m/e 461 [(M+H)⁺, calcd for C₂₇H₃₃N₄O₃: 461]; 99% purity based on HPLC.

### Example 279

### (S)-6-AMINO-2-{3-[4-(PYRIDIN-4-YLMETHOXY)-PHENYL]-UREIDO}-HEXANOIC ACID P-TOLYLAMIDE

The title compound was prepared as described in the method of Example 262 with a final coupling with 4-Bromomethyl-pyridine. ¹H NMR (400 MHz, DMSO-d6) δ 10.06 (s, 1H), 8.64 (m, 2H), 8.57-8.55 (m, 1H), 7.64 (br s, 3H), 7.56-7.53 (m, 2H), 7.49 (d, J=8.42 Hz, 2H), 7.29 (d, J=8.99 Hz, 2H), 7.11 (d, J=8.27 Hz, 2H), 6.91 (d, J=12.42 Hz, 2H), 6.44 (t, J=7.96 Hz, 1H), 5.18 (m, 2H), 4.37 (dd, J=7.53, 13.84 Hz, 1H), 2.80-2.75 (m, 2H), 2.25 (s, 3H), 1.75-1.68 (m, 1H), 1.63-1.52 (m, 3H), 1.44-1.29 (m, 2H); low resolution mass spectrum (ES) m/e 462 [(M+H)⁺, calcd for C₂₈H₃₂N₅O₃: 462]; 95.4% purity based on HPLC.

### Example 280

### (S)-{4-[(5-AMINO-1-P-TOLYLCARBAMOYL-PENTYLAMINO)-METHYL]-PHENYL}-CARBAMIC ACID

Intermediate #2 of Example 1 was coupled to 1-Chloromethyl-4-isocyanato-benzene as described for Intermediate #3 of Example 1 and quenched with water. Purification by HPLC produced the title compound. ¹H NMR (500 MHz, DMSO-d6) δ 10.08 (s, 1H), 8.69 (s, 1H), 7.68 (br s, 3H), 7.50 (d, J=8.45 Hz, 2H), 7.33 (d, J=8.52 Hz, 2H), 7.16 (d, J=8.47 Hz, 2H), 7.11 (d, J=8.46 Hz, 2H), 6.53 (d, J=8.26 Hz, 1H), 5.03 (br s, 1H), 4.40-4.36 (m, 3H), 2.82-2.75 (m, 2H), 2.25 (s, 3H), 1.77-1.70 (m, 1H), 1.64-1.52 (m, 3H), 1.44-1.30 (m, 2H); low resolution mass spectrum (ES) m/e 385 [(M+H)⁺, calcd for C₂₁H₂₉N₄O₃: 385]; 98.9% purity based on HPLC.

### Example 281

### 2-[1-(4-AMINO-BUTYL)-3-(4-BENZYLOXY-PHENYL)-UREIDO]-N-(1H-INDOL-4-YL)-ACETAMIDE

[(4-tert-Butoxycarbonylamino-butyl)-(9H-fluoren-9-ylmethoxycarbonyl)-amino]-acetic acid was coupled to 1H-Indol-4-ylamine, and then to 1-Benzyloxy-4-isocyanato-benzene as described in the method of Example 2. Purification by HPLC produced the title compound. ¹H NMR (400 MHz, DMSO-d6) δ 9.66 (s, 1H); 8.32 (s, 1H); 7.62 (m, 4H); 7.48-7.24 (m, 9H); 7.14 (d, 1H, J=8.1 Hz); 7.00 (t, 1H, J=7.9 Hz); 6.90 (d, 2H, J=9.1 Hz); 6.68 (s, 1H); 5.03 (s, 2H); 4.25 (s, 2H); 3.42 (t, 2H, J=6.2 Hz); 2.81 (dd, 2H, J=6.1 Hz, J=11.9 Hz); 1.58 (br s, 4H); low resolution mass spectrum (ES) m/e 486 [(M+H)⁺, calcd for C₂₈H₃₂N₅O₃: 486]; 90% purity based on HPLC.

### Example 282

### 2-[1-(4-AMINO-BUTYL)-3-(4-BENZYLOXY-PHENYL)-UREIDO]-N-(4-TERT-BUTYL-PHENYL)-ACETAMIDE

[(4-tert-Butoxycarbonylamino-butyl)-(9H-fluoren-9-ylmethoxycarbonyl)-amino]-acetic acid was coupled to 4-t-butyl phenylamine, and then to 1-Benzyloxy-4-isocyanato-benzene as described in the method of Example 2. Purification by HPLC produced the title compound. ¹H NMR (400 MHz, DMSO-d6) δ 9.90 (s, 1H); 8.25 (s, 1H); 7.63 (s, 3H); 7.50 (d, 2H, J=8.7 Hz); 7.39 (m, 4H); 7.31 (ddd, 5H, J=2.4 Hz, J=4.1 Hz, J=8.7 Hz); 6.89 (d, 2H, J=9.1 Hz); 5.03 (s, 2H); 4.10 (s, 2H); 2.80 (dd, 2H, J=6.0 Hz, J=11.3 Hz); 1.56 (s, 4H); 1.25 (s, 9H); low resolution mass spectrum (ES) m/e 503 [(M+H)⁺, calcd for C₃₀H₃₉N₄O₃: 503]; 98% purity based on HPLC.

### Example 283

### (S)-6-AMINO-2-[3-(4-BENZYLOXY-PHENYL)-UREIDO]-HEXANOIC ACID INDAN-5-YLAMIDE

The title compound was prepared as described in the method of Example 2 with an initial coupling to Indan-5-ylamine. ¹H NMR (400 MHz, DMSO-d6) δ 7.47 (br, 1H), 7.42-7.21 (m, 8H), 7.12 (d, J=8.1 Hz, 1H), 6.87 (d, J=9.2 Hz, 2H), 5.00 (s, 2H), 4.32 (dd, J=5.7, 7.9 Hz, 1H), 2.77 (quintet, J=7.2, 13.8, 12.8 Hz, 6H), 1.97 (quintet, J=7.2, 11.0, 11.4 Hz, 2H), 1.76-1.48 (m, 4H), 1.41-1.25 (m, 2H); low resolution mass spectrum (ES) m/e 487 [(M+H)⁺, calcd for C₂₉H₃₅N₄O₃: 487]; 96% purity based on HPLC.

### Example 284

### (S)-6-AMINO-2-{3-[4-(2,3-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,3-difluoro-benzene. ¹H NMR (500 MHz, DMSO-d6) δ 10.07 (s, 1H), 8.56 (s, 1H), 7.65 (br s, 3H), 7.49 (d, J=−8.42 Hz, 2H), 7.43 (dt, J=1.30, 9.82 Hz, 1H), 7.35 (dd, J=6.45, 7.30 Hz, 1H), 7.30 (d, J=8.88 Hz, 2H), 7.23 (dt, J=0.99, 8.92 Hz, 1H), 7.11 (d, J=8.54 Hz, 2H), 6.92 (d, J=8.92 Hz, 2H), 6.44 (d, J=8.30 Hz, 1H), 5.12 (s, 2H), 4.38 (dt, J=5.83, 8.09 Hz, 1H), 2.81-2.75 (m, 2H), 2.25 (s, 3H), 1.76-1.69 (m, 1H), 1.63-1.51 (m, 3H), 1.44-1.31 (m, 2H); low resolution mass spectrum (ES) m/e 497 [(M+H)⁺, calcd for C₂₇H₃₁F₂N₄O₃: 497]; 85.4% purity based on HPLC.

### Example 285

### (S)-6-AMINO-2-{3-[4-(2,4-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,4-difluoro-benzene. ¹H NMR (500 MHz, DMSO-d6) δ 10.07 (s, 1H), 8.57 (s, 1H), 7.67 (br s, 3H), 7.59 (dd, J=8.56, 15.32 Hz, 1H), 7.49 (d, J=8.42 Hz, 2H), 7.32-7.27 (m, 3H), 7.13-7.10 (m, 3H), 6.91 (d, J=8.96 Hz, 2H), 6.45 (d, J=8.35 Hz, 1H), 5.03 (s, 2H), 4.37 (dt, J=5.66, 8.16 Hz, 1H), 2.81-2.75 (m, 2H), 2.25 (s, 3H), 1.76-1.69 (m, 1H), 1.63-1.51 (m, 3H), 1.44-1.31 (m, 2H); low resolution mass spectrum (ES) m/e 497 [(M+H)⁺, calcd for C₂₇H₃₁F₂N₄O₃: 497]; 97.3% purity based on HPLC.

### Example 286

### (S)-6-AMINO-2-{3-[4-(2,5-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,5-difluoro-benzene. ¹H NMR (500 MHz, DMSO-d6) δ 10.07 (s, 1H), 8.58 (s, 1H), 7.66 (br s, 3H), 7.49 (d, J=−8.41 Hz, 2H), 7.38 (ddd, J=3.20, 5.62, 8.85 Hz, 1H), 7.33-7.29 (m, 3H), 7.27-7.22 (m, 1H), 7.11 (d, J=8.46 Hz, 2H), 6.92 (d, J=8.95 Hz, 2H), 6.45 (d, J=8.28 Hz, 1H), 5.06 (s, 2H), 4.38 (dt, J=5.79, 8.11 Hz, 1H), 2.81-2.75 (m, 2H), 2.25 (s, 3H), 1.76-1.69 (m, 1H), 1.63-1.52 (m, 3H), 1.44-1.31 (m, 2H); low resolution mass spectrum (ES) m/e 497 [(M+H)⁺, calcd for C₂₇H₃₁F₂N₄O₃: 497]; 97.3% purity based on HPLC.

### Example 287

### (S)-6-AMINO-2-{3-[4-(2,6-DIFLUORO-BENZYLOXY)-PHENYL]-UREIDO}-HEXANOIC ACID P-TOLYLAMIDE

The title compound was prepared as described in the method of Example 248 with a final coupling to 1-Bromomethyl-2,6-difluoro-benzene: ¹H NMR (500 MHz, DMSO-d6) δ 10.07 (s, 1H), 8.57 (s, 1H), 7.65 (br s, 3H), 7.54-7.48 (m, 3H), 7.30 (d, J=8.91 Hz, 2H), 7.21-7.15 (m, 3H), 7.12 (d, J=−8.50 Hz, 2H), 6.91 (d, J=8.94 Hz, 2H), 6.44 (d, J=8.26 Hz, 1H), 5.03 (s, 2H), 4.38 (dt, J=5.59, 8.03 Hz, 1H), 2.81-2.75 (m, 2H), 2.25 (s, 3H), 1.76-1.69 (m, 1H), 1.63-1.51 (m, 3H), 1.43-1.32 (m, 2H); low resolution mass spectrum (ES) m/e 497 [(M+H)⁺, calcd for C₂₇H₃₁F₂N₄O₃: 497]; 81.7% purity based on HPLC.

### Example 288

### (S)-6-AMINO-2-{3-[4-(3,4-DIFLUORO-BENZYLOXY)-PHENYL]-UREIDO}-HEXANOIC ACID P-TOLYLAMIDE

The title compound was prepared as described in Example 248 with the final coupling completed with 1-Bromomethyl-3,4-difluoro-benzene: ¹H NMR (500 MHz, DMSO-d6) δ 10.07 (s, 1H), 8.56 (s, 1H), 7.67 (br s, 3H), 7.52-7.41 (m, 4H), 7.28 (d, J=9.03 Hz, 3H), 7.11 (d, J=8.39 Hz, 2H), 6.89 (d, J=9.05 Hz, 2H), 6.44 (d, J=8.30 Hz, 1H), 5.02 (s, 2H), 4.37 (dt, J=5.86, 8.17 Hz, 1H), 2.81-2.75 (m, 2H), 2.25 (s, 3H), 1.77-1.69 (m, 1H), 1.63-1.51 (m, 3H), 1.42-1.30 (m, 2H); low resolution mass spectrum (ES) m/e 497 [(M+H)⁺, calcd for C₂₇H₃₁F₂N₄O₃: 497]; 93.1% purity based on HPLC.

### Example 289

### (S)-1-[5-AMINO-1-(4-METHYL-PIPERAZINE-1-CARBONYL)-PENTYL]-3-(4-BENZYLOXY-PHENYL)-UREA

The title compound was prepared as described in the method of Example 2 with an initial coupling to 1-Methyl-piperazine. ¹H NMR (400 MHz, DMSO-d6) δ 7.42-7.26 (m, 5H), 7.22 (d, J=9.4 Hz, 2H), 6.87 (d, J=9.4 Hz, 2H), 5.00 (s, 3H), 4.63 (dd, J=5.2, 7.8 Hz, 1H), 3.38-2.83 (br, 4H), 2.80-2.69 (m, 5H), 1.68-1.21 (m, 6H), CH₂ obscured by water peak; low resolution mass spectrum (ES) m/e 454 [(M+H)⁺, calcd for C₂₅H₃₆N₅O₃: 453]; 99% purity based on HPLC.

### Example 290

### (S)-1-[5-AMINO-1-(PIPERIDINE-1-CARBONYL)-PENTYL]-3-(4-BENZYLOXY-PHENYL)-UREA

The title compound was prepared as described in the method of Example 2 with an initial coupling to Piperidine. ¹H NMR (400 MHz, DMSO-d6) δ 7.41-7.25 (m, 5H), 7.21 (d, J=9.4 Hz, 2H), 6.86 (d, J=8.9 Hz, 2H), 4.99 (s, 2H), 4.63 (dd, J=4.6, 8.3 Hz, 1H), 3.52-3.29 (m, 4H), 2.73 (t, J=7.4 Hz, 2H), 1.63-1.22 (m, 12H); low resolution mass spectrum (ES) m/e 439 [(M+H)⁺, calcd for C₂₅H₃₅N₄O₃: 438.6]; 99% purity based on HPLC.

### Example 291

### (S)-6-AMINO-2-[3-(4-BENZYLOXY-PHENYL)-UREIDO]-HEXANOIC ACID ISOPROPYLAMIDE

The title compound was prepared as described in the method of Example 2 with an initial coupling to Isopropylamine. ¹H NMR (400 MHz, DMSO-d6) δ 7.39-7.22 (m, 5H), 7.19 (d, J=8.9 Hz, 2H), 6.84 (d, J=8.9 Hz, 2H), 4.97 (s, 2H), 4.06 (dd, J=5.9, 7.4 Hz, 1H), 3.77 (m, 1H), 2.70 (t, J=7.6 Hz, 2H), 1.61-1.12 (m, 6H), 1.00 (t, J=6.3 Hz, 6H); low resolution mass spectrum (ES) m/e 413 [(M+H)⁺, calcd for C₂₃H₃₃N₄O₃: 412]; 97% purity based on HPLC.

### Example 292

### (S)-6-AMINO-2-(3-{4-[(METHYL-P-TOLYL-AMINO)-METHYL]-PHENYL}-UREIDO)-HEXANOIC ACID P-TOLYLAMIDE

The title compound was prepared as described in the method of Example 251. ¹H NMR (500 MHz, DMSO-d6) δ 10.49 (s, 1H), 9.30 (br, 2H), 8.13 (s, 1H), 7.71 (br s, 3H), 7.46 (dd, J=2.68, 8.57 Hz, 4H), 7.29 (d, J=8.56 Hz, 2H), 7.22-7.15 (m, 6H), 4.06 (br s, 2H), 3.83 (br s, 1H), 3.22 (s, 3H), 2.77-2.73 (m, 2H), 2.32 (s, 3H), 2.27 (s, 3H), 1.95-1.24 (m, 6H); low resolution mass spectrum (ES) m/e 488 [(M+H)⁺, calcd for C₂₉H₃₈N₅O₂: 488]; 91.7% purity based on HPLC.

### Example 293

### (4-AMINO-BUTYL)-(P-TOLYLCARBAMOYL-METHYL)-CARBAMIC ACID 9H-FLUOREN-9-YLMETHYL ESTER

[(4-tert-Butoxycarbonylamino-butyl)-(9H-fluoren-9-ylmethoxycarbonyl)-amino]-acetic acid was coupled to 4-t-butyl phenylamine as described in the method of Example 6. Purification by HPLC produced the title compound. ¹H NMR (400 MHz, DMSO-d6) δ 10.04 (s, 0.54H); 9.88 (s, 0.43H); 7.91 (d, J=7.44 Hz, 0.85H); 7.84 (d, J=7.59 Hz, 1.05H); 7.71-7.61 (m, 2.75H); 7.59 (d, J=7.52 Hz, 1H); 7.53 (d, J=8.34 Hz, 1H); 7.47-7.40 (m, 1.62H); 7.35 (dd, J=7.28, 14.6 Hz, 1.85H); 7.18-7.05 (m, 3H); 4.45 (d, J=5.60 Hz, 1H); 4.31 (t, J=5.53 Hz, 0.63H); 4.24-4.07 (m, 3H); 3.93 (br s, 1H); 3.03 (t, J=6.98 Hz, 1.23H); 2.89-2.72 (m, 1.2H); 2.71-2.59 (m, 0.98H); 2.27 (s, 1.68H); 1.37 (s, 1.37H); 1.63-1.41 (m, 2H); 1.40-1.16 (m, 2H). Low resolution mass spectrum (ES) m/e 458 [(M+H)⁺, calcd for C₂₈H₃₂N₃O₃: 458]; 100% purity based on HPLC.

### Example 294

### [5-AMINO-1-(6-METHYL-PYRIDIN-3-YLCARBAMOYL)-PENTYL]-CARBAMIC ACID 9H-FLUOREN-9-YLMETHYL ESTER

The title compound was prepared as described in the method of Example 6 with an initial coupling to 6-Methyl-pyridin-3-ylamine. 1H NMR (400 MHz, DMSO-d6) δ 10.04 (s, 0.59H); 9.87 (s, 0.41H); 7.90 (d, J=7.46 Hz, 0.83H); 7.83 (d, J=7.57 Hz, 1H); 7.73-7.59 (m, 3H); 7.57 (d, J=7.51 Hz, 1H); 7.53 (d, J=8.38 Hz, 1H); 7.42 (t, J=7.58, 1.42H); 7.39-7.27 (m, 1.63H); 7.23-6.99 (m, 2.52H); 4.44 (d, J=5.59 Hz, 0.83H); 4.30 (t, J=5.28 Hz, 0.54H); 4.23-4.06 (m, 2.46H); 3.92 (br s, 0.86H); 3.02 (t, J=6.81 Hz, 1H); 2.87-2.74 (m, 1H); 2.70-2.57 (m, 1H); 1.86-1.62 (m, 4H); 1.62-1.47 (m, 2H); 1.47-1.1 (m, 6H); Low resolution mass spectrum (ES) m/e 526.25 [(M+H)⁺, calcd for C₃₃H₃₉N₃O₃: 526.30]; 99.5% purity based on HPLC.

### Example 295

### (4-AMINO-BUTYL)-[(2-METHYL-1H-INDOL-5-YLCARBAMOYL)-METHYL]-CARBAMIC ACID 9H-FLUOREN-9-YLMETHYL ESTER

[(4-tert-Butoxycarbonylamino-butyl)-(9H-fluoren-9-ylmethoxycarbonyl)-amino]-acetic acid was coupled to 4-t-butyl phenylamine as described in the method of Example 6. Purification by HPLC produced the title compound. ¹H NMR (400 MHz, DMSO-d6) δ 10.82 and 10.79 (two singlets, 1H); 9.86 (s, 0.58H); 9.67 (s, 0.45H); 7.91 (d, J=7.47 Hz, 1H); 7.83 (d, J=7.53 Hz, 1H); 7.71-7.52 (m, 5H); 7.43 (t, J=7.36 Hz, 1H); 7.39-7.28 (m, 2H); 7.23-7.01 (m, 3H); 6.04 (d, J=15.1 Hz, 1H); 4.44 (d, J=5.59 Hz, 1H); 4.30 (t, J=5.33 Hz, 0.68H); 4.18 (br s, 1.64H); 4.13 (br s, 1.36H); 3.93 (br s, 1H); 3.041 (t, J=6.85 Hz, 1H); 2.88-2.76 (m, 1H); 2.73-2.58 (m, 1H); 2.35 (s, 1.74H); 2.33 (s, 1.30H); 1.57 (br s, 2H); 2.12 (m, 2H). Low resolution mass spectrum (ES) m/e 497 [(M+H)⁺, calcd for C₃₀H₃₃N₄O₃: 497]; 97.4% purity based on HPLC.

### Example 296

### (S)-5-(2-AMINO-ACETYLAMINO)-2-[3-(4-BENZYLOXY-PHENYL)-UREIDO]-PENTANOIC ACID (2-METHYL-1H-INDOL-5-YL)-AMIDE

1H-Indol-5-ylamine was coupled to (S)-5-tert-Butoxycarbonylamino-2-(9H-fluoren-9-ylmethoxycarbonylamino)-pentanoic acid as described in the method of Example 6. Coupling to tert-Butoxycarbonylamino-acetic acid, and then to 1-Benzyloxy-4-isocyanato-benzene as described in the method of Example 2 provided the title compound. ¹H NMR (400 MHz, DMSO-d6) δ 7.67 (d, J=2.2 Hz, 1H), 7.43-7.27 (m, 6H), 7.24 (d, J=8.8 Hz, 2H), 7.17 (d, J=8.8 Hz, 1H), 7.09 (dd, J=2.2, 8.3 Hz, 1H), 6.87 (d, J=8.8 Hz, 2H), 5.00 (s, 2H), 4.35 (dd, J=5.9, 7.5 Hz, 1H), 3.47 (s, 2H), 3.13 (t, J=7.2, 6.4 Hz, 2H), 2.32 (s, 3H), 1.79-1.39 (m, 4H); low resolution mass spectrum (ES) m/e 543 [(M+H)⁺, calcd for C₃₀H₃₅N₆O₄: 543]; 93% purity based on HPLC.

### Example 297

### (S)-5-(2-AMINO-ACETYLAMINO)-2-[3-(9H-FLUOREN-2-YL)-UREIDO]-PENTANOIC ACID (2-METHYL-1H-INDOL-5-YL)-AMIDE

1H-Indol-5-ylamine was coupled to (S)-5-tert-Butoxycarbonylamino-2-(9H-fluoren-9-ylmethoxycarbonylamino)-pentanoic acid as described in the method of Example 6. Coupling to tert-Butoxycarbonylamino-acetic acid, and then to 2-Isocyanato-9H-fluorene as described in the method of Example 2 provided the title compound. ¹H NMR (400 MHz, DMSO-d6) δ 7.77-7.66 (m, 4H), 7.50 (d, J=7.5 Hz, 1H), 7.35-7.27 (m, 2H), 7.22 (d, J=7.5 Hz, 1H), 7.17 (d, J=8.8 Hz, 1H), 7.11 (dd, J=2.0, 8.3 Hz, 1H), 4.41 (dd, J=5.9, 7.5 Hz, 1H), 3.83 (s, 2H), 3.48 (s, 2H), 3.16 (t, J=7.5, 7.0 Hz, 2H), 2.33 (s, 3H), 1.81-1.43 (m, 4H); low resolution mass spectrum (ES) m/e 525 [(M+H)⁺, calcd for C₃₀H₃₃N₆O₃: 525]; 91% purity based on HPLC.

### Example 298

### (S)-6-(2-AMINO-ACETYLAMINO)-2-[3-(9H-FLUOREN-2-YL)-UREIDO]-HEXANOIC ACID (2-METHYL-1H-INDOL-5-YL)-AMIDE

1H-Indol-5-ylamine was coupled to (S)-6-tert-Butoxycarbonylamino-2-(9H-fluoren-9-ylmethoxycarbonylamino)-hexanoic acid as described in the method of Example 6. Coupling to tert-Butoxycarbonylamino-acetic acid, and then to 2-Isocyanato-9H-fluorene as described in the method of Example 2 provided the title compound. ¹H NMR (400 MHz, DMSO-d6) δ 7.77-7.66 (m, 4H), 7.50 (d, J=7.2 Hz, 1H), 7.35-7.27 (m, 3H), 7.24-7.15 (m, 2H), 7.11 (dd, J=2.2, 8.6 Hz, 1H), 4.39 (dd, J=5.5, 7.9 Hz, 1H), 3.84 (s, 2H), 3.47 (s, 2H), 3.11 (t, J=6.8 Hz, 2H), 2.33 (s, 3H), 1.82-1.27 (m, 6H); low resolution mass spectrum (ES) m/e 539 [(M+H)⁺, calcd for C₃₁H₃₄N₆O₃: 539]; 90% purity based on HPLC.

### Example 299

### (S)-6-AMINO-2-{3-[4-(PYRIDIN-2-YLMETHOXY)-PHENYL]-UREIDO}-HEXANOIC ACID P-TOLYLAMIDE

The title compound was prepared as described in the method of Example 262 with a final coupling to 2-Bromomethyl-pyridine. ¹H NMR (400 MHz, DMSO-d6) δ 10.07 (s, 1H), 8.60-8.57 (m, 2H), 7.90-7.86 (m, 1H), 7.66 (br s, 3H), 7.57-7.53 (m, 1H), 7.49 (d, J=8.41 Hz, 2H), 7.40-7.37 (m, 1H), 7.29 (d, J=9.00 Hz, 2H), 7.11 (d, J=8.45 Hz, 2H), 6.91 (d, J=9.05 Hz, 2H), 6.45 (br, 1H), 5.13 (s, 2H), 4.37 (dd, J=−7.90, 13.50 Hz, 1H), 2.82-2.74 (m, 2H), 2.25 (s, 3H), 1.77-1.68 (m, 1H), 1.64-1.30 (m, 5H); low resolution mass spectrum (ES) m/e 462 [(M+H)⁺, calcd for C₂₆H₃₂N₅O₃: 461]; 88.7% purity based on HPLC.

### Example 300

### (S)-6-AMINO-2-[3-(4-METHOXY-PHENYL)-UREIDO]-HEXANOIC ACID P-TOLYLAMIDE

The title compound was prepared as described in the method of Example 2 with a final coupling to 1-Isocyanato-4-methoxy-benzene. ¹H NMR (400 MHz, DMSO-d6) δ 10.06 (s, 1H), 8.57 (s, 1H), 7.72 (br s, 3H), 7.50 (d, J=8.43 Hz, 2H), 7.28 (d, J=9.05 Hz, 2H), 7.11 (d, J=8.37 Hz, 2H), 6.81 (d, J=9.06 Hz, 2H), 6.46 (d, J=8.26 Hz, 1H), 4.37 (dd, J=8.04, 13.53 Hz, 1H) 2.82-2.74 (m, 2H), 2.25 (s, 3H), 1.77-1.68 (m, 1H), 1.64-1.51 (m, 3H), 1.46-1.30 (m, 2H); low resolution mass spectrum (ES) m/e 385 [(M+H)⁺, calcd for C₂₁H₂₉N₄O₃: 385]; 95.3% purity based on HPLC.

### Example 301

### (S)-6-AMINO-2-{3-[4-(4-METHYL-BENZYLOXY)-PHENYL]-UREIDO}-HEXANOIC ACID P-TOLYLAMIDE

The title compound was prepared as described in the method of Example 248 with a final coupling to 1-Bromomethyl-4-methyl-benzene. ¹H NMR (500 MHz, DMSO-d6) δ 10.03 (s, 1H), 8.98 (s, 1H), 8.29 (s, 1H), 7.64 (br s, 3H), 7.48 (d, J=8.42 Hz, 2H), 7.11 (d, J=8.49 Hz, 2H), 7.08-7.03 (m, 5H), 6.90 (d, J=2.61 Hz, 1H), 6.66 (d, J=8.61 Hz, 1H), 6.29 (d, J=8.33 Hz, 1H) 4.33 (dd, J=−8.04, 13.67 Hz, 1H), 3.75 (s, 2H), 2.80-2.73 (m, 2H), 2.25 (s, 3H), 2.24 (s, 3H), 1.73-1.66 (m, 1H), 1.59-1.50 (m, 3H), 1.40-1.29 (m, 2H); low resolution mass spectrum (ES) m/e 475 [(M+H)⁺, calcd for C₂₈H₃₅N₄O₃: 475]; 98.1% purity based on HPLC.

### Example 302

### (S)-6-AMINO-2-[3-(4-BENZYLOXY-PHENYL)-UREIDO]-HEXANOIC ACID (FURAN-2-YLMETHYL)-AMIDE

The title compound was prepared as described in the method of Example 2 with an initial coupling to C-Furan-2-yl-methylamine. ¹H NMR (400 MHz, DMSO-d6) δ 7.54 (br s, 1H), 7.43-7.27 (m, 5H), 7.23 (d, J=8.6 Hz, 2H), 6.88 (d, J=9.0 Hz, 2H), 5.01 (s, 2H), 4.29-4.24 (br, 2H), 4.19 (dd, J=5.7, 7.7 Hz, 1H), 2.73 (t, J=7.7 Hz, 2H), 1.69-1.41 (m, 4H), 1.36-1.19 (m, 2H), low resolution mass spectrum (ES) m/e 451 [(M+H)⁺, calcd for C₂₅H₃₁N₄O₄: 450]; 100% purity based on HPLC.

### Example 303

### (S)-6-AMINO-2-[3-(4-BENZYLOXY-PHENYL)-UREIDO]-HEXANOIC ACID BENZOTHIAZOL-2-YLAMIDE

The title compound was prepared as described in the method of Example 2 with an initial coupling to Benzothiazol-2-ylamine. ¹H NMR (400 MHz, DMSO-d6) δ 7.96 (d, J=7.2 Hz, 1H), 7.74 (d, J=7.2 Hz, 1H), 7.48-7.21 (m, 10H), 6.88 (d, J=9.0 Hz, 2H), 5.01 (s, 2H), 4.49 (dd, J=5.9, 7.9 Hz, 1H), 2.77 (t, J=7.7 Hz, 2H), 1.87-1.49 (m, 4H), 1.48-1.29 (m, 2H); low resolution mass spectrum (ES) m/e 504 [(M+H)⁺, calcd for C₂₇H₃₀N₅O₃S: 503]; 92% purity based on HPLC.

### Example 304

### (S)—N-(4-AMINOMETHYL-BENZYL)-2-[3-(4-BENZYLOXY-PHENYL)-UREIDO]-2-PHENYL-ACETAMIDE

(S)-[(9H-Fluoren-9-ylmethoxycarbonylamino)]-phenyl-acetic acid was coupled to (4-Aminomethyl-benzyl)-carbamic acid tert-butyl ester, and then to 1-Benzyloxy-4-isocyanato-benzene as described in the method of Example 2. Purification by HPLC produced the title compound. ¹H NMR (400 MHz, DMSO-d6) δ 8.91 (t, J=5.7 Hz, 1H), 7.44-7.26 (m, 12H), 7.23 (d, J=9.4 Hz, 2H), 7.16 (d, J=8.3 Hz, 2H), 6.87 (d, J=8.8 Hz, 2H), 5.35 (s, 1H), 5.00 (s, 2H), 4.28 (d, J=5.5 Hz, 2H), 3.95 (s, 2H); low resolution mass spectrum (ES) m/e 495 [(M+H)⁺, calcd for C₃₀H₃₁N₄O₃: 495]; 90% purity based on HPLC.

### Example 305

### (S)—N-(4-AMINOMETHYL-PHENYL)-2-[3-(4-BENZYLOXY-PHENYL)-UREIDO]-2-PHENYL-ACETAMIDE

(S)-[(9H-Fluoren-9-ylmethoxycarbonylamino)]-phenyl-acetic acid was coupled to (4-Amino-benzyl)-carbamic acid tert-butyl ester, and then to 1-Benzyloxy-4-isocyanato-benzene as described in the method of Example 2. Purification by HPLC produced the title compound. ¹H NMR (400 MHz, DMSO-d6) δ 10.51 (s, 1H), 7.59 (d, J=6.4 Hz, 2H), 7.46 (d, J=7.5 Hz, 2H), 7.42-7.26 (m, 10H), 7.23 (d, J=9.2 Hz, 2H), 6.87 (d, J=9.2 Hz, 2H), 5.48 (s, 1H), 5.00 (s, 2H), 3.93 (s, 2H); low resolution mass spectrum (ES) m/e 481 [(M+H)⁺, calcd for C₂₉H₂₉N₄O₃: 481]; 99% purity based on HPLC.

### Example 306

### (S)-3-(2-AMINO-ACETYLAMINO)-2-[3-(4-BENZYLOXY-PHENYL)-UREIDO]-N-(4-METHYL-CYCLOHEXYL)-PROPIONAMIDE

4-Methyl-cyclohexylamine was coupled to 3-tert-Butoxycarbonylamino-2-(9H-fluoren-9-ylmethoxycarbonylamino)-propionic acid as described in the method of Example 6. Coupling to tert-Butoxycarbonylamino-acetic acid, and then to 1-Benzyloxy-4-isocyanato-benzene as described in the method of Example 2 produced the title compound. ¹H NMR (400 MHz, DMSO-d6) δ 8.59 (s, 1H); 8.23 (t, J=5.60 Hz, 1H); 7.93 (br s, 3H); 7.45-7.21 (m, 6H); 6.66 (d, J=9 Hz, 2H); 6.34 (d, J=8 Hz, 1H); 5.00 (s, 2H); 4.37 (dd, J=6.03; 13.87 Hz; 1H); 3.8 (br s, 1H); 3.47 (br s, 2H); 3.38 (dddd, J=7.56, 13.62; 13.38, 2H); 1.79-1.11 (m, 9H); 0.87 (d, J=6.6 Hz, 3H); low resolution mass spectrum (ES) m/e 492 [(M+H)⁺, calcd for C₂₆H₃₆N₅O₄: 482]; 95% purity based on HPLC.

### Example 307

### (S)-6-AMINO-2-[3-(4-BENZYLOXY-PHENYL)-UREIDO]-HEXANOIC ACID (1H-INDOL-7-YL)-AMIDE

The title compound was prepared as described in the method of Example 2 with an initial coupling reaction to 1H-Indol-7-ylamine. ¹H NMR (400 MHz, DMSO-d6) δ 10.69 (s, 1H); 9.90 (s, 1H); 8.57 (s, 1H); 7.65 (br s, 3H); 7.56-7.10 (m, 12H); 6.94 (t, 2H, J=7.8 Hz); 6.89 (d, 2H, J=8.9 Hz); 6.50 (d, 1H, J=7.9 Hz); 6.44 (br s, 1H); 5.02 (s, 2H); 4.52 (dd, 1H, J=7.3; 13.3 Hz); 2.79 (br s, 2H); 1.91-1.28 (m, 6H); low resolution mass spectrum (ES) m/e 486 [(M+H)⁺, calcd for C₂₈H₃₂N₅O₃: 485]; 87% purity based on HPLC.

### Example 308

### (±)-4-(2-AMINO-ACETYL)-PIPERAZINE-1,2-DICARBOXYLIC ACID 1-[(4-BENZYLOXY-PHENYL)-AMIDE]2-P-TOLYLAMIDE

Piperazine-1,2-dicarboxylic acid 1-(9H-fluoren-9-ylmethyl) ester was coupled to 4-methyl phenylamine, and then to tert-Butoxycarbonylamino-acetic acid as described in the method of Example 26. Purification by HPLC produced the title compound. ¹H NMR (400 MHz, DMSO-d6) δ 9.95 (d, J=7.5 Hz, 1H), 7.51-7.26 (m, 9H), 7.10 (dd, J=8.8, 11.4 Hz, 2H), 6.91 (dd, J=6.3, 9.0 Hz, 2H), 5.04 (s, 2H), 4.96-4.91 (br, 0.5H), 4.86-4.80 (br, 0.5H), 4.72 (d, J=13.1 Hz, 0.5H), 4.25-3.60 (m, 5H), 3.32-3.20 (m, 2H), 3.05-2.92 (m, 1H), 2.25 (s, 1.5H), 2.23 (s, 1.5H); low resolution mass spectrum (ES) m/e 502 [(M+H)⁺, calcd for C₂₈H₃₂N₅O₄: 502]; 100% purity based on HPLC.

### Example 309

### (S)-2-[3-(4-BENZYLOXY-PHENYL)-UREIDO]-6-HYDROXY-HEXANOIC ACID P-TOLYLAMIDE

The title compound was prepared as described in the method of Example 2 with an initial coupling to (S)-2-(9H-Fluoren-9-ylmethoxycarbonylamino)-6-hydroxy-hexanoic acid. ¹H NMR (400 MHz, DMSO-d6) 10.00 (s, 1H), 8.46 (s, 1H), 7.48 (d, J=8.2 Hz, 2H), 7.44-7.23 (m, 7H), 7.09 (d, J=8.6 Hz, 2H), 6.88 (d, J=9.2 Hz, 2H), 6.33 (d, J=8.2 Hz, 1H), 5.02 (s, 2H), 4.35 (m, 1H), 3.37 (dd, J=6.3, 11.4 Hz, 2H), 2.24 (s, 3H), 1.75-1.25 (m, 6H); low resolution mass spectrum (ES) m/e 462 [(M+H)⁺, calcd for C₂₇H₃₂N₃O₄: 462]; 100% purity based on HPLC.

### Example 310

### [(2-AMINO-PHENYLCARBAMOYL)-METHYL]-CARBAMIC ACID 9H-FLUOREN-9-YLMETHYL ESTER

(9H-Fluoren-9-ylmethoxycarbonylamino)-acetic acid was coupled to (2-Amino-phenyl)-carbamic acid tert-butyl ester as described in the method of Example 6. Purification by HPLC provided the title compound. ¹H NMR (400 MHz, DMSO-d₆) δ 9.19 (s, 1H), 7.90 (d, J=7.2 Hz, 2H), 7.73 (d, J=7.2 Hz, 2H), 7.59 (t, J=5.8 Hz, 1H), 7.43 (t, J=7.4 Hz, 2H), 7.35 (t, J=7.6 Hz, 2H), 7.12 (d, J=8.0, 1H), 6.94 (t, J=7.7 Hz, 1H), 6.73 (d, J=7.6 Hz, 1H), 6.57 (t, J=7.0 Hz, 1H), 4.33 (d, J=6.8 Hz, 2H), 4.25 (t, J=6.8 Hz, 1H); low resolution mass spectrum (ES) m/e 388 [(M+H)⁺, calcd for C₂₃H₂₂N₃O₃: 388]; 94% purity based on HPLC.

### Example 311

### {[2-(2-AMINO-ACETYLAMINO)-PHENYLCARBAMOYL]-METHYL}-CARBAMIC ACID 9H-FLUOREN-9-YLMETHYL ESTER

The compound of Example 310 was coupled to tert-Butoxycarbonylamino-acetic acid as described in the method of Example 6 to produce the title compound. ¹H NMR (400 MHz, DMSO-d₆) δ 9.95 (s, 1H), 9.61 (s, 1H), 8.23 (br s, 3H), 7.91 (d, J=7.6 Hz, 2H), 7.68-7.77 (m, 3H), 7.58-7.65 (m, 2H), 4.35 (d, J=7.2 Hz, 2H), 4.27 (t, J=6.6 Hz, 1H), 3.81-3.93 (m, 4H); low resolution mass spectrum (ES) m/e 445 [(M+H)⁺, calcd for C₂₅H₂₅N₄O₄: 445]; 96% purity based on HPLC.

### Example 312

### {[2-(3-AMINO-PROPIONYLAMINO)-PHENYLCARBAMOYL]-METHYL}-CARBAMIC ACID 9H-FLUOREN-9-YLMETHYL ESTER

The compound of Example 310 was coupled to 3-tert-Butoxycarbonylamino-propionic acid as described in the method of Example 6 to produce the title compound. ¹H NMR (400 MHz, DMSO-d₆) δ 9.78 (s, 1H), 9.52 (s, 1H), 7.88-7.98 (m, 5H), 7.71-7.77 (m, 3H), 7.63 (d, J=8.0 Hz, 1H), 7.55 (d, J=8.0 Hz, 1H), 7.44 (t, J=7.6 Hz, 2H), 7.35 (t, J=7.4 Hz, 2H), 7.15-7.21 (m, 2H), 4.35 (d, J=6.8 Hz, 2H), 4.27 (t, J=6.6 Hz, 1H), 3.88 (d, J=6.0 Hz, 2H), 3.07-3.14 (m, 2H), 2.80 (t, J=6.6 Hz, 2H); low resolution mass spectrum (ES) m/e 459 [(M+H)⁺, calcd for C₂₆H₂₇N₄O₄: 459]; 94% purity based on HPLC.

### Example 313

### (S)-[(6-AMINO-HEXYLCARBAMOYL)-PHENYL-METHYL]-CARBAMIC ACID 9H-FLUOREN-9-YLMETHYL ESTER

(S)-[(9H-Fluoren-9-ylmethoxycarbonylamino)]-phenyl-acetic acid was coupled to (6-Amino-hexyl)-carbamic acid tert-butyl ester as described in the method of Example 6. Purification by HPLC produced the title compound. 1H NMR (400 MHz, DMSO-d6) δ 7.86 (d, J=7.5 Hz, 2H), 7.72 (d, J=7.5 Hz, 2H), 7.44-7.24 (m, 9H), 5.15 (s, 1H), 4.29-4.12 (m, 3H), 3.02 (m, 2H), 2.69 (t, J=7.9 Hz, 2H), 1.51-1.28 (m, 4H), 1.27-1.08 (m, 4H); Low resolution mass spectrum (ES) m/e 472.2 [(M+H)⁺, calcd for C29H33N3O3: 472.6]; 98% purity based on HPLC.

### Example 314

### (S)-[(4-AMINOMETHYL-BENZYLCARBAMOYL)-PHENYL-METHYL]-CARBAMIC ACID 9H-FLUOREN-9-YLMETHYL ESTER

(S)-[(9H-Fluoren-9-ylmethoxycarbonylamino)]-phenyl-acetic acid was coupled to (4-Aminomethyl-benzyl)-carbamic acid tert-butyl ester as described in the method of Example 6. Purification by HPLC produced the title compound. 1H NMR (400 MHz, DMSO-d6) 7.87 (d, J=7.9 Hz, 2H), 7.74 (d, J=7.9 Hz, 2H), 7.48-7.24 (m, 11H), 7.15 (d, J=8.5 Hz, 2H), 5.24 (s, 1H), 4.33-4.13 (m, 5H), 3.95 (s, 2H); Low resolution mass spectrum (ES) m/e 492.2 [(M+H)⁺, calcd for C31H29N3O3: 492.6]; 100% purity based on HPLC.

### Example 315

### (S)-[(4-AMINOMETHYL-PHENYLCARBAMOYL)-PHENYL-METHYL]-CARBAMIC ACID 9H-FLUOREN-9-YLMETHYL ESTER

(S)-[(9H-Fluoren-9-ylmethoxycarbonylamino)]-phenyl-acetic acid was coupled to (4-Amino-benzyl)-carbamic acid tert-butyl ester as described in the method of Example 6. Purification produced the title compound. 1H NMR (400 MHz, DMSO-d6) 10.41 (s, 1H), 7.87 (d, J=8.1 Hz, 2H), 7.74 (d, J=7.5 Hz, 2H), 7.59 (d, J=7.9 Hz, 2H), 7.50 (d, J=7.5 Hz, 2H), 7.43-7.25 (m, 9H), 5.38 (s, 1H), 4.29-4.16 (m, 3H), 3.94 (s, 2H); Low resolution mass spectrum (ES) m/e 478.3 [(M+H)⁺, calcd for C30H27N3O3: 478.6]; 100% purity based on HPLC.

### Example 316

### (S)-6-AMINO-2-[3-(4-BENZYLOXY-PHENYL)-UREIDO]-HEXANOIC ACID (5-METHYL-2-PHENYL-2H-PYRAZOL-3-YL)-AMIDE

The title compound was prepared as described in the method of Example 2 with an initial coupling to 5-Methyl-2-phenyl-2H-pyrazol-3-ylamine. ¹H NMR (400 MHz, DMSO-d6) 7.45-7.26 (m, 10H), 7.24 (d, J=9.0 Hz, 2H), 6.89 (d, J=9.0 Hz, 2H), 6.19 (s, 1H), 5.01 (s, 2H), 4.25 (dd, J=3.9, 8.1 Hz, 1H), 2.75-2.63 (m, 2H), 2.18 (s, 3H), 1.67-1.39 (m, 4H), 1.28-1.13 (m, 2H); Low resolution mass spectrum (ES) m/e 527.2 [(M+H)⁺, calcd for C30H34N6O3: 527.6]; 100% purity based on HPLC.

### Example 317

### (S)-6-AMINO-2-[3-(4-BENZYLOXY-PHENYL)-UREIDO]-HEXANOIC ACID (4-IMIDAZOL-1-YL-PHENYL)-AMIDE

The title compound was prepared as described in the method of Example 2 with an initial coupling to 4-Imidazol-1-yl-phenylamine. 1H NMR (400 MHz, DMSO-d6) 9.34 (s, 1H), 8.09 (s, 1H), 7.81 (d, J=8.8 Hz, 2H), 7.75-7.67 (m, 3H), 7.42-7.22 (m, 7H), 6.88 (d, J=9.0 Hz, 2H), 5.01 (s, 2H), 4.34 (dd, J=5.7, 8.3 Hz, 1H), 2.76 (t, J=7.0 Hz, 2H), 1.82-1.25 (m, 6H); Low resolution mass spectrum (ES) m/e 513.2 [(M+H)⁺, calcd for C29H32N6O3: 513.6]; 100% purity based on HPLC.

### Example 318

### (S)-1-[5-AMINO-1-(MORPHOLINE-4-CARBONYL)-PENTYL]-3-(4-BENZYLOXY-PHENYL)-UREA

The title compound was prepared as described in the method of Example 2 with an initial coupling to Morpholine. 1H NMR (400 MHz, DMSO-d6) 7.43-7.18 (m, 7H), 6.87 (d, J=8.8 Hz, 2H), 5.00 (s, 2H), 4.62 (dd, J=4.6, 8.6 Hz, 0.8H), 4.13 (dd, J=5.0, 8.3 Hz, 0.4H), 3.58-3.36 (m, 8H), 2.79-2.69 (m, 2H), 1.68-1.19 (m, 6H); Low resolution mass spectrum (ES) m/e 441.2 [(M+H)⁺, calcd for C24H32N4O4: 441.5]; 75% purity based on HPLC.

### Example 319

### (S)—N-(6-AMINO-HEXYL)-2-[3-(4-BENZYLOXY-PHENYL)-UREIDO]-2-PHENYL-ACETAMIDE

(S)-[(9H-Fluoren-9-ylmethoxycarbonylamino)]-phenyl-acetic acid was coupled to (6-Amino-hexyl)-carbamic acid tert-butyl ester then coupled to 1-Benzyloxy-4-isocyanato-benzene as described in the method of Example 2. Purification produced the title compound. 1H NMR (400 MHz, DMSO-d6) 7.46-7.17 (m, 12H), 6.86 (d, J=8.8 Hz, 2H), 5.26 (s, 1H), 5.00 (s, 2H), 3.02 (dd, J=7.0, 12.1 Hz, 2H), 2.69 (t, J=7.7 Hz, 2H), 1.49-1.10 (m, 8H); Low resolution mass spectrum (ES) m/e 475.1 [(M+H)⁺, calcd for C28H34N4O3: 474.6]; 97% purity based on HPLC.

### Example 320

### (R)—N-(6-AMINO-HEXYL)-2-[3-(4-BENZYLOXY-PHENYL)-UREIDO]-2-PHENYL-ACETAMIDE

(R)-[(9H-Fluoren-9-ylmethoxycarbonylamino)]-phenyl-acetic acid was coupled to (6-Amino-hexyl)-carbamic acid tert-butyl ester then coupled to 1-Benzyloxy-4-isocyanato-benzene as described in the method of Example 2. Purification produced the title compound. 1H NMR (400 MHz, DMSO-d6) 7.42-7.19 (m, 12H), 6.87 (d, J=8.8 Hz, 2H), 5.27 (s, 1H), 5.00 (s, 2H), 3.09-2.96 (m, 2H), 2.75-2.63 (m, 2H), 1.51-1.28 (m, 4H), 1.28-1.08 (m, 4H); Low resolution mass spectrum (ES) m/e 475.1 [(M+H)⁺, calcd for C28H34N4O3: 474.6]; 99% purity based on HPLC.

### Example 321

### (S)-6-AMINO-2-[3-(4-BENZYLOXY-PHENYL)-UREIDO]-HEXANOIC ACID INDAN-4-YLAMIDE

The title compound was prepared as described in the method of Example 2 with an initial coupling to Indan-4-ylamine. 1H NMR (400 MHz, DMSO-d6) 7.96 (d, J=7.2 Hz, 1H), 7.74 (d, J=7.2 Hz, 1H), 7.48-7.21 (m, 10H), 6.88 (d, J=9.0 Hz, 2H), 5.01 (s, 2H), 4.49 (dd, J=5.9, 7.9 Hz, 1H), 2.77 (t, J=7.7 Hz, 2H), 1.87-1.49 (m, 4H), 1.48-1.29 (m, 2H); Low resolution mass spectrum (ES) m/e 487.1 [(M+H)⁺, calcd for C29H34N4O3: 487.6]; 92% purity based on HPLC.

### Example 322

### (S)-5-(2-AMINO-ACETYLAMINO)-2-[3-(4-BENZYLOXY-PHENYL)-UREIDO]-PENTANOIC ACID P-TOLYLAMIDE

4-Methyl-phenylamine was coupled to 5-tert-Butoxycarbonylamino-2-(9H-fluoren-9-ylmethoxycarbonylamino)-pentanoic acid as described in the method of Example 6. Coupling to tert-Butoxycarbonylamino-acetic acid, and then to 1-Benzyloxy-4-isocyanato-benzene as described in the method of Example 2 produced the title compound. 1H NMR (400 MHz, DMSO-d6) δ 7.45 (d, J=8.3 Hz, 2H), 7.42-7.26 (m, 5H), 7.24 (d, J=9.3 Hz, 2H), 7.10 (d, J=8.1 Hz, 2H), 6.87 (d, J=9.3 Hz, 2H), 5.00 (s, 2H), 4.33 (t, J=7.5 Hz, 1H), 3.47 (s, 2H), 3.13 (t, J=6.8 Hz, 2H), 2.23 (s, 3H), 1.78-1.35 (m, 4H); Low resolution mass spectrum (ES) m/e 504.1 [(M+H)⁺, calcd for C₂₈H₃₃N₅O₄: 504.6]; 97% purity based on HPLC.

### Example 323

### 2-[1-(4-AMINO-BUTYL)-3-(4-BENZYLOXY-PHENYL)-UREIDO]-N-(4-CYCLOHEXYL-PHENYL)-ACETAMIDE

[(4-tert-Butoxycarbonylamino-butyl)-(9H-fluoren-9-ylmethoxycarbonyl)-amino]-acetic acid was coupled to 4-cyclohexyl phenylamine then to 1-Benzyloxy-4-isocyanato-benzene as described in the method of Example 2. Purification by HPLC produced the title compound. Low resolution mass spectrum (ES) m/e 529 [(M+H)⁺, calcd for C₃₂H₄₁N₄O₃: 529]; 98% purity based on HPLC.

### Example 324

### 2-[1-(4-AMINO-BUTYL)-3-(4-STYRYL-PHENYL)-UREIDO]-N-(4-CYCLOHEXYL-PHENYL)-ACETAMIDE

[(4-tert-Butoxycarbonylamino-butyl)-(9H-fluoren-9-ylmethoxycarbonyl)-amino]-acetic acid was coupled to 4-cyclohexyl phenylamine then to 1-Isocyanato-4-styryl-benzene as described in the method of Example 2. Purification by HPLC produced the title compound. Low resolution mass spectrum (ES) m/e 525 [(M+H)⁺, calcd for C₃₃H₄₁N₄O₂: 525]; 99% purity based on HPLC.

### Example 325

### ({2-[2-(9H-FLUOREN-9-YLMETHOXYCARBONYLAMINO)-ACETYLAMINO]-BENZYLCARBAMOYL}-METHYL)-CARBAMIC ACID TERT-BUTYL ESTER

(9H-Fluoren-9-ylmethoxycarbonylamino)-acetic acid was coupled to (2-Amino-benzyl)-carbamic acid tert-butyl ester as described in the method of Example 6, and then coupled to tert-Butoxycarbonylamino-acetic acid as described for Intermediate #1 of Example 1. Purification by HPLC produced the title compound. Low resolution mass spectrum (ES) m/e 560 [(M+H)⁺, calcd for C₃₁H₃₅N₄O₆: 560]; 79% purity based on HPLC.

### Scheme (II) Compounds

### Example 326

### (S)-6-AMINO-2-[2-(BIPHENYL-4-YLOXY)-ACETYLAMINO]-HEXANOIC ACID P-TOLYLAMIDE

Prepared by solid-phase as described in Scheme 2: Low resolution mass spectrum (ES) m/e 446 [(M+H)⁺, calcd for C₂₇H₃₂N₃O₃: 446]; 90% purity based on HPLC.

### Example 327

### (S)-6-AMINO-2-[2-(4-IODO-PHENOXY)-ACETYLAMINO]-HEXANOIC ACID P-TOLYLAMIDE

Prepared by solid-phase as described in Scheme 2: Low resolution mass spectrum (ES) m/e 496 [(M+H)⁺, calcd for C₂₁H₂₇IN₃O₃: 496]; 93% purity based on HPLC.

### Example 328

### (S)-6-AMINO-2-(2-PHENOXY-ACETYLAMINO)-HEXANOIC ACID P-TOLYLAMIDE

Prepared by solid-phase as described in Scheme 2. Low resolution mass spectrum (ES) m/e 370 [(M+H)⁺, calcd for C₂₁H₂₈N₃O₃: 470]; 97% purity based on HPLC.

### Example 329

### (S)-6-AMINO-2-[2-(4-FLUORO-PHENOXY)-ACETYLAMINO]-HEXANOIC ACID P-TOLYLAMIDE

Prepared by solid-phase as described in Scheme 2. Low resolution mass spectrum (ES) m/e 388 [(M+H)⁺, calcd for C₂₁H₂₇FN₃O₃: 388]; 98% purity based on HPLC.

### Example 330

### (S)-6-AMINO-2-[2-(3-CHLORO-PHENOXY)-ACETYLAMINO]-HEXANOIC ACID P-TOLYLAMIDE

Prepared by solid-phase as described in Scheme 2. Low resolution mass spectrum (ES) m/e 404 [(M+H)⁺, calcd for C₂₁H₂₇ClN₃O₃: 404]; 95% purity based on HPLC.

### Example 331

### (S)-6-AMINO-2-[2-(4-CHLORO-PHENOXY)-ACETYLAMINO]-HEXANOIC ACID P-TOLYLAMIDE

Prepared by solid-phase as described in Scheme 2. Low resolution mass spectrum (ES) m/e 404 [(M+H)⁺, calcd for C₂₁H₂₇ClN₃O₃: 404]; 98% purity based on HPLC.

### Example 332

### (S)-6-AMINO-2-[2-(3-TRIFLUOROMETHYL-PHENOXY)-ACETYLAMINO]-HEXANOIC ACID P-TOLYLAMIDE

Prepared by solid-phase as described in Scheme 2. Low resolution mass spectrum (ES) m/e 438 [(M+H)⁺, calcd for C₂₂H₂₇F₃N₃O₃: 438]; 94% purity based on HPLC.

### Example 333

### (S)-6-AMINO-2-[2-(4-PHENOXY-PHENOXY)-ACETYLAMINO]-HEXANOIC ACID P-TOLYLAMIDE

Prepared by solid-phase as described in Scheme 2. Low resolution mass spectrum (ES) m/e 462 [(M+H)⁺, calcd for C₂₇H₃₁N₃O₄: 462]; 88% purity based on HPLC.

### Example 334

### (S)-6-AMINO-2-(2-PHENETHYLAMINO-ACETYLAMINO)-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 85% purity based on HPLC.

### Example 335

### (S)-6-AMINO-2-{2-[2-(1H-INDOL-3-YL)-ETHYLAMINO]-ACETYLAMINO}-HEXANOIC ACID P-TOLYLAMIDE

Prepared by solid-phase as described in Scheme 2. Low resolution mass spectrum (ES) m/e 436 [(M+H)⁺, calcd for C₂₅H₃₄N₅O₂: 436]; 78% purity based on HPLC.

### Example 336

### (S)-6-AMINO-2-[2-(INDAN-2-YLAMINO)-ACETYLAMINO]-HEXANOIC ACID P-TOLYLAMIDE

Prepared by solid-phase as described in Scheme 2. Low resolution mass spectrum (ES) m/e 409 [(M+H)⁺, calcd for C₂₄H₃₃N₄O₂: 409]; 93% purity based on HPLC.

### Example 337

### (S)-6-AMINO-2-(2-BENZYLAMINO-ACETYLAMINO)-HEXANOIC ACID P-TOLYLAMIDE

Prepared by solid-phase as described in Scheme 2. Low resolution mass spectrum (ES) m/e 383 [(M+H)⁺, calcd for C₂₂H₃₁N₄O₂: 383]; 81% purity based on HPLC.

### Example 338

### (S)-6-AMINO-2-[2-(2-METHYL-BENZYLAMINO)-ACETYLAMINO]-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 91% purity based on HPLC.

### Example 339

### (S)-6-AMINO-2-[2-(3-METHYL-BENZYLAMINO)-ACETYLAMINO]-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 86% purity based on HPLC.

### Example 340

### (S)-6-AMINO-2-[2-(4-METHYL-BENZYLAMINO)-ACETYLAMINO]-HEXANOIC ACID P-TOLYLAMIDE

Prepared by solid-phase as described in Scheme 2. Low resolution mass spectrum (ES) m/e 397 [(M+H)⁺, calcd for C₂₃H₃₃N₄O₂: 397]; 88% purity based on HPLC.

### Example 341

### (S)-6-AMINO-2-[2-(2-METHOXY-BENZYLAMINO)-ACETYLAMINO]-HEXANOIC ACID P-TOLYLAMIDE

Prepared by solid-phase as described in Scheme 2. Low resolution mass spectrum (ES) m/e 413 [(M+H)⁺, calcd for C₂₃H₃₃N₄O₃: 413]; 83% purity based on HPLC.

### Example 342

### (S)-6-AMINO-2-[2-(3-METHOXY-BENZYLAMINO)-ACETYLAMINO]-HEXANOIC ACID P-TOLYLAMIDE

Prepared by solid-phase as described in Scheme 2. Low resolution mass spectrum (ES) m/e 413 [(M+H)⁺, calcd for C₂₃H₃₃N₄O₃: 413]; 82% purity based on HPLC.

### Example 343

### (S)-6-AMINO-2-[2-(4-METHOXY-BENZYLAMINO)-ACETYLAMINO]-HEXANOIC ACID P-TOLYLAMIDE

Prepared by solid-phase as described in Scheme 2. Low resolution mass spectrum (ES) m/e 413 [(M+H)⁺, calcd for C₂₃H₃₃N₄O₃: 413]; 86% purity based on HPLC.

### Example 344

### (S)-6-AMINO-2-[2-(2-FLUORO-BENZYLAMINO)-ACETYLAMINO]-HEXANOIC ACID P-TOLYLAMIDE

Prepared by solid-phase as described in Scheme 2. Low resolution mass spectrum (ES) m/e 401 [(M+H)⁺, calcd for C₂₂H₃₀FN₄O₂: 401]; 90% purity based on HPLC.

### Example 345

### (S)-6-AMINO-2-[2-(3-FLUORO-BENZYLAMINO)-ACETYLAMINO]-HEXANOIC ACID P-TOLYLAMIDE

Prepared by solid-phase as described in Scheme 2. Low resolution mass spectrum (ES) m/e 401 [(M+H)⁺, calcd for C₂₂H₃₀FN₄O₂: 401]; 85% purity based on HPLC.

### Example 346

### (S)-6-AMINO-2-[2-(4-FLUORO-BENZYLAMINO)-ACETYLAMINO]-HEXANOIC ACID P-TOLYLAMIDE

Prepared by solid-phase as described in Scheme 2. Low resolution mass spectrum (ES) m/e 401 [(M+H)⁺, calcd for C₂₂H₃₀FN₄O₂: 401]; 87% purity based on HPLC.

### Example 347

### (S)-6-AMINO-2-[2-(2-CHLORO-BENZYLAMINO)-ACETYLAMINO]-HEXANOIC ACID P-TOLYLAMIDE

Prepared by solid-phase as described in Scheme 2. Low resolution mass spectrum (ES) m/e 417 [(M+H)⁺, calcd for C₂₂H₃₀ClN₄O₂: 417]; 87% purity based on HPLC.

### Example 348

### (S)-6-AMINO-2-[2-(3-CHLORO-BENZYLAMINO)-ACETYLAMINO]-HEXANOIC ACID P-TOLYLAMIDE

Prepared by solid-phase as described in Scheme 2. Low resolution mass spectrum (ES) m/e 417 [(M+H)⁺, calcd for C₂₂H₃₀ClN₄O₂: 417]; 90% purity based on HPLC.

### Example 349

### (S)-6-AMINO-2-[2-(4-CHLORO-BENZYLAMINO)-ACETYLAMINO]-HEXANOIC ACID P-TOLYLAMIDE

Prepared by solid-phase as described in Scheme 2. Low resolution mass spectrum (ES) m/e 417 [(M+H)⁺, calcd for C₂₂H₃₀ClN₄O₂: 417]; 85% purity based on HPLC.

### Example 350

### (S)-6-AMINO-2-[2-(2-TRIFLUOROMETHYL-BENZYLAMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₃H₃₀F₃N₄O₂: 451]; 87% purity based on HPLC.

### Example 351

### (S)-6-AMINO-2-[2-(3-TRIFLUOROMETHYL-BENZYLAMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₃H₃₀F₃N₄O₂: 451]; 84% purity based on HPLC.

### Example 352

### (S)-6-AMINO-2-[2-(4-TRIFLUOROMETHYL-BENZYLAMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₃H₃₀F₃N₄O₂: 451]; 86% purity based on HPLC.

### Example 353

### (S)-6-AMINO-2-[2-(BENZYL-METHYL-AMINO)-ACETYLAMINO]-HEXANOIC ACID P-TOLYLAMIDE

Prepared by solid-phase as described in Scheme 2: Low resolution mass spectrum (ES) m/e 397 [(M+H)⁺, calcd for C₂₃H₃₃N₄O₂: 397]; 95% purity based on HPLC.

### Example 354

### (S)-6-AMINO-2-[2-(CYCLOHEXYL-PHENYL-AMINO)-ACETYLAMINO]-HEXANOIC ACID P-TOLYLAMIDE

Prepared by solid-phase as described in Scheme 2: Low resolution mass spectrum (ES) m/e 451 [(M+H)⁺, calcd for C₂₇H₃₉N₄O₂: 451]; 90% purity based on HPLC.

### Example 355

### (S)-6-AMINO-2-(2-M-TOLYLAMINO-ACETYLAMINO)-HEXANOIC ACID P-TOLYLAMIDE

Prepared by solid-phase as described in Scheme 2: Low resolution mass spectrum (ES) m/e 383 [(M+H)⁺, calcd for C₂₂H₃₁N₄O₂: 383]; 95% purity based on HPLC.

### Example 356

### (S)-6-AMINO-2-(2-P-TOLYLAMINO-ACETYLAMINO)-HEXANOIC ACID P-TOLYLAMIDE

Prepared by solid-phase as described in Scheme 2: Low resolution mass spectrum (ES) m/e 383 [(M+H)⁺, calcd for C₂₂H₃₁N₄O₂: 383]; 89% purity based on HPLC.

### Example 357

### (S)-6-AMINO-2-[2-(2-METHOXY-PHENYLAMINO)-ACETYLAMINO]-HEXANOIC ACID P-TOLYLAMIDE

Prepared by solid-phase as described in Scheme 2: Low resolution mass spectrum (ES) m/e 399 [(M+H)⁺, calcd for C₂₂H₃₁N₄O₃: 399]; 84% purity based on HPLC.

### Example 358

### (S)-6-AMINO-2-[2-(3-METHOXY-PHENYLAMINO)-ACETYLAMINO]-HEXANOIC ACID P-TOLYLAMIDE

Prepared by solid-phase as described in Scheme 2: Low resolution mass spectrum (ES) m/e 399 [(M+H)⁺, calcd for C₂₂H₃₁N₄O₃: 399]; 87% purity based on HPLC.

### Example 359

### (S)-6-AMINO-2-[2-(2-FLUORO-PHENYLAMINO)-ACETYLAMINO]-HEXANOIC ACID P-TOLYLAMIDE

Prepared by solid-phase as described in Scheme 2: Low resolution mass spectrum (ES) m/e 387 [(M+H)⁺, calcd for C₂₁H₂₈FN₄O₂: 387]; 88% purity based on HPLC.

### Example 360

### (S)-6-AMINO-2-[2-(3-FLUORO-PHENYLAMINO)-ACETYLAMINO]-HEXANOIC ACID P-TOLYLAMIDE

Prepared by solid-phase as described in Scheme 2: Low resolution mass spectrum (ES) m/e 387 [(M+H)⁺, calcd for C₂₁H₂₈FN₄O₂: 387]; 92% purity based on HPLC.

### Example 361

### (S)-6-AMINO-2-[2-(4-FLUORO-PHENYLAMINO)-ACETYLAMINO]-HEXANOIC ACID P-TOLYLAMIDE

Prepared by solid-phase as described in Scheme 2: Low resolution mass spectrum (ES) m/e 387 [(M+H)⁺, calcd for C₂₁H₂₈FN₄O₂: 387]; 89% purity based on HPLC.

### Example 362

### (S)-6-AMINO-2-[2-(3-CHLORO-PHENYLAMINO)-ACETYLAMINO]-HEXANOIC ACID P-TOLYLAMIDE

Prepared by solid-phase as described in Scheme 2: Low resolution mass spectrum (ES) m/e 403 [(M+H)⁺, calcd for C₂₁H₂₈ClN₄O₂: 403]; 92% purity based on HPLC.

### Example 363

### (S)-6-AMINO-2-[2-(4-CHLORO-PHENYLAMINO)-ACETYLAMINO]-HEXANOIC ACID P-TOLYLAMIDE

Prepared by solid-phase as described in Scheme 2: Low resolution mass spectrum (ES) m/e 403 [(M+H)⁺, calcd for C₂₁H₂₈ClN₄O₂: 403]; 90% purity based on HPLC.

### Example 364

### (S)-6-AMINO-2-[2-(3-TRIFLUOROMETHYL-PHENYLAMINO)-ACETYLAMINO]-HEXANOIC ACID P-TOLYLAMIDE

Prepared by solid-phase as described in Scheme 2: Low resolution mass spectrum (ES) m/e 437 [(M+H)⁺, calcd for C₂₂H₂₈F₃N₄O₂: 438]; 96% purity based on HPLC.

### Example 365

### (S)-6-AMINO-2-[2-(4-TRIFLUOROMETHYL-PHENYLAMINO)-ACETYLAMINO]-HEXANOIC ACID P-TOLYLAMIDE

Prepared by solid-phase as described in Scheme 2: Low resolution mass spectrum (ES) m/e 437 [(M+H)⁺, calcd for C₂₂H₂₈F₃N₄O₂: 438]; 82% purity based on HPLC.

### Example 366

### (S)-6-AMINO-2-[2-(2-PHENOXY-PHENYLAMINO)-ACETYLAMINO]-HEXANOIC ACID P-TOLYLAMIDE

Prepared by solid-phase as described in Scheme 2: Low resolution mass spectrum (ES) m/e 461 [(M+H)⁺, calcd for C₂₇H₃₃N₄O₃: 461]; 85% purity based on HPLC.

### Example 367

### (S)-6-AMINO-2-[2-(3-PHENOXY-PHENYLAMINO)-ACETYLAMINO]-HEXANOIC ACID P-TOLYLAMIDE

Prepared by solid-phase as described in Scheme 2: Low resolution mass spectrum (ES) m/e 461 [(M+H)⁺, calcd for C₂₇H₃₃N₄O₃: 461]; 91% purity based on HPLC.

### Example 368

### (S)-6-AMINO-2-[2-(4-PHENOXY-PHENYLAMINO)-ACETYLAMINO]-HEXANOIC ACID P-TOLYLAMIDE

Prepared by solid-phase as described in Scheme 2: Low resolution mass spectrum (ES) m/e 461 [(M+H)⁺, calcd for C₂₇H₃₃N₄O₃: 461]; 83% purity based on HPLC.

### Example 369

### (S)-6-AMINO-2-[2-(INDAN-5-YLAMINO)-ACETYLAMINO]-HEXANOIC ACID P-TOLYLAMIDE

Prepared by solid-phase as described in Scheme 2: Low resolution mass spectrum (ES) m/e 409 [(M+H)⁺, calcd for C₂₄H₃₃N₄O₂: 409]; 85% purity based on HPLC.

### Example 370

### (S)-3-(9H-FLUOREN-9-YLMETHOXYCARBONYLAMINO)-N-P-TOLYL-SUCCINAMIC ACID

Prepared by solid-phase as described in Scheme 2: Low resolution mass spectrum (ES) m/e 445 [(M+H)⁺, calcd for C₂₆H₂₅N₂O₅: 445]; 95.2% purity based on HPLC.

### Example 371

### (R)-3-(9H-FLUOREN-9-YLMETHOXYCARBONYLAMINO)-N-P-TOLYL-SUCCINAMIC ACID

Prepared by solid-phase as described in Scheme 2: Low resolution mass spectrum (ES) m/e 445 [(M+H)⁺, calcd for C₂₆H₂₅N₂O₅: 445]; 94.8% purity based on HPLC.

### Example 372

### (S)-4-(9H-FLUOREN-9-YLMETHOXYCARBONYLAMINO)-4-P-TOLYLCARBAMOYL-BUTYRIC ACID

Prepared by solid-phase as described in Scheme 2: Low resolution mass spectrum (ES) m/e 459 [(M+H)⁺, calcd for C₂₇H₂₇N₂O₅: 459]; 99% purity based on HPLC.

### Example 373

### (R)-4-(9H-FLUOREN-9-YLMETHOXYCARBONYLAMINO)-4-P-TOLYLCARBAMOYL-BUTYRIC ACID

Prepared by solid-phase as described in Scheme 2: Low resolution mass spectrum (ES) m/e 459 [(M+H)⁺, calcd for C₂₇H₂₇N₂O₅: 459]; 99.2% purity based on HPLC.

### Example 374

### (S)-(3-HYDROXY-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 431 [(M+H)⁺, calcd for C₂₆H₂₇N₂O₄: 431]; 86.6% purity based on HPLC.

### Example 375

### (R)-(2-HYDROXY-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 417 [(M+H)⁺, calcd for C₂₅H₂₅N₂O₄: 417]; 98.2% purity based on HPLC.

### Example 376

### (2+/−,3S)-(2-HYDROXY-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 431 [(M+H)⁺, calcd for C₂₆H₂₇N₂O₄: 431]; 96.1% purity based on HPLC.

### Example 377

### (2+/−,3R)-(2-HYDROXY-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 431 [(M+H)⁺, calcd for C₂₆H₂₇N₂O₄: 431]; 99.3% purity based on HPLC.

### Example 378

### (S)-[2-(1H-INDOL-3-YL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 516 [(M+H)⁺, calcd for C₃₃H₃₀N₃O₃: 516]; 88.2% purity based on HPLC.

### Example 379

### (R)-[2-(1H-INDOL-3-YL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 516 [(M+H)⁺, calcd for C₃₃H₃₀N₃O₃: 516]; 96.7% purity based on HPLC.

### Example 380

### (R)-[2-(4-HYDROXY-PHENYL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 493 [(M+H)⁺, calcd for C₃₁H₂₉N₂O₄: 493]; 98.5% purity based on HPLC.

### Example 381

### (S)-(2-THIOPHEN-2-YL-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 483 [(M+H)⁺, calcd for C₂₉H₂₇N₂O₄S: 483]; 88.2% purity based on HPLC.

### Example 382

### (R)-(3-CARBAMOYL-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 458 [(M+H)⁺, calcd for C₂₇H₂₈N₃O₄: 458]; 98.5% purity based on HPLC.

### Example 383

### (R)-(3-METHYLSULFANYL-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 461 [(M+H)⁺, calcd for C₂₇H₂₉N₂O₃S: 461]; 86.1% purity based on HPLC.

### Example 384

### (R)-[2-(3H-IMIDAZOL-4-YL)-1-P-TOLYLCARBAMOYL-ETHYL]-CARBAMIC ACID 9H-FLUOREN-9-YLMETHYL ESTER

Prepared by solid-phase as described in Scheme 2: Low resolution mass spectrum (ES) m/e 467 [(M+H)⁺, calcd for C₂₈H₂₇F₃N₄O₃: 467]; 96.2% purity based on HPLC.

### Example 385

### (S)-(2-PYRIDIN-3-YL-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 478 [(M+H)⁺, calcd for C₃₀H₂₈N₃O₃: 478]; 98.9% purity based on HPLC.

### Example 386

### (S)-6-AMINO-2-(3,4-DIMETHOXY-BENZENESULFONYLAMINO)-HEXANOIC ACID P-TOLYLAMIDE

Prepared by solid-phase as described in Scheme 2: Low resolution mass spectrum (ES) m/e 436 [(M+H)⁺, calcd for C₂₁H₃₀N₃O₅S: 436]; 100% purity based on HPLC.

### Example 387

### (S)-6-AMINO-2-BENZENESULFONYLAMINO-HEXANOIC ACID P-TOLYLAMIDE

Prepared by solid-phase as described in Scheme 2: Low resolution mass spectrum (ES) m/e 376 [(M+H)⁺, calcd for C₁₉H₂₅N₃O₃S: 376]; 100% purity based on HPLC.

### Example 388

### (S)-6-AMINO-2-(4-BENZENESULFONYL-THIOPHENE-2-SULFONYLAMINO)-HEXANOIC ACID P-TOLYLAMIDE

Prepared by solid-phase as described in Scheme 2: Low resolution mass spectrum (ES) m/e 522 [(M+H)⁺, calcd for C₂₃H₂₈N₃O₅S₃: 522]; 85% purity based on HPLC.

### Example 389

### (S)-6-AMINO-2-(5-BENZENESULFONYL-THIOPHENE-2-SULFONYLAMINO)-HEXANOIC ACID P-TOLYLAMIDE

Prepared by solid-phase as described in Scheme 2: Low resolution mass spectrum (ES) m/e 522 [(M+H)⁺, calcd for C₂₃H₂₈N₃O₅S₃: 522]; 88% purity based on HPLC.

### Example 390

### (S)-6-AMINO-2-(4-BUTOXY-BENZENESULFONYLAMINO)-HEXANOIC ACID P-TOLYLAMIDE

Prepared by solid-phase as described in Scheme 2; Low resolution mass spectrum (ES) m/e 448 [(M+H)⁺, calcd for C₂₃H₃₄N₃O₄S: 448]; 92% purity based on HPLC.

### Example 391

### (S)-6-AMINO-2-(4-CHLORO-BENZENESULFONYLAMINO)-HEXANOIC ACID P-TOLYLAMIDE

Prepared by solid-phase as described in Scheme 2: Low resolution mass spectrum (ES) m/e 410 [(M+H)⁺, calcd for C₁₉H₂₅N₃O₃S: 410]; 100% purity based on HPLC.

### Example 392

### (S)-6-AMINO-2-(3,4-DICHLORO-BENZENESULFONYLAMINO)-HEXANOIC ACID P-TOLYLAMIDE

Prepared by solid-phase as described in Scheme 2: Low resolution mass spectrum (ES) m/e 444 [(M+H)⁺, calcd for C₁₉H₂₄Cl₂N₃O₃S: 444]; 94% purity based on HPLC.

### Example 393

### (S)-6-AMINO-2-(2,4-DICHLORO-BENZENESULFONYLAMINO)-HEXANOIC ACID P-TOLYLAMIDE

Prepared by solid-phase as described in Scheme 2: Low resolution mass spectrum (ES) m/e 444 [(M+H)⁺, calcd for C₁₉H₂₄Cl₂N₃O₃S: 444]; 86% purity based on HPLC.

### Example 394

### (S)-6-AMINO-2-(3,5-DICHLORO-BENZENESULFONYLAMINO)-HEXANOIC ACID P-TOLYLAMIDE

Prepared by solid-phase as described in Scheme 2: Low resolution mass spectrum (ES) m/e 444 [(M+H)⁺, calcd for C₁₉H₂₄Cl₂N₃O₃S: 444]; 93% purity based on HPLC.

### Example 395

### (S)-6-AMINO-2-(2,5-DICHLORO-THIOPHENE-3-SULFONYLAMINO)-HEXANOIC ACID P-TOLYLAMIDE

Prepared by solid-phase as described in Scheme 2; Low resolution mass spectrum (ES) m/e 450 [(M+H)⁺, calcd for C₁₇H₂₂Cl₂N₃O₃S: 450]; 88% purity based on HPLC.

### Example 396

### (S)-6-AMINO-2-(TOLUENE-4-SULFONYLAMINO)-HEXANOIC ACID P-TOLYLAMIDE

Prepared by solid-phase as described in Scheme 2: Low resolution mass spectrum (ES) m/e 390 [(M+H)⁺, calcd for C₂₀H₂₈N₃O₃S: 390]; 100% purity based on HPLC.

### Example 397

### (S)-6-AMINO-2-(4-TRIFLUOROMETHYL-BENZENESULFONYLAMINO)-HEXANOIC ACID P-TOLYLAMIDE

Prepared by solid-phase as described in Scheme 2: Low resolution mass spectrum (ES) m/e 444 [(M+H)⁺, calcd for C₂₀H₂₅F₃N₃O₃S: 444]; 91% purity based on HPLC.

### Example 398

### (S)-6-AMINO-2-(BENZO[1,2,5]OXADIAZOLE-4-SULFONYLAMINO)-HEXANOIC ACID P-TOLYLAMIDE

Prepared by solid-phase as described in Scheme 2: Low resolution mass spectrum (ES) m/e 418 [(M+H)⁺, calcd for C₁₉H₂₄N₅O₄S: 418]; 92% purity based on HPLC.

### Example 399

### (S)-6-AMINO-2-(THIOPHENE-3-SULFONYLAMINO)-HEXANOIC ACID P-TOLYLAMIDE

Prepared by solid-phase as described in Scheme 2: Low resolution mass spectrum (ES) m/e 382 [(M+H)⁺, calcd for C₁₇H₂₄N₃O₃S₂: 382]; 98% purity based on HPLC.

### Example 400

### (R)-(3-AMINO-1-P-TOLYLCARBAMOYL-PROPYL)-CARBAMIC ACID 9H-FLUOREN-9-YLMETHYL ESTER

Prepared by solid-phase as described in Scheme 2: Low resolution mass spectrum (ES) m/e 430 [(M+H)⁺, calcd for C₂₆H₂₈N₃O₃: 430]; 100% purity based on HPLC.

### Example 401

### (R)-(2-AMINO-1-P-TOLYLCARBAMOYL-ETHYL)-CARBAMIC ACID 9H-FLUOREN-9-YLMETHYL ESTER

Prepared by solid-phase as described in Scheme 2: Low resolution mass spectrum (ES) m/e 416 [(M+H)⁺, calcd for C₂₅H₂₆N₃O₃: 416]; 100% purity based on HPLC.

### Example 402

### (S)-5-(9H-FLUOREN-9-YLMETHOXYCARBONYLAMINO)-5-P-TOLYLCARBAMOYL-PENTANOIC ACID

Prepared by solid-phase as described in Scheme 2: Low resolution mass spectrum (ES) m/e 473 [(M+H)⁺, calcd for C₂₈H₂₈N₂O₅: 473]; 91% purity based on HPLC.

### Example 403

### (S)-(5-AMINO-1-P-TOLYLCARBAMOYL-PENTYL)-CARBAMIC ACID BENZYL ESTER

Prepared by solid-phase as described in Scheme 2: Low resolution mass spectrum (ES) m/e 370 [(M+H)⁺, calcd for C₂₁H₂₈N₃O₃: 370]; 100% purity based on HPLC.

### Example 404

### In Vitro Activity—Calf and Human Polymerase-Alpha Inhibition

Calf polymerase alpha and human polymerase alpha investigations were performed with a kit available from Replizyme Ltd (Heslington, York, UK). Percent inhibition results from single point screening are provided in Table 1 and dose responses are provided in Table 2.

TABLE 1
% Inhibition at 10 μM (Calf Polymerase alpha)

| Compound | % Inhibition |
|---|---|
| 210 | 104 |
| 305 | 104 |
| 142 | 103 |
| 117 | 102 |
| 119 | 102 |
| 121 | 102 |
| 145 | 102 |
| 146 | 102 |
| 163 | 102 |
| 166 | 102 |
| 262 | 102 |
| 81 | 101 |
| 278 | 101 |
| 321 | 101 |
| 120 | 100 |
| 125 | 100 |
| 366 | 100 |
| 105 | 100 |
| 173 | 99 |
| 126 | 98 |
| 212 | 97 |
| 33 | 95 |
| 40 | 95 |
| 204 | 94 |
| 94 | 93 |
| 324 | 92.5 |
| 74 | 92 |
| 98 | 89 |
| 206 | 87 |
| 247 | 87 |
| 108 | 84 |
| 124 | 79 |
| 189 | 76 |
| 103 | 72 |
| 50 | 71 |
| 319 | 71 |
| 176 | 68 |
| 249 | 67 |
| 240 | 63 |
| 268 | 62.7 |
| 110 | 62.5 |
| 225 | 62 |
| 248 | 61 |
| 172 | 60 |
| 250 | 57 |
| 92 | 56.5 |
| 261 | 55 |
| 70 | 50 |
| 246 | 46.6 |
| 127 | 45 |
| 313 | 43 |
| 264 | 41 |
| 199 | 39.7 |
| 184 | 38 |
| 264 | 35 |
| 187 | 33.5 |
| 263 | 31 |
| 281 | 30.5 |
| 53 | 22 |
| 223 | 22 |
| 294 | 19 |
| 181 | 18.5 |
| 20 | 18 |
| 114 | 17 |
| 297 | 16.5 |
| 299 | 16 |
| 279 | 15 |
| 22 | 14 |
| 52 | 13 |
| 21 | 12 |
| 302 | 5 |
| 243 | 0 |

As shown in Table 1, the compounds of this disclosure inhibit calf polymerase alpha at a physiologically relevant concentration of only 10 μM. Thus, these compounds could be used to treat a hyperproliferative disease or disorder by inhibiting polymerase alpha.

TABLE 2
IC₅₀ against Calf and Human Polymerase Activity

| Compound | Calf IC₅₀ (μM) | Human IC₅₀ (μM) |
|---|---|---|
| 119 | 1.5 | 1.5 |
| 125 | 2.8 | 3 |
| 228 | 5.5 | NT* |
| 368 | 6 | NT |
| 172 | 7.3 | NT |
| 199 | 7.9 | NT |
| 281 | 9.2 | NT |
| 246 | 9.8 | 8 |
| 2 | 13 | NT |
| 264 | 16 | NT |
| 297 | 26 | 10 |
| 299 | >30 | 68 |
| 302 | >30 | 72 |

*NT means not tested.

After several doses of a compound were tested, the IC₅₀ was calculated, which represents the concentration of compound that inhibits 50% of the measurable polymerase activity in Huh-7 cells as compared to an untreated control. As shown in Table 2, the IC₅₀s of the test compounds varied from about 1.5 μM to >30 μM for calf polymerase and 1.5 μM to about 72 μM for human polymerase.

### Example 405

### In Vitro Activity—Polymerase-Alpha and Polymerase Gamma Inhibition

To test for selectivity, an enzymatic assay of activity in human polymerase alpha and human polymerase gamma was performed using a homotemplate assay format (Replizyme Ltd., Heslington, York, UK). The IC₅₀ was measured by testing four different concentrations of a selected compound as compared to an untreated control. The dose response results for each polymerase type are provided in Table 3.

TABLE 3
IC₅₀ against Human Polymerase alpha and gamma

| Compound | Alpha IC₅₀ (μM) | Gamma IC₅₀ (μM) |
|---|---|---|
| 173 | <4 | 16 |
| 297 | <4 | 13 |
| 2 | <4 | 27 |
| 199 | ~4 | 32 |

As is evident from Table 3, the test compounds showed greater activity against polymerase alpha as compared to polymerase gamma (i.e., about 4-fold to about 8-fold greater activity). Still, these compounds showed good activity against polymerase gamma—having IC₅₀s that ranged from about 16 μM to about 32 μM for human polymerase. In addition, the compounds of this disclosure lack inhibitory activity against polymerase beta (data not shown). Thus, the selective nature of these inhibitors indicates that toxicity or severe side effects may be minimized at a therapeutically effective dose.

### Example 406

### Cytotoxicity Huh-7 Cells

Cytotoxicity of compounds for use in treating hyperproliferative diseases or disorders was tested in Huh-7 cells using tetrazolium salt MTS combined with the electron acceptor PMS in a 96-well format. Six doses of each compound was tested, starting at about 1.2 μM to 0.4 μM serially diluted in a final 1% DMSO solution in DMEM/F12 media. Terfenadine was used as a positive control. Cells were seeded at 2.5×10⁵ cells/mL in each well of the 96-well plates in a final volume of 0.1 mL, and then incubated at 37° C. in the presence of 5% CO₂ overnight. Compounds (in media) were added the following day and the treated cells were incubated another 3-days at 37° C. in the presence of 5% CO₂. After the 3-day incubation, 20 μL of MTS/PMS (3-(4,5-dimethylthiazol-2-yl)-5-(3-carboxymethoxy-phenyl)-2-(4-sulfophenyl)-2H-tetrazolium) was added to each well. After about a one to four hour incubation of MTS/PMS at 37° C. and 5% CO₂—that is, until an OD₄₉₀ absorbance of ≧0.6 for the wells containing cells with media only, the level of cytotoxicity at each concentration was calculated. The level of compound required to cause 50% of maximum cytotoxicity was determined using curve fitting and is denoted as the CC₅₀. Dose response results from the cytotoxicity assay (CC₅₀) are provided in Table 4 in comparison with IC₅₀ data (from Table 2)

TABLE 4
IC₅₀ and CC₅₀ for Selected Compounds

| Compound | Calf IC₅₀ (μM) | Huh-7 CC₅₀ (μM) |
|---|---|---|
| 119 | 1.5 | 12.2 |
| 125 | 2.8 | 9.7 |
| 228 | 5.5 | 36.7 |
| 368 | 6 | 58 |
| 172 | 7.3 | 26 |
| 199 | 7.9 | 53 |
| 281 | 9.2 | NT |
| 246 | 9.8 | 45.9 |
| 2 | 13 | 22.2 |
| 264 | 16 | 26.6 |
| 297 | 26 | 9.9 |
| 299 | >30 | >100 |
| 302 | >30 | >100 |

Table 4 shows that the compounds of this disclosure are effective at inhibiting polymerase at doses well below those doses that would result in cytotoxicity and, therefore, would be expected to be effective at clinically relevant doses.

### Example 407

### Activity in Cancer Cell Lines

Compounds 105 and 173 were tested in single and multiple doses in the National Cancer Institute (NCI) In Vitro Cell Line Screening Project (IVCLSP). Compounds were solubilized in DMSO at 400-fold the desired final maximum test concentration and stored frozen prior to use.

Cell Preparation: All human tumor cell lines of the cancer screening panel were grown in RPMI 1640 medium containing 5% fetal bovine serum and 2 mM L-glutamine. Cells were inoculated into 96 well microtitre plates in 100 μL media at plating densities ranging from 5000 to 40000 cells/well depending on the doubling times of individual cell lines. After cell inoculation, the microtitre plates were incubated at 37° C., 5% CO₂, 95% air, and 100% relative humidity for 24 h prior to addition of drug.

Assay: After 24 hours, two plates of each cell line were fixed in situ with TCA, to represent a measurement of the cell population for each cell line at the time of drug addition (Tz). At the time of drug addition, an aliquot of frozen drug concentrate was thawed and diluted to twice the desired maximum test concentration of 100 μM with complete medium containing 50 μg/mL gentamicin. Additional four, 10-fold or ½ log serial dilutions were made to provide a total of five drug concentrations plus control. Aliquots of 100 μL of these different drug dilutions were added to the appropriate microtitre well already containing 100 μL of medium, resulting in the required final drug concentration.

Following drug addition, the plates were incubated for an additional 48 h at 37° C., 5% CO₂, 95% air, and 100% humidity. For adherent cells, the assay was terminated by the addition of cold TCA. Cells were fixed in situ by the gentle addition of 50 μL of cold 50% (w/v) TCA (final concentration, 10% TCA) and incubated for 60 minutes at 4° C. The supernatant was discarded, and the plates were washed five times with tap water and air dried. Sulforodamine B (SRB) solution (100 μL) at 0.4% (w/v) in 1% acetic acid was added to each well, and plates were incubated for 10 minutes at room temperature. After staining, unbound dye was removed by washing 5 times with 1% acetic acid and the plates were air dried. Bound stain was subsequently solubilized with 10 mM trizma base, and the absorbance was read on an automated plate reader at a wavelength of 515 nm. For suspension cells, the methodology was the same except that the assay was terminated by fixing cells at the bottom of the wells by gently adding 50 μL of 80% TCA (final concentration, 16% TCA). Using the seven absorbance measurements [time zero, (Tz), control growth (C), and test growth in the presence of drug at the five concentration levels (Ti)], the percentage growth was calculated at each of the drug concentration levels. Percentage growth was calculated as follows:

[(Ti−Tz)/(C−Tz)]×100 for concentrations for which Ti>=Tz;

[(Ti−Tz)/Tz]×100 for concentrations for which Ti<Tz.

Three dose response parameters were calculated for each drug for each cell line. Growth inhibition of 50% (GI50) was calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) was calculated from Ti=Tz. The LC50 (concentration of drug resulting in a 50% reduction in the measured protein at the end of the drug treatment as compared to the beginning) indicating a net loss of cells following treatment was calculated from [(Ti−Tz)/Tz]×100=−50. Values were calculated for each of these three parameters if the level of activity was reached; however, if the effect was not reached or was exceeded, the value for that parameter was expressed as greater or less than the maximum or minimum concentration tested. Results are shown in Table 5.

TABLE 5
Results for NCI Dose Response Testing in 60 Cancer Cell Lines (all units in μM)

| | Compound 105 | | | Compound 173 | | |
|---|---|---|---|---|---|---|
| Cell Line | GI50 | TGI | LC50 | GI50 | TGI | LC50 |

Leukemia

| | | | | | | |
|---|---|---|---|---|---|---|
| CCRF-CEM | 1.69 | 3.31 | 6.47 | 1.97 | 5.15 | >100 |
| HL-60(TB) | 1.54 | 3.47 | NA | 1.77 | 3.79 | NA |
| K-562 | 1.39 | 3.32 | NA | 1.67 | NA | >100 |
| MOLT-4 | 1.66 | 3.42 | NA | 1.93 | 4.43 | 17.5 |
| RPMI-8226 | 1.71 | 3.78 | NA | 1.59 | 4.87 | >100 |
| SR | 0.403 | NA | >100 | 1.30 | 3.41 | NA |

Non-Small Cell Lung Cancer

| | | | | | | |
|---|---|---|---|---|---|---|
| A549/ATCC | 1.77 | 23.5 | >100 | 1.55 | 3.45 | 7.66 |
| EKVX | 21.0 | 43.5 | 90.0 | 1.19 | 46.2 | >100 |
| HOP-62 | 2.18 | 3.87 | 6.85 | 1.88 | 3.79 | NA |
| HOP-92 | 1.34 | 3.08 | 7.1 | 1.37 | 5.13 | 38.5 |
| NCI-H226 | 22.2 | 44.4 | 89.2 | 1.62 | 9.56 | 45.6 |
| NCI-H23 | NA | 1.95 | 5.47 | 3.45 | 19.6 | >100 |
| NCI-H322M | 13.5 | 30.7 | 69.9 | 2.54 | 12.0 | 39.8 |
| NCI-H460 | 1.13 | 2.47 | 5.37 | 1.86 | 3.43 | 6.33 |
| NCI-H522 | 2.1 | 4.43 | NA | 4.15 | 23.5 | >100 |

Colon Cancer

| | | | | | | |
|---|---|---|---|---|---|---|
| HCC-2998 | 1.39 | 2.88 | 5.96 | 2.22 | 4.88 | 17.5 |
| HCT-116 | 1.53 | 3.35 | 7.30 | 2.16 | 4.40 | NA |
| HCT-15 | 11.1 | 24.4 | 53.9 | 23.9 | 87.8 | >100 |
| HT29 | 0.733 | NA | >100 | 3.64 | >100 | >100 |
| KM12 | 14.8 | 28.7 | 55.7 | 1.31 | 2.57 | 5.07 |
| SW-620 | 2.49 | 6.24 | 21.7 | 1.67 | 3.19 | 6.12 |

CNS (Central Nervous System) Cancer

| | | | | | | |
|---|---|---|---|---|---|---|
| SF-268 | 17.0 | 31.5 | 58.3 | 7.37 | 21.8 | 51.0 |
| SF-295 | 3.74 | 19.0 | 65.3 | 0.341 | 2.07 | 5.17 |
| SF-539 | 1.48 | 2.89 | 5.63 | 1.92 | 4.48 | 11.9 |
| SNB-19 | 1.60 | 3.01 | 5.68 | 1.59 | 3.21 | NA |
| SNB-75 | 1.29 | 2.78 | 5.97 | 1.61 | 4.65 | >100 |
| U251 | 4.74 | 17.3 | 48.3 | 1.49 | 2.80 | 5.30 |

Melanoma

| | | | | | | |
|---|---|---|---|---|---|---|
| LOX IMVI | 1.81 | 3.41 | 6.42 | 1.40 | 2.70 | 5.20 |
| MALME-3M | 15.6 | 37.4 | 89.5 | 1.55 | 3.57 | 8.23 |
| M14 | 1.77 | 3.40 | 6.55 | 3.43 | 11.4 | >100 |
| SK-MEL-28 | 12.3 | 24.8 | 50.2 | 1.43 | 2.79 | 5.43 |
| SK-MEL-5 | 1.54 | 2.94 | NA | 1.03 | 26.5 | 68.3 |
| UACC-257 | 1.75 | 3.70 | NA | ND | ND | ND |
| UACC-62 | 13.7 | 28.2 | 58.2 | 1.15 | 2.74 | 6.52 |

Ovarian Cancer

| | | | | | | |
|---|---|---|---|---|---|---|
| IGROV1 | NA | 3.11 | 6.97 | 3.56 | 17.9 | 81.0 |
| OVCAR-3 | 16.1 | 30.1 | 56.3 | 1.31 | 9.94 | 31.6 |
| OVCAR-4 | 1.77 | 4.25 | >100 | 9.30 | 68.8 | >100 |
| OVCAR-5 | 1.85 | 3.46 | 6.5 | 1.92 | 3.55 | 6.57 |
| SK-OV-3 | 1.85 | 3.33 | 5.99 | 1.61 | 3.08 | 5.88 |

Renal Cancer

| | | | | | | |
|---|---|---|---|---|---|---|
| 786-0 | 1.33 | 2.66 | 5.30 | 3.42 | 11.1 | 91.1 |
| A498 | 0.116 | 0.665 | 15.5 | 1.99 | 8.26 | 37.9 |
| ACHN | 15.7 | 30.3 | 58.5 | 12.2 | 25.8 | 54.7 |
| CAKI-1 | 19.8 | 43.9 | 97.2 | 0.135 | 34.0 | >100 |
| SN12C | 13.8 | 29.5 | 62.9 | 1.17 | 2.41 | 5.00 |
| TK-10 | 14.1 | 34.1 | 82.9 | 3.59 | 17.6 | 55.4 |
| UO-31 | 1.58 | 3.27 | 6.76 | >100 | >100 | >100 |

Prostate Cancer

| | | | | | | |
|---|---|---|---|---|---|---|
| PC-3 | 1.73 | 3.33 | 6.41 | 2.78 | 11.0 | 44.5 |
| DU-145 | 15.8 | 31.2 | 61.6 | 6.94 | 20.0 | 46.3 |

Breast Cancer

| | | | | | | |
|---|---|---|---|---|---|---|
| MCF7 | 0.546 | 1.91 | 4.75 | 1.45 | 2.77 | 5.29 |
| NCI/ADR-RES | <0.01 | 1.1 | 30.5 | >100 | >100 | >100 |
| MDA-MB-231/ATCC | 11.1 | 26.8 | 64.7 | 1.26 | 2.57 | 5.24 |
| HS 578T | 2.93 | 16.7 | 48.7 | 1.64 | 4.77 | 32.7 |
| MDA-MB-435 | 4.27 | 21.7 | 87.1 | 1.98 | 6.21 | 24.3 |
| BT-549 | 25.3 | 50.6 | >100 | 11.7 | 26.9 | 61.6 |
| T-47D | 1.45 | 3.08 | NA | 1.70 | 3.78 | 8.41 |
| MDA-MB-468 | 1.46 | 3.27 | NA | 1.66 | 3.72 | 8.31 |

NA = Not Available;
ND = Not Done

### Example 408

### Activity in Cancer Cell Lines

Compounds were solubilized at 10 μM in DMSO and were stored at −20° C. Test compounds were initially tested at a single concentration of 10 μM and a subset of test compounds were further tested in selected cell lines using a six concentration dose response curve. Tamoxifen citrate was purchased from Sigma and used as a positive control compound.

Cell Preparation Cancer cell lines were passaged in T-75 flasks prior to use in the assay in cell culture medium recommended by the ATCC (American Type Culture Collection; Table 6). On the day preceding the assay, the cells were split 1:2 to assure they were in an exponential growth phase at the time of compound treatment. Total cell and viability quantification was performed using a hemocytometer and Trypan Blue dye exclusion. Cell viability was greater than 95% for cells to be utilized in the assay. The cells were resuspended at the appropriate cell per mL (Table 6) in tissue culture medium and added to the microtitre plates in a volume of 100 μL. Colon cell lines were incubated overnight at 37° C./5% CO₂ to allow for cell adherence to the plates.

TABLE 6
Cancer Cell Culture Medium and Cell Densities

| | | | Cell Density |
|---|---|---|---|
| Cell | Cancer | | (96 well plate/ |
| Line | Type | Cell Culture Medium* | per well) |
| CEM-SS | Leukemia | RPMI1640, 10% FBS, | 1 × 10⁵ |
| | | 2 mM L-glutamine | |
| Jurkat | Leukemia | RPMI1640, 10% FBS, | 1 × 10⁵ |
| | | 2 mM L-glutamine | |
| THP-1 | Leukemia | RPMI1640, 10% FBS, | 1 × 10⁵ |
| | | 2 mM L-glutamine | |
| HL-60 | Leukemia | IMDM, 10% FBS, | 1 × 10⁵ |
| | | 4 mM L-glutamine | |
| K562 | Leukemia | IMDM, 10% FBS, | 1 × 10⁵ |
| | | 2 mM L-glutamine | |
| Caco-2 | Colon | EMDM, 20% FBS, | 5 × 10⁴ |
| | | 2 mM L-glutamine, | |
| | | 0.1 mM NEAA, | |
| | | 1 mM sodium pyruvate | |
| HT-29 | Colon | McCoys 5A, 10% FBSm | 7.5 × 10⁴ |
| | | 2 mM L-glutamine | |
| DLD-1 | Colon | RPMI1640, 10% FBS, | 7.5 × 10⁴ |
| | | 2 mM L-glutamine | |
| HCT-116 | Colon | RPMI1640, 10% FBS, | 1 × 10⁵ |
| | | 2 mM L-glutamine | |
| HCT-15 | Colon | RPMI1640, 10% FBS, | 1 × 10⁵ |
| | | 2 mM L-glutamine | |

*All medium contained 100 μg/mL streptomycin and 100 U/mL penicillin

Plate Format: Each plate contains cell control wells (cells only), drug calorimetric control wells (drug only) as well as experimental wells (drug plus cells). Samples were tested in triplicate at a concentration of 10 μM and five half-logarithmic dilutions.

Cell Viability by XTT: Following incubation at 37° C. in a 5% CO₂ incubator for four days, the test plates were stained with the tetrazolium dye XTT (2,3-bis(2-methoxy-4-nitro-5-sulfophenyl)-5-[(phenylamino)carbonyl]-2H-tetrazolium hydroxide). XTT-tetrazolium was metabolized by the mitochondrial enzymes of metabolically active cells to a soluble formazan product. XTT solution was prepared daily as a stock of 1 mg/mL in RPMI1640. Phenazine methosulfate (PMS) solution was prepared at 0.15 mg/mL in phosphate buffered saline (PBS) and stored in the dark at −20° C. XTT/PMS stock was prepared immediately before use by adding 40 μL of PMS per mL of XTT solution. Fifty (50) microlitres of XTT/PMS was added to each well of the plate and the plate was reincubated for 4 hours at 37° C. Plates were sealed with adhesive plate sealers and shaken gently or inverted several times to mix the soluble formazan product and the plate was read spectrophotometrically at 450/650 nm with a Molecular Devices Vmax plate reader. Raw data was collected from the Softmax Pro 4.6 software and imported into a Microsoft Excel 2003 spreadsheet for analysis. Tamoxifen citrate was evaluated in parallel with the test compounds as a control compound and yielded TC₅₀ values ranging from 15.4 to 53.6 μM in the four cancer cell lines evaluated in dose response. An Average Response Factor (ARF) was calculated for each compound by averaging the ten Percentage of Control values for the cell lines. Also shown are the range for the same 10 cell lines. Deviations below 100% control indicate activity. Results are shown in Tables 7, 8 and 9. The concentration of each tested compound is 10 μM. The entries in Table 7 are compared to the TC₅₀ concentration for tamoxifen citrate. These tests were performed at ImQuest BioSciences Inc. (7340 Executive Way, Suite R, Frederick, Md. 21704, USA).

TABLE 7
Single Dose Results for Selected Compounds and Cell Lines

| | Percentage of Cell Control | | | | | |
|---|---|---|---|---|---|---|
| Compound | Caco-2 | HT29 | HL-60 | THP-1 | HCT-15 | Jurkat |
| Tamoxifen | 17.9 | 12.7 | 20.4 | 22.4 | 59.1 | 19 |
| Citrate | | | | | | |
| (TC₅₀, μM) | | | | | | |
| 50 | 8.3 | 27.7 | 91.7 | 89.7 | 114.3 | 4.6 |
| 105 | 0 | 13.8 | 19.6 | 0 | 85.6 | 23.5 |
| 210 | 2.4 | 0 | 0.4 | 0 | 71.8 | 9.3 |
| 262 | 21.6 | 63.8 | 53.1 | 49.4 | 75 | 3.1 |

TABLE 8
Average Response Factors (ARF) for 5 Leukemia and 5 Colon
Cancer Cell Lines (Average Percentage of Control)

| Compound | ARF | Range |
|---|---|---|
| 50 | 77.7 | 4.6 to 122 |
| 70 | 94.5 | 61.1 to 115.5 |
| 81 | 94.2 | 61.3 to 116.5 |
| 98 | 90.8 | 53.8 to 126.6 |
| 105 | 53.4 | 0 to 110.8 |
| 117 | 86.5 | 35.7 to 125.7 |
| 120 | 88.4 | 40.4 to 117.1 |
| 173 | 76.8 | 4.3 to 108.6 |
| 189 | 89.0 | 66.6 to 107.6 |
| 204 | 89.3 | 70.1 to 116.4 |
| 206 | 92.9 | 81.4 to 107.6 |
| 210 | 41.1 | 0 to 94.0 |
| 212 | 94.2 | 52.7 to 118.6 |
| 366 | 100.4 | 64.8 to 121.1 |
| 324 | 95.8 | 69.3 to 109.9 |
| 262 | 69.4 | 3.1 to 121.1 |
| 247 | 85.6 | 28.5 to 104.3 |
| 305 | 79.5 | 30.9 to 105.9 |
| 319 | 95.6 | 80.0 to 116.4 |
| 321 | 88.0 | 55.9 to 109.3 |

TABLE 9
Dose Response Results for Selected Compounds and Cell Lines

Activity (TC₅₀, μM)

| Compound | Caco-2 | HT29 | THP-1 | Jurkat |
|---|---|---|---|---|
| Tamoxifen Citrate | 21.5 | 53.6 | 22.4 | 15.4 |
| 50 | 9.4 | ND | ND | ND |
| 105 | 8.6 | 8.5 | 6.7 | 2.5 |
| 210 | 10.0 | 9.8 | 7.7 | 5.4 |
| 262 | 8.7 | ND | 7.9 | 5.5 |

Although the foregoing disclosure has been described in some detail to facilitate understanding, it will be apparent that certain changes and modifications may be practiced within the scope of the appended claims. Accordingly, the described embodiments are to be considered as illustrative and not restrictive, and this disclosure is not to be limited to the details given herein, but may be modified within the scope and equivalents of the appended claims.

All literature and patent references cited throughout the application are incorporated by reference into the application for all purposes.

1. A method for treating a hyperproliferative disease or disorder, comprising administering to a subject in need thereof a therapeutically effective amount of a compound having a structure of formula (III): 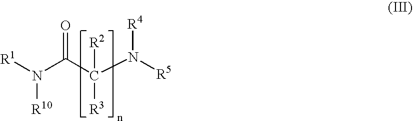
or a stereoisomer, prodrug or pharmaceutically acceptable salt thereof, wherein:
n is 1-5;
R¹ is selected from (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;
R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;
R², R³, R⁴, and R⁹ are (i) independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;
or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ and R⁹ are as defined above;
or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R⁹ are as defined above;
or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R², R³ and R⁹ are as defined above;
and wherein at least one, but not more than two of R², R³, R⁴ and R⁵, is hydrogen, provided that R¹ is not an amino acid when R⁴ and R⁵ are both H.

2. The method of claim 1, wherein R² is H, R⁴ is H, n is 1, and R⁵ is selected from —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, and —SO₂R⁹.

3. The method of claim 2, wherein the R⁹ substituent of R⁵ is selected from alkyl substituted with one or more of the same or different substituted or unsubstituted aryl groups, (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups.

4. The method of claim 3, wherein the R⁹ substituent of R⁵ is selected from —(CH₂)ₘ—R¹¹ and —R¹¹ where m is an integer in the range from 1 to 4 and R¹¹ is selected from (C₅-C₁₈) aryl optionally substituted with one or more of the same or different R¹⁰ groups, (C₆-C₂₀), arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups.

5. The method of claim 4, wherein R¹¹ is selected from (C₅-C₁₈) aryl optionally substituted with one or more of the same or different R¹⁰ groups.

6. The method of claim 5, wherein, R¹¹ is selected from the following: 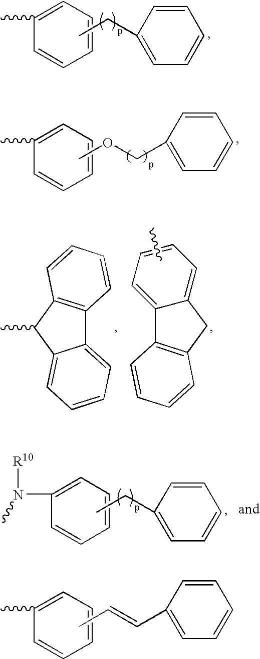
wherein p is an integer in the range of 0 to 4 and wherein each phenyl group is optionally substituted with 1 to 3 of the same or different R¹⁰ groups.

7. The method of claim 1, wherein R² is H, R⁴ is H, n is 1, and R⁵ is selected from —C(═O)R⁹, —CO₂R⁹, —C(═O)NR¹⁰R⁹, and —C(═O)(NR¹⁰)SO₂R⁹.

8. The method of claim 1, wherein R³ is —(CR⁹R¹⁰)ᵣNR⁹R¹⁰ wherein r is an integer in the range of 1 to 10 and each R⁹ and R¹⁰ is independently selected.

9. The method of claim 1, wherein R¹ is (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups.

10. The method of claim 1 wherein the hyperproliferative disease or disorder is a benign or malignant neoplasm.

11. The method of claim 1 wherein the hyperproliferative disease or disorder is leukemia.

12. A method for treating a hyperproliferative disease or disorder, comprising administering to a subject in need thereof a therapeutically effective amount of a compound having a structure of formula (IV): 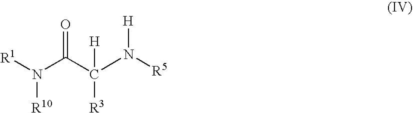
or a stereoisomer, prodrug or pharmaceutically acceptable salt thereof, wherein:
R¹, R³ and R⁹ are each independently selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, or (C₅-C₂₀) heteroarylalkenyl optionally substituted with one or more of the same or different R¹⁰ groups; provided that R¹ and R³ are not hydrogen;
R⁵ is selected from —C(═O)NR¹⁰R⁹, —C(═O)(NR¹⁰)SO₂R⁹, —C(═S)NR¹⁰R⁹, or —C(═NR¹⁰)NR¹⁰R⁹; and
each R¹⁰ is independently selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, or (C₅-C₂₀) heteroarylalkenyl.

13. The method of claim 12, wherein R³ is —(CR⁹R¹⁰)ᵣNR⁹R¹⁰ wherein r is an integer in the range of 1 to 10 and each R⁹ and R¹⁰ is independent.

14. The method of claim 12, wherein, wherein the R⁹ substituent of R⁵ is selected from alkyl substituted with one or more of the same or different substituted or unsubstituted aryl groups, (C₅-C₁₈) aryl optionally substituted with one or more of the same or different R¹⁰ groups, (C₆-C₂₀) arylalkyl optionally substituted with one or more of the same or different R¹⁰ groups, (C₄-C₁₂) heteroaryl optionally substituted with one or more of the same or different R¹⁰ groups, and (C₅-C₂₀) heteroarylalkyl optionally substituted with one or more of the same or different R¹⁰ groups.

15. The method of claim 12, wherein the hyperproliferative disease or disorder is a benign or malignant neoplasm.

16. The method of claim 12, wherein the hyperproliferative disease or disorder is leukemia.

17. The method of claim 12, wherein the compound is administered parenterally.

18. The method of claim 17, wherein the parenteral administration is intravenous, intra-arterial, or intratumoral.

19. A method for treating a hyperproliferative disease or disorder, comprising administering to a subject in need thereof a therapeutically effective amount of a compound having a structure of formula (I): 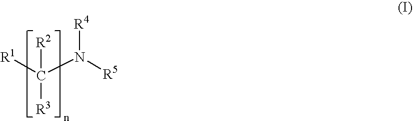
or a stereoisomer, prodrug or pharmaceutically acceptable salt thereof, wherein:
n is 1-5;
R¹ is selected from —C(═O)R⁹, —C(═S)R⁹, —C(═NR¹⁰)R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —S(═O)R⁹, —SO₂R⁹, provided that R⁹ is not H, and that R⁹ does not form an ester with the carbonyl group to which it is bonded when R¹ is —C(═O)R⁹;
(i) R², R³ and R⁴ are each independently the same or different substituent as defined for R⁹; or (ii) R² and R³ taken together with the carbon atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R⁴ is selected from R⁹; or (iii) R³ and R⁴ taken together with the carbon atom and N atom to which they are bonded, respectively, form a five- to seven-membered saturated or unsaturated ring that optionally includes one or more of the same or different heteroatoms selected from O, N, S and that is optionally substituted at one or more ring carbon or heteroatom with the same or different R¹⁰ substituent, and R² is selected from R⁹; or (iv) R⁴ and R⁵ taken together with the N atom to which they are bonded form a four- to seven-membered saturated or unsaturated ring that optionally includes one or more of the same or different heteroatoms selected from O, N, S and that is optionally substituted at one or more ring carbon or heteroatom with the same or different R¹⁰ substituent, and R² and R³ are selected from R⁹;
R⁵ is selected from H, —C(═O)R⁹, —C(═S)R⁹, —C(═NR¹⁰)R⁹, —CO₂R⁹, —C(═O)NR¹⁰R⁹, —C(═O)(NR¹⁰)SO₂R⁹, —C(═S)NR¹⁰R⁹, —C(═NR¹⁰)NR¹⁰R⁹, —OR⁹, —SR⁹, —NR¹⁰R⁹, —S(═O)R⁹, —SO₂R⁹;
R⁹ is selected from H, (C₁-C₁₀) alkyl optionally substituted with one or more of the same or different R¹⁰ groups, (C₂-C₁₀) alkenyl optionally substituted with one or more of the same or different R¹⁰ groups, (C₂-C₁₀) alkynyl optionally substituted with one or more of the same or different R¹⁰ groups, (C₁-C₁₀) alkyleno optionally substituted with one or more of the same or different R¹⁰ groups, (C₁-C₁₀) alkyldiyl optionally substituted with one or more of the same or different R¹⁰ groups, (C₅-C₁₈) aryl optionally substituted with one or more of the same or different R¹⁰ groups, (C₆-C₂₀) arylalkyl optionally substituted with one or more of the same or different R¹⁰ groups, (C₆-C₂₀) arylalkenyl optionally substituted with one or more of the same or different R¹⁰ groups, (C₁-C₁₀) heteroalkyl optionally substituted with one or more of the same or different R¹⁰ groups, (C₂-C₁₀) heteroalkenyl optionally substituted with one or more of the same or different R¹⁰ groups, (C₂-C₁₀) heteroalkynyl optionally substituted with one or more of the same or different R¹⁰ groups, (C₁-C₁₀) heteroalkyleno optionally substituted with one or more of the same or different R¹⁰ groups, (C₁-C₁₀) heteroalkyldiyl optionally substituted with one or more of the same or different R¹⁰ groups, (C₄-C₁₂) heteroaryl optionally substituted with one or more of the same or different R¹⁰ groups, (C₅-C₂₀) heteroarylalkyl optionally substituted with one or more of the same or different R¹⁰ groups, and (C₅-C₂₀) heteroarylalkenyl optionally substituted with one or more of the same or different R¹⁰ groups;
R¹⁰ is selected from H, (C₁-C₁₀) alkyl, (C₂-C₁₀) alkenyl, (C₅-C₁₈) aryl, (C₆-C₂₀) arylalkyl, (C₆-C₂₀) arylalkenyl, (C₁-C₁₀) heteroalkyl, (C₂-C₁₀) heteroalkenyl, (C₄-C₁₂) heteroaryl, (C₅-C₂₀) heteroarylalkyl, (C₅-C₂₀) heteroarylalkenyl;
and wherein at least one, but not more than two, of R², R³, R⁴ and R⁵ is hydrogen, provided that R¹ is not an amino acid when R⁴ and R⁵ are both H.
